(19) United States
(12) Patent Application Publication
(10) Pub. No.: US 2025/0277029 A1
(43) Pub. Date: Sep. 4, 2025

(54) CANNABINOID TYPE 1 RECEPTOR BINDING PROTEINS AND USES THEREOF

(71) Applicant: AMGEN INC., Thousand Oaks, CA (US)

(72) Inventors: Irwin CHEN, San Francisco, CA (US); Su CHONG, Oxnard, CA (US); Seog Joon HAN, Simi Valley, CA (US); Kirill OXENOID, Vancouver (CA); Weihsu Claire CHEN, Vancouver (CA); Venkata Nihal PASUMARTHI, Coquitlam (CA); John Bergen, Port Coquitlam (CA); Wadim Matochko, Burnaby (CA); Dawn Weishuhn, Surrey (CA); Serina LI, Vancouver (CA); Murielle Marie VENIANT-ELLISON, Thousand Oaks, CA (US); Elizabeth KILLION, Thousand Oaks, CA (US); Shu-Chen LU, Thousand Oaks, CA (US)

(73) Assignee: AMGEN INC., Thousand Oaks, CA (US)

(21) Appl. No.: 19/037,790

(22) PCT Filed: Jul. 26, 2023
(86) PCT No.: PCT/US2023/071024

§ 371 Date: Jan. 27, 2025

Related U.S. Application Data
(60) Provisional application No. 63/392,891, filed on Jul. 28, 2022.

(51) Int. Cl. C07K 16/28; A61K 39/00; A61P 3/04

(52) U.S. Cl. CPC C07K 16/28; A61P 3/04; A61K 2039/505; C07K 2317/565; C07K 2317/76; C07K 2317/92

(57) ABSTRACT

Described herein are anti-CB1 (e.g., huCB1) antibodies and antigen binding fragments, compositions comprising the same and their use for the treatment of various disorders or diseases such as obesity and its comorbidities, chronic kidney disease, liver disease (e.g., NAFLD), and NASH.

### FIELD OF THE INVENTION

The present disclosure relates to cannabinoid type 1 receptor (CB1) binding proteins and use thereof for the treatment of diseases or disorders.

### INCORPORATION-BY-REFERENCE OF MATERIAL SUBMITTED ELECTRONICALLY

Incorporated by reference in its entirety herein is an amino acid sequence listing submitted concurrently herewith and identified as follows: 7027 kilobyte XML document named “10129-WO01-SEC Sequence Listing.xml,” created on Jul. 20, 2023.

### CROSS-REFERENCE TO RELATED APPLICATIONS

This application claims the benefit of U.S. Provisional Application No. 63/392,891, filed on Jul. 28, 2022.

### BACKGROUND

The cannabinoid receptor 1 (CB1) is a G-protein coupled receptor (GPCR) that is expressed in the central nervous system (CNS) and peripheral organs such as liver, skeletal muscle, adipose tissue and endocrine pancreas. (Cinar R. et al., Pharmacology & Therapeutics 208 (2020) 107477.) CB1 receptors are expressed at a lower level in peripheral organs compared to the CNS and the receptors are involved in regulating metabolic homeostasis in both systems. Due to its role in energy homeostasis, there have been longstanding efforts in developing CB1 receptor antagonists and/or inverse agonists for the treatment of obesity and its comorbidities. (See e.g., Cinar R. et al., Pharmacology & Therapeutics 208 (2020) 107477; Thomas Murphy and Bernard Le Foll, Biomolecules 2020, 10, 855.)

In clinical trials, the small molecule CB1 receptor antagonist/inverse agonist rimonabant (also known as SR141716) reduced bodyweight in overweight people and significantly improved multiple cardiometabolic parameters such as waist circumference, hemoglobin Alc, HDL, plasma cholesterol and triglycerides. Rimonabant was approved as an anti-obesity drug in Europe in 2006. However, the CNS-penetrance of rimonabant also induces severe psychiatric side effects such as anxiety, depression, and suicidality, which led to its withdrawal in 2008. Additional efforts have been made in the development of peripherally restricted small molecule CB1 receptor antagonists (e.g., Tam et al, J. Clin. Invest. (2010) 120:2953-66; US2011/0144157), but success in this area remains to be seen.

Because antibodies do not appreciably cross the blood brain barrier and penetrate the CNS, scientists have hypothesized that CB1 antagonist antibodies with rimonabant-like potencies may confer the beneficial effects on metabolism (e.g., treating obesity and its comorbidities) without the CNS-related side effects. However, the discovery and engineering of potent antibody antagonists of CB1 have shown to be difficult in the field. For example, potencies of antagonist CB1 antibodies described in the field are in the double or triple digit nanomolar range (See e.g., WO2014/210025, WO2015/148948 and WO2019/211665); in comparison, the potency of rimonabant is in the single digit nanomolar range. (See e.g., Congy C. et al., FEBS Letters 350 (1994) 240-244; Bauer M. et al., The Journal of Biological Chemistry, 287(44) (2016) 36944-36967). In addition, binding affinities of those antibodies to CB1 (e.g., huCB1) are inferior to that of rimonabant.

Several factors are considered to contribute to this difficulty. It has generally been difficult to raise antibodies against GPCRs with small extracellular loops such as CB1 via immunization (see e.g., Jo Migyeong and Jung Sang Take, Experimental & Molecular Medicine (2016) 48, e207). In addition, many CB1 antibodies raised in animal campaigns tend to be non-functional. Further, potencies and/or affinity of antagonist antibodies isolated from animal campaigns are typically not sufficiently high to meet the potency and/or affinity requirement (e.g., comparable to or better than rimonabant). Although affinity maturation can be used to improve the potency of antagonist antibodies, it has been challenging in employing this strategy for CB1 antibodies due to the lack of stable and high-quality preparations of conformationally-relevant CB1 protein.

There remains a need in the field for identifying CB1 antagonist antibodies with rimonabant-like potencies and affinities that can achieve therapeutic effects while avoid CNS-related adverse effects.

### SUMMARY

The present application relates to antagonist or inverse agonist anti-CB1 (e.g., huCB1) antibodies and antigen binding fragments having high potencies (e.g., potencies comparable to or better than that of rimonabant). The antibodies disclosed herein have much higher potencies compared to previously known anti-CB1 antibodies and are suitable for safe and effective treatment of various diseases such as obesity and its comorbidities, chronic kidney disease, non-alcoholic steatohepatitis (NASH), and nonalcoholic fatty liver disease (NAFLD). Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
E1: An isolated antibody that binds to huCB1, the antibody comprises a HV and a LV, wherein a) the HV comprises HCDR1, HCDR2 and HCDR3, wherein HCDR1 comprises the amino acid sequence of RGGDYWX1 (SEQ ID NO: 530), wherein X1 is A, S, or G; HCDR2 comprises the amino acid sequence of HX2YX3X4GX5TX6YNPX7X8X9X10 (SEQ ID NO: 531), wherein X2 is I or V; X3 is H, Y, or Q; X4 is E, T, or S; X5 is K, S, or Q; X6 is A, K or N; X7 is N, S, K, or R; X8 is F or L; X9 is E or K; and X10 is G, D, S, or N; and HCDR3 comprises the amino acid sequence of X11YDX12X13X14GX15SYYYYGMDV (SEQ ID NO: 532), wherein X11 is D, E, or N; X12 is A, I, P, T, or V; X13 is F, L, or V; X14 is S or T; and X15 is H, N, or Y; and b) the LV is of a VK1/O2/JK4, a VK2/A19/JK1, or a VK3/A27/JK1 germline.
E2 The antibody of E1, wherein a) HCDR1 comprises the amino acid sequence of RGGDYWX1 (SEQ ID NO: 530), wherein X1 is A or S; HCDR2 comprises the amino acid sequence of HX2YX3X4GX5TX6YNPX7X8X9X10 (SEQ ID NO: 531), wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N; and HCDR3 comprises the amino acid sequence of X11YDX12X13X14GX15SYYYYGMDV (SEQ ID NO: 532), wherein X11 is D, E, or N; X12 is A, I, P, or V; X13 is L or V; X14 is S or T; and X15 is H or Y; and b) the LV is of a VK2/A19/JK1 germline.
E3 The antibody of E2, wherein X4 is T, X15 is Y.
E4 The antibody of any one of E1-E3, wherein b) the LV comprises LCDR1, LCDR2 and LCDR3, and wherein LCDR1 comprises the amino acid sequence RSSQSLLX16SX17GX18NYX19D (SEQ ID NO: 533), wherein X16 is H, S, or T; X17 is S, T, or Y; X18 is A, N, I or Y; and X19 is L or V; LCDR2 comprises the amino acid sequence of X20GSNRA (SEQ ID NO: 534), wherein X20 is L or Q; and LCDR3 comprises the amino acid sequence of X21QAX22X23X24PRT (SEQ ID NO: 535), wherein X21 is M or R; X22 is L, I, R, or V; X23 is Q, E, T, A, or G; and X24 is T, I, L, or Q.
E5 The antibody E4, wherein X24 is L.
E6. The antibody of E1, wherein a) HCDR1 comprises the amino acid sequence of RGGDYWX1 (SEQ ID NO: 530), wherein X1 is A, G or S; HCDR2 comprises the amino acid sequence of HX2YX3X4GX5TX6YNPX7X8X9X10 (SEQ ID NO: 531), wherein X2 is I or V; X3 is H, Y, or Q; X4 is T or S; X5 is K, S or Q; X6 is A, K or N; X7 is N, S, K, or R; X8 is F or L; X9 is E or K; and X10 is G, D, S, or N; and HCDR3 comprises the amino acid sequence of X11YDX12X13X14GX15SYYYYGMDV (SEQ ID NO: 532), wherein X11 is D or N; X12 is A, I, or T; X13 is F, L, or V; X14 is S or T; and X15 is H, N or Y; and b) the LV is of a VK1/O2/JK4 or a VK3/A27/JK1 germline.
E7. The antibody of claim E6, wherein a) X1 is A or G; X3 is Q or Y, X4 is T, X8 is F, X14 is T; and X15 is Y.
E8. The antibody of E6 or E7, wherein b) the LV is of a VK1/O2/JK4 germline.
E9. The antibody of E6 or E7, wherein b) the LV is of a VK1/O2/JK4 germline and comprises light chain LCDR1, LCDR2 and LCDR3, and wherein LCDR1 comprises the amino acid sequence of RASQSISNYLN (SEQ ID NO: 132), RASQSIISYLN (SEQ ID NO: 150), or RASQSISSYLN (SEQ ID NO: 186); LCDR2 comprises the amino acid sequence of AASSLHS (SEQ ID NO: 133) or AASSLRS (SEQ ID NO: 151); and LCDR3 comprises the amino acid sequence of QQYQSYPLT (SEQ ID NO: 134) or QQYSNYPLT (SEQ ID NO: 152); or
b) the LV is of a VK3/A27/JK1 germline and comprises light chain LCDR1, LCDR2 and LCDR3, and wherein LCDR1 comprises the amino acid sequence of RASQSVSSYLG (SEQ ID NO: 168); LCDR2 comprises the amino acid sequence of GASSRAT (SEQ ID NO: 169); and LCDR3 comprises the amino acid sequence of QQYGSSPRT (SEQ ID NO: 170).
E10. The antibody of E9, wherein LCDR1 comprises the amino acid sequence of RASQSISNYLN (SEQ ID NO: 132), LCDR2 comprises the amino acid sequence of AASSLHS (SEQ ID NO: 133), and LCDR3 comprises the amino acid sequence of QQYQSYPLT (SEQ ID NO: 134).
E11. The antibody of E6, wherein a) HCDR1 comprises the amino acid sequence of RGGDYWX1 (SEQ ID NO: 530), wherein X1 is A or G, HCDR2 comprises the amino acid sequence of HX2YX3X4GX5TX6YNPX7X8X9X10 (SEQ ID NO: 531), wherein X2 is I or V; X3 is Y or Q; X4 is T; X5 is S; X6 is K or N; X7 is S or R; X8 is F; X9 is K; and X10 is G or D; and HCDR3 comprises the amino acid sequence of X11YDX12X13X14GX15SYYYYGMDV (SEQ ID NO: 532), wherein X11 is N; X12 is T; X13 is L or V; X14 is T; and X15 is Y, and b) the LV is of a VK1/O2/JK4 germline.
E12. The antibody of E11, wherein a) HCDR1 comprises the amino acid sequence of RGGDYWA (SEQ ID NO: 414) or RGGDYWG (SEQ ID NO: 408); HCDR2 comprises the amino acid sequence of HVYYTGSTKYNPSFKD (SEQ ID NO: 427), HVYYTGSTNYNPRFKD (SEQ ID NO: 445), HIYQTGSTNYNPRFKG (SEQ ID NO: 415), or HVYQTGSTKYNPSFKD (SEQ ID NO: 409); and HCDR3 comprises the amino acid sequence of NYDTLTGYSYYYYGMDV (SEQ ID NO: 410) or NYDTVTGYSYYYYGMDV (SEQ ID NO: 446).
E13. The antibody of E11 or E12, wherein b) the LV comprises LCDR1, LCDR2 and LCDR3, and wherein LCDR1 comprises the sequence of RASQSISSYLN (SEQ ID NO: 405); LCDR2 comprises the sequence of X39ARX40LX41S (SEQ ID NO: 536), wherein X39 is N, S, K or G; X40 is R, K, L or A; and X41 is A, G or S; and LCDR3 comprises the sequence of QQX42X43X44X45PX46T (SEQ ID NO: 537), wherein X42 is Y or F, X43 is R, A, S, G, or Y; X44 is S, K, R or H; X45 is S, L, F, Y, P, or M; and X46 is L, I or V.
E14. The antibody of E13, wherein X42 is Y.
E15. An isolated antibody that binds to huCB1, the antibody comprises a HV and a LV, wherein a) the HV comprises CDRH1, CDRH2 and CDRH3, wherein CDRH1 comprises the amino acid sequence of RGGDYWS (SEQ ID NO: 1); CDRH2 comprises the amino acid sequence of HX25YX26X27GX28TX29YNPX30X31X32X33 (SEQ ID NO: 538), wherein X25 is I or V; X26 is Y or Q; X27 is A, E, K, or T; X28 is S or Q; X29 is A, E, K, or T; X30 is N or S; X31 is F or L, X32 is K or R; and X33 is G, S, or N; and CDRH3 comprises the amino acid sequence of X34YDX35X36X37GYSYYYYGX38DV (SEQ ID NO: 539), wherein X34 is D or G; X35 is A, I, T, or V; X36 is L or absent; X37 is S or T; and X38 is M or L; and b) the LV is of a VK2/A19/JK1 germline.
E16. The antibody of E15, wherein a) X26 is Y, X34 is G, X36 is absent, and X37 is S.
E17. The antibody of E15 or E16, wherein b) the LV comprises LCDR1, LCDR2 and LCDR3, wherein LCDR1 comprises the amino acid sequence of RSSQSLLHRSGYNYLD (SEQ ID NO: 257); LCDR2 comprises the amino acid sequence of LGSNRAS (SEQ ID NO: 258) or QGSNRAS (SEQ ID NO: 264); and LCDR3 comprises the amino acid sequence of MQSLQTPRT (SEQ ID NO: 259), RQSVALPRT (SEQ ID NO: 271), or RQARALPRT (SEQ ID NO: 265).
E18. The antibody of E1-E14, wherein the antibody comprises a HCDR1, HCDR2, HCDR3, LCDR1, LCDR2, or LCDR3, or a set of HCDR1, HCDR2, HCDR3, LCDR1, LCDR2, and LCDR3 of any one of LIBC523596-1, LIBC523603-1, LIBC523661-1, LIBC523670-1, LIBC523748-1, LIBC523760-1, LIBC523797-1, LIBC523813-1, LIBC523815-1, LIBC523844-1, LIBC523857-1, LIBC523862-1, LIBC523868-1, LIBC523969-1, LIBC524049-1, LIBC527906-1, LIBC527814-1, LIBC527997-1, LIBC528141-1, LIBC527912-1, LIBC528116-1, LIBC527879-1, LIBC527919-1, LIBC527984-1, LIBC527968-1, LIBC528169-1, LIBC528131-1, LIBC527827-1, LIBC527869-1, and LIBC528148-1, LIBC680562-1, LIBC680574-1, LIBC680773-1, LIBC680800-1, LIBC680812-1, LIBC681593-1, LIBC681594-1, and LIBC681737-1.
E19. The antibody of E15-E17, wherein the antibody comprises a HCDR1, HCDR2, HCDR3, LCDR1, LCDR2, or LCDR3 or a set of HCDR1, HCDR2, HCDR3, LCDR1, LCDR2, and LCDR3 of any one of LIBC673948-1, LIBC673952-1, LIBC673965-1, LIBC673982-1, LIBC673972-1, LIBC674024-1, LIBC674035-1, LIBC674043-1, LIBC674090-1, LIBC674002-1, LIBC674153-1, LIBC674200-1, LIBC674214-1, LIBC674216-1, LIBC674229-1, LIBC674235-1, LIBC674257-1, and LIBC674276-1.
E20. The antibody of E18 or E19, wherein the antibody comprises a HCDR1, HCDR2, HCDR3, LCDR1, LCDR2, or LCDR3 or a set of HCDR1, HCDR2, HCDR3, LCDR1, LCDR2, and LCDR3 of any one of LIBC680574, LIBC528116, LIBC673952, LIBC523797, LIBC527814, LIBC527997, LIBC527919, LIBC523661 and LIBC524049.
E21. The antibody of any one of E1-E20, wherein the amino acid at position 83 of the HV is N and the amino acid at position 85 of the HV is Y.
E22. The antibody of E21, wherein the amino acid at position 94 of the LV is S.
E23. The antibody of E19, wherein the amino acid at position 83 of the HV is N, the amino acid at position 85 of the HV is Y, and the amino acid at position 79 of the HV is R.
E24. The antibody of E23, the amino acid at position 94 of the LV is S, and the amino acid at position 76 is D.
E25. The antibody of any one of E1-E24, wherein HCDR2 of the antibody comprises 5 or fewer mutations, or 3 or fewer mutations, compared to HVYYTGSTNYNPRFKD (SEQ ID NO: 136).
E26. The antibody of any one of E1-E24, wherein HCDR2 of the antibody comprises 5 or fewer mutations compared to HVYYTGSTKYNPNFKG (SEQ ID NO: 8), and optionally, HCDR3 of the antibody comprises 4 or fewer mutations compared to DYDILTGYSYYYYGMDV (SEQ ID NO: 9).
E27. The antibody of any one of E1-E26, wherein the antibody comprises a HV and/or LV of any one of LIBC523596-1, LIBC523603-1, LIBC523661-1, LIBC523670-1, LIBC523748-1, LIBC523760-1, LIBC523797-1, LIBC523813-1, LIBC523815-1, LIBC523844-1, LIBC523857-1, LIBC523862-1, LIBC523868-1, LIBC523969-1, LIBC524049-1, LIBC527906-1, LIBC527814-1, LIBC527997-1, LIBC528141-1, LIBC527912-1, LIBC528116-1, LIBC527879-1, LIBC527919-1, LIBC527984-1, LIBC527968-1, LIBC528169-1, LIBC528131-1, LIBC527827-1, LIBC527869-1, and LIBC528148-1, LIBC680562-1, LIBC680574-1, LIBC680773-1, LIBC680800-1, LIBC680812-1, LIBC681593-1, LIBC681594-1, LIBC681737-1, LIBC673948-1, LIBC673952-1, LIBC673965-1, LIBC673982-1, LIBC673972-1, LIBC674024-1, LIBC674035-1, LIBC674043-1, LIBC674090-1, LIBC674002-1, LIBC674153-1, LIBC674200-1, LIBC674214-1, LIBC674216-1, LIBC674229-1, LIBC674235-1, LIBC674257-1, and LIBC674276-1.
E28. The antibody of E27, wherein the antibody comprises a HV and/or LV of any one of LIBC680574, LIBC528116, LIBC673952, LIBC523797, LIBC527814, LIBC527997, LIBC527919, LIBC523661 and LIBC524049.
E29. An isolated antibody that binds to huCB1 and comprises a HCDR1, HCDR2, HCDR3, LCDR1, LCDR2, or LCDR3 or a set of HCDR1, HCDR2, HCDR3, LCDR1, LCDR2, and LCDR3 of LIBC529593, LIBC560340, or LIBC560657.
E30. The antibody of E29, wherein the antibody comprises a HV and/or LV of LIBC529593, LIBC560340, or LIBC560657.
E31. The antibody of any one of E1-E30, wherein the antibody is a monoclonal antibody, optionally, the monoclonal antibody is a chimeric antibody, a humanized antibody, or a human antibody.
E32. The monoclonal antibody of E31, wherein the antibody is an antagonist and/or an inverse agonist antibody of huCB1.
E33. The antibody of E31 or E32, wherein the antibody has a binding affinity for huCB1 that is at least 3 times higher compared to 10D10N35Y.
E34. The antibody of E32 or E33, wherein the antibody has an IC50 of 10 nM or less, such as 5 nM or less, 3 nM or less or 1 nM or less, as determined using a cell-based cAMP assay.
E35. An isolated monoclonal antibody that binds to huCB1, wherein the antibody is an antagonist and/or inverse agonist antibody of huCB1 and has an IC50 of 10 nM or less, or 8 nM or less, as determined using a cell-based cAMP assay.
E36. The antibody of E35, wherein the antibody binds to the extracellular loop 2 of huCB1 or to a region comprised within the extracellular loop 2 of huCB1.
E37. The antibody of any one of E34-E36, wherein the cell-based cAMP assay is a cell-based cAMP assay with CP55,940.
E38. The antibody of any one of E31-E37, wherein the monoclonal antibody is a human IgG1, IgG2, IgG3, or IgG4 antibody, preferably, a human IgG1 antibody.
E39. The antibody of E38, wherein the antibody comprises a mutation at amino acid position N297, such as N297G, according to EU numbering in its heavy chain.
E40. The antibody of E39, wherein the antibody further comprises R292C and V302C mutations according to EU numbering in its heavy chain.
E41. The antibody of any one of E37-E40, wherein the antibody comprises mutations at amino acid positions M252, S254, and T256, preferably M252Y, S254T, and T256E, according to EU numbering in its heavy chain.
E42. A method for treating a subject in need of antagonizing or inverse agonizing the CB1 receptor, the method comprises administering the antibody of any one of E1-E41 to the subject.
E43. A method for treating a disease or disorder in a subject responsive to antagonizing or inverse agonizing the CB1 receptor, the method comprises administering to the subject the antibody of any one of E1-E41.
E44. The method of E42 or E43, wherein the administration results in one or more of reduced body weight, reduced appetite, improved metabolic parameters, reduced blood glucose levels, reduced insulin levels, reduced triglyceride levels, reduced kidney injury, reduced kidney fibrosis, reduced kidney inflammation, and improved kidney function.
E45. The method of E43, wherein the disease or disorder is selected from obesity, diabetes, dyslipidemia, metabolic diseases, liver disease, fibrosis, non-alcoholic steatohepatitis (NASH), primary biliary cirrhosis, renal disease, kidney fibrosis, chronic kidney disease, IgA nephropathy, osteoporosis, atherosclerosis, cardiovascular disease, cancer, and inflammatory disease.
E46. The antibody of any one of E1-E41 for use in the treatment of a disease or disorder in a subject, wherein the disease or disorder is selected from obesity, diabetes, dyslipidemia, metabolic diseases, fibrosis, liver disease, NASH, primary biliary cirrhosis, renal disease, kidney fibrosis, chronic kidney disease, IgA nephropathy, osteoporosis, atherosclerosis, cardiovascular disease, cancer, and inflammatory disease.
E47. The antibody according to E46, wherein the use results in one or more of reduced body weight, reduced appetite, improved metabolic parameters, reduced blood glucose levels, reduced insulin levels, reduced triglyceride levels, reduced kidney injury, reduced kidney fibrosis, reduced kidney inflammation, and improved kidney function.
E48. The method of E45 or the antibody of E46, wherein the disease or disorder is obesity, optionally, the subject has a BMI of at least 27 kg/m² or the subject has a BMI of at least 27 kg/m².
E49. The method of any one of E42-45 or the antibody of E47 or E48, wherein the subject is a human.
E50. An isolated polynucleotide that encodes the antibody of any one of E1-E41.
E51. An expression vector comprising the polynucleotide of E50.
E52. A host cell comprising the expression vector of E51.
E53. The antibody of E5 or E15, wherein the amino acid at position 32 according to AHo numbering in the heavy chain is an R.

### BRIEF DESCRIPTION OF THE FIGURES

FIGS. 1A and 1B show the binding profile of top affinity-matured cognate variants.

FIGS. 2A and 2B show the binding profile of top affinity-matured LC-swapped variants.

FIG. 3A shows HV CDR and FR3 sequence alignment of top cognate affinity-matured variants. FIG. 3B shows LV CDR and FR3 sequence alignment of top cognate affinity-matured variants.

FIG. 4A shows HV CDR and FR3 sequence alignment of top LC-swapped affinity-matured variants. FIG. 4B shows LV CDR sequence alignment of top LC-swapped affinity-matured variants.

FIG. 5A shows antagonist of exemplary affinity-matured antibodies. FIG. 5B shows inverse agonist activity of exemplary affinity-matured antibodies.

FIGS. 6A and 6B show reduction of body weight and foot intake by an exemplary affinity-matured antibody in obese human CB1 knock in mice. FIG. 6C shows reduction of fat mass, liver triglyceride and insulin levels of the antibody. FIG. 6D shows impact on kidney injury, fibrosis and inflammatory markers of an exemplary affinity-matured antibody.

FIG. 7 shows HV and LV CDR sequence alignment of top further LC-swapped affinity-matured variants.

FIG. 8A shows HV CDR and FR3 sequence alignment of top further cognate affinity-matured variants. FIG. 8B shows LV CDR sequence alignment of top further cognate affinity-matured variants.

FIG. 9 shows antagonist activity of antibodies isolated from mouse antibody campaigns.

FIG. 10A shows reduction of body weight by exemplary further affinity-matured antibodies in obese human CB1 knock in mice. FIG. 10B shows the impact on food intake of the antibodies. FIG. 10C shows the impact on fat mass of the antibodies and FIG. 10D shows the impact on liver weight of the antibodies.

FIG. 11A shows treatment with an exemplary anti-CB1 antibody increases energy expenditure and decreases respiratory exchange ratio in an animal model (mice). FIG. 11B shows treatment with an exemplary anti-CB1 antibody increases insulin sensitivity in an animal model.

### DETAILED DESCRIPTION

Cannabinoid receptor 1 (CB1) is a G-protein coupled receptor for endogenous cannabinoids such as 2-arachidonoylglycerol (2-AG) and the receptor mediates many cannabinoid-induced effects (e.g., food intake). Activation of the receptor leads to a decrease of intracellular cyclic AMP (cAMP) concentration and an increase of mitogen-activated protein kinase (MAP kinase) concentration. (Gerard C. et al., Biochem. J. 279:129-134(1991)). The human CB1 (huCB1) receptor is a polypeptide of 472 amino acids (UniProtKB/Swiss-Prot: P21554) and is encoded by the CNR1 gene. The amino acid sequence of the huCB1 is listed below (SEQ ID NO: 559). huCB1 is a membrane protein that has four extracellular regions (amino acid residues 1-116, 176-187, 256-273, and 366-377 of SEQ ID NO: 559), four cytoplasmic regions (amino acid residues 143-154, 213-232, 300-344, and 400-472 of SEQ ID NO: 559) and seven transmembrane domains (amino acid residues 117-142, 155-175, 188-212, 233-255, 274-299, 345-365, and 378-399 of SEQ ID NO: 559).

```
MKSILDGLAD TTFRTITTDL LYVGSNDIQY EDIKGDMASK LGYFPQKFPL
                60 70 80 90 100
TSFRGSPFQE KMTAGDNPQL VPADQVNITE FYNKSLSSEK ENEENIQCGE
              110 120 130 140 150
NFMDIECFMV LNPSQQLAIA VLSLTLGTFT VLENLLVLCV ILHSRSLRCR
              160 170 180 190 200
PSYHFIGSLA VADLLGSVIF VYSFIDFHVF HRKDSRNVFL FKLGGVTASF
              210 220 230 240 250
TASVGSLFLT AIDRYISIHR PLAYKRIVTR PKAVVAFCLM WTIAIVIAVL
              260 270 280 290 300
PLLGWNCEKL QSVCSDIFPH IDETYLMFWI GVTSVLLLFI VYAYMYILWK
              310 320 330 340 350
AHSHAVRMIQ RGTQKSIIIH TSEDGKVQVT RPDQARMDIR LAKTIVLILV
              360 370 380 390 400
VLIICWGPLL AIMVYDVFGK MNKLIKTVFA FCSMLCLLNS TVNPIIYALR
              410 420 430 440 450
SKDLRHAFRS MFPSCEGTAQ PLDNSMGDSD CLHKHANNAA SVHRAAESCI
              460 470
KSTVKIAKVT MSVSTDTSAE AL
```

CB1 receptors are expressed in the CNS and peripheral systems and are involved in regulating energy homeostasis. The receptor has been a target for the development of treatments for obesity and its comorbidities. In view of the experiences with rimonabant, the field has focused on developing peripherally acting CB1 receptor antagonists or inverse agonists to confer the beneficial effects on metabolism without the CNS-related side effects.

Antibodies against the CB1 receptor can serve as peripherally acting antagonists or inverse agonists because they do not appreciably penetrate the CNS. Efforts have been made to identify antagonist or inverse agonist anti-CB1 antibodies that have rimonabant-like potency to achieve therapeutic effects without the side effect profile. The 10D10 antibody was a lead antagonist anti-CB1 antibody isolated from Xenomouse antibody campaigns but its potency was significantly lower than that of rimonabant. (WO 2014/210205, the disclosure of which is incorporated by reference in its entirety). Multiple rounds of affinity maturations were carried out to improve the potency of the antibody. Although several 10D10 affinity matured variants isolated from the earlier affinity maturation campaigns had improved potency compared to 10D10, none of the variants met the design goal of rimonabant-like potency. The lack of stable, high-quality preparations of conformationally-relevant CB1 protein was believed to have contributed to the inability in isolating anti-CB1 antibodies with rimonabant-like potency. In addition, the 10D10 antibody and its earlier affinity matured variants had suboptimal manufacturability (e.g., low expression levels and instability).

The evolution of technical sophistication in generating high-quality, soluble preparations of complex membrane proteins culminated with the ability to formulate huCB1 into nanodisc (ND) or styrene maleic acid lipoparticles (SMALP) (Luna V. et al., European Polymer Journal 109 (2018) 483-488; Lavington S. and Watts A, Biophysical Reviews, vol. 12 (2020)1287-1302). In contrast to detergent-solubilized cell lysates prepared from huCB1-expressing cells, both CB1-ND and -SMALP are purified reagents that are stable after multiple freeze/thaw cycles and compatible with long-term storage. Furthermore, they can be added to binding experiments at user-defined concentrations of huCB1. The availability of CB1-ND and -SMALP enabled the isolation of high potency antagonist or inverse agonist anti-CB1 antibodies disclosed herein.

The present application relates to antagonist or inverse agonist anti-CB1 antibodies and antigen binding fragments, in particular anti-huCB1 antibodies and antigen binding fragments, with potencies that are comparable to or better than that of rimonabant. Antibodies that inhibit CB1 cause an increase of cAMP, which can be measured using in vitro cAMP assays such as a cell-based cAMP assay. As exemplified in the examples, potencies of the antibodies and antigen binding fragments disclosed herein are in the single digit nanomolar range, e.g., they have an IC 50 of less than about 10 nM in a cell-based cAMP assay (see Examples 5, 8 and 9). As shown in the examples, the high potency antibodies and antigen binding fragments disclosed herein reduced body weight, food intake and multiple metabolic parameters in obese animal models (e.g., reduce body fat, insulin level and liver triglyceride levels, Example 7), and increased energy expenditure, lipid oxidation and insulin sensitivity in animal models while had minimal brain exposure, indicating that they can enable safe and effective therapeutic treatment of various diseases and dis orders (e.g., obesity and its comorbidities, chronic kidney disease, NAFLD, NASH). In contrast, antagonist or inverse agonist antibodies with potency values lower than that of rimonabant are not considered to be adequate for such therapeutic purposes. Furthermore, the anti-CB1 antibodies and antigen binding fragments disclosed herein are stable and express well for manufacture purpose. These high potency anti-CB1 antibodies and antigen binding fragments are described and exemplified herein.

### Definitions

The present invention provides antagonist or inverse agonist antibodies against human CB1. An “antibody” is a protein that comprises an antigen-binding fragment that specifically binds to an antigen, and a scaffold or framework portion that allows the antigen-binding fragment to adopt a conformation that promotes binding of the antibody to the antigen. As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL or LV) and a single immunoglobulin light chain constant domain (CL or LC). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH or HV), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu ( ), delta (A), gamma (7), alpha (a), and epsilon (F), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains.

In a human antibody, CH1 means a region having the amino acid sequence at positions 118 to 215 of the EU index or EU numbering system, which is based on the sequential numbering of the first human IgG1 sequenced (i.e., the “EU antibody”) (Edelman et al., Proc Natl Acad Sci USA, 63(1): 78-85 (1969)). A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a κ chain of a human antibody, CL represents a region having the amino acid sequence at positions 108 to 214 of the EU index. In a X chain, CL represents a region having the amino acid sequence at positions 108 to 215.

A “variable domain” refers to the variable region of the antibody light chain (VL or LV) or the variable region of the antibody heavy chain (VH or HV), either alone or in combination. As known in the art, the variable regions of the heavy and light chains each consist of four framework regions (FR) connected by three complementarity determining regions (CDRs), and contribute to the formation of the antigen-binding site of antibodies. From N-terminus to C-terminus, naturally-occurring light and heavy chain variable regions both typically conform with the following order of these elements: FR1, CDR1, FR2, CDR2, FR3, CDR3 and FR4.

The “Complementarity Determining Regions” (CDRs) of an antibody can be defined according to Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). AbM definition of CDRs is a compromise between Kabat and Chothia and uses Oxford Molecular's AbM antibody modeling software (Accelrys®). The identity of the amino acid residues in a particular antibody that make up a CDR can be determined using methods well known in the art.

The term “antigen-binding fragment” refers to a molecule that derived from an antibody and retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, and (v) a dAb fragment (Ward et al., 1989 Nature 341:544-546), which consists of a VH domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988) and Huston et al., 1988, Proc. Natl. Acad. Sci. USA 85:5879-5883.

“About” or “approximately,” when used in connection with a measurable numerical variable, refers to the indicated value of the variable and to all values of the variable that are within the experimental error of the indicated value (e.g. within the 95% confidence interval for the mean) or ±10% of the indicated value, whichever is greater. Numeric ranges are inclusive of the numbers defining the range.

The term “isolated molecule” (where the molecule is, for example, a polypeptide, a polynucleotide, an antibody, or antigen-binding fragment) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

Unless indicated otherwise, throughout the present specification and claims, the numbering of the amino acid residues in an immunoglobulin heavy chain or light chain is according to the AHo numbering scheme as described in Honegger and Pluckthun, J. Mol. Biol. 309(3):657-670; 2001. In some embodiments, the EU numbering scheme as described in Edelman et al., Proc. Natl. Acad. USA, Vol. 63: 78-85 (1969) is used when referring to the position of an amino acid with an immunoglobulin constant region. Both numbering schemes are well known in the art.

An “antagonist” refers to an agent that binds to a protein (e.g., a receptor) and inter alia, partially or totally inhibits, blocks, decreases, prevents, delays, inactivates, desensitizes, or down regulates the signaling activity of a ligand of the protein (e.g., agonist of the receptor). An inverse agonist is an agent that causes the opposite effect of an agonist, for example, it decreases basal activity of a receptor.

### Anti-CB1 Antibodies and Antigen Binding Fragments

Disclosed herein are antibodies and antigen binding fragments against huCB1 (SEQ ID NO: 559) and are antagonist and/or inverse agonist of huCB1. Potencies of the antibodies and antigen binding fragments are comparable to or better than that of rimonabant, which can enable them to achieve therapeutic effects (e.g., treating obesity, chronic kidney disease, liver disease) without CNS-related side effects. In some embodiments, the antibodies and antigen binding fragments have an IC50 in the single digit nanomolar range as measured by a cell-based cAMP assay. For example, in some embodiments, the IC50 of the antibodies and antigen binding fragments is less than about 10 nM as measured by a cell-based cAMP assay. In some embodiments, the IC50 of the antibodies and antigen binding fragments is less than about 5 nM as measured by a cell-based cAMP assay. In some embodiments, the IC50 of the antibodies and antigen binding fragments is less than about 3 nM or less than about 1 nM as measured by a cell-based cAMP assay.

Anti-CB1 antibodies and antigen binding fragments disclosed herein were identified from affinity maturation of 10D10 (e.g., Examples 1-4 and 9). The 10D10 antibody was isolated from previous antibody campaigns (WO2014/210205) and the heavy and light chain CDR sequences and heavy and light chain variable region sequences of 10D10 are listed in Tables 7 and 8. The yeast display library designs for affinity maturation were guided by next generation sequencing wherein amino acids at specific HV and LV positions of 10D10 were optimized to improve binding affinity and potency. The 10D10 LC belongs to the VK2 germline and as disclosed herein, only the HC of the antibody binds to the CB1 receptor (Examples 1 and 14). LC-swapped affinity matured antibodies (i.e., swapping the 10D10 LC with light chains of VK1 or VK3 germline that have improved manufacturability) were also isolated. Heavy and light chain consensus CDR sequences of the affinity matured antibodies and antigen binding fragments are summarized in Table 35. Thus, in one aspect, disclosed herein is an antibody or antigen binding fragment thereof that binds to huCB1 and comprises a heavy chain variable region (HV) and a light chain variable region (LV), wherein
a) the HV comprises HCDR1, HCDR2 and HCDR3, wherein HCDR1 comprises the amino acid sequence of RGGDYWX1 (SEQ ID NO: 530), wherein X1 is A, S, or G; HCDR2 comprises the amino acid sequence of HX2YX3X4GX5TX6YNPX7X8X9X10 (SEQ ID NO: 531), wherein X2 is I or V; X3 is H, Y, or Q; X4 is E, T, or S; X5 is K, S, or Q; X6 is A, K or N; X7 is N, S, K, or R; X8 is F or L; X9 is E or K; and X10 is G, D, S, or N; and HCDR3 comprises the amino acid sequence of X11YDX12X13X14GX15SYYYYGMDV (SEQ ID NO: 532), wherein X11 is D, E, or N; X12 is A, I, P, T, or V; X13 is F, L, or V; X14 is S or T; and X15 is H, N, or Y; and
b) the LV is of or derived from a VK1/O2/JK4, a VK2/A19/JK1, or a VK3/A27/JK1 germline.

As used herein, a variable region sequence (such as a variable region or CDR amino acid sequence) “of a germline” or “derived from a germline” refers to that sequence having structural features that are characteristic of the specified germline, rather than another germline. In order for a host organism to generate an antibody that binds to an antigen, rearranged immunoglobulin germline genes (of a B cell of the host organism) must initially encode an initial antibody with a three-dimensional structure and biochemical properties capable of interreacting with that antigen. Each host organism has a defined repertoire of immunoglobulin germline genes that may be rearranged to produce initial antibodies. Through the process of somatic hypermutation, the host organism can select for a mutation or collection of mutations in the genes encoding the antibody that further enhance its interaction with the antigen. Accordingly, an antibody sequence “of” or “derived from” a specified germline may have variable region sequences that contain differences compared to that germline. However, the antibody sequence will contain structural features, such as particular amino acids, CDR lengths and/or conformation characteristic of a specified germline, indicating that the specified germline is the closest germline for that sequence to be associated with. This interaction can be analogized to a hand (antigen) being fitted by a glove (germline antibody). The initial structures defined by the germline genes must be sufficient for the glove to fit the hand in the first place, which may then be tailored to fit with even more precision. Since the specific combination of rearranged immunoglobulin germline genes that encoded the antibody must have specified a three-dimensional structure (glove) that interacted with the antigen (hand), the germline identity of an antibody will be understood to define structural features of paratope that interact with the antigen. As light chain of the CB1 antibodies disclosed herein does not make direct contact with the antigen, the light chain germline identity will be understood to define structural features of the light chains for pairing with the heavy chains for paratope interaction with the antigen.

Repertoires of germline genes that are typically utilized for generating antibodies, including Homo sapiens and Mus musculus are sequences, and are known and may be accessed, for example, from IMGT/GENE-DB (Giudicelli V., et al. “IMGT/GENE-DB: a comprehensive database for human and mouse immunoglobulin and T cell receptor genes.” Nucleic Acids Res., 33: D256-D261 (2005)). A sequence of an antibody may be aligned against germline gene repertoires, for example using an algorithm such as Basic Local Alignment Search Tool (BLAST) under default settings, to determine which host organism germline gene segment (or segments) that antibody is of.

In some embodiments, the antibody or antigen binding fragment thereof comprises a) HCDR1 comprising the sequence of RGGDYWX1 (SEQ ID NO: 530), wherein X1 is A or S; HCDR2 comprising the sequence of HX2YX3X4GX5TX6YNPX7X8X9X10 (SEQ ID NO: 531), wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N, and HCDR3 comprising the sequence of X11YDX12X13X14GX15SYYYYGMDV (SEQ ID NO: 532), wherein X11is D, E, or N; X12 is A, I, P, or V; X13 is L or V; X14 is S or T; and X15 is H or Y. In some embodiments, the antibody or antigen binding fragment thereof comprises a) HCDR1 comprising the sequence of SEQ ID NO: 530, wherein X1 is A or S; HCDR2 comprising the sequence SEQ ID NO: 531, wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N, and HCDR3 comprising the sequence of SEQ ID NO: 532, wherein X11 is D, E, or N; X12 is A, I, P, or V; X13 is L or V; X14 is T; and X15 is Y. In various embodiments, wherein the HCDR1, HCDR2 and HCDR3 comprise the sequence defined above, the LV is of or derived from a VK1/O2/JK4, a VK2/A19/JK1, or a VK3/A27/JK1 germline. In some embodiments, the LV is of or derived from a VK2/A19/JK1 germline.

In some embodiments, the antibody or antigen binding fragment thereof comprises a) HCDR1 comprising the sequence of SEQ ID NO: 530, wherein X1 is A or S; HCDR2 comprising the sequence of SEQ ID NO: 531, wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N, and HCDR3 comprising the sequence of SEQ ID NO: 532, wherein X11 is D, E, or N; X12 is A, I, P, or V; X13 is L or V; X14 is S or T; and X15 is H or Y, and b) the LV comprises LCDR1, LCDR2 and LCDR3, wherein LCDR1 comprises the amino acid sequence RSSQSLLX16SX17GX18NYX19D (SEQ ID NO: 533), wherein X16 is H, S, or T; X17 is S, T, or Y; X18 is A, N, I or Y; and X19 is L or V; LCDR2 comprises the amino acid sequence of X20GSNRA (SEQ ID NO: 534), wherein X20 is L or Q; and LCDR3 comprises the amino acid sequence of X21QAX22X23X24PRT (SEQ ID NO: 535), wherein X21 is M or R; X22 is L, I, R, or V; X23 is Q, E, T, A, or G; and X24 is T, I, L, or Q.

In some embodiments, the antibody or antigen binding fragment thereof comprises a) HCDR1 comprising the sequence of SEQ ID NO: 530, wherein X1 is A or S; HCDR2 comprising the sequence SEQ ID NO: 531, wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N, and HCDR3 comprising the sequence of SEQ ID NO: 532, wherein X11 is D, E, or N; X12 is A, I, P, or V; X13 is L or V; X14 is T; and X15 is Y, and b) LCDR1 comprises the sequence SEQ ID NO: 533, wherein X16 is H, S, or T; X17 is S, T, or Y; X18 is A, N, I or Y; and X19 is L or V; LCDR2 comprises the sequence of SEQ ID NO: 534, wherein X20 is L or Q; and LCDR3 comprises the sequence of SEQ ID NO: 535, wherein X21 is M or R; X22 is L, I, R, or V; X23 is Q, E, T, A, or G; and X24 is T, I, L, or Q.

In some embodiments, the antibody or antigen binding fragment thereof comprises a) HCDR1 comprising the sequence of SEQ ID NO: 530, wherein X1 is A or S; HCDR2 comprising the sequence of SEQ ID NO: 531, wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N, and HCDR3 comprising the sequence of SEQ ID NO: 532, wherein X11 is D, E, or N; X12 is A, I, P, or V; X13 is L or V; X14 is S or T; and X15 is H or Y, and b) LCDR1 comprises the sequence SEQ ID NO: 533, wherein X16 is H, S, or T; X17 is S, T, or Y; X18 is A, N, I or Y; and X19 is L or V; LCDR2 comprises the sequence of SEQ ID NO: 534, wherein X20 is L or Q; and LCDR3 comprises the sequence of SEQ ID NO: 535, wherein X21 is M or R; X22 is L, I, R, or V; X23 is Q, E, T, A, or G; and X24 is L.

In some embodiments, the antibody or antigen binding fragment thereof comprises a) HCDR1 comprising the sequence of SEQ ID NO: 530, wherein X1 is A or S; HCDR2 comprising the sequence SEQ ID NO: 531, wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N, and HCDR3 comprising the sequence of SEQ ID NO: 532, wherein X11 is D, E, or N; X12 is A, I, P, or V; X13 is L or V; X14 is T; and X15 is Y, and b) LCDR1 comprises the amino acid sequence SEQ ID NO: 533, wherein X16 is H, S, or T; X17 is S, T, or Y; X18 is A, N, I or Y; and X19 is L or V; LCDR2 comprises the amino acid sequence of SEQ ID NO: 534, wherein X20 is L or Q; and LCDR3 comprises the amino acid sequence of SEQ ID NO: 535, wherein X21 is M or R; X22 is L, I, R, or V; X23 is Q, E, T, A, or G; and X24 is L.

In some embodiments, the antibody or antigen binding fragment thereof comprises a) HCDR1 comprising the sequence of SEQ ID NO: 530, wherein X1 is A, G or S; HCDR2 comprising the sequence of SEQ ID NO: 531, wherein X2 is I or V; X3 is H, Y, or Q; X4 is T or S; X5 is K, S or Q; X6 is A, K or N; X7 is N, S, K, or R; X8 is F or L; X9 is E or K; and X10 is G, D, S, or N; and HCDR3 comprising the sequence of SEQ ID NO: 532, wherein X11 is D or N; X12 is A, I, or T; X13 is F, L, or V; X14 is S or T; and X15 is H, N or Y. In some embodiments, the antibody or antigen binding fragment thereof comprises a) HCDR1 comprising the sequence of SEQ ID NO: 530, wherein X1 is A or G; HCDR2 comprising the sequence of SEQ ID NO: 531, wherein X2 is I or V; X3 is Y or Q; X4 is T; X5 is K, S or Q; X6 is A, K or N; X7 is N, S, K, or R; X8 is F; X9 is E or K; and X10 is G, D, S, or N; and HCDR3 comprising the sequence of SEQ ID NO: 532, wherein X11is D or N; X12 is A, I, or T; X13 is F, L, or V; X14 is T; and X15 is Y. In some embodiments, the antibody or antigen binding fragment thereof comprises a) HCDR1 comprising the sequence of SEQ ID NO: 530, wherein X1 is A or G; HCDR2 comprising the sequence of SEQ ID NO: 531, wherein X2 is I or V; X3 is Y or Q; X4 is T; X5 is S; X6 is K or N; X7 is S or R; X8 is F; X9 is K; and X10 is G or D; and HCDR3 comprising the sequence of SEQ ID NO: 532, wherein X11 is N; X12 is T; X13 is L or V; X14 is T; and X15 is Y. In various embodiments, wherein the HCDR1, HCDR2 and HCDR3 comprise the sequence defined above, the LV is of or derived from a VK1/O2/JK4 or a VK3/A27/JK1 germline. In some embodiments, the LV is of or derived from a VK1/O2/JK4 germline.

In some embodiments, the antibody or antigen binding fragment thereof comprises a) HCDR1 comprising the sequence of SEQ ID NO: 530, wherein X1 is A, G or S; HCDR2 comprising the sequence of SEQ ID NO: 531, wherein X2 is I or V; X3 is H, Y, or Q; X4 is T or S; X5 is K, S or Q; X6 is A, K or N; X7 is N, S, K, or R; X8 is F or L; X9 is E or K; and X10 is G, D, S, or N; and HCDR3 comprising the sequence of SEQ ID NO: 532, wherein X11 is D or N; X12 is A, I, or T; X13 is F, L, or V; X14 is S or T; and X15 is H, N or Y, and b) the LV is of or derived from a VK1/O2/JK4 or a VK3/A27/JK1 germline, preferably, the LV is of or derived from a VK1/O2/JK4 germline.

In some embodiments, the antibody or antigen binding fragment thereof comprises a) HCDR1 comprising the sequence of SEQ ID NO: 530, wherein X1 is A or G; HCDR2 comprising the sequence of SEQ ID NO: 531, wherein X2 is I or V; X3 is Y or Q; X4 is T; X5 is K, S or Q; X6 is A, K or N; X7 is N, S, K, or R; X8 is F; X9 is E or K; and X10 is G, D, S, or N; and HCDR3 comprising the sequence of SEQ ID NO: 532, wherein X11 is D or N; X12 is A, I, or T; X13 is F, L, or V; X14 is T; and X15 is Y, and b) the LV is of or derived from a VK1/O2/JK4 germline.

In some embodiments, the antibody or antigen binding fragment thereof comprises a) HCDR1 comprising the sequence of SEQ ID NO: 530, wherein X1 is A, G or S; HCDR2 comprising the sequence of SEQ ID NO: 531, wherein X2 is I or V; X3 is H, Y, or Q; X4 is T or S; X5 is K, S or Q; X6 is A, K or N; X7 is N, S, K, or R; X8 is F or L; X9 is E or K; and X10 is G, D, S, or N; and HCDR3 comprising the sequence of SEQ ID NO: 532, wherein X11 is D or N; X12 is A, I, or T; X13 is F, L, or V; X14 is S or T; and X15 is H, N or Y, and b) the LV is of or derived from a VK1/O2/JK4 and comprises LCDR1, LCDR2, and LCDR3, wherein LCDR1 comprises the amino acid sequence of RASQSISNYLN (SEQ ID NO: 132), RASQSIISYLN (SEQ ID NO: 150), or RASQSISSYLN (SEQ ID NO: 186); LCDR2 comprises the amino acid sequence of AASSLHS (SEQ ID NO: 133) or AASSLRS (SEQ ID NO: 151); and LCDR3 comprises the amino acid sequence of QQYQSYPLT (SEQ ID NO: 134) or QQYSNYPLT (SEQ ID NO: 152); or the VL is of a VK3/A27/JK1 germline and comprises light chain LCDR1, LCDR2 and LCDR3, and wherein LCDR1 comprises the amino acid sequence of RASQSVSSYLG (SEQ ID NO: 168); LCDR2 comprises the amino acid sequence of GASSRAT (SEQ ID NO: 169); and LCDR3 comprises the amino acid sequence of QQYGSSPRT (SEQ ID NO: 170).

In some embodiments, the antibody or antigen binding fragment thereof comprises a) HCDR1 comprising the sequence of SEQ ID NO: 530, wherein X1 is A, G or S; HCDR2 comprising the sequence of SEQ ID NO: 531, wherein X2 is I or V; X3 is H, Y, or Q; X4 is T or S; X5 is K, S or Q; X6 is A, K or N; X7 is N, S, K, or R; X8 is F or L; X9 is E or K; and X10 is G, D, S, or N; and HCDR3 comprising the sequence of SEQ ID NO: 532, wherein X11 is D or N; X12 is A, I, or T; X13 is F, L, or V; X14 is S or T; and X15 is H, N or Y, and b) LCDR1 comprising the sequence of RASQSISNYLN (SEQ ID NO: 132), RASQSIISYLN (SEQ ID NO: 150), or RASQSISSYLN (SEQ ID NO: 186); LCDR2 comprising the sequence of AASSLHS (SEQ ID NO: 133) or AASSLRS (SEQ ID NO: 151); and LCDR3 comprising the sequence of QQYQSYPLT (SEQ ID NO: 134) or QQYSNYPLT (SEQ ID NO: 152); preferably, b) LCDR1 comprising the sequence of RASQSISNYLN (SEQ ID NO: 132); LCDR2 comprising the sequence of AASSLHS (SEQ ID NO: 133); and LCDR3 comprising the sequence of QQYQSYPLT (SEQ ID NO: 134).

In some embodiments, the antibody or antigen binding fragment thereof comprises a) HCDR1 comprising the sequence of SEQ ID NO: 530, wherein X1 is A or G; HCDR2 comprising the sequence of SEQ ID NO: 531, wherein X2 is I or V; X3 is Y or Q; X4 is T; X5 is K, S or Q; X6 is A, K or N; X7 is N, S, K, or R; X8 is F; X9 is E or K; and X10 is G, D, S, or N; and HCDR3 comprising the sequence of SEQ ID NO: 532, wherein X11 is D or N; X12 is A, I, or T; X13 is F, L, or V; X14 is T; and X15 is Y, and b) the LV is of or derived from a VK1/O2/JK4 germline and comprises LCDR1, LCDR2, and LCDR3, wherein LCDR1 comprises the amino acid sequence of RASQSISNYLN (SEQ ID NO: 132), RASQSIISYLN (SEQ ID NO: 150), or RASQSISSYLN (SEQ ID NO: 186); LCDR2 comprises the amino acid sequence of AASSLHS (SEQ ID NO: 133) or AASSLRS (SEQ ID NO: 151); and LCDR3 comprises the amino acid sequence of QQYQSYPLT (SEQ ID NO: 134) or QQYSNYPLT (SEQ ID NO: 152); preferably, b) LCDR1 comprising the sequence of RASQSISNYLN (SEQ ID NO: 132); LCDR2 comprising the sequence of AASSLHS (SEQ ID NO: 133); and LCDR3 comprising the sequence of QQYQSYPLT (SEQ ID NO: 134).

In some embodiments, the antibody or antigen binding fragment thereof comprises a) HCDR1 comprising the sequence of SEQ ID NO: 530, wherein X1 is A or G; HCDR2 comprising the sequence of SEQ ID NO: 531, wherein X2 is I or V; X3 is Y or Q; X4 is T; X5 is S; X6 is K or N; X7 is S or R; X8 is F; X9 is K; and X10 is G or D; and HCDR3 comprising the sequence of SEQ ID NO: 532, wherein X11 is N; X12 is T; X13 is L or V; X14 is T; and X15 is Y, and b) the LV is of or derived from a VK1/O2/JK4 germline. In some embodiments, the antibody or antigen binding fragment thereof comprises a) HCDR1 comprising the amino acid sequence of RGGDYWA (SEQ ID NO: 414) or RGGDYWG (SEQ ID NO: 408); HCDR2 comprising the amino acid sequence of HVYYTGSTKYNPSFKD (SEQ ID NO: 427), HVYYTGSTNYNPRFKD (SEQ ID NO: 445), HIYQTGSTNYNPRFKG (SEQ ID NO: 415), or HVYQTGSTKYNPSFKD (SEQ ID NO: 409); and HCDR3 comprising the amino acid sequence of NYDTLTGYSYYYYGMDV (SEQ ID NO: 410) or NYDTVTGYSYYYYGMDV (SEQ ID NO: 446, and b) the LV is of or derived from a VK1/O2/JK4 germline.

In some embodiments, the antibody or antigen binding fragment thereof comprises a) HCDR1 comprising the sequence of SEQ ID NO: 530, wherein X1 is A or G; HCDR2 comprising the sequence of SEQ ID NO: 531, wherein X2 is I or V; X3 is Y or Q; X4 is T; X5 is S; X6 is K or N; X7 is S or R; X8 is F; X9 is K; and X10 is G or D; and HCDR3 comprising the sequence of SEQ ID NO: 532, wherein X11 is N; X12 is T; X13 is L or V; X14 is T; and X15 is Y, and b) the LV is of or derived from a VK1/O2/JK4 germline comprising LCDR1, LCDR2 and LCDR3, wherein LCDR1 comprises the sequence of RASQSISSYLN (SEQ ID NO: 405); LCDR2 comprises the sequence of X39ARX40LX41S (SEQ ID NO: 536), wherein X39 is N, S, K or G; X40 is R, K, L or A; and X41 is A, G or S; and LCDR3 comprises the sequence of QQX42X43X44X45PX46T (SEQ ID NO: 537), wherein X42 is Y or F, X43 is R, A, S, G, or Y; X44 is S, K, R or H; X45 is S, L, F, Y, P, or M; and X46 is L, I or V. In some embodiments, X42 is Y and X1-X15, X39-X41, and X43-X44 are as defined above.

In some embodiments, the antibody or antigen binding fragment thereof comprises a) HCDR1 comprising the amino acid sequence of RGGDYWA (SEQ ID NO: 414) or RGGDYWG (SEQ ID NO: 408); HCDR2 comprising the amino acid sequence of HVYYTGSTKYNPSFKD (SEQ ID NO: 427), HVYYTGSTNYNPRFKD (SEQ ID NO: 445), HIYQTGSTNYNPRFKG (SEQ ID NO: 415), or HVYQTGSTKYNPSFKD (SEQ ID NO: 409); and HCDR3 comprising the amino acid sequence of NYDTLTGYSYYYYGMDV (SEQ ID NO: 410) or NYDTVTGYSYYYYGMDV (SEQ ID NO: 446, and b) LCDR1 comprises the sequence of RASQSISSYLN (SEQ ID NO: 405); LCDR2 comprises the sequence of X39ARX40LX41S (SEQ ID NO: 536), wherein X39 is N, S, K or G; X40 is R, K, L or A; and X41 is A, G or S; and LCDR3 comprises the sequence of QQX42X43X44X45PX46T (SEQ ID NO: 537), wherein X42 is Y or F, X43 is R, A, S, G, or Y; X44 is S, K, R or H; X45 is S, L, F, Y, P, or M; and X46 is L, I or V. In some embodiments, X42 is Y and X39-X41 and X43-X44 are as defined above.

In certain embodiments, the anti-CB1 antibodies or antigen binding fragments disclosed herein comprise a heavy chain variable region comprising a HCDR1, HCDR2, and HCDR3, and a light chain variable region comprising a LCDR1, LCDR2, and LCDR3 from any of the anti-CB1 antibodies described herein. Thus, in some embodiments, described herein is an anti-CB1 antibody or antigen binding fragment thereof comprising a HCDR1, HCDR2, HCDR3, LCDR1, LCDR2, or LCDR3 of any one of LIBC523596-1, LIBC527906-1, LIBC523603-1, LIBC527814-1, LIBC523661-1, LIBC527997-1, LIBC523670-1, LIBC528141-1, LIBC523748-1, LIBC527912-1, LIBC523760-1, LIBC528116-1, LIBC523797-1, LIBC527879-1, LIBC523813-1, LIBC527919-1, LIBC523815-1, LIBC527984-1, LIBC523844-1, LIBC527968-1, LIBC523857-1, LIBC528169-1, LIBC523862-1, LIBC528131-1, LIBC523868-1, LIBC527827-1, LIBC523969-1, LIBC527869-1, LIBC524049-1, LIBC528148-1, LIBC680562-1, LIBC680574-1, LIBC680773-1, LIBC680800-1, LIBC680812-1, LIBC681593-1, LIBC681594-1, and LIBC681737-1. In some embodiments, the anti-CB1 antibody or antigen binding fragment thereof comprises a HCDR1, HCDR2, and HCDR3, and a LCDR1, LCDR2, and LCDR3 of any one of these antibodies. In some embodiments, the anti-CB1 antibody or antigen binding fragment thereof comprises a HCDR1, HCDR2, and HCDR3, and a LCDR1, LCDR2, and LCDR3 of any one of LIBC523661-1, LIBC523797-1, LIBC524049-1, LIBC527814-1, LIBC527997-1, LIBC528116, LIBC527919-1, and LIBC680574-1. HV and LV CDR sequences of these antibodies are listed in Tables 7, 10 and 26.

In some embodiments, the anti-CB1 antibody or antigen binding fragment thereof comprises a HV or a LV of any one of LIBC523596-1, LIBC527906-1, LIBC523603-1, LIBC527814-1, LIBC523661-1, LIBC527997-1, LIBC523670-1, LIBC528141-1, LIBC523748-1, LIBC527912-1, LIBC523760-1, LIBC528116-1, LIBC523797-1, LIBC527879-1, LIBC523813-1, LIBC527919-1, LIBC523815-1, LIBC527984-1, LIBC523844-1, LIBC527968-1, LIBC523857-1, LIBC528169-1, LIBC523862-1, LIBC528131-1, LIBC523868-1, LIBC527827-1, LIBC523969-1, LIBC527869-1, LIBC524049-1, LIBC528148-1, LIBC680562-1, LIBC680574-1, LIBC680773-1, LIBC680800-1, LIBC680812-1, LIBC681593-1, LIBC681594-1, and LIBC681737-1. In some embodiments, the anti-CB1 antibody or antigen binding fragment thereof comprises a HV and a LV of any one of LIBC523596-1, LIBC527906-1, LIBC523603-1, LIBC527814-1, LIBC523661-1, LIBC527997-1, LIBC523670-1, LIBC528141-1, LIBC523748-1, LIBC527912-1, LIBC523760-1, LIBC528116-1, LIBC523797-1, LIBC527879-1, LIBC523813-1, LIBC527919-1, LIBC523815-1, LIBC527984-1, LIBC523844-1, LIBC527968-1, LIBC523857-1, LIBC528169-1, LIBC523862-1, LIBC528131-1, LIBC523868-1, LIBC527827-1, LIBC523969-1, LIBC527869-1, LIBC524049-1, LIBC528148-1, LIBC680562-1, LIBC680574-1, LIBC680773-1, LIBC680800-1, LIBC680812-1, LIBC681593-1, LIBC681594-1, and LIBC681737-1. HV and LV sequences of these antibodies are listed in Tables 8, 11 and 27.

In some embodiments, the anti-CB1 antibody or antigen binding fragment thereof comprises a HV or LV of any one of LIBC523661-1, LIBC523797-1, LIBC524049-1, LIBC527814-1, LIBC527997-1, LIBC528116, LIBC527919-1, and LIBC680574-1. In some embodiments, the anti-CB1 antibody or antigen binding fragment thereof comprises a HV and LV of any one of LIBC523661-1, LIBC523797-1, LIBC524049-1, LIBC527814-1, LIBC527997-1, LIBC528116, LIBC527919-1, and LIBC680574-1.

Certain mutations in HC_FR3 de-loop of 10D10 that are associated with improved binding to CB1 were included in the affinity maturation library design (Example 1). In addition, all tested sequences identified after affinity maturation included the R94S germline reversion of a LC FR3 non-consensus mutation (Examples 2 and 3). Further, R32 (AHo numbering) that is adjacent to HCDR1 is also involved in epitope interaction. Thus, in some embodiments of the antibodies and antigen binding fragments described above, the amino acid at HV position 83 (AHo numbering scheme) is N and the amino acid at HV position 85 (AHo numbering scheme) is Y. In some embodiments of the antibodies and antigen binding fragments described above, the amino acid at HV position 83 is N, the amino acid at HV position 85 is Y, and the amino acid at LV position 94 is S. In some embodiments, of the antibodies and antigen binding fragments described above the amino acid at HV position 32 is R.

In various embodiments, the antibodies and antigen binding fragments disclosed herein are antagonist and/or inverse agonist of huCB1, for example, the antibodies and binding fragments inhibit signaling of huCB1. Potencies of the antibodies and antigen binding fragments are in the nanomolar or subnanomolar range, for example, the antibodies and antigen binding fragments have an IC50 of less than about 10 nM, less than about 5 nM, less than about 3 nM or less than about 1 nM, as measured in a cell-based cAMP assay. In some embodiments, potencies of the antibodies and antigen binding fragments disclosed herein are comparable to or better than that of rimonabant (e.g., at least two-fold, three-fold, five-fold, eight-fold, or ten-fold better than that of rimonabant).

In another aspect, disclosed herein is an antibody or antigen binding fragment thereof that binds to huCB1 and comprises a heavy chain variable region (HV) and a light chain variable region (LV), wherein
a) the VH comprises HCDR1, HCDH2 and HCDR3, wherein CDRH1 comprises the amino acid sequence of RGGDYWS (SEQ ID NO: 1); CDRH2 comprises the sequence of HX25YX26X27GX28TX29YNPX30X31X32X33 (SEQ ID NO: 538), wherein X25 is I or V; X26 is Y or Q; X27 is A, E, K, or T; X28 is S or Q; X29 is A, E, K, or T; X30 is N or S; X31 is F or L, X32 is K or R; and X33 is G, S, or N; and CDRH3 comprises the sequence of X34YDX35X36X37GYSYYYYGX38DV (SEQ ID NO: 539), wherein X34 is D or G; X35 is A, I, T, or V; X36 is L or absent; X37 is S or T; and X38 is M or L; and
b) the VL is of or derived from a VK2/A19/JK1 germline.

In some embodiments, the antibody or antigen binding fragment thereof comprises a) CDRH1 comprising the sequence of RGGDYWS (SEQ ID NO: 1); CDRH2 comprising the sequence of HX25YX26X27GX28TX29YNPX30X31X32X33 (SEQ ID NO: 538), wherein X25 is I or V; X26 is Y; X27 is A, E, K, or T; X28 is S or Q; X29 is A, E, K, or T; X30 is N or S; X31 is F or L, X32 is K or R; and X33 is G, S, or N; and CDRH3 comprising the sequence of X34YDX35X36X37GYSYYYYGX38DV (SEQ ID NO: 539), wherein X34 is G; X35 is A, I, T, or V; X36 is absent; X37 is S; and X38 is M or L, and b) the VL is of or derived from a VK2/A19/JK1 germline.

In some embodiments, the antibody or antigen binding fragment thereof comprises a) CDRH1 comprising the sequence of RGGDYWS (SEQ ID NO: 1); CDRH2 comprising the sequence of HX25YX26X27GX28TX29YNPX30X31X32X33 (SEQ ID NO: 538), wherein X25 is I or V; X26 is Y or Q; X27 is A, E, K, or T; X28 is S or Q; X29 is A, E, K, or T; X30 is N or S; X31 is F or L, X32 is K or R; and X33 is G, S, or N; and CDRH3 comprising the sequence of X34YDX35X36X37GYSYYYYGX38DV (SEQ ID NO: 539), wherein X34 is D or G; X35 is A, I, T, or V; X36 is L or absent; X37 is S or T; and X38 is M or L, and b) the VL is of or derived from a VK2/A19/JK1 germline and comprises LCDR1, LCDR2, and LCDR3, wherein LCDR1 comprises the sequence of RSSQSLLHRSGYNYLD (SEQ ID NO: 257); LCDR2 comprises the sequence of X20GSNRA (SEQ ID NO: 534), wherein X20 is L or Q; and LCDR3 comprises X47QX48X49X50X51PRT (SEQ ID NO: 540), wherein X47 is M or R; X48 is A or S; X49 is L, R, or V; X50 is Q or A; and X51 is T or L.

In some embodiments, the antibody or antigen binding fragment thereof comprises a) CDRH1 comprising the sequence of RGGDYWS (SEQ ID NO: 1); CDRH2 comprising the sequence of HX25YX26X27GX28TX29YNPX30X31X32X33 (SEQ ID NO: 538), wherein X25 is I or V; X26 is Y; X27 is A, E, K, or T; X28 is S or Q; X29 is A, E, K, or T; X30 is N or S; X31 is F or L, X32 is K or R; and X33 is G, S, or N; and CDRH3 comprising the sequence of X34YDX35X36X37GYSYYYYGX38DV (SEQ ID NO: 539), wherein X34 is G; X35 is A, I, T, or V; X36 is absent; X37 is S; and X38 is M or L, and b) LCDR1 comprising the sequence of RSSQSLLHRSGYNYLD (SEQ ID NO: 257); LCDR2 comprising the sequence of X20GSNRA (SEQ ID NO: 534), wherein X20 is L or Q; and LCDR3 comprising X47QX48X49X50X51PRT (SEQ ID NO: 540), wherein X47 is M or R; X48 is A or S; X49 is L, R, or V; X50 is Q or A; and X51 is T or L.

In some embodiments, the antibody or antigen binding fragment thereof comprises a) CDRH1 comprising the sequence of RGGDYWS (SEQ ID NO: 1); CDRH2 comprising the sequence of HX25YX26X27GX28TX29YNPX30X31X32X33 (SEQ ID NO: 538), wherein X25 is I or V; X26 is Y or Q; X27 is A, E, K, or T; X28 is S or Q; X29 is A, E, K, or T; X30 is N or S; X31 is F or L, X32 is K or R; and X33 is G, S, or N; and CDRH3 comprising the sequence of X34YDX35X36X37GYSYYYYGX38DV (SEQ ID NO: 539), wherein X34 is D or G; X35 is A, I, T, or V; X36 is L or absent; X37 is S or T; and X38 is M or L, and b) LCDR1 comprising the amino acid sequence of RSSQSLLHRSGYNYLD (SEQ ID NO: 257); LCDR2 comprising the amino acid sequence of LGSNRAS (SEQ ID NO: 258) or QGSNRAS (SEQ ID NO: 264); and LCDR3 comprising the amino acid sequence of MQSLQTPRT (SEQ ID NO: 259), RQSVALPRT (SEQ ID NO: 271), or RQARALPRT (SEQ ID NO: 265).

In some embodiments, the antibody or antigen binding fragment thereof comprises a) CDRH1 comprising the sequence of RGGDYWS (SEQ ID NO: 1); CDRH2 comprising the sequence of HX25YX26X27GX28TX29YNPX30X31X32X33 (SEQ ID NO: 538), wherein X25 is I or V; X26 is Y; X27 is A, E, K, or T; X28 is S or Q; X29 is A, E, K, or T; X30 is N or S; X31 is F or L, X32 is K or R; and X33 is G, S, or N; and CDRH3 comprising the sequence of X34YDX35X36X37GYSYYYYGX38DV (SEQ ID NO: 539), wherein X34 is G; X35 is A, I, T, or V; X36 is absent; X37 is S; and X38 is M or L, and b) LCDR1 comprising the amino acid sequence of RSSQSLLHRSGYNYLD (SEQ ID NO: 257); LCDR2 comprising the amino acid sequence of LGSNRAS (SEQ ID NO: 258) or QGSNRAS (SEQ ID NO: 264); and LCDR3 comprising the amino acid sequence of MQSLQTPRT (SEQ ID NO: 259), RQSVALPRT (SEQ ID NO: 271), or RQARALPRT (SEQ ID NO: 265).

In certain embodiments, the anti-CB1 antibodies or antigen binding fragments disclosed herein comprise a heavy chain variable region comprising a HCDR1, HCDR2, and HCDR3, and a light chain variable region comprising a LCDR1, LCDR2, and LCDR3 from any of the anti-CB1 antibodies described herein. For example, in some embodiments, the anti-CB1 antibody or antigen binding fragment thereof comprises a HCDR1, HCDR2, HCDR3, LCDR1, LCDR2, or LCDR3 of any one of LIBC673948-1, LIBC673952-1, LIBC673965-1, LIBC673982-1, LIBC673972-1, LIBC674024-1, LIBC674035-1, LIBC674043-1, LIBC674090-1, LIBC674002-1, LIBC674153-1, LIBC674200-1, LIBC674214-1, LIBC674216-1, LIBC674229-1, LIBC674235-1, LIBC674257-1, and LIBC674276-1. In some embodiments, the anti-CB1 antibody or antigen binding fragment thereof comprises a HCDR1, HCDR2, and HCDR3, and a LCDR1, LCDR2, and LCDR3 of any one of LIBC673948-1, LIBC673952-1, LIBC673965-1, LIBC673982-1, LIBC673972-1, LIBC674024-1, LIBC674035-1, LIBC674043-1, LIBC674090-1, LIBC674002-1, LIBC674153-1, LIBC674200-1, LIBC674214-1, LIBC674216-1, LIBC674229-1, LIBC674235-1, LIBC674257-1, and LIBC674276-1. HV and LV CDR sequences of these antibodies are listed in Table 30.

In some embodiments, the anti-CB1 antibody or antigen binding fragment thereof comprises a HV or a LV of any one of LIBC673948-1, LIBC673952-1, LIBC673965-1, LIBC673982-1, LIBC673972-1, LIBC674024-1, LIBC674035-1, LIBC674043-1, LIBC674090-1, LIBC674002-1, LIBC674153-1, LIBC674200-1, LIBC674214-1, LIBC674216-1, LIBC674229-1, LIBC674235-1, LIBC674257-1, and LIBC674276-1. In some embodiments, the anti-CB1 antibody or antigen binding fragment thereof comprises a HV and a LV of any one of LIBC673948-1, LIBC673952-1, LIBC673965-1, LIBC673982-1, LIBC673972-1, LIBC674024-1, LIBC674035-1, LIBC674043-1, LIBC674090-1, LIBC674002-1, LIBC674153-1, LIBC674200-1, LIBC674214-1, LIBC674216-1, LIBC674229-1, LIBC674235-1, LIBC674257-1, and LIBC674276-1. HV and LV sequences of these antibodies are listed in Table 31.

In some embodiments, the anti-CB1 antibody or antigen binding fragment thereof comprises a HCDR1, HCDR2, HCDR3, LCDR1, LCDR2, or LCDR3 of any one of LIBC529593-1, LIBC560340-1 and LIBC560657-1. In some embodiments, the anti-CB1 antibody or antigen binding fragment thereof comprises a HCDR1, HCDR2, and HCDR3, and a LCDR1, LCDR2, and LCDR3 of any one of LIBC529593-1, LIBC560340-1 and LIBC560657-1. HV and LV CDR sequences of the antibodies are listed in Table 19. For example, the antibody or antigen binding fragment thereof comprises a) HCDR1 comprising the amino acid sequence of RGGDYWS (SEQ ID NO: 474) or RGGDYWN (SEQ ID NO: 480); HCDR2 comprising the amino acid sequence of HIYYSGSTNYNPSLRS (SEQ ID NO: 475), HIYYSGSKNYNPSLKS (SEQ ID NO: 481), or HIYYTGTKYYNPSLKS (SEQ ID NO: 487), and HCDR3 comprising the amino acid sequence of DYDILYGYSYYYYGLDV (SEQ ID NO: 476), or GYDSSGYSYYYYGMDV (SEQ ID NO: 475), and b) LCDR1 comprising the amino acid sequence of RSSQSLLHRSGYNYLD (SEQ ID NO: 471), RSSQSLLYSNGHNFLD (SEQ ID NO: 477), or RSSQSLLYSNGHNYLD (SEQ ID NO: 483), LCDR2 comprises the amino acid sequence of LGSNRAS (SEQ ID NO: 472) or LGSNRAP (SEQ ID NO: 484), and LCDR3 comprises the amino acid sequence of MQSLQTPRT (SEQ ID NO: 473) or MQALQTPRT (SEQ ID NO: 479).

In some embodiments, the antibody or antigen binding fragment thereof comprises a HV or LV of any one of LIBC529593-1, LIBC560340-1 and LIBC560657-1. In some embodiments, the antibody or antigen binding fragment thereof comprises a HV and a LV of any one of LIBC529593-1, LIBC560340-1 and LIBC560657-1. HV and LV sequences of the antibodies are listed in Table 20.

In some embodiments, the anti-CB1 antibody or antigen binding fragment thereof comprises amino acid mutations in the HV CDRs compared to a reference antibody. For example, in some embodiments, the antibody or antigen binding fragment thereof comprises a) a HV comprising a HCDR1, HCDR2 and HCDR3, wherein HCDR2 of the antibody comprises 5 or fewer mutations compared to HVYYTGSTNYNPRFKD (SEQ ID NO: 136). In some embodiments, HCDR2 of the antibody or antigen binding fragment thereof comprises 4 or fewer mutations compared to HVYYTGSTNYNPRFKD (SEQ ID NO: 136). In some embodiments, HCDR2 of the antibody or antigen binding fragment thereof comprises 5 or fewer mutations compared to HVYYTGSTNYNPRFKD (SEQ ID NO: 136) and HCDR3 of the antibody or antigen binding fragment thereof comprises 3 or fewer mutations compared to NYDTVTGYSYYYYGMDV (SEQ ID NO: 137). In some embodiments, HCDR2 of the antibody or antigen binding fragment thereof comprises 4 or fewer mutations compared to HVYYTGSTNYNPRFKD (SEQ ID NO: 136) and HCDR3 of the antibody or antigen binding fragment thereof comprises 3 or fewer mutations compared to NYDTVTGYSYYYYGMDV (SEQ ID NO: 137). In such embodiments, the HCDR1 comprises the amino acid sequence of RGGDYWX1 (SEQ ID NO: 530), wherein X1 is A, S, or G, preferably A or S, and the LV of the antibody or antigen binding fragment thereof is of or derived from a VK1/O2/JK4, a VK2/A19/JK1, or a VK3/A27/JK1 germline. Preferably the LV is of or derived from a VK1/O2/JK4 or a VK2/A19/JK1 germline, more preferably a VK1/O2/JK4 germline. Exemplary sequences of LV sequences of or derived from the germlines are described above and in Tables 8, 11, 27 and 31.

In some embodiments, the antibody or antigen binding fragment thereof comprises a) a HV comprising a HCDR1, HCDR2 and HCDR3, wherein HCDR2 of the antibody or antigen binding fragment thereof comprises 5 or fewer mutations compared to HVYYTGSTKYNPNFKG (SEQ ID NO: 8). In some embodiments, HCDR2 of the antibody or antigen binding fragment thereof comprises 4 or fewer mutations compared to HVYYTGSTKYNPNFKG (SEQ ID NO: 8). In some embodiments, HCDR2 of the antibody or antigen binding fragment thereof comprises 5 or fewer mutations compared to HVYYTGSTKYNPNFKG (SEQ ID NO: 8) and HCDR3 of the antibody or antigen binding fragment thereof comprises 3 or fewer mutations compared to DYDILTGYSYYYYGMDV (SEQ ID NO: 9). In some embodiments, HCDR2 of the antibody or antigen binding fragment thereof comprises 4 or fewer mutations compared to HVYYTGSTKYNPNFKG (SEQ ID NO: 8) and HCDR3 of the antibody or antigen binding fragment thereof comprises 3 or fewer mutations compared to DYDILTGYSYYYYGMDV (SEQ ID NO: 9). In such embodiments, the HCDR1 comprises the amino acid sequence of RGGDYWX1 (SEQ ID NO: 530), wherein X1 is A, S, or G, preferably A or S, and the LV of the antibody or antigen binding fragment thereof is of or derived from a VK1/O2/JK4 or a VK2/A19/JK1 germline, preferably a VK2/A19/JK1 germline. Exemplary sequences of LV sequences of or derived from the germlines are described above and in Tables 8, 11, 20, 27 and 31.

In some embodiments, the anti-CB1 antibody or antigen binding fragment thereof comprises a heavy chain variable region comprising a sequence that is at least 90% identical, or at least 95% identical (e.g., at least 96%, at least 97%, or at least 98% identical) to the sequence of SEQ ID NO: 228. The LV of the antibody or antigen binding fragment thereof is of or derived from a VK1/O2/JK4, a VK2/A19/JK1, or a VK3/A27/JK1 germline. Preferably the LV is of or derived from a VK1/O2/JK4 or a VK2/A19/JK1 germline, more preferably a VK1/O2/JK4 germline. In some embodiments, the anti-CB1 antibody or antigen binding fragment thereof comprises a heavy chain variable region comprising a sequence that is at least 90% identical, or at least 95% identical (e.g., at least 96%, at least 97%, or at least 98% identical) to the sequence of SEQ ID NO: 100. The LV of the antibody or antigen binding fragment thereof is of or derived from a VK1/O2/JK4 or a VK2/A19/JK1 germline, preferably a VK2/A19/JK1 germline. Exemplary sequences of LV sequences of or derived from the germlines are described above and in Tables 8, 11, 20, 27 and 31.

The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, WI). The computer algorithm GAP is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:
Algorithm: Needleman et al. 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

As described above, the anti-CB1 antibody or antigen binding fragment thereof disclosed herein can contain mutations in HC_FR3 region compared to 10D10. For antibody or antigen biding fragment thereof that comprises HCDR1 of SEQ ID NO: 1, HCDR2 comprising SEQ ID NO: 538 and HCDR3 of SEQ ID NO: 539, in addition to mutations at HV position 83 and/or 85, the amino acid at HV position 79 may be R. In some embodiments, the amino acid at HV position 83 of the antibody is N and the amino acid at HV position 85 of the antibody is Y. In some embodiments, the amino acid at position 79 of the antibody or antigen binding fragment thereof is R, the amino acid at HV position 83 of the antibody or antigen binding fragment thereof is N and the amino acid at HV position 85 of the antibody or antigen binding fragment thereof is Y. In some embodiments of the antibodies described above, the amino acid at HV position of the antibody or antigen binding fragment thereof 83 is N, the amino acid at HV position 85 of the antibody or antigen binding fragment thereof is Y, and the amino acid at LV position 94 of the antibody or antigen binding fragment thereof is S. In some embodiments, the amino acid at position 79 of the antibody or antigen binding fragment thereof is R, the amino acid at HV position 83 of the antibody or antigen binding fragment thereof is N and the amino acid at HV position 85 of the antibody or antigen binding fragment thereof is Y, and the amino acid at LV position 94 of the antibody or antigen binding fragment thereof is S.

In certain preferred embodiments, the anti-CB1 antibody or antigen binding fragment thereof comprises a HCDR1, HCDR2, HCDR3, LCDR1, LCDR2 or LCDR3 of any one of LIBC528116-1, LIBC680574-1, LIBC673952-1, LIBC523797-1, LIBC527814-1, LIBC527997-1, LIBC527919-1, LIBC523661-1 and LIBC524049-1. For example, the anti-CB1 antibody or antigen binding fragment thereof comprises a set of heavy and light chain CDRs of any one of the following:
(a) HCDR1, HCDR2 and HCDR3 comprise the amino acid sequence of SEQ ID NOS: 135-137, respectively and LCDR1, LCDR2 and LCDR3 comprise the amino acid sequence of SEQ ID NOS: 138-140, respectively;
(b) HCDR1, HCDR2 and HCDR3 comprise the amino acid sequence of SEQ ID NOS: 426-428, respectively and LCDR1, LCDR2 and LCDR3 comprise the amino acid sequence of SEQ ID NOS: 423-425, respectively;
(c) HCDR1, HCDR2 and HCDR3 comprise the amino acid sequence of SEQ ID NOS: 266-268, respectively and LCDR1, LCDR2 and LCDR3 comprise the amino acid sequence of SEQ ID NOS: 263-235, respectively
(d) HCDR1, HCDR2 and HCDR3 comprise the amino acid sequence of SEQ ID NOS: 129-131, respectively and LCDR1, LCDR2 and LCDR3 comprise the amino acid sequence of SEQ ID NOS: 132-134, respectively;
(e) HCDR1, HCDR2 and HCDR3 comprise the amino acid sequence of SEQ ID NOS: 159-161, respectively and LCDR1, LCDR2 and LCDR3 comprise the amino acid sequence of SEQ ID NOS: 162-164, respectively;
(f) HCDR1, HCDR2 and HCDR3 comprise the amino acid sequence of SEQ ID NOS: 207-209, respectively and LCDR1, LCDR2 and LCDR3 comprise the amino acid sequence of SEQ ID NOS: 210-212, respectively;
(g) HCDR1, HCDR2 and HCDR3 comprise the amino acid sequence of SEQ ID NOS: 1-3, respectively and LCDR1, LCDR2 and LCDR3 comprise the amino acid sequence of SEQ ID NOS: 4-6, respectively; and
(h) HCDR1, HCDR2 and HCDR3 comprise the amino acid sequence of SEQ ID NOS: 55-57, respectively and LCDR1, LCDR2 and LCDR3 comprise the amino acid sequence of SEQ ID NOS: 58-60, respectively.

In some preferred embodiments, the antibody or antigen binding fragment thereof comprises a HV comprising an amino acid sequence selected from SEQ ID NO: 228, SEQ ID DO: 460, SEQ ID NO: 368, SEQ ID NO: 100, SEQ ID NO: 236, SEQ ID NO: 226, SEQ ID NO: 252, SEQ ID NO: 98, and SEQ ID NO: 116. In some preferred embodiments, the antibody or antigen binding fragment thereof comprises a LV comprising an amino acid sequence selected from SEQ ID NO: 227, SEQ ID NO: 459, SEQ ID NO: 367, SEQ ID NO: 99, SEQ ID NO: 235, SEQ ID NO: 225, SEQ ID NO: 251, SEQ ID NO: 97, and SEQ ID NO: 115. In some preferred embodiments, the antibody or antigen binding fragment thereof comprises a HV and LV selected from any of the following: i) a HV comprising the amino acid sequence of SEQ ID NO: 228, a LV comprising the amino acid sequence of SEQ ID NO: 227; ii) a HV comprising the amino acid sequence of SEQ ID NO: 460, a LV comprising the amino acid sequence of SEQ ID NO: 459; iii) a HV comprising the amino acid sequence of SEQ ID NO: 368, a LV comprising the amino acid sequence of SEQ ID NO: 367; iv) a HV comprising the amino acid sequence of SEQ ID NO: 100, a LV comprising the amino acid sequence of SEQ ID NO: 99; v) a HV comprising the amino acid sequence of SEQ ID NO: 236, a LV comprising the amino acid sequence of SEQ ID NO: 235; vi) a HV comprising the amino acid sequence of SEQ ID NO: 226, a LV comprising the amino acid sequence of SEQ ID NO: 225; vii) a HV comprising the amino acid sequence of SEQ ID NO: 252, a LV comprising the amino acid sequence of SEQ ID NO: 251; viii) a HV comprising the amino acid sequence of SEQ ID NO: 98, a LV comprising the amino acid sequence of SEQ ID NO: 97; and ix) a HV comprising the amino acid sequence of SEQ ID NO: 116, a LV comprising the amino acid sequence of SEQ ID NO: 115.

In various embodiments, the antibodies or antigen binding fragments thereof disclosed herein have a higher binding affinity for CB1 (e.g., huCB1) compared to 10D10 LC N35Y (i.e., 10D10 that contains N to Y substitution at LC amino acid position 35). In some embodiments, the antibodies or antigen binding fragments thereof have a binding affinity for CB1 that is at least three times higher compared to 10D10 LC N35Y. In some embodiments, the antibodies or antigen binding fragments thereof have a binding affinity for CB1 that is at least five times higher (e.g., at least eight time higher, at least ten times higher, at least twelve times higher, at least fifteen times higher, or at least twenty times higher) compared to 10D10 LC N35Y.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method. Binding to CB1 (e.g., huCB1) can be measured using these techniques and an antigen source (e.g., CB1-ND, CB1-SMALP, or CB1 expressing cells (e.g., 293T cells).

Antibody or antigen binding fragment that specifically bind an antigen may have an equilibrium dissociation constant (K_{D})≤1×10⁻⁶ M. The antibody or antigen binding fragment specifically binds antigen with “high affinity” when the K_{D} is ≤3×10⁻⁸ M. In some embodiments, the anti-CB1 antibodies or antigen binding fragments bind to target antigen (e.g., huCB1) with a K_{D} of ≤100 nM (e.g., less than about 90 nM, 70 nM, 50 nM, 30 nM, 20 nM, 10 nM, or a range defined by any two of the foregoing values). In some embodiments, the anti-CB1 antibodies or binding fragments bind to CB1 (e.g., huCB1 such as huCB1-ND) with a dissociation constant (K_{D}) of less than about 70 nM-10 nM (e.g., about 60 nM-20 nM, or about 40 nM-10 nM), as determined using Octet® systems and huCB1-ND. In some embodiments, the antibodies or antigen binding fragments bind to CB1 (e.g., hu-CB1) with a KD of ≤1×10⁻⁸ M (e.g., ≤1×10⁻⁹ M, ≤5×10⁻¹ M, or ≤1×10⁻¹⁰ M) as determined using a KinExA assay and huCB1-SMAP or CB1 expressing cells.

In various embodiments, the anti-CB1 antibodies and antigen binding fragments disclosed herein are antagonist and/or inverse agonist anti-huCB1 antibodies and antigen binding fragments, for example, they inhibit signaling of huCB1. Potencies of the antibodies and antigen binding fragments are in single digit nanomolar range or subnanomolar range, for example, they have an IC50 of less than about 10 nM, less than about 5 nM, less than about 3 nM or less than about 1 nM as measured in a cell-based cAMP assay (e.g., a cell-based cAMP assay in the presence of CP 55,940). In some embodiments, the anti-CB1 antibodies and antigen binding fragments disclosed herein have a potency comparable to that of rimonabant. In some embodiments, the anti-CB1 antibodies and antigen binding fragments disclosed herein have a potency greater than that of rimonabant (e.g., at least two-fold, three-fold, five-fold, eight-fold, or ten-fold greater than that of rimonabant). In various embodiments, potency can be measured using a cell-based cMAP assay.

The 10D10 antibody binds to the extracellular loop 2 region of huCB1 (i.e., amino acid residues 256 to 273 of SEQ ID NO: 559) (WO2014210205). The anti-CB1 antibodies and antigen binding fragments disclosed herein bind to the same region on huCB1 as they are affinity matured variants of the 10D10 antibody. Thus, in some embodiments, the anti-CB1 antibodies and antigen binding fragments disclosed herein bind to the extracellular loop 2 region of huCB1 and have an IC50 of less than about 10 nM, less than about 5 nM, less than about 3 nM, or less than about 1 nM, as measured in a cell-based cAMP assay (e.g., a cell-based cAMP assay in the presence of CP 55,940).

The anti-CB1 antibodies of the invention can comprise any immunoglobulin constant region. The term “constant region,” used interchangeably herein with “constant domain” refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, SgG3, QgG4, IgA1, IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes. Examples of human immunoglobulin light chain constant region sequences are shown in Table 1:

TABLE 1
Exemplary Human Immunoglobulin Light Chain Constant Regions

| | SEQ | |
|---|---|---|
| | ID | |
| Designation | NO: | CL Domain Amino Acid Sequence |
| Human | 513 | GQPKANPTVTLFPPSSEELQANKATLVCLISDFYPGAVTVAWKADGSP |
| lambda v1 | | VKAGVETTKPSKQSNNKYAASSYLSLTPEQWKSHRSYSCQVTHEGST |
| | | VEKTVAPTECS |
| Human | 514 | GQPKAAPSVTLFPPSSEELQANKATLVCLISDFYPGAVTVAWKADSSP |
| lambda v2 | | VKAGVETTTPSKQSNNKYAASSYLSLTPEQWKSHRSYSCQVTHEGSTV |
| | | EKTVAPTECS |
| Human | 515 | QPKAAPSVTLFPPSSEELQANKATLVCLISDFYPGAVTVAWKADSSPV |
| lambda v3 | | KAGVETTTPSKQSNNKYAASSYLSLTPEQWKSHRSYSCQVTHEGSTVE |
| | | KTVAPTECS |
| Human | 516 | GQPKAAPSVTLFPPSSEELQANKATLVCLISDFYPGAVTVAWKADSSP |
| lambda v4 | | VKAGVETTTPSKQSNNKYAASSYLSLTPEQWKSHKSYSCQVTHEGST |
| | | VEKTVAPTECS |
| Human | 517 | GQPKAAPSVTLFPPSSEELQANKATLVCLVSDFYPGAVTVAWKADGSP |
| lambda v5 | | VKVGVETTKPSKQSNNKYAASSYLSLTPEQWKSHRSYSCRVTHEGST |
| | | VEKTVAPAECS |
| Human kappa | 518 | TVAAPSVFIFPPSDEQLKSGTASVVCLLNNFYPREAKVQWKVDNALQS |
| v1 | | GNSQESVTEQDSKDSTYSLSSTLTLSKADYEKHKVYACEVTHQGLSSP |
| | | VTKSFNRGEC |
| Human kappa | 519 | RTVAAPSVFIFPPSDEQLKSGTASVVCLLNNFYPREAKVQWKVDNALQ |
| v2 | | SGNSQESVTEQDSKDSTYSLSSTLTLSKADYEKHKVYACEVTHQGLSS |
| | | PVTKSFNRGEC |

The heavy chain constant region of the anti-CB1 antibodies of the invention can be, for example, an alpha-, delta-, epsilon-, gamma-, or mu-type heavy chain constant region, e.g., a human alpha-, delta-, epsilon-, gamma-, or mu-type heavy chain constant region. In some embodiments, the anti-CB1 antibodies comprise a heavy chain constant region from an IgG1, IgG2, IgG3, or IgG4 immunoglobulin, such as a human IgG1, IgG2, IgG3, or IgG4 immunoglobulin. In one embodiment, the anti-CB1 antibody comprises a heavy chain constant region from a human IgG1 immunoglobulin. In such embodiments, the human IgG1 immunoglobulin constant region may comprise one or more mutations to prevent glycosylation and/or half life of the antibody as described in more detail herein. In another embodiment, the anti-CB1 antibody comprises a heavy chain constant region from a human IgG2 immunoglobulin. In yet another embodiment, the anti-CB1 antibody comprises a heavy chain constant region from a human IgG4 immunoglobulin. Examples of human IgG1, IgG2, and IgG4 heavy chain constant region sequences are shown below in Table 2.

TABLE 2
Exemplary Human Immunoglobulin Heavy Chain Constant Regions

| | SEQ | |
|---|---|---|
| | ID | |
| Ig isotype | NO: | Heavy Chain Constant Region Amino Acid Sequence |
| Human IgG1z | 520 | ASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human IgG1za | 521 | ASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| Human IgG1f | 522 | ASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human IgG1fa | 523 | ASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| Human IgG1z | 524 | ASTKGPSVFPLAPSSKSTSGGTAALGCLVKDYFPEPVTVSWNSGALTSGVHTFPAV |
| aglycosylated v1 | | LQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVH |
| | | NAKTKPREEQYGSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human IgG1z | 525 | ASTKGPSVFPLAPSSKSTSGGTAALGCLVKDYFPEPVTVSWNSGALTSGVHTFPAV |
| aglycosylated v2 | | LQSSGLYSLSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWYVDGVEVH |
| | | NAKTKPCEEQYGSTYRCVSVLTVLHQDWLNGKEYKCKVSNKALPAPIEKTISKAKG |
| | | QPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| Human IgG1z | 526 | ASTKGPSVFPLAPSSKSTSGGTAALGCLVKDYFPEPVTVSWNSGALTSGVHTFPAV |
| aglycosylated | | LQSSGLYSLSSVVTVPSSSLGTQTYICNVNHKPSNTKVDKKVEPKSCDKTHTCPPCP |
| v2_half life | | APELLGGPSVFLFPPKPKDTLYITREPEVTCVVVDVSHEDPEVKFNWYVDGVEVHN |
| extended | | AKTKPCEEQYGSTYRCVSVLTVLHQDWLNGKEYKCKVSNKALPAPIEKTISKAKGQ |
| | | PREPQVYTLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| huIgG1z_half life | 527 | ASTKGPSVFPLAPSSKSTSGGTAALGCLVKDYFPEPVTVSWNSGALTSGVHTFPAV |
| extended | | LQSSGLYSLSSVVTVPSSSLGTQTYICNVNHKPSNTKVDKKVEPKSCDKTHTCPPCP |
| | | APELLGGPSVFLFPPKPKDTLYITREPEVTCVVVDVSHEDPEVKFNWYVDGVEVHN |
| | | AKTKPREEQYNSTYRVVSVLTVLHQDWLNGKEYKCKVSNKALPAPIEKTISKAKGQ |
| | | PREPQVYTLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| Human IgG2 | 528 | ASTKGPSVFPLAPCSRSTSESTAALGCLVKDYFPEPVTVSWNSGALTSGVHTFPAVL |
| | | QSSGLYSLSSVVTVPSSNFGTQTYTCNVDHKPSNTKVDKTVERKCCVECPPCPAPP |
| | | VAGPSVFLFPPKPKDTLMISRTPEVTCVVVDVSHEDPEVQFNWYVDGVEVHNAKT |
| | | KPREEQFNSTFRVVSVLTVVHQDWLNGKEYKCKVSNKGLPAPIEKTISKTKGQPRE |
| | | PQVYTLPPSREEMTKNQVSLTCLVKGFYPSDIAVEWESNGQPENNYKTTPPMLDS |
| | | DGSFFLYSKLTVDKSRWQQGNVFSCSVMHEALHNHYTQKSLSLSPGK |
| Human IgG4 | 529 | ASTKGPSVFPLAPCSRSTSESTAALGCLVKDYFPEPVTVSWNSGALTSGVHTFPAVL |
| | | QSSGLYSLSSVVTVPSSSLGTKTYTCNVDHKPSNTKVDKRVESKYGPPCPSCPAPEFL |
| | | GGPSVFLFPPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK |

Each of the light chain variable regions and each of the heavy chain variable regions disclosed herein (Tables 8, 11, 20, 27 and 31) may be attached to the above light chain constant regions (Table 1) and heavy chain constant regions (Table 2) to form complete antibody light and heavy chains, respectively. In some embodiments, each of the light chain variable regions and each of the heavy chain variable regions disclosed in Tables 8, 11, 20, 27 and 31 are attached to SEQ ID NO: 518 or 519 and SEQ ID NO: 525, 526, or 527 to form complete antibody light and heavy chains, respectively. In some embodiments, each of the light chain variable regions and each of the heavy chain variable regions disclosed in Tables 8, 11, 20, 27 and 31 are attached to SEQ ID NO: 518 and SEQ ID NO: 525, 526, or 527 to form complete antibody light and heavy chains, respectively. In some embodiments, each of the light chain variable regions and each of the heavy chain variable regions disclosed in Tables 8, 11, 20, 27 and 31 are attached SEQ ID NO: 518 and SEQ ID NO: 525 to form complete antibody light and heavy chains, respectively. In some embodiments, the monoclonal antibody comprises a heavy chain having the amino acid sequence of SEQ ID NO: 562 and a light chain having the amino acid sequence of SEQ ID NO: 563. In some embodiments, the monoclonal antibody comprises a heavy chain having the amino acid sequence of SEQ ID NO: 562 with the amino acid residue K at the C-terminal end deleted and a light chain having the amino acid sequence of SEQ ID NO: 563. Further, each of the so generated heavy and light chain sequences may be combined to form a complete antibody structure. It should be understood that the heavy chain and light chain variable regions provided herein can also be attached to other constant domains having different sequences than the exemplary sequences listed above.

The anti-CB1 antibodies or antigen binding fragments of the invention can be monoclonal antibodies, polyclonal antibodies, recombinant antibodies, human antibodies, humanized antibodies, chimeric antibodies, or multispecific antibodies or antigen-binding fragments thereof. In certain embodiments, the anti-CB1 antibody is a monoclonal antibody. In such embodiments, the anti-CB1 antibody may be a chimeric antibody, a humanized antibody, or a fully human antibody having a human immunoglobulin constant domain. In these and other embodiments, the anti-CB1 antibody is a human IgG1 (e.g., IgG1z), IgG2, IgG3, or IgG4 antibody. Thus, the anti-CB1 antibody may, in some embodiments, have a human IgG1, IgG2, IgG3, or IgG4 constant domain. In one embodiment, the anti-CB1 antibody is a monoclonal human IgG1 antibody, preferably, a monoclonal human IgG1z antibody. In another embodiment, the anti-CB1 antibody is a monoclonal human IgG2 antibody. In yet another embodiment, the anti-CB1 antibody is a monoclonal human IgG4 antibody.

The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from an animal after completion of the immunization schedule. The spleen cells can be immortalized using any technique known in the art, e.g., by fusing them with myeloma cells to produce hybridomas. See, for example, Antibodies; Harlow and Lane, Cold Spring Harbor Laboratory Press, 1st Edition, e.g. from 1988, or 2nd Edition, e.g. from 2014. Myeloma cells for use in hybridoma-producing fusion procedures preferably are non-antibody-producing, have high fusion efficiency, and enzyme deficiencies that render them incapable of growing in certain selective media, which support the growth of only the desired fused cells (hybridomas). Examples of suitable cell lines for use in fusions with mouse cells include, but are not limited to, Sp-20, P3-X63/Ag8, P3-X63-Ag8.653, NS1/1.Ag 4 1, Sp210-Agl4, FO, NSO/U, MPC-11, MPC11-X45-GTG 1.7 and 5194/5XXO Bul. Examples of suitable cell lines used for fusions with rat cells include, but are not limited to, R210.RCY3, Y3-Ag 1.2.3, IR983F and 4B210. Other cell lines useful for cell fusions are U-266, GM1500-GRG2, LICR-LON-HMy2 and UC729-6. Additional exemplary methods for isolating monoclonal antibodies include screening plasma B cells from an animal after completion of the immunization schedule. (See e.g., Pedrioli A. and Oxenius A., Trends in Immunology, 2021, 42(12):1148-1158). Properties of monoclonal antibodies isolated by these methods can be optimized (e.g., to improve binding) using in vitro affinity maturation techniques such as yeast display-based affinity maturation known in the art. (see e.g., Cherf, G. M. and Cochran, J. R., Yeast Surface Display: Methods, Protocols, and Applications. 2015, 155-175).

In some embodiments, the anti-CB1 antibodies or antigen-binding fragments of the invention are chimeric or humanized antibodies or antigen-binding fragments thereof based upon the CDR and variable region sequences of the antibodies described herein. A chimeric antibody is an antibody composed of protein segments from different antibodies that are covalently joined to produce functional immunoglobulin light or heavy chains or binding fragments thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567 and Morrison et al., 1985, Proc. Natl. Acad. Sci. USA 81:6851-6855, both of which are hereby incorporated by reference in their entireties.

Generally, the goal of making a chimeric antibody is to create a chimera in which the number of amino acids from the intended species is maximized. One example is the “CDR-grafted” antibody, in which the antibody comprises one or more CDRs from a particular species or belonging to a particular antibody class or subclass, while the remainder of the antibody chain(s) is/are identical with or homologous to a corresponding sequence in antibodies derived from another species or belonging to another antibody class or subclass. CDR grafting is described, for example, in U.S. Pat. Nos. 6,180,370, 5,693,762, 5,693,761, 5,585,089, and 5,530,101. For use in humans, the variable region or selected CDRs from a rodent or rabbit antibody often are grafted into a human antibody, replacing the naturally-occurring variable regions or CDRs of the human antibody.

One useful type of chimeric antibody is a “humanized” antibody. Generally, a humanized antibody is produced from a monoclonal antibody raised initially in a non-human animal, such as a rodent or rabbit. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent or rabbit variable region for the corresponding regions of a human antibody (see, e.g., U.S. Pat. Nos. 5,585,089, and 5,693,762; Jones et al., 1986, Nature 321:522-525; Riechmann et al., 1988, Nature 332:323-27; and Verhoeyen et al., 1988, Science 239:1534-1536).

In one aspect, the CDRs of the light and heavy chain variable regions of the antibodies provided herein (see, Tables 7, 10, 19, 26 and 30) are grafted to framework regions (FRs) from antibodies from the same, or a different, phylogenetic species. For example, the CDRs of the heavy and light chain variable regions listed in Tables 7, 10, 19, 26 and 30 can be grafted to consensus human FRs. To create consensus human FRs, FRs from several human heavy chain or light chain amino acid sequences may be aligned to identify a consensus amino acid sequence. Alternatively, the grafted variable regions from the one heavy or light chain may be used with a constant region that is different from the constant region of that particular heavy or light chain as disclosed herein.

In certain embodiments, the anti-CB1 antibodies or antigen-binding fragments of the invention are fully human antibodies or antigen-binding fragments thereof. A “fully human antibody” is an antibody that comprises variable and constant regions derived from or indicative of human germline immunoglobulin sequences. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

The transgenic mice described above, referred to as “HuMab” mice, contain a human immunoglobulin gene minilocus that encodes unrearranged human heavy (mu and gamma) and kappa light chain immunoglobulin sequences, together with targeted mutations that inactivate the endogenous mu and kappa chain loci (Lonberg et al., 1994, Nature 368:856-859). Accordingly, the mice exhibit reduced expression of mouse IgM and kappa proteins and in response to immunization, the introduced human heavy and light chain transgenes undergo class switching and somatic mutation to generate high affinity human IgG kappa monoclonal antibodies (Lonberg and Huszar, 1995, Intern. Rev. Immunol. 13: 65-93; Harding and Lonberg, 1995, Ann. N.Y Acad. Sci. 764:536-546). The preparation of HuMab mice is described in detail in Taylor et al., 1992, Nucleic Acids Research 20:6287-6295; Chen et al., 1993, International Immunology 5:647-656; Tuaillon et al., 1994, J. Immunol. 152:2912-2920; Lonberg et al., 1994, Nature 368:856-859; Lonberg, 1994, Hand book of Exp. Pharmacology 113:49-101; Taylor et al., 1994, International Immunology 6:579-591; Lonberg and Huszar, 1995, Intern. Rev. Immunol. 13:65-93; Harding and Lonberg, 1995, Ann. N.Y Acad. Sci. 764:536-546; Fishwild et al., 1996, Nature Biotechnology 14:845-851; the foregoing references are hereby incorporated by reference in their entireties. One particular transgenic mouse line suitable for generation of fully human anti-CB1 antibodies is the XenoMouse® transgenic mouse line described in U.S. Pat. Nos. 6,114,598; 6,162,963; 6,833,268; 7,049,426; 7,064,244; Green et al., 1994, Nature Genetics 7:13-21; Mendez et al., 1997, Nature Genetics 15:146-156; Green and Jakobovitis, 1998, J. Ex. Med, 188:483-495; Green, 1999, Journal of Immunological Methods 231:11-23; Kellerman and Green, 2002, Current Opinion in Biotechnology 13, 593-597, all of which are hereby incorporated by reference in their entireties. Human-derived antibodies can also be generated using phage display techniques. Phage display is described in e.g., Dower et al., WO 91/17271, McCafferty et al., WO 92/01047, and Caton and Koprowski, 1990, Proc. Natl. Acad. Sci. USA, 87:6450-6454, each of which is incorporated herein by reference in its entirety.

In certain embodiments, the anti-CB1 antibodies and antigen-binding fragments of the invention may comprise one or more mutations or modifications to a constant region. For example, the heavy chain constant regions or the Fc regions of the anti-CB1 antibodies may comprise one or more amino acid substitutions that affect the glycosylation, effector function, and/or Fcγ receptor binding of the antibody. The term “Fe region” refers to the C-terminal region of an immunoglobulin heavy chain, which may be generated by papain digestion of an intact antibody. The Fc region of an immunoglobulin generally comprises two constant domains, a CH2 domain and a CH3 domain, and optionally comprises a CH4 domain. In certain embodiments, the Fc region is an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as Clq binding, complement-dependent cytotoxicity (CDC), Fc receptor binding, antibody-dependent cell-mediated cytotoxicity (ADCC), and phagocytosis. In other embodiments, the Fc region may be modified to reduce or eliminate effector function and/or improve half-life as described in further detail below.

In some embodiments, the anti-CB1 antibodies of the invention comprise one or more amino acid substitutions in the Fc region to reduce effector function. An amino acid substitution in an amino acid sequence is typically designated herein with a one-letter abbreviation for the amino acid residue in a particular position, followed by the numerical amino acid position relative to an original sequence of interest, which is then followed by the one-letter abbreviation for the amino acid residue substituted in. For example, “C220S” symbolizes a substitution of a cysteine residue by a serine residue at amino acid position 220, relative to the original sequence of interest. Exemplary amino acid substitutions (according to the EU numbering scheme) that can reduce effector function include, but are not limited to, C220S, C226S, C229S, E233P, L234A, L234V, V234A, L234F, L235A, L235E, G237A, P238S, S267E, H268Q, N297A, N297G, V309L, E318A, L328F, A330S, A331S, P331S, or combinations of any of the foregoing.

Glycosylation can contribute to the effector function of antibodies, particularly IgG1 antibodies. Thus, in some embodiments, the anti-CB1 antibodies of the invention may comprise one or more amino acid substitutions that affect the level or type of glycosylation of the antibodies. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In some embodiments, glycosylation of the anti-CB1 antibodies described herein is decreased or eliminated by removing one or more glycosylation sites, e.g., from the Fc region of the antibody. In some embodiments, the anti-CB1 antibody is an aglycosylated human monoclonal antibody, e.g., an aglycosylated human IgG1 monoclonal antibody. Amino acid substitutions that eliminate or alter N-linked glycosylation sites can reduce or eliminate N-linked glycosylation of the antibody. In certain embodiments, the anti-CB1 antibodies described herein comprise a heavy chain mutation at position N297 (according to the EU numbering scheme), such as N297Q, N297A, or N297G. In some embodiments, the anti-CB1 antibodies of the invention comprise an Fc region from a human IgG1 antibody with a mutation at position N297. In one particular embodiment, the anti-CB1 antibodies of the invention comprise an Fc region from a human IgG1 antibody with a N297G mutation. For instance, in some embodiments, the anti-CB1 antibodies of the invention comprise a heavy chain constant region comprising the sequence of SEQ ID NO: 524.

To improve the stability of molecules comprising a N297 mutation, the Fc region of the anti-CB1 antibodies may be further engineered. For instance, in some embodiments, one or more amino acids in the Fc region are substituted with cysteine to promote disulfide bond formation in the dimeric state. Residues corresponding to V259, A287, R292, V302, L306, V323, or 1332 (according to the EU numbering scheme) of an IgG1 Fc region may thus be substituted with cysteine. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and I332C. In certain embodiments, the anti-CB1 antibodies described herein comprise an Fc region from a human IgG1 antibody with mutations R292C and V302C. In such embodiments, the Fc region may also comprise a N297 mutation, such as a N297G mutation. In some embodiments, the anti-CB1 antibodies of the invention comprise a heavy chain constant region comprising the sequence of SEQ ID NO: 525.

Modifications of the anti-CB1 antibodies of the invention to increase serum half-life (e.g., in vivo half-life) also may desirable. One approach for achieving increased serum half-life is by amino acid mutations in certain positions in the constant domain such as those described in WO2002060919. Such mutations increase the affinity of the antibody for the FcRn and its serum half-life. In some embodiments, the anti-CB1 antibodies described herein comprise an Fc region from a human IgG1 antibody with one or more mutations at amino acid positions 251-256 (according to the EU numbering scheme). In some embodiments, the Fc region comprises one or more mutations at positions 252, 254 and 256. In some embodiments, the Fc region comprises M252Y, S254T, and T256E (according to the EU numbering scheme). In some embodiments, the anti-CB1 antibodies of the invention comprise a heavy chain constant region comprising the sequence of SEQ ID NO: 527.

In some embodiments, Fc region of the anti-CB1 antibodies described herein may comprises modifications that alter glycosylation, stability and serum half-life of the antibodies. For example, in some embodiments, the anti-CB1 antibodies described herein comprise an Fc region from a human IgG1 antibody with mutations R292C and V302C, a N297 mutation, such as a N297G mutation (according to the EU numbering scheme), and may also comprise one or more mutations at positions 252, 254 and 256, such as M252Y, S254T, and T256E (according to the EU numbering scheme). In some embodiments, the anti-CB1 antibodies of the invention comprise a heavy chain constant region comprising the sequence of SEQ ID NO: 526.

It is envisioned that the anti-CB1 antibodies and antigen binding fragments described herein do not cross-react with (i.e., essentially do not recognize or bind to) human cannabinoid receptor type 2 (CB2). Human CB2 is a G protein-coupled receptor from the cannabinoid receptor family and is encoded by the CNR2 gene. Human CB2 contains 360 amino acids (UniProtKB—P34972, SEQ ID NO: 560) and shares about 44% sequence similarity with huCB1. The anti-CB1 antibodies and antigen binding fragments described herein also do not cross-react with mouse CB1 protein (UniProtKB—P47746, SEQ ID NO: 561).

The present invention includes one or more polynucleotides or nucleic acids encoding the anti-CB1 antibodies or antigen-binding fragments described herein. In addition, the present invention encompasses vectors comprising the nucleic acids, host cells or cell lines comprising the nucleic acids, and methods of making the anti-CB1 antibodies and antigen-binding fragments of the invention. The nucleic acids comprise, for example, polynucleotides that encode all or part of an antibody or antigen-binding fragment, for example, one or both chains of an antibody of the invention, or a fragment, derivative, or variant thereof, PCR primers or sequencing primers for identifying, analyzing, mutating or amplifying a polynucleotide encoding a polypeptide, anti-sense oligonucleotides for inhibiting expression of a polynucleotide, and complementary sequences of the foregoing. The nucleic acids can be any length as appropriate for the desired use or function, and can comprise one or more additional sequences, for example, regulatory sequences, and/or be part of a larger nucleic acid, for example, a vector. Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention can be derived from human sources as well as non-human species.

Relevant amino acid sequences from an immunoglobulin or region thereof (e.g., variable region, Fc region, etc.) or polypeptide of interest may be determined by direct protein sequencing, and suitable encoding nucleotide sequences can be designed according to a universal codon table. Alternatively, genomic or cDNA encoding monoclonal antibodies or binding fragments thereof of the invention can be isolated and sequenced from cells producing such antibodies using conventional procedures (e.g., by using oligonucleotide probes that are capable of binding specifically to genes encoding the heavy and light chains of the monoclonal antibodies).

The present invention also includes vectors comprising one or more nucleic acids encoding one or more components of the antibodies or antigen-binding fragments of the invention (e.g., variable regions, light chains, and heavy chains). The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the variable region polypeptide sequences listed in Tables 8, 11, 20, 27, and 31. In certain embodiments, a signal peptide having the amino acid sequence of MDMRVPAQLLGLLLLWLRGARC (SEQ ID NO: 541) is fused to the amino terminus of any of the variable region polypeptide sequences in Tables 8, 11, 20, 27, and 31. In other embodiments, a signal peptide having the amino acid sequence of MAWALLLLTLLTQGTGSWA (SEQ ID NO: 542) is fused to the amino terminus of any of the variable region polypeptide sequences in Tables 8, 11, 20, 27, and 31. In still other embodiments, a signal peptide having the amino acid sequence of MTCSPLLLTLLIHCTGSWA (SEQ ID NO: 543) is fused to the amino terminus of any of the variable region polypeptide sequences in Tables 8, 11, 20, 27, and 31. Other suitable signal peptide sequences that can be fused to the amino terminus of the variable region polypeptide sequences described herein include: MEAPAQLLFLLLLWLPDTTG (SEQ ID NO: 544), MEWTWRVLFLVAAATGAHS (SEQ ID NO: 545), METPAQLLFLLLLWLPDTTG (SEQ ID NO: 546), METPAQLLFLLLLWLPDTTG (SEQ ID NO: 547), MKHLWFFLLLVAAPRWVLS (SEQ ID NO: 548), MEWSWVFLFFLSVTTGVHS (SEQ ID NO: 549), MDIRAPTQLLGLLLLWLPGAKC (SEQ ID NO: 550), MDIRAPTQLLGLLLLWLPGARC (SEQ ID NO: 551), MDTRAPTQLLGLLLLWLPGATF (SEQ ID NO: 552), MDTRAPTQLLGLLLLWLPGARC (SEQ ID NO: 553), METGLRWLLLVAVLKGVQC (SEQ ID NO: 554), METGLRWLLLVAVLKGVQCQE (SEQ ID NO: 555), MDMRAPTQLLGLLLLWLPGARC (SEQ ID NO: 556), MKILILGIFLFLCSTPAWA (SEQ ID NO: 557), and MRTLAILAAILLVALQAQA (SEQ ID NO: 558). Other signal or secretory peptides are known to those of skill in the art and may be fused to any of the variable region polypeptide chains listed in Tables 8, 11, 20, 27, and 31, for example, to facilitate or optimize expression in particular host cells.

Typically, expression vectors used in the host cells to produce the anti-CB1 antibodies and antigen-binding fragments of the invention will contain sequences for plasmid maintenance and for cloning and expression of exogenous nucleotide sequences encoding the components of the antibodies and antigen-binding fragments. Such sequences, collectively referred to as “flanking sequences,” in certain embodiments will typically include one or more of the following nucleotide sequences: a promoter (e.g., a promoter suitable for a host cell), one or more enhancer sequences (e.g., a viral enhancer sequence), an origin of replication (e.g., plasmid origin for bacteria or viral origins for mammalian system), a transcriptional termination sequence, a complete intron sequence containing a donor and acceptor splice site, a sequence encoding a signal sequence for polypeptide secretion, a ribosome binding site (e.g., a Shine-Dalgarno sequence (prokaryotes) or a Kozak sequence (eukaryotes)), a polyadenylation sequence, a polylinker region for inserting the nucleic acid encoding the polypeptide to be expressed, and a selectable marker element (e.g., sequences that encode proteins conferring resistance to antibiotics or complement auxotrophic deficiencies of host cells). Franking sequences and vectors are well known in the art. For example, expression vectors are commercially available. In addition, desired modifications may be made to commercially available expression vectors or constructed using sequences known in the art. Nucleic acids encoding the different components of the anti-CB1 antibodies or binding fragments (e.g., HV and/or LV) may be inserted into proper sites of the same vector or different vectors for expression in a host cell.

A “host cell” refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated nucleic acid of the invention, preferably operably linked to at least one expression control sequence (e.g. promoter or enhancer), is a “recombinant host cell.” Methods for transforming or transfecting vectors into a host cell is well known in the art.

Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antibodies and antigen-binding fragments can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antibodies and antigen-binding fragments from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. CHO cells are preferred host cells in some embodiments for expressing the anti-CB1 antibodies and antigen-binding fragments of the invention.

Host cells are transformed or transfected with the above-described nucleic acids or vectors for production of anti-CB1 antibodies or antigen-binding fragments and are cultured in conventional nutrient media modified as appropriate for inducing promoters, selecting transformants, or amplifying the genes encoding the desired sequences. Thus, the present invention also provides a method for producing an anti-CB1 antibody or antigen-binding fragment described herein comprising culturing a host cell comprising one or more expression vectors described herein in a culture medium under conditions permitting expression of the antibody or antigen-binding fragment encoded by the one or more expression vectors; and recovering the antibody or antigen-binding fragment from the culture medium or host cell.

Upon culturing the host cells, the antibody or antigen-binding fragment can be produced intracellularly, in the periplasmic space, or directly secreted into the medium. If the antibody or antigen-binding fragment is produced intracellularly, as a first step, the particulate debris, either host cells or lysed fragments, is removed, for example, by centrifugation or ultrafiltration. The antibody or antigen-binding fragment can be purified using methods known in the art, for example, hydroxyapatite chromatography, cation or anion exchange chromatography, or preferably affinity chromatography, using the antigen(s) of interest or protein A or protein G as an affinity ligand. Protein A can be used to purify proteins that include polypeptides that are based on human γ1, γ2, or γ4 heavy chains (Lindmark et al., J. Immunol. Meth. 62: 1-13, 1983). Protein G is recommended for all mouse isotypes and for human 73 (Guss et al., EMBO J. 5: 1567-1575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a CH3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular antibody or antigen-binding fragment to be recovered.

### Compositions

Also disclosed herein are compositions comprising the anti-CB1 antibodies or antigen binding fragments. Suitable compositions include those that comprise the anti-CB1 antibodies or antigen binding fragments described and exemplified herein and one or more pharmaceutically acceptable excipients. “Pharmaceutically-acceptable” refers to molecules, and compounds that are non-toxic to human recipients at the dosages and concentrations employed and/or do not produce allergic or adverse reactions when administered to humans. Pharmaceutical compositions of the invention include, but are not limited to, liquid, frozen, and lyophilized compositions.

In some embodiments, the pharmaceutical composition may contain materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, buffers (such as acetate buffer, Tris-HCl, citrate buffer, phosphate buffer or buffers containing other organic acids); bulking agents or fillers such as monosaccharides; disaccharides (e.g., sucrose); and other carbohydrates; proteins (such as serum albumin, gelatin or immunoglobulins); amino acids (e.g., glycine, glutamine, asparagine, histidine, arginine, or lysine), coloring and diluting agents; emulsifying agents; hydrophilic polymers (such as polyvinylpyrrolidone); chelating agents (such as ethylenediamine tetraacetic acid (EDTA)); complexing agents (such as caffeine, polyvinylpyrrolidone, beta-cyclodextrin or hydroxypropyl-beta-cyclodextrin); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol, sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company.

In some embodiments, the composition disclosed herein is a liquid composition such as an aqueous solution. In some embodiments, the composition comprises a buffering agent, a tonicity or bulking agent, a surfactant, and has a pH in the range of from about 4.5 to about 7.5. Optionally, the composition further comprises a salt and/or a preservative.

In some embodiments, the pH of the composition is in the range of from about 4.5 to about 7.0, for example, from about 4.5 to about 6.5, or from about 4.8 to about 5.5, or alternatively about 5.0. Examples of buffers that are suitable for a pH within this range include acetate (e.g., sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the composition.

A tonicity agent, which may also stabilize the antibody or antigen-binding fragment, may be included in the composition. Exemplary tonicity agents include polyols, such as sucrose, mannitol, or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the formulation to reduce aggregation of the formulated antibody or antigen-binding fragment and/or minimize the formation of particulates in the formulation and/or reduce adsorption. Exemplary surfactants include nonionic surfactants such as polysorbates (e.g. polysorbate 20 or polysorbate 80) or poloxamers (e.g. poloxamer 188). Exemplary concentrations of surfactant may range from about 0.001% to about 0.5%, or from about 0.005% to about 0.2%, or alternatively from about 0.004% to about 0.01% w/v.

In one embodiment, the composition contains the above-identified agents (i.e. antibody or antigen-binding fragment, buffer, polyol and surfactant) and is essentially free of one or more preservatives, such as benzyl alcohol, phenol, m-cresol, chlorobutanol and benzethonium chloride. In another embodiment, a preservative may be included in the formulation, e.g., at concentrations ranging from about 0.1% to about 2%, or alternatively from about 0.5% to about 1%. In one embodiment, the composition may further comprise a salt such as sodium chloride or one or more other pharmaceutically acceptable excipients. One or more other pharmaceutically acceptable carriers, excipients or stabilizers such as those described in REMINGTON'S PHARMACEUTICAL SCIENCES, 18th Edition, (A. R. Genrmo, ed.), 1990, Mack Publishing Company, may be included in the formulation provided that they do not adversely affect the desired characteristics of the formulation.

The formulations to be used for in vivo administration must be sterile. The compositions of the invention may be sterilized by conventional, well-known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

In some embodiments, the composition comprising the anti-CB1 antibodies or antigen binding fragment and one or more pharmaceutically acceptable excipients is a solid composition such as a lyophilized cake. The process of lyophilization is often employed to stabilize polypeptides for long-term storage, particularly when the polypeptide is relatively unstable in liquid compositions. A lyophilization cycle is usually composed of three steps: freezing, primary drying, and secondary drying (see Williams and Polli, Journal of Parenteral Science and Technology, Volume 38, Number 2, pages 48-59, 1984). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use. The standard reconstitution practice for lyophilized material is to add back a volume of pure water (typically equivalent to the volume removed during lyophilization), although dilute solutions of antibacterial agents are sometimes used in the production of pharmaceuticals for parenteral administration (see Chen, Drug Development and Industrial Pharmacy, Volume 18: 1311-1354, 1992).

Excipients have been noted in some cases to act as stabilizers for freeze-dried products (see Carpenter et al., Volume 74: 225-239, 1991). For example, known excipients include polyols (including mannitol, sorbitol and glycerol); sugars (including glucose and sucrose); and amino acids (including alanine, glycine and glutamic acid). In addition, polyols and sugars are also often used to protect polypeptides from freezing- and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

The formulations of the invention may be designed to be short-acting, fast-releasing, long-acting, or sustained-releasing as described herein. Thus, the pharmaceutical formulations may also be formulated for controlled release or for slow release. Suitable examples of sustained-release preparations include semipermeable matrices of solid hydrophobic polymers containing the antibody or antigen-binding fragment, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions. Excipients and methods for making sustained-release compositions are described in e.g., REMINGTON'S PHARMACEUTICAL SCIENCES, 18th Edition, (A. R. Genrmo, ed.), 1990.

Compositions comprising the anti-CB1 antibodies or antigen binding fragments and one or more pharmaceutically acceptable excipients described above can be administered by any suitable means, including parenteral administration. Parenteral administration includes intravenous, intraarterial, intraperitoneal, intramuscular, intradermal or subcutaneous administration. Preferably, the dosing is given by injections, most preferably subcutaneous, intramuscular, or intravenous injections. Other administration methods are contemplated, including topical, particularly transdermal, transmucosal, rectal, oral or local administration e.g., through a catheter placed close to the desired site.

### Use

Disclosed herein also includes the use of the anti-CB1 antibodies or antigen binding fragments or compositions comprising the same for inhibiting, reducing or neutralizing the amount, activity or signaling of the CB1 receptor (e.g., huCB1). The antibodies and antigen binding fragments of the invention are useful for the treatment, prevention and/or amelioration of any disease or disorder associated with or mediated by CB1 expression or activity, or treatable by blocking the interaction between CB1 and a CB1 ligand (e.g., a cannabinoid) or otherwise inhibiting CB1 activity and/or signaling, and/or promoting receptor internalization and/or decreasing cell surface receptor number. In various embodiments, the CB1 is a peripheral CB1.

Exemplary disorders, diseases and conditions that may be treated by the anti-CB1 antibodies or antigen-binding fragments described herein include obesity and comorbidities such as syndromic obesities including Prader-Willi syndrome, Alstrom syndrome, Bardet-Biedel syndrome (BBS), Albright Hereditary Osteodystrophy (AHO), and SIM1 deletion syndrome; diabetes and related complications (e.g., abnormal plasma glucose, insulin, and/or resistin levels); dyslipidemia (e.g., abnormal HDL, plasma cholesterol and/or triglyceride levels); liver diseases such as, for example, non-alcoholic steatohepatitis (NASH), non-alcoholic fatty liver disease (NAFLD), and primary biliary cirrhosis; fibrosis, for example, kidney fibrosis; chronic kidney disease (CKD) such as CKD driven by metabolic derangements; IgA nephropathy; renal disease; metabolic diseases, osteoporosis, atherosclerosis, inflammatory disease, cardiovascular disease, cancer, pain, systemic sclerosis, multiple sclerosis spasticity, glaucoma, and nicotine addiction.

Obesity refers to abnormal or excessive fat accumulation that may impair health of an individual and may be measured or classified by body mass index (BMI). BMI is defined as a person's weight (e.g., kilograms) divided by the square of his height in meters (e.g., kg/m²).

Thus, in one aspect, disclosed herein is a method for treating a disease or disorder in a subject responsive of antagonizing or inverse agonizing the CB1 receptor (e.g., huCB1), or in a subject in need of antagonizing or inverse agonizing the CB1 receptor, the method comprises administering the antibodies or antigen binding fragments described above. In some embodiments, the antibodies or antigen binding fragments are potent antagonist and/or inverse agonist of the CB1 receptor, for example they have a potency (IC50) of less than about 10 nM as measured in a cell-based cAMP assay (e.g., less than about 8 nM, less than about 5 nM, less than about 3 nM, or less than about 1 nM). In some embodiments, the antibodies or antigen binding fragments have an IC50 less than about 10 nM (e.g., less than about 5 nM, less than about 3 nM, or less than about 1 nM) as measured in a cell-based cAMP assay (e.g., a cell-based cAMP assay in the presence of CP 55,940). In some embodiments, the antibodies or antigen binding fragments comprise a heavy and/or a light chain CDR as described above. CDR sequences of various exemplary antibodies are listed in Tables 7, 10, 19, 26, and 30. In some embodiments, the antibodies or antigen binding fragments comprise a heavy and/or light chain variable region as described above. In other embodiments, the antibodies comprise a heavy and/or light chain as described above. Heavy and light chain variable region sequences of exemplary antibodies are listed in Tables 8, 11, 20, 27 and 31. Exemplary heavy and light chain constant region sequences are listed in Tables 1 and 2. In various embodiments, the antibodies or antigen binding fragments is administered in an effective amount for treating the disorder or disease (e.g., cause a potential benefit or therapeutic effect). In various embodiments, the CB1 receptor is a peripheral CB1 receptor.

The terms “subject in need” or those “in need of treatment” includes those suffering from the disorder or disease, as well as those in which the disorder or disease has not yet clinically manifested. The “subject” includes human and other mammalian subjects that receive either prophylactic or therapeutic treatment. In preferred embodiments, the subject is human.

In some embodiments, the disorder or disease is obesity, diabetes, dyslipidemia, metabolic diseases, fibrosis, liver disease such as non-alcoholic steatohepatitis (NASH) or NAFLD, primary biliary cirrhosis, renal disease, kidney fibrosis, chronic kidney disease (e.g. chronic kidney disease driven by metabolic derangements), IgA nephropathy, osteoporosis, atherosclerosis, cardiovascular disease, cancer, or inflammatory disease. In some embodiments, the disorder or disease is obesity. In some embodiments, the subject being treated has chronic kidney disease such as chronic kidney disease driven by metabolic derangements or kidney injury. In some embodiments, the subject being treated has obesity with a BMI of 27 kg/m² or higher. In some embodiments, the subject being treated has obesity with a BMI of 30 kg/m² or higher. In other embodiments, the disorder or disease is a liver disease (e.g., NASH).

In some embodiments, the administration or treatment results in one or more of the following in the subject: reduced body weight, reduced appetite, improved metabolic parameters, reduced blood glucose levels (e.g., reduced fasting blood glucose levels), reduced insulin levels, reduced HbA1c levels, reduced blood lipid levels (e.g., reduced HDL, cholesterol and/or triglyceride levels), reduced kidney injury, fibrosis, inflammation, and improved kidney function.

### Kits

In some embodiments, disclosed herein is a kit comprising compositions comprising the anti-CB1 antibodies or antigen binding fragments and one or more pharmaceutically acceptable excipients described above. For example, such a kit includes a composition comprising anti-CB1 antibodies or antigen binding fragments described herein such as an aqueous or lyophilized composition described above, packaged in a container such as a sealed bottle, vessel, single-use or multi-use vial, prefilled syringe, or prefilled injection device. A suitable vehicle or carrier may be provided, such as water for injection or physiological saline solution, for lyophilized compositions. Other materials common in formulations for parenteral administration may also be included. In one aspect, the composition is packaged in a unit dosage form. The kit may further include a device suitable for administering the formulation according to a specific route of administration. Preferably, the kit further contains a label that describes use of the antibodies or antigen binding fragments or compositions comprising the same.

All of the references cited herein, including patents, patent applications, literature publications, and the like, are hereby incorporated in their entireties by reference.

The following examples are given merely to illustrate the present invention and not in any way to limit its scope.

### EXAMPLES

### Example 1. Deep Mutational Scan and Design of Affinity Maturation Libraries

Materials and Methods: CB1-ND preparation. For nanodisc preparation, CB1 construct described in Tian H. et al., Cell, 167(3) (2016) 750-762 was expressed with a histidine purification tag in Sf9 cells and purified using affinity and size exclusion chromatography in HEPES buffer containing 0.05% DDM; 0.01% CHS and 10 uM ligand-1981202 #5. POPC lipid was solubilized with the help of 100 mM sodium cholate and mixed with MSP1D1 scaffold protein and CB1 at the molar ratio of 480:8:1. Nanodisc formation was achieved by incubation with SM2 BioBeads for 1 h at 4° C.

Binding to CB1-ND and empty-ND. The propagation and induction of yeast cells displaying antibodies for binding experiments have been described by Luna V. et al. (Luna V. et al., European Polymer Journal 109 (2018) 483-488). At least 2×10⁵ yeast cells displaying IgGs were removed and washed with 1× phosphate-buffered saline (no Mg2+, no Ca2+) supplemented with 5 mg/mL bovine serum albumin (PBS-B). His-tagged huCB1-ND or empty-ND was added to the desired concentration and incubated with the yeast cells at 25° C. for 1 hour. The cells were washed with PBS-B and then incubated with an allophycocyanin (APC)-conjugated anti-His antibody to detect binding and a phycoerythrin (PE)-conjugated anti-huFc antibody to detect IgG surface display. The cells were washed with PBS-B before flow cytometry analysis using a FACS-Canto (BD Biosciences). APC and PE median fluorescence intensities (MFI) were recorded.

FACS library sorting. At least 10⁷ cells were incubated with antigen as described above. Following fluorescent antibody staining, the cells were sorted using a FACS Aria (BD Biosciences) using the appropriate settings for allophycocyanin and phycoerythrin detection.

The anti-CB1 antibody 10D10 was previously generated by immunizing Xenomouse with HEK293 cells expressing human CB1. (See WO2014210205). Although affinity maturation is a common strategy employed to improve the potency of antagonist antibodies, previous efforts of affinity maturing 10D10 failed to make antibodies with sufficient potency (e.g., potency in the single digit nM range in a cell-based cAMP assay) to produce desired therapeutic effects (e.g., reduce body weight) without CNS-related side effects. A lack of high-quality, soluble preparations of complex membrane proteins such as CB1 was considered to be a major contributor of previous failures. The development of CB1 nanodisc (ND) and CB1 styrene maleic acid lipoparticles (SMALP) greatly improved the quality of soluble preparations of CB1. (Luna V. et al., European Polymer Journal 109 (2018) 483-488). Both CB1-ND and -SMALP are purified reagents that are stable after multiple freeze/thaw cycles and compatible with long-term storage. Furthermore, these reagents can be added to binding experiments at user-defined concentrations of huCB1. The availability of these reagents enabled additional affinity maturation efforts.

Deep mutational scanning of the antibody was carried out to inform the design of affinity maturation libraries. Deep mutational scanning entails generating comprehensive libraries of point mutations, performing binding selections under different conditions, and evaluating changes in mutation frequencies after selections with high-throughput sequencing. (See e.g., Araya, C. L. and Fowler, D. M., Trends Biotechnol. 2011 September; 29(9): 435-442). Identification of the subset of mutations associated with improved and specific huCB1 binding enables focused combinatorial library designs for affinity maturation without the benefit of a high-resolution antibody-antigen co-crystal structure.

Fifty-three and thirty-seven two positions within the 10D10 heavy chain (HC) and light chain (LC), respectively, were chosen for single-site saturation mutagenesis. Point mutation IgG-yeast libraries were generated wherein the comprehensive set of 10D10 point mutations in the heavy and light chain CDRs and the heavy chain (HC) and light chain FR3 de-loops were split into four libraries (2 each for heavy and light chain). Each library was sorted using FACS for surface expression, and separately against huCB1-ND at two different stringencies and against empty-ND to assess non-specific binding. The lower-stringency huCB1-ND binding gate collected all cells exhibiting at least 10D10-like binding (10-20% of the population), whereas the higher-stringency gate collected only the top binders (1.5-6% of the population). After FACS, the plasmids from the sorted pools were harvested using a Zymoprep II kit (Zymo Research) and amplicons were generated by minimal PCR amplification of the recovered plasmids.

Illumina MiSeq was used to rank 1710 point mutations by comprehensively assessing changes in the frequencies of mutations within the sort gates vs their starting frequencies (freq) within an expression-sorted reference pool. Binding enrichment (EF) factors for each mutation were calculated based on NGS analysis and using the expression-sorted pools as reference samples (EF=freq_{bind}/freq_{reference (expression)}). The enrichment factors were then used to generate sequence-binding fitness maps for each mutation in the CDRs and FR3 de loop. Mutations that resulted in better, comparable, and worse binding to CB1 compared to the 10D10 antibody were referred to as beneficial, neutral, and deleterious mutations, respectively. Based on the fitness map, many changes within HCDR1 and HCDR3 were deleterious, indicating that these regions already seem well-optimized and may have less need and/or potential for optimization. A number of mutations in HCDR2 and HC_FR3 de loop appeared to drive improved binding. The data from the library containing LCDR1 and LCDR2 mutations indicated a lack of beneficial mutations in these regions. Overall, binding fitness maps suggested that the HCDRs were more promising areas to diversify for affinity maturation to improve binding.

Based on experience, an ideal position to diversify for affinity maturation typically contains changes to both deleterious and beneficial mutations. To identify the most promising mutations at a single position to be included in combinatorial library designs, each neutral or beneficial mutation for huCB1-ND binding was also assessed for binding to empty-ND. The most promising mutations were enriched for huCB1-ND binding and depleted for empty ND binding (vs the 10D10 parent). Mutations exhibiting positive enrichment factors against both huCB1-ND and empty-ND were discarded from consideration for library designs.

Two NGS-guided HC yeast libraries were designed and constructed for affinity maturation. Each library was designed to explore separate CDR loop regions and to keep the combinatorial theoretical diversities manageable for coverage by a routine yeast library transformation. Tables 3-4 below summarize diversification strategies for the HC libraries. Combinations of the desired mutations were fully explored within discrete regions of 10D10 HC by introducing multiple degenerate codons during library gene synthesis to construct an initial set of two yeast-displayed IgG HC variant libraries paired with the parent LC of 10D10. Each degenerate codon at a specific position was chosen to maximize the representation of the desired mutations within the encoded theoretical diversity

TABLE 3
Design of 10D10 H1H3 library 1*

| 10D10 residue | Location | Diversification strategy |
|---|---|---|
| S30 | Extended HCDR1 | S, N |
| R32 | Extended HCDR1 | R, K |
| S42 | Extended HCDR1 | S, A, G, T |
| D109 | HCDR3 | D, E, N, K |
| I112 | HCDR3 | I, T, V, P, Q, A |
| L113 | HCDR3 | L, F, V |
| T114 | HCDR3 | T, S |
| Y116 | HCDR3 | Y, H, N |

*Theoretical combinatorial diversity: 6.9E3 (paired with 10D10 LC)

TABLE 4
Design of 10D10 H2/de loop library 2*

| 10D10 residue | location | Diversification strategy |
|---|---|---|
| Y57 | HCDR2 | Y, H |
| I58 | HCDR2 | I, V |
| Y60 | HCDR2 | Y, H, Q, stop |
| S65 | HCDR2 | S, E, T, A, K, stop |
| S67 | HCDR2 | S, Q |
| N69 | HCDR2 | N, A, K |
| S73 | HCDR2 | S, K, N, R |
| L74 | HCDR2 | L, F |
| K75 | HCDR2 | K, E |
| S76 | HCDR2 | S, G, N, D |
| D83 | de loop (HC_FR3) | D, K, E, N |
| D85 | de loop (HC_FR3) | S, Y |

*Theoretical combinatorial diversity: 3.0E5 (paired with 10D10 LC)

One NGS-guided yeast library was designed and constructed for LC optimization to improve manufacturability of 10D10. It was believed that the long HCDR3 and LCDR1 may contribute to the poor expression and aggregation propensities of earlier 10D10 mutants, and the design of LC libraries incorporated strategies to improve these properties (e.g., reducing hydrophobicity at key solvent-exposed LC residues in LCDR1). As the fitness maps of the LC mutations showed a lack of beneficial mutations, the LC library design included mutations exhibiting 10D10-like binding enrichment against CB1-ND and depletion against empty ND. Table 5 below summarizes diversification strategies for the LC optimization library. Combinations of the desired mutations were fully explored within discrete regions of 10D10 LC by introducing multiple degenerate codons during library gene synthesis to construct a yeast-displayed IgG LC variant library paired with the parent 10D10 HC. Each degenerate codon at a specific position was chosen to maximize the representation of the desired set of mutations within the encoded theoretical diversity. This library was used in the cognate affinity maturation described below (Example 2).

TABLE 5
Design of 10D10 LC optimization library**

| 10D10 residue | Location | Diversification strategy |
|---|---|---|
| H33 | LCDR1 | H, S, T |
| N35 | LCDR1 | N, S*, T*, Y# |
| Y38 | LCDR1 | Y, A*, E*, I*, N*, F |
| L41 | LCDR1 | L, V |
| L58 | LCDR2 | L, Q* |
| R94 | LC FR3 | R, S |
| M107 | LCDR3 | M, R |
| L110 | LCDR3 | L, F, I, R, V, C, G, S |
| Q111 | LCDR3 | Q, E, K, R, T, A, G, P |
| T135 | LCDR3 | T, I, L, Q, K, P |

*Amino acids with reduced hydrophobicity vs corresponding 10D10 residue
**Theoretical combinatorial diversity: 5.0E5 (paired with 10D10 HC)

The 10D10 LC belongs to the VK2 germline. It was known in the art that LCs of germlines such as VK1 or VK3 confer better biophysical properties and better manufacturability (see e.g., Ewert S. et al., J. Mol. Biol., (2003)325(3):531-53). In a second strategy for LC optimization, swapping out the 10D10 LC with LCs of a different germline (e.g., VK1 or VK3) predicted to have more favorable biophysical properties was explored. Both low-resolution X-ray and CryoEM structures of mAb/antigen complex showed that only the HC loops of 10D10 interact with the CB1 receptor. These results indicated that it may indeed be possible to swap out the 10D10 VK2 LC for another LC belonging to a germline (e.g., VK1 or VK3) predicted to have superior biophysical properties to improve manufacturability. To this end, the 10D10 HC was paired with a diverse human LC library, screened and sorted for binders that retained equivalent binding as 10D10. Next, those that possess LCs related to the 10D10 LC optimization design described above were removed from the positive clones. The process resulted in yeast clones that exhibit equivalent CB1 binding as 10D10 but contain LCs from VK1 and VK3 germlines, which demonstrated the feasibility of a LC swap strategy. The top 6 clones obtained from the LC swap are listed in Table 6 below. The six LCs were then expanded to a set of 22 LCs to remedy germline derivations within the FR regions of four of the clones.

TABLE 6
Six new LCs that can replace 10D10 LC without impacting CB1 binding

| | SEQ | | SEQ | | SEQ | | LC |
|---|---|---|---|---|---|---|---|
| NAME | NO. | LCDR1 | NO. | LCDR2 | NO. | LCDR3 | Germline |
| LIBC488095-1 | 495 | RASQSVSSSYLA | 496 | GASSRAT | 497 | QQGYSNPRT | VK3\|A27/JK3 |
| LIBC488333-1 | 498 | RASQSIISYLN | 499 | AASSLRS | 500 | QQYSNYPLT | VK1\|O2/JK4 |
| LIBC488370-1 | 501 | RASQSVSNYLA | 502 | DASNRAT | 503 | QQYGSLPRT | VK3\|L6/JK2 |
| LIBC488554-1 | 504 | RASQSVSSYLG | 505 | GASSRAT | 506 | QQYGSSPRT | VK3\|A27/JK1 |
| LIBC488452-1 | 507 | RASQSVSSYLA | 508 | DASNRAT | 509 | QQYGSLPRT | VK3\|A11/JK2 |
| LIBC488537-1 | 510 | RASQSISNYLN | 511 | AASSLHS | 512 | QQYQSYPLT | VK1\|O2/JK4 |
| 10D10 | 94 | RSSQSLLHSNGYN | 95 | LGSNRAS | 96 | MQALQTPRT | VK2\|A19/JK1 |
| | | YLD | | | | | |

### Example 2. Cognate Affinity Maturation

Materials and methods: GFP-fused huCB1-SMALP was prepared as described in Luna V. et al., European Polymer Journal 109 (2018) 483-488.

The preparation of yeast cells for binding experiments and measurement of binding to GFP-fused huCB1-SMALP by flow cytometry were carried out as described in Luna V. et al., European Polymer Journal 109 (2018) 483-488, except an AlexaFluor647-conjugated anti-huFab antibody was used to detect IgG surface display. The median fluorescence intensities (MFI) for GFP (binding) and AlexaFluor647 (display) were recorded.

For the nanodisc binding experiments, a binding/display ratio for each tested yeast clone was calculated by dividing the APC MFI by the PE MFI. The binding/display ratio of each clone was further normalized to that of 10D10 LC N35Y control to calculate a “relative binding, -fold change” ratio enabling comparison of yeast clones. Specific binding to huCB1-ND over empty-ND was assessed by dividing the huCB1-ND binding/display ratio by the empty-ND binding/display ratio. For SMALP binding experiments, each clone's GFP MFI after background subtraction was normalized to that of 10D10 LC N35Y control to compare binding of yeast clones.

The two HC libraries and the one LC optimization library designed and constructed in Example 1 were used in cognate affinity maturation of 10D10. The two HC libraries were separately enriched by FACS for improved binders relative to 10D10 following established methods (see e.g., Boder, E.T. and K. D. Wittrup, Yeast surface display for directed evolution of protein expression, affinity, and stability. Methods Enzymol, 2000. 328: p. 430-44; and Chao, G., et al., Isolating and engineering human antibodies using yeast surface display. Nat Protoc, 2006. 1(2): p. 755-68). The LC optimization library was enriched for cells exhibiting 10D10-like binding signals. The surviving HC and LC mutations after enrichment were recovered from the yeast pools by zymoprep and PCR, and pools of HCs and LCs were mixed by design during yeast transformation to construct a new set of shuffled HC/LC libraries (mutH1H3/mutLC, mutH2/mutLC and mutH1H2H3/mutLC). Higher-stringency FACS enrichments utilizing successively lower concentrations of huCB1-ND were implemented on the chain-shuffled libraries. Clones from the enriched shuffled libraries were screened in two rounds for top clone selection. In the first round, approximately 600 clones from the three libraries were screened for clones that bind to huCB1-ND better than the 10D10 LC N35Y control (10D10 with N35Y in the LC).

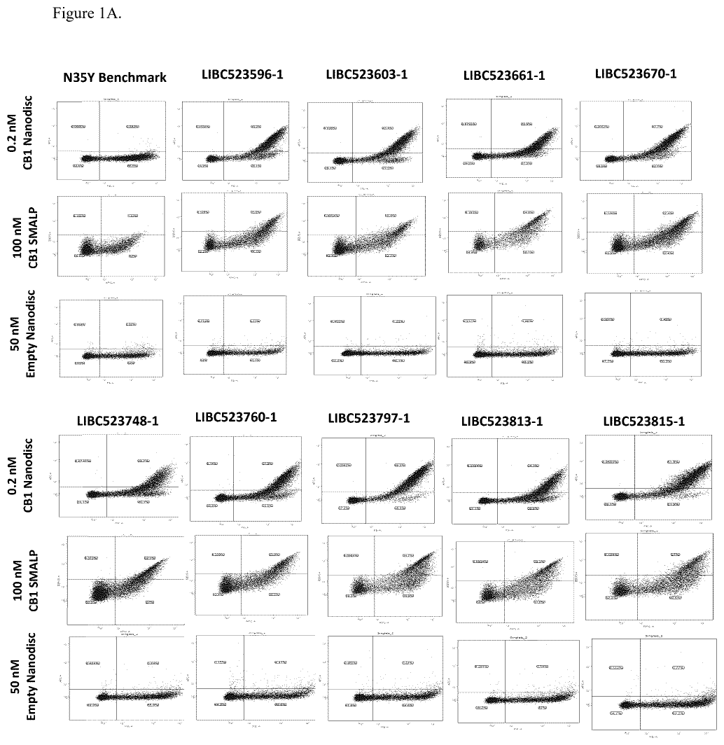

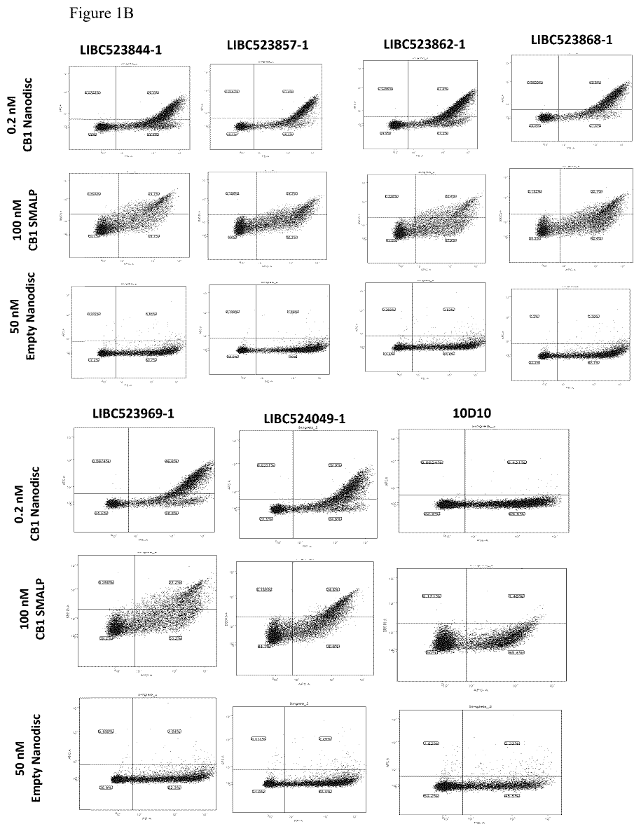

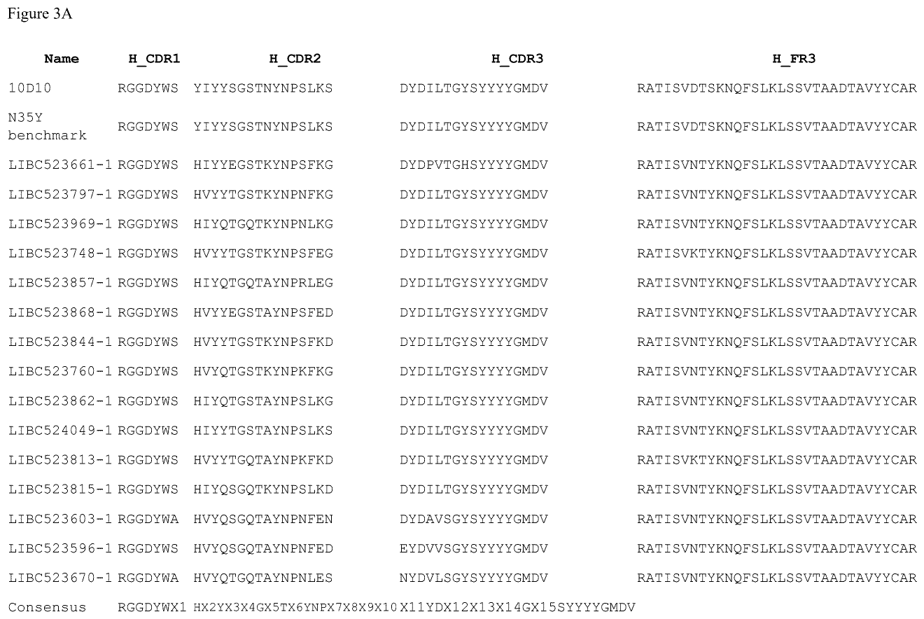

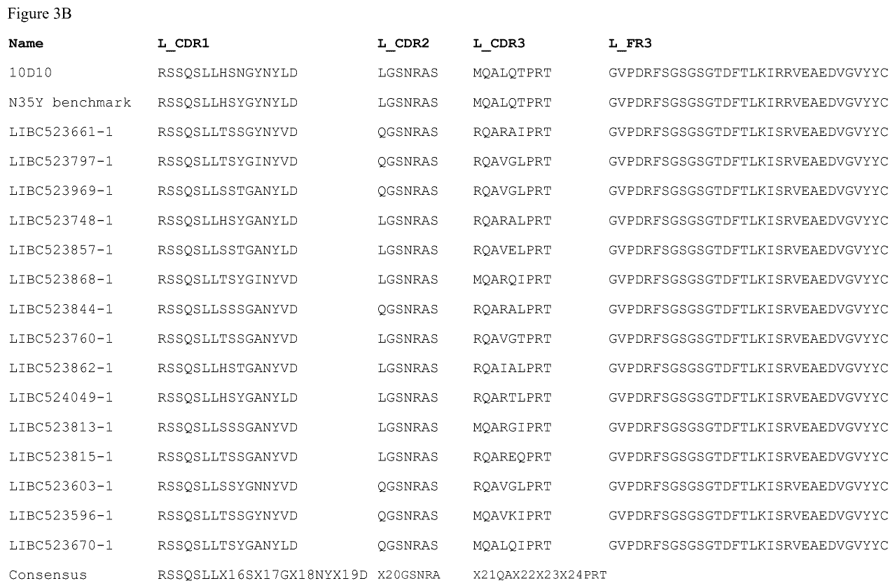

In the second round of screening, the 200 best binding clones from the first round were re-tested for higher-stringency binding to 200 μM huCB1-ND, 100 nM GFP-tagged huCB1-SMALP and against empty-ND for nonspecific binding. After ranking positive clones by CB1-specific binding, those with N-linked glycosylation and unpaired Cys Tier 1 hotspots were removed and those with predicted Tier 1.5 deamidation and isomerization sites other than DT were de-prioritized. In addition, positive clones with R94S fixes for the LC FR3 consensus violation were prioritized. Positive clones possessing the HC D83K FR3 mutation believed to be associated with enhanced anti-CB1 function were also prioritized if they satisfied the minimum threshold for binding improvement. Furthermore, clones with a reduction in hydrophobicity at key residues in LCDR1 and LCDR2 were prioritized. Tables 7 and 8 below summarize the sequence information of the top 15 cognate affinity matured 10D10 variants. FIGS. 1A and 1B show the flow cytometry binding profiles of the 15 variants; binding data of the variants are shown in in Table 9 below. All top clones exhibited significantly improved binding to huCB1-ND and huCB1-SMALP while maintaining minimal binding to empty-ND (Table 9). In addition, Y57H within HCDR2, and D83N and S85Y within the HC FR3 de loop were observed in all top clones (FIG. 3A). None of the top 15 binders contain predicted hotspots that pose the highest risk for chemical modification during therapeutic antibody manufacturing and storage.

TABLE 7
CDR sequence of top 15 affinity-matured cognate 10D10 variants

| | Seq | | Seq | | Seq | |
|---|---|---|---|---|---|---|
| Name | No. | CDR1 | No. | CDR2 | No. | CDR3 |
| LIBC523661- | 1 | RGGDYWS | 2 | HIYYEGSTKYNPSFKG | 3 | DYDPVTGHSYYYYGMDV |
| 1_HC huCB1 HV | | | | | | |
| LIBC523661- | 4 | RSSQSLLTSSGYNYVD | 5 | QGSNRAS | 6 | RQARAIPRT |
| 1_LC huCB1 LV | | | | | | |
| LIBC523797- | 7 | RGGDYWS | 8 | HVYYTGSTKYNPNFKG | 9 | DYDILTGYSYYYYGMDV |
| 1_HC huCB1 HV | | | | | | |
| LIBC523797- | 10 | RSSQSLLTSYGINYVD | 11 | QGSNRAS | 12 | RQAVGLPRT |
| 1_LC huCB1 LV | | | | | | |
| LIBC523969- | 13 | RGGDYWS | 14 | HIYQTGQTKYNPNLKG | 15 | DYDILTGYSYYYYGMDV |
| 1_HC huCB1 HV | | | | | | |
| LIBC523969- | 16 | RSSQSLLSSTGANYLD | 17 | QGSNRAS | 18 | RQAVGLPRT |
| 1_LC huCB1 LV | | | | | | |
| LIBC523748- | 19 | RGGDYWS | 20 | HVYYTGSTKYNPSFEG | 21 | DYDILTGYSYYYYGMDV |
| 1_HC huCB1 HV | | | | | | |
| LIBC523748- | 22 | RSSQSLLHSYGANYLD | 23 | LGSNRAS | 24 | RQARALPRT |
| 1_LC huCB1 LV | | | | | | |
| LIBC523857- | 25 | RGGDYWS | 26 | HIYQTGQTAYNPRLEG | 27 | DYDILTGYSYYYYGMDV |
| 1_HC huCB1 HV | | | | | | |
| LIBC523857- | 28 | RSSQSLLSSTGANYLD | 29 | LGSNRAS | 30 | RQAVELPRT |
| 1_LC huCB1 LV | | | | | | |
| LIBC523868- | 31 | RGGDYWS | 32 | HVYYEGSTAYNPSFED | 33 | DYDILTGYSYYYYGMDV |
| 1_HC huCB1 HV | | | | | | |
| LIBC523868- | 34 | RSSQSLLTSYGINYVD | 35 | LGSNRAS | 36 | MQARQIPRT |
| 1_LC huCB1 LV | | | | | | |
| LIBC523844- | 37 | RGGDYWS | 38 | HVYYTGSTKYNPSFKD | 39 | DYDILTGYSYYYYGMDV |
| 1_HC huCB1 HV | | | | | | |
| LIBC523844- | 40 | RSSQSLLSSSGANYVD | 41 | QGSNRAS | 42 | RQARALPRT |
| 1_LC huCB1 LV | | | | | | |
| LIBC523760- | 43 | RGGDYWS | 44 | HVYQTGSTKYNPKFKG | 45 | DYDILTGYSYYYYGMDV |
| 1_HC huCB1 HV | | | | | | |
| LIBC523760- | 46 | RSSQSLLTSSGANYVD | 47 | LGSNRAS | 48 | RQAVGTPRT |
| 1_LC huCB1 LV | | | | | | |
| LIBC523862- | 49 | RGGDYWS | 50 | HIYQTGSTAYNPSLKG | 51 | DYDILTGYSYYYYGMDV |
| 1_HC huCB1 HV | | | | | | |
| LIBC523862- | 52 | RSSQSLLHSTGANYVD | 53 | LGSNRAS | 54 | RQAIALPRT |
| 1_LC huCB1 LV | | | | | | |
| LIBC524049- | 55 | RGGDYWS | 56 | HIYYTGSTAYNPSLKS | 57 | DYDILTGYSYYYYGMDV |
| 1_HC huCB1 HV | | | | | | |
| LIBC524049- | 58 | RSSQSLLHSYGANYLD | 59 | LGSNRAS | 60 | RQARTLPRT |
| 1_LC huCB1 LV | | | | | | |
| LIBC523813-1 + | 61 | RGGDYWS | 62 | HVYYTGQTAYNPKFKD | 63 | DYDILTGYSYYYYGMDV |
| VK2 germline | | | | | | |
| fix_HC huCB1 | | | | | | |
| HV | | | | | | |
| LIBC523813-1 + | 64 | RSSQSLLSSSGANYVD | 65 | LGSNRAS | 66 | MQARGIPRT |
| VK2 germline | | | | | | |
| fix_LC huCB1 | | | | | | |
| LV | | | | | | |
| LIBC523815- | 67 | RGGDYWS | 68 | HIYQSGQTKYNPSLKD | 69 | DYDILTGYSYYYYGMDV |
| 1_HC huCB1 HV | | | | | | |
| LIBC523815- | 70 | RSSQSLLTSSGANYVD | 71 | LGSNRAS | 72 | RQAREQPRT |
| 1_LC huCB1 LV | | | | | | |
| LIBC523603- | 73 | RGGDYWA | 74 | HVYQSGQTAYNPNFEN | 75 | DYDAVSGYSYYYYGMDV |
| 1_HC huCB1 HV | | | | | | |
| LIBC523603- | 76 | RSSQSLLSSYGNNYVD | 77 | QGSNRAS | 78 | RQAVGLPRT |
| 1_LC huCB1 LV | | | | | | |
| LIBC523596-1 | 79 | RGGDYWS | 80 | HVYQSGQTAYNPNFED | 81 | EYDVVSGYSYYYYGMDV |
| HV | | | | | | |
| LIBC523596-1 | 82 | RSSQSLLTSSGYNYVD | 83 | QGSNRAS | 84 | MQAVKIPRT |
| LV | | | | | | |
| LIBC523670-1 + | 85 | RGGDYWA | 86 | HVYQTGQTAYNPNLES | 87 | NYDVLSGYSYYYYGMDV |
| VK2 germline | | | | | | |
| fix_HV | | | | | | |
| LIBC523670-1 + | 88 | RSSQSLLTSYGANYLD | 89 | QGSNRAS | 90 | MQALQIPRT |
| VK2 germline | | | | | | |
| fix_LV | | | | | | |
| 10D10 HV | 91 | RGGDYWS | 92 | YIYYSGSTNYNPSLKS | 93 | DYDILTGYSYYYYGMDV |
| 10D10 LV | 94 | RSSQSLLHSNGYNYLD | 95 | LGSNRAS | 96 | MQALQTPRT |

TABLE 8
Variable region sequence of top 15 cognate affinity-matured 10D10 variants

| | Seq | | Seq | |
|---|---|---|---|---|
| Name | No. | LC LV | No. | HC HV |
| LIBC523661- | 97 | DIVMTQSPLSLPVTPGEPASISCRSSQSLLTSSGYNYVDW | 98 | QVQLQESGPGLVKPSQTLSLTCTVSGGSIRRGGDYWSWIRQHPGK |
| 1 | | YLQKPGQSPQLLIYQGSNRASGVPDRFSGSGSGTDFTLKI | | GLEWIGHIYYEGSTKYNPSFKGRATISVNTYKNQFSLKLSSVTAADT |
| | | SRVEAEDVGVYYCRQARAIPRTFGQGTKVEIKR | | AVYYCARDYDPVTGHSYYYYGMDVWGQGTTVTVSS |
| LIBC523797- | 99 | DIVMTQSPLSLPVTPGEPASISCRSSQSLLTSYGINYVDW | 100 | QVQLQESGPGLVKPSQTLSLTCTVSGGSIRRGGDYWSWIRQHPGK |
| 1 | | YLQKPGQSPQLLIYQGSNRASGVPDRFSGSGSGTDFTLKI | | GLEWIGHVYYTGSTKYNPNFKGRATISVNTYKNQFSLKLSSVTAADT |
| | | SRVEAEDVGVYYCRQAVGLPRTFGQGTKVEIKR | | AVYYCARDYDILTGYSYYYYGMDVWGQGTTVTVSS |
| LIBC523969- | 101 | DIVMTQSPLSLPVTPGEPASISCRSSQSLLSSTGANYLDW | 102 | QVQLQESGPGLVKPSQTLSLTCTVSGGSIRRGGDYWSWIRQHPGK |
| 1 | | YLQKPGQSPQLLIYQGSNRASGVPDRFSGSGSGTDFTLKI | | GLEWIGHIYQTGQTKYNPNLKGRATISVNTYKNQFSLKLSSVTAADT |
| | | SRVEAEDVGVYYCRQAVGLPRTFGQGTKVEIKR | | AVYYCARDYDILTGYSYYYYGMDVWGQGTTVTVSS |
| LIBC523748- | 103 | DIVMTQSPLSLPVTPGEPASISCRSSQSLLHSYGANYLDW | 104 | QVQLQESGPGLVKPSQTLSLTCTVSGGSIRRGGDYWSWIRQHPGK |
| 1 | | YLQKPGQSPQLLIYLGSNRASGVPDRFSGSGSGTDFTLKI | | GLEWIGHVYYTGSTKYNPSFEGRATISVKTYKNQFSLKLSSVTAADT |
| | | SRVEAEDVGVYYCRQARALPRTFGQGTKVEIKR | | AVYYCARDYDILTGYSYYYYGMDVWGQGTTVTVSS |
| LIBC523857- | 105 | DIVMTQSPLSLPVTPGEPASISCRSSQSLLSSTGANYLDW | 106 | QVQLQESGPGLVKPSQTLSLTCTVSGGSIRRGGDYWSWIRQHPGK |
| 1 | | YLQKPGQSPQLLIYLGSNRASGVPDRFSGSGSGTDFTLKI | | GLEWIGHIYQTGQTAYNPRLEGRATISVNTYKNQFSLKLSSVTAADT |
| | | SRVEAEDVGVYYCRQAVELPRTFGQGTKVEIKR | | AVYYCARDYDILTGYSYYYYGMDVWGQGTTVTVSS |
| LIBC523868- | 107 | DIVMTQSPLSLPVTPGEPASISCRSSQSLLTSYGINYVDW | 108 | QVQLQESGPGLVKPSQTLSLTCTVSGGSIRRGGDYWSWIRQHPGK |
| 1 | | YLQKPGQSPQLLIYLGSNRASGVPDRFSGSGSGTDFTLKI | | GLEWIGHVYYEGSTAYNPSFEDRATISVNTYKNQFSLKLSSVTAADT |
| | | SRVEAEDVGVYYCMQARQIPRTFGQGTKVEIKR | | AVYYCARDYDILTGYSYYYYGMDVWGQGTTVTVSS |
| LIBC523844- | 109 | DIVMTQSPLSLPVTPGEPASISCRSSQSLLSSSGANYVDW | 110 | QVQLQESGPGLVKPSQTLSLTCTVSGGSIRRGGDYWSWIRQHPGK |
| 1 | | YLQKPGQSPQLLIYQGSNRASGVPDRFSGSGSGTDFTLKI | | GLEWIGHVYYTGSTKYNPSFKDRATISVNTYKNQFSLKLSSVTAADT |
| | | SRVEAEDVGVYYCRQARALPRTFGQGTKVEIKR | | AVYYCARDYDILTGYSYYYYGMDVWGQGTTVTVSS |
| LIBC523760- | 111 | DIVMTQSPLSLPVTPGEPASISCRSSQSLLTSSGANYVDW | 112 | QVQLQESGPGLVKPSQTLSLTCTVSGGSIRRGGDYWSWIRQHPGK |
| 1 | | YLQKPGQSPQLLIYLGSNRASGVPDRFSGSGSGTDFTLKI | | GLEWIGHVYQTGSTKYNPKFKGRATISVNTYKNQFSLKLSSVTAADT |
| | | SRVEAEDVGVYYCRQAVGTPRTFGQGTKVEIKR | | AVYYCARDYDILTGYSYYYYGMDVWGQGTTVTVSS |
| LIBC523862- | 113 | DIVMTQSPLSLPVTPGEPASISCRSSQSLLHSTGANYVD | 114 | QVQLQESGPGLVKPSQTLSLTCTVSGGSIRRGGDYWSWIRQHPGK |
| 1 | | WYLQKPGQSPQLLIYLGSNRASGVPDRFSGSGSGTDFTL | | GLEWIGHIYQTGSTAYNPSLKGRATISVNTYKNQFSLKLSSVTAADT |
| | | KISRVEAEDVGVYYCRQAIALPRTFGQGTKVEIKR | | AVYYCARDYDILTGYSYYYYGMDVWGQGTTVTVSS |
| LIBC524049- | 115 | DIVMTQSPLSLPVTPGEPASISCRSSQSLLHSYGANYLDW | 116 | QVQLQESGPGLVKPSQTLSLTCTVSGGSIRRGGDYWSWIRQHPGK |
| 1 | | YLQKPGQSPQLLIYLGSNRASGVPDRFSGSGSGTDFTLKI | | GLEWIGHIYYTGSTAYNPSLKSRATISVNTYKNQFSLKLSSVTAADTA |
| | | SRVEAEDVGVYYCRQARTLPRTFGQGTKVEIKR | | VYYCARDYDILTGYSYYYYGMDVWGQGTTVTVSS |
| LIBC523813- | 117 | DIVMTQSPLSLPVTPGEPASISCRSSQSLLSSSGANYVDW | 118 | QVQLQESGPGLVKPSQTLSLTCTVSGGSIRRGGDYWSWIRQHPGK |
| 1 + VK2 | | YLQKPGQSPQLLIYLGSNRASGVPDRFSGSGSGTDFTLKI | | GLEWIGHVYYTGQTAYNPKFKDRATISVKTYKNQFSLKLSSVTAADT |
| germline | | SRVEAEDVGVYYCMQARGIPRTFGQGTKVEIKR | | AVYYCARDYDILTGYSYYYYGMDVWGQGTTVTVSS |
| fix | | | | |
| LIBC523815- | 119 | YLQKPGQSPQLLIYLGSNRASGVPDRFSGSGSGTDFTLKI | 120 | QVQLQESGPGLVKPSQTLSLTCTVSGGSIRRGGDYWSWIRQHPGK |
| 1 | | DIVMTQSPLSLPVTPGEPASISCRSSQSLLTSSGANYVDW | | GLEWIGHIYQSGQTKYNPSLKDRATISVNTYKNQFSLKLSSVTAADT |
| | | SRVEAEDVGVYYCRQAREQPRTFGQGTKVEIKR | | AVYYCARDYDILTGYSYYYYGMDVWGQGTTVTVSS |
| LIBC523603- | 121 | DIVMTQSPLSLPVTPGEPASISCRSSQSLLSSYGNNYVDW | 122 | QVQLQESGPGLVKPSQTLSLTCTVSGGSIRRGGDYWAWIRQHPGK |
| 1 | | YLQKPGQSPQLLIYQGSNRASGVPDRFSGSGSGTDFTLKI | | GLEWIGHVYQSGQTAYNPNFENRATISVNTYKNQFSLKLSSVTAAD |
| | | SRVEAEDVGVYYCRQAVGLPRTFGQGTKVEIKR | | TAVYYCARDYDAVSGYSYYYYGMDVWGQGTTVTVSS |
| LIBC523596- | 123 | DIVMTQSPLSLPVTPGEPASISCRSSQSLLTSSGYNYVDW | 124 | QVQLQESGPGLVKPSQTLSLTCTVSGGSIRRGGDYWSWIRQHPGK |
| 1 | | YLQKPGQSPQLLIYQGSNRASGVPDRFSGSGSGTDFTLKI | | GLEWIGHVYQSGQTAYNPNFEDRATISVNTYKNQFSLKLSSVTAAD |
| | | SRVEAEDVGVYYCMQAVKIPRTFGQGTKVEIKR | | TAVYYCAREYDVVSGYSYYYYGMDVWGQGTTVTVSS |
| LIBC523670- | 125 | DIVMTQSPLSLPVTPGEPASISCRSSQSLLTSYGANYLDW | 126 | QVQLQESGPGLVKPSQTLSLTCTVSGGSIRRGGDYWAWIRQHPGK |
| 1 + VK2 | | YLQKPGQSPQLLIYQGSNRASGVPDRFSGSGSGTDFTLKI | | GLEWIGHVYQTGQTAYNPNLESRATISVNTYKNQFSLKLSSVTAAD |
| germline | | SRVEAEDVGVYYCMQALQIPRTFGQGTKVEIKR | | TAVYYCARNYDVLSGYSYYYYGMDVWGQGTTVTVSS |
| fix | | | | |
| 10D10 | 127 | DIVMTQSPLSLPVTPGEPASISCRSSQSLLHSNGYNYLDW | 128 | QVQLQESGPGLVKPSQTLSLTCTVSGGSIRRGGDYWSWIRQHPGK |
| | | YLQKPGQSPQLLIYLGSNRASGVPDRFSGSGSGTDFTLKI | | GLEWIGYIYYSGSTNYNPSLKSRATISVDTSKNQFSLKLSSVTAADTA |
| | | RRVEAEDVGVYYCMQALQTPRTFGQGTKVEIKR | | VYYCARDYDILTGYSYYYYGMDVWGQGTTVTVSS |

TABLE 9
Top 15 affinity-matured cognate 10D10 variants: binding information

| | | | | | | 100 nM- | |
|---|---|---|---|---|---|---|---|
| | | 100 nM- | Specific | | | huCB1- | Specific |
| | | huCB1- | huCB1 | | | SMALP | huCB1 |
| | 0.2 nM- | SMALP | binding | | 0.2 nM- | Binding | binding |
| | huCB1-ND | Binding | (CB1 | | huCB1-ND | (normalized | (CB1 |
| | (Relative to | (normalized | ND/Empty | | (Relative to | to | ND/Empty |
| Name | 10D10N35Y) | to 10D10) | ND ratio)) | Name | 10D10N35Y) | 10D10) | ND ratio)) |
| 10D10 | 0.83 | 1.00 | 1.32 | LIBC523760-1 | 4.19 | 5.70 | 7.21 |
| N35Y | 1.00 | 0.95 | 1.35 | LIBC523862-1 | 5.07 | 3.97 | 13.55 |
| benchmark | | | | | | | |
| LIBC523661-1 | 4.03 | 8.00 | 9.32 | LIBC524049-1 | 5.01 | 5.51 | 9.50 |
| LIBC523797-1 | 8.49 | 7.01 | 24.45 | LIBC523813-1 | 3.38 | 6.32 | 5.79 |
| LIBC523969-1 | 4.53 | 5.69 | 12.81 | LIBC523815-1 | 6.15 | 4.55 | 14.78 |
| LIBC523748-1 | 4.41 | 6.12 | 5.33 | LIBC523603-1 | 6.05 | 4.05 | 19.35 |
| LIBC523857-1 | 4.05 | 4.71 | 13.91 | LIBC523596-1 | 4.92 | 4.19 | 20.63 |
| LIBC523868-1 | 6.28 | 4.21 | 13.26 | LIBC523670-1 | 7.46 | 5.42 | 23.04 |
| LIBC523844-1 | 3.78 | 6.69 | 8.58 | | | | |

### Example 3. Light Chain Swapped Affinity Maturation

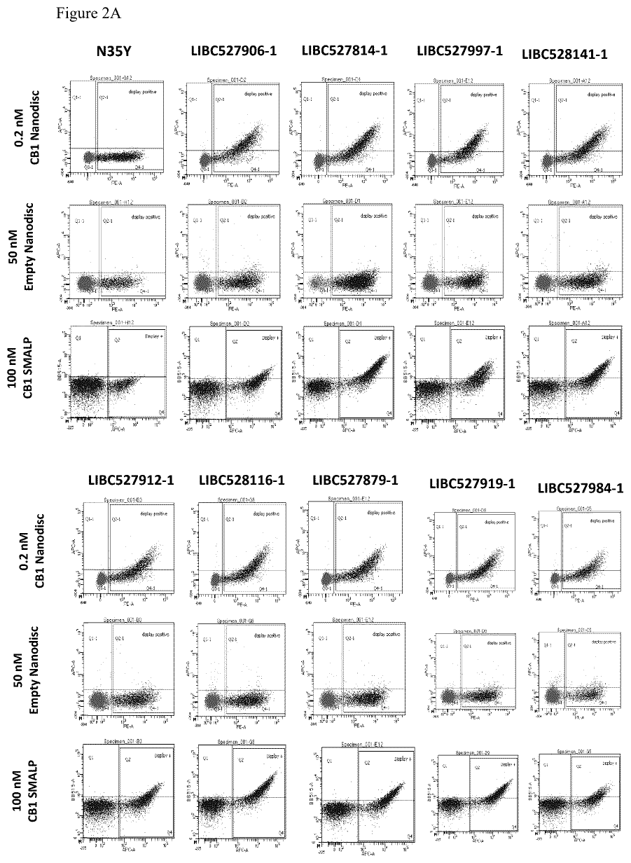

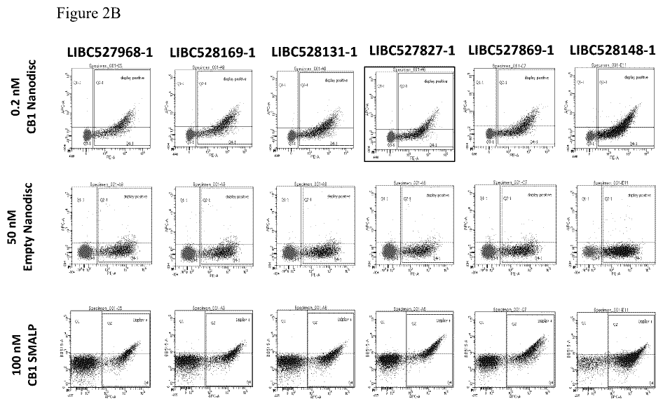

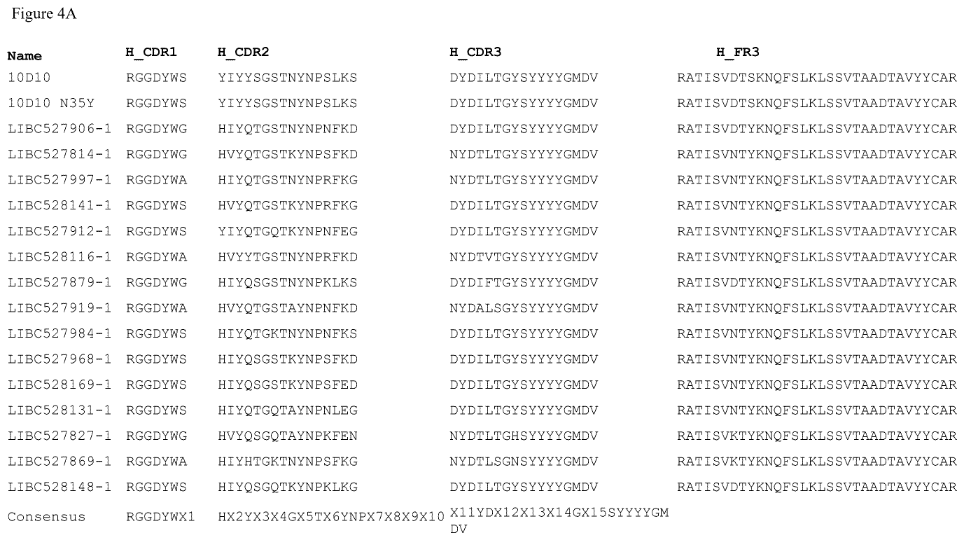

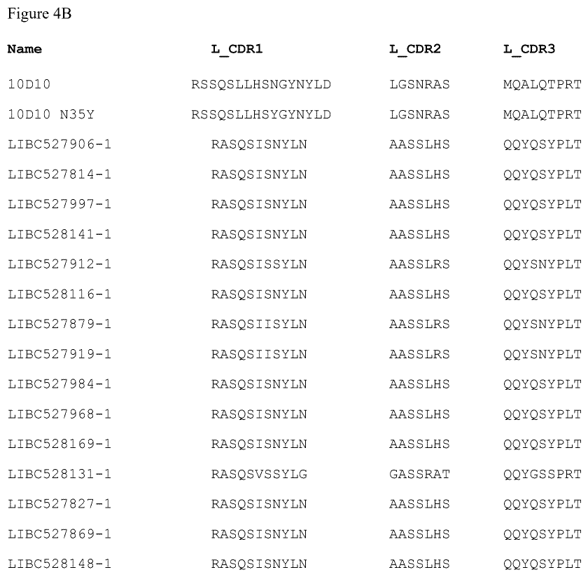

In a parallel arm of affinity maturation, the 22 VK1 and VK3 LCs obtained in Example 1 were transformed with 10D10 mutant HC genes or gene fragments extracted from yeast cells displaying improved binding variants obtained after FACS enrichment of H1H3 and H2/de-loop libraries (Example 2). The three LC-swapped libraries (mutH1H3/VK LCs, mutH2/VK LCs, and mutH1H2H3/VK LCs) for affinity maturation were enriched for the most improved binders using higher-stringency FACS enrichments. Clones isolated from the LC-swapped libraries were screened in two rounds for top clone selection. The screening and selection criteria for LC-swapped 10D10 affinity matured variants were the same as those for the cognate affinity maturation described in Example 2 except that prioritization of sequences with a reduction in hydrophobicity in LCDR1 and LCDR2 was not used. A total of 600 positive clones entered the first round of screening and 200 positive clones entered the second round. The 200 positive clones were then ranked for CB1 specific binding to obtain the final 15 top LC-swapped binders. CDR and variable region sequence of the top 15 LC-swapped affinity-matured 10D10 variant are shown in Tables 10 and 11 blow. Table 12 below summarizes the binding data of the top 15 LC-swapped affinity matured 10D10 variants. FIGS. 2A and 2B show the flow cytometry binding profiles of these variants. Similar to the cognate affinity matured variants, all LC-swapped top clones exhibit significantly improved binding to huCB1-ND and huCB1-SMALP while maintaining the minimal binding to empty-ND compared to 10D10 LC N35Y (Table 12). In addition, Y57H within HCDR2, and D83N and S85Y mutations within the HC FR3 de loop were observed in the top 15 LC-swapped 10D10 affinity maturation variants (FIG. 4A). Furthermore, VK1 LCs were heavily represented among the top LC-swapped 10D10 variants, with only one variant having a VK3 LC (Table 13). The R94S germline reversion was imposed in all VK LC sequences of the top variants if not present in the original sequence.

TABLE 10
CDR sequence of top 15 affinity-matured LC-swapped 10D10 variants

| | Seq | | Seq | | Seq | |
|---|---|---|---|---|---|---|
| Name | No. | CDR1 | No. | CDR2 | No. | CDR3 |
| LIBC527997- | 129 | RGGDYWA | 130 | HIYQTGSTNYNPRFKG | 131 | NYDTLTGYSYYYYGMDV |
| 1_HC huCB1 | | | | | | |
| HV | | | | | | |
| LIBC527997- | 132 | RASQSISNYLN | 133 | AASSLHS | 134 | QQYQSYPLT |
| 1_LC huCB1 LV | | | | | | |
| LIBC528116-1 + | 135 | RGGDYWA | 136 | HVYYTGSTNYNPRFKD | 137 | NYDTVTGYSYYYYGMDV |
| VK1 germline | | | | | | |
| fix(es)_HC | | | | | | |
| huCB1 HV | | | | | | |
| LIBC528116-1 + | 138 | RASQSISNYLN | 139 | AASSLHS | 140 | QQYQSYPLT |
| VK1 germline | | | | | | |
| fix(es)_LC | | | | | | |
| huCB1 LV | | | | | | |
| LIBC528169-1 + | 141 | RGGDYWS | 142 | HIYQSGSTKYNPSFED | 143 | DYDILTGYSYYYYGMDV |
| VK1 germline | | | | | | |
| fix(es)_HC | | | | | | |
| huCB1 HV | | | | | | |
| LIBC528169-1 + | 144 | RASQSISNYLN | 145 | AASSLHS | 146 | QQYQSYPLT |
| VK1 germline | | | | | | |
| fix(es)_LC | | | | | | |
| huCB1 LV | | | | | | |
| LIBC527879- | 147 | RGGDYWG | 148 | HIYQSGSTNYNPKLKS | 149 | DYDIFTGYSYYYYGMDV |
| 1_HC huCB1 | | | | | | |
| HV | | | | | | |
| LIBC527879- | 150 | RASQSIISYLN | 151 | AASSLRS | 152 | QQYSNYPLT |
| 1_LC huCB1 LV | | | | | | |
| LIBC528141- | 153 | RGGDYWS | 154 | HVYQTGSTKYNPRFKG | 155 | DYDILTGYSYYYYGMDV |
| 1_HC huCB1 | | | | | | |
| HV | | | | | | |
| LIBC528141- | 156 | RASQSISNYLN | 157 | AASSLHS | 158 | QQYQSYPLT |
| 1_LC huCB1 LV | | | | | | |
| LIBC527814- | 159 | RGGDYWG | 160 | HVYQTGSTKYNPSFKD | 161 | NYDTLTGYSYYYYGMDV |
| 1_HC huCB1 | | | | | | |
| HV | | | | | | |
| LIBC527814- | 162 | RASQSISNYLN | 163 | AASSLHS | 164 | QQYQSYPLT |
| 1_LC huCB1 LV | | | | | | |
| LIBC528131- | 165 | RGGDYWS | 166 | HIYQTGQTAYNPNLEG | 167 | DYDILTGYSYYYYGMDV |
| 1_HC huCB1 | | | | | | |
| HV | | | | | | |
| LIBC528131- | 168 | RASQSVSSYLG | 169 | GASSRAT | 170 | QQYGSSPRT |
| 1_LC huCB1 LV | | | | | | |
| LIBC527984-1 + | 171 | RGGDYWS | 172 | HIYQTGKTNYNPNFKS | 173 | |
| VK1 germline | | | | | | |
| fix(es)_HC | | | | | | |
| huCB1 HV | | | | | | |
| LIBC527984-1 + | 174 | RASQSISNYLN | 175 | AASSLHS | 176 | QQYQSYPLT |
| VK1 germline | | | | | | |
| fix(es)_LC | | | | | | |
| huCB1 LV | | | | | | |
| LIBC527906-1 + | 177 | RGGDYWG | 178 | HIYQTGSTNYNPNFKD | 179 | DYDILTGYSYYYYGMDV |
| VK1 germline | | | | | | |
| fix(es)_HC | | | | | | |
| huCB1 HV | | | | | | |
| LIBC527906-1 + | 180 | RASQSISNYLN | 181 | AASSLHS | 182 | QQYQSYPLT |
| VK1 germline | | | | | | |
| fix(es)_LC | | | | | | |
| huCB1 LV | | | | | | |
| LIBC527912- | 183 | RGGDYWS | 184 | YIYQTGQTKYNPNFEG | 185 | DYDILTGYSYYYYGMDV |
| 1_HC huCB1 | | | | | | |
| HV | | | | | | |
| LIBC527912- | 186 | RASQSISSYLN | 187 | AASSLRS | 188 | QQYSNYPLT |
| 1_LC huCB1 LV | | | | | | |
| LIBC528148-1 + | 189 | RGGDYWS | 190 | HIYQSGQTKYNPKLKG | 191 | DYDILTGYSYYYYGMDV |
| VK1 germline | | | | | | |
| fix(es)_HC | | | | | | |
| huCB1 HV | | | | | | |
| LIBC528148-1 + | 192 | RASQSISNYLN | 193 | AASSLHS | 194 | QQYQSYPLT |
| VK1 germline | | | | | | |
| fix(es)_LC | | | | | | |
| huCB1 LV | | | | | | |
| LIBC527968-1 + | 195 | RGGDYWS | 196 | HIYQSGSTKYNPSFKD | 197 | DYDILTGYSYYYYGMDV |
| VK1 germline | | | | | | |
| fix(es)_HC | | | | | | |
| huCB1 HV | | | | | | |
| LIBC527968-1 + | 198 | RASQSISNYLN | 199 | AASSLHS | 200 | QQYQSYPLT |
| VK1 germline | | | | | | |
| fix(es)_LC | | | | | | |
| huCB1 LV | | | | | | |
| LIBC527869-1 + | 201 | RGGDYWA | 202 | HIYHTGKTNYNPSFKG | 203 | NYDTLSGNSYYYYGMDV |
| VK1 germline | | | | | | |
| fix(es)_HC | | | | | | |
| huCB1 HV | | | | | | |
| LIBC527869-1 + | 204 | RASQSISNYLN | 205 | AASSLHS | 206 | QQYQSYPLT |
| VK1 germline | | | | | | |
| fix(es)_LC | | | | | | |
| huCB1 LV | | | | | | |
| LIBC527919- | 207 | RGGDYWA | 208 | HVYQTGSTAYNPNFKD | 209 | NYDALSGYSYYYYGMDV |
| 1_HC huCB1 | | | | | | |
| HV | | | | | | |
| LIBC527919- | 210 | RASQSIISYLN | 211 | AASSLRS | 212 | QQYSNYPLT |
| 1_LC huCB1 LV | | | | | | |
| LIBC527827-1 + | 213 | RGGDYWG | 214 | HVYQSGQTAYNPKFEN | 215 | NYDTLTGHSYYYYGMDV |
| VK1 germline | | | | | | |
| fix(es)_HC | | | | | | |
| huCB1 HV | | | | | | |
| LIBC527827-1 + | 216 | RASQSISNYLN | 217 | AASSLHS | 218 | QQYQSYPLT |
| VK1 germline | | | | | | |
| fix(es)_LC | | | | | | |
| huCB1 LV | | | | | | |
| 10D10 HV | 91 | RGGDYWS | 92 | YIYYSGSTNYNPSLKS | 93 | DYDILTGYSYYYYGMDV |
| 10D10 LV | 94 | RSSQSLLHSNGYNYLD | 95 | LGSNRAS | 96 | MQALQTPRT |
| 10D10N35Y HV | 219 | RGGDYWS | 220 | YIYYSGSTNYNPSLKS | 221 | DYDILTGYSYYYYGMDV |
| 20D10N35Y LV | 222 | RSSQSLLHSYGYNYLD | 223 | LGSNRAS | 224 | MQALQTPRT |

TABLE 11
Variable region sequence of top 15 LC-swapped 10D10 variants

| | Seq | | Seq | |
|---|---|---|---|---|
| Name | No. | LC LV | No. | HC HV |
| LIBC527997- | 225 | DIQMTQSPSSLSASVGDRVTITCRASQSISNYLNWYQQK | 226 | QVQLQESGPGLVKPSQTLSLTCTVSGGSIRRGGDYWAWIRQHPG |
| 1 | | PGKAPKLLIYAASSLHSGVPSRFSGSGSGTDFTLTISSLQPE | | KGLEWIGHIYQTGSTNYNPRFKGRATISVNTYKNQFSLKLSSVTAA |
| | | DFASYYCQQYQSYPLTFGQGTKVEIKR | | DTAVYYCARNYDTLTGYSYYYYGMDVWGQGTTVTVSS |
| LIBC528116- | 227 | DIQLTQSPSTLSASVGDRVTITCRASQSISNYLNWYQQKP | 228 | QVQLQESGPGLVKPSQTLSLTCTVSGGSIRRGGDYWAWIRQHPG |
| 1 + VK1 | | GKAPKLLIYAASSLHSGVPSRFSGSGSGTDFTLTISSLQPE | | KGLEWIGHVYYTGSTNYNPRFKDRATISVNTYKNQFSLKLSSVTAA |
| germline | | DFASYYCQQYQSYPLTFGQGTKVEIKR | | DTAVYYCARNYDTVTGYSYYYYGMDVWGQGTTVTVSS |
| fix(es) | | | | |
| LIBC528169- | 229 | DIQLTQSPSTLSASVGDRVTITCRASQSISNYLNWYQQKP | 230 | QVQLQESGPGLVKPSQTLSLTCTVSGGSIRRGGDYWSWIRQHPG |
| 1 + VK1 | | GKAPKLLIYAASSLHSGVPSRFSGSGSGTDFTLTISSLQPE | | KGLEWIGHIYQSGSTKYNPSFEDRATISVNTYKNQFSLKLSSVTAA |
| germline | | DFASYYCQQYQSYPLTFGQGTKVEIKR | | DTAVYYCARDYDILTGYSYYYYGMDVWGQGTTVTVSS |
| fix(es) | | | | |
| LIBC527879- | 231 | DIQLTQSPSSLSASVGDRVTITCRASQSIISYLNWYQQKP | 232 | QVQLQESGPGLVKPSQTLSLTCTVSGGSIRRGGDYWGWIRQHPG |
| 1 | | GKAPKLLIYAASSLRSGVPSRFSGSGSGTDFTLTISSLQPED | | KGLEWIGHIYQSGSTNYNPKLKSRATISVDTYKNQFSLKLSSVTAA |
| | | FATYYCQQYSNYPLTFGGGTRVEIKR | | DTAVYYCARDYDIFTGYSYYYYGMDVWGQGTTVTVSS |
| LIBC528141- | 233 | DIQLTQSPSTLSASVGDRVTITCRASQSISNYLNWYQQKP | 234 | QVQLQESGPGLVKPSQTLSLTCTVSGGSIRRGGDYWSWIRQHPG |
| 1 | | GKAPKLLIYAASSLHSGVPSRFSGSGSGTDFTLTISSLQPE | | KGLEWIGHVYQTGSTKYNPRFKGRATISVNTYKNQFSLKLSSVTAA |
| | | DFASYYCQQYQSYPLTFGQGTKVEIKR | | DTAVYYCARDYDILTGYSYYYYGMDVWGQGTTVTVSS |
| LIBC527814- | 235 | DIQMTQSPSTLSASVGDRVTITCRASQSISNYLNWYQQK | 236 | QVQLQESGPGLVKPSQTLSLTCTVSGGSIRRGGDYWGWIRQHPG |
| 1 | | PGKAPKLLIYAASSLHSGVPSRFSGSGSGTDFTLTISSLQPE | | KGLEWIGHVYQTGSTKYNPSFKDRATISVNTYKNQFSLKLSSVTAA |
| | | DFASYYCQQYQSYPLTFGQGTKVEIKR | | DTAVYYCARNYDTLTGYSYYYYGMDVWGQGTTVTVSS |
| LIBC528131- | 237 | EIVLTQSPATLSLSPGERATLSCRASQSVSSYLGWYQQKP | 238 | QVQLQESGPGLVKPSQTLSLTCTVSGGSIRRGGDYWSWIRQHPG |
| 1 | | GQAPRLLIYGASSRATGIPDRFSGSGSGTDFTLTISRLEPE | | KGLEWIGHIYQTGQTAYNPNLEGRATISVNTYKNQFSLKLSSVTAA |
| | | DFAVYYCQQYGSSPRTFGQGTKVEIKR | | DTAVYYCARDYDILTGYSYYYYGMDVWGQGTTVTVSS |
| LIBC527984- | 239 | DIQMTQSPSTLSASVGDRVTITCRASQSISNYLNWYQQK | 240 | QVQLQESGPGLVKPSQTLSLTCTVSGGSIRRGGDYWSWIRQHPG |
| 1 + VK1 | | PGKAPKLLIYAASSLHSGVPSRFSGSGSGTDFTLTISSLQPE | | KGLEWIGHIYQTGKTNYNPNFKSRATISVNTYKNQFSLKLSSVTAA |
| germline | | DFASYYCQQYQSYPLTFGQGTKVEIKR | | DTAVYYCARDYDILTGYSYYYYGMDVWGQGTTVTVSS |
| fix(es) | | | | |
| LIBC527906- | 241 | DIQLTQSPSTLSASVGDRVTITCRASQSISNYLNWYQQKP | 242 | QVQLQESGPGLVKPSQTLSLTCTVSGGSIRRGGDYWGWIRQHPG |
| 1 + VK1 | | GKAPKLLIYAASSLHSGVPSRFSGSGSGTDFTLTISSLQPE | | KGLEWIGHIYQTGSTNYNPNFKDRATISVDTYKNQFSLKLSSVTAA |
| germline | | DFASYYCQQYQSYPLTFGQGTKVEIKR | | DTAVYYCARDYDILTGYSYYYYGMDVWGQGTTVTVSS |
| fix(es) | | | | |
| LIBC527912- | 243 | DIQLTQSPSSLSASVGDRVTITCRASQSISSYLNWYQQKP | 244 | QVQLQESGPGLVKPSQTLSLTCTVSGGSIRRGGDYWSWIRQHPG |
| 1 | | GKAPKLLIYAASSLRSGVPSRFSGSGSGTDFTLTISSLQPED | | KGLEWIGYIYQTGQTKYNPNFEGRATISVNTYKNQFSLKLSSVTAA |
| | | FATYYCQQYSNYPLTFGGGTRVEIKR | | DTAVYYCARDYDILTGYSYYYYGMDVWGQGTTVTVSS |
| LIBC528148- | 245 | DIQLTQSPSTLSASVGDRVTITCRASQSISNYLNWYQQKP | 246 | QVQLQESGPGLVKPSQTLSLTCTVSGGSIRRGGDYWSWIRQHPG |
| 1 + VK1 | | GKAPKLLIYAASSLHSGVPSRFSGSGSGTDFTLTISSLQPE | | KGLEWIGHIYQSGQTKYNPKLKGRATISVDTYKNQFSLKLSSVTAA |
| germline | | DFASYYCQQYQSYPLTFGQGTKVEIKR | | DTAVYYCARDYDILTGYSYYYYGMDVWGQGTTVTVSS |
| fix(es) | | | | |
| LIBC527968- | 247 | DIQLTQSPSTLSASVGDRVTITCRASQSISNYLNWYQQKP | 248 | QVQLQESGPGLVKPSQTLSLTCTVSGGSIRRGGDYWSWIRQHPG |
| 1 + VK1 | | GKAPKLLIYAASSLHSGVPSRFSGSGSGTDFTLTISSLQPE | | KGLEWIGHIYQSGSTKYNPSFKDRATISVNTYKNQFSLKLSSVTAA |
| germline | | DFASYYCQQYQSYPLTFGQGTKVEIKR | | DTAVYYCARDYDILTGYSYYYYGMDVWGQGTTVTVSS |
| fix(es) | | | | |
| LIBC527869- | 249 | DIQLTQSPSTLSASVGDRVTITCRASQSISNYLNWYQQKP | 250 | QVQLQESGPGLVKPSQTLSLTCTVSGGNIRRGGDYWAWIRQHP |
| 1 + VK1 | | GKAPKLLIYAASSLHSGVPSRFSGSGSGTDFTLTISSLQPE | | GKGLEWIGHIYHTGKTNYNPSFKGRATISVKTYKNQFSLKLSSVTA |
| germline | | DFASYYCQQYQSYPLTFGQGTKVEIKR | | ADTAVYYCARNYDTLSGNSYYYYGMDVWGQGTTVTVSS |
| fix(es) | | | | |
| LIBC527919- | 251 | DIQLTQSPSSLSASVGDRVTITCRASQSIISYLNWYQQKP | 252 | QVQLQESGPGLVKPSQTLSLTCTVSGGSIRRGGDYWAWIRQHPG |
| 1 | | GKAPKLLIYAASSLRSGVPSRFSGSGSGTDFTLTISSLQPED | | KGLEWIGHVYQTGSTAYNPNFKDRATISVNTYKNQFSLKLSSVTA |
| | | FATYYCQQYSNYPLTFGGGTRVEIKR | | ADTAVYYCARNYDALSGYSYYYYGMDVWGQGTTVTVSS |
| LIBC527827- | 253 | DIQMTQSPSTLSASVGDRVTITCRASQSISNYLNWYQQK | 254 | QVQLQESGPGLVKPSQTLSLTCTVSGGSIRRGGDYWGWIRQHPG |
| 1 + VK1 | | PGKAPKLLIYAASSLHSGVPSRFSGSGSGTDFTLTISSLQPE | | KGLEWIGHVYQSGQTAYNPKFENRATISVKTYKNQFSLKLSSVTA |
| germline | | DFASYYCQQYQSYPLTFGQGTKVEIKR | | ADTAVYYCARNYDTLTGHSYYYYGMDVWGQGTTVTVSS |
| fix(es) | | | | |
| 10D1 | 127 | DIVMTQSPLSLPVTPGEPASISCRSSQSLLHSNGYNYLDW | 128 | QVQLQESGPGLVKPSQTLSLTCTVSGGSIRRGGDYWSWIRQHPG |
| | | YLQKPGQSPQLLIYLGSNRASGVPDRFSGSGSGTDFTLKI | | KGLEWIGYIYYSGSTNYNPSLKSRATISVDTSKNQFSLKLSSVTAAD |
| | | RRVEAEDVGVYYCMQALQTPRTFGQGTKVEIKR | | TAVYYCARDYDILTGYSYYYYGMDVWGQGTTVTVSS |
| 10D10 | 255 | DIVMTQSPLSLPVTPGEPASISCRSSQSLLHSYGYNYLDW | 256 | QVQLQESGPGLVKPSQTLSLTCTVSGGSIRRGGDYWSWIRQHPG |
| N35Y | | YLQKPGQSPQLLIYLGSNRASGVPDRFSGSGSGTDFTLKI | | KGLEWIGYIYYSGSTNYNPSLKSRATISVDTSKNQFSLKLSSVTAAD |
| | | RRVEAEDVGVYYCMQALQTPRTFGQGTKVEIKR | | TAVYYCARDYDILTGYSYYYYGMDVWGQGTTVTVSS |

TABLE 12
Top 15 LC-swapped 10D10 variants: Binding Information

| | 100 nM-huCB1-GFP- | | |
|---|---|---|---|
| | SMALP Binding | 0.200 nM-huCB1- | 50.0 nM-Empty |
| | (normalized to | Nanodisc (Relative to | Nanodisc (Relative |
| Name | 10D10N35Y) | 10D10N35Y) | to 10D10N35Y) |
| 10D10 | 1.00 | 0.68 | 0.81 |
| 10D10 N35Y | 1.00 | 1.00 | 1.00 |
| LIBC527906-1 | 3.79 | 8.02 | 0.51 |
| LIBC527814-1 | 6.44 | 6.49 | 0.61 |
| LIBC527997-1 | 6.63 | 6.38 | 0.44 |
| LIBC528141-1 | 8.31 | 6.20 | 0.49 |
| LIBC527912-1 | 4.32 | 5.95 | 0.51 |
| LIBC528116-1 | 5.80 | 5.91 | 0.77 |
| LIBC527879-1 | 3.24 | 5.75 | 0.73 |
| LIBC527919-1 | 4.15 | 5.22 | 0.64 |
| LIBC527984-1 | 4.85 | 5.21 | 0.61 |
| LIBC527968-1 | 4.65 | 4.86 | 0.64 |
| LIBC528169-1 | 4.58 | 4.82 | 0.68 |
| LIBC528131-1 | 3.68 | 4.47 | 0.63 |
| LIBC527827-1 | 4.99 | 4.31 | 1.16 |
| LIBC527869-1 | 3.61 | 4.15 | 0.97 |
| LIBC528148-1 | 3.38 | 5.80 | 0.27 |

TABLE 13
Top 15 LC-swapped 10D10 variants germline information

| Name | VH germline | VL Germline |
|---|---|---|
| 10D10 | VH4\|4-31/D3\|3-9\|RF2/JH6 | VK2\|A19/JK1 |
| 10D10 N35Y | VH4\|4-31/D3\|3-9\|RF2/JH6 | VK2\|A19/JK1 |
| LIBC527906-1 | VH4\|4-31/D3\|3-9\|RF2/JH6 | VK1\|O2/JK1 |
| LIBC527814-1 | VH4\|4-31/D3\|3-9\|RF2/JH6 | VK1\|O2/JK1 |
| LIBC527997-1 | VH4\|4-31/D3\|3-9\|RF2/JH6 | VK1\|O2/JK1 |
| LIBC528141-1 | VH4\|4-31/D3\|3-9\|RF2/JH6 | VK1\|O2/JK1 |
| LIBC527912-1 | VH4\|4-31/D3\|3-9\|RF2/JH6 | VK1\|O2/JK4 |
| LIBC528116-1 | VH4\|4-31/D3\|3-9\|RF2/JH6 | VK1\|O2/JK1 |
| LIBC527879-1 | VH4\|4-31/D3\|3-9\|RF2/JH6 | VK1\|O2/JK4 |
| LIBC527919-1 | VH4\|4-31/D3\|3-9\|RF2/JH6 | VK1\|O2/JK4 |
| LIBC527984-1 | VH4\|4-31/D3\|3-9\|RF2/JH6 | VK1\|O2/JK1 |
| LIBC527968-1 | VH4\|4-31/D3\|3-9\|RF2/JH6 | VK1\|O2/JK1 |
| LIBC528169-1 | VH4\|4-31/D3\|3-9\|RF2/JH6 | VK1\|O2/JK1 |
| LIBC528131-1 | VH4\|4-31/D3\|3-9\|RF2/JH6 | VK3\|A27/JK1 |
| LIBC527827-1 | VH4\|4-31/D3\|3-9\|RF2/JH6 | VK1\|O2/JK1 |
| LIBC527869-1 | VH4\|4-31/D3\|3-9\|RF2/JH6 | VK1\|O2/JK1 |
| LIBC528148-1 | VH4\|4-31/D3\|3-9\|RF2/JH6 | VK1\|O2/JK1 |

### Example 4 Cross Reactivity and Specificity of Affinity Matured Antibodies

The 10D10Dam antibody does not bind to the mouse CB (muCB1) or the human CB2 (huCB2) protein (WO2014/210205). Because all the antibodies isolated in Examples 2 and 3 are variants of 10D 10, they are not expected to bind to huCB1 or muCB1. Binding activity of exemplary antibodies against muCB1 and huCB2 confirms that there were no cross-reactivity against these two proteins (Table 14).

TABLE 14
No cross-reactivity against muCB1 and huCB2

Fold over mock

| Name | huCB2 | muCB1 |
|---|---|---|
| LIBC527814-1 | 0.9 | 1.2 |
| LIBC527919-1 | 0.9 | 1.0 |
| LIBC527997-1 | 0.8 | 1.0 |
| LIBC528116-1 | 0.9 | 0.8 |
| LIBC523661-1 | 0.8 | 0.7 |
| LIBC523797-1 | 0.9 | 0.8 |
| LIBC524049-1 | 1.0 | 0.8 |
| 10D10 HC D83K/LC | 0.7 | 0.7 |
| N35Y (26970-1) | | |
| 10D10 | 0.9 | 0.9 |
| Positive control | 16.5 | 7.9 |
| Negative control | 1.1 | 1.1 |

### Example 5 Characterization of Affinity Maturation Variants

The top clones isolated from Examples 2 and 3 above were cloned and expressed as IgG1 SEFL2 mAbs and then tested in a cAMP functional assay to measure antagonist activity or inverse agonist activity.

### Antagonist and Inverse Agonist Activity

Antagonist activity. Stably transfected CB1 CHO cells were serum starved overnight before the assay at 37° C./5% CO₂ and using DMEM (Sigma Cat #D5671) with 0.5% FBS, 25 mM HEPES. On the day of assay, CB1 antibodies and Rimonabant were titrated 1 in 4 at 4 times final concentration using DMEM (Sigma Cat #D5671) with 0.1% BSA (from PE kit), 25 mM HEPES, the titrated antibodies were transferred to the 384 well assay plate at a volume of 7.5 μL per well. The cells were harvested using Accutase (Sigma) and neutralized with DMEM (Sigma Cat #D5671) with 0.5% FBS, 25 mM HEPES. These cells were pelleted and resuspended to 1.33×10⁶ cells/mL using DMEM (Sigma Cat #D5671) with 0.1% BSA (from PE kit), 25 mM HEPES. The anti cAMP antibody from the Lance Ultra cAMP Detection kit (Perkin Elmer; TRF0263) was added to the cells at a 1 in 150 ratio of antibody to cell volume, 3-Isobutyl-1-methylxanthine (IBMX; Sigma) was also added to the cells at a final concentration of 1 mM. The cells were transferred to the assay plate at a volume of 15 μL per well or 20 000 cells per well. Forskolin and CP55,940 cocktail was diluted to 4 times final concentration using DMEM (Sigma Cat #D5671) with 0.1% BSA (from PE kit), 25 mM HEPES and transferred to the assay plate at a volume of 7.5 μL per well. The assay plate was incubated at 37° C./5% CO₂ for ½ hour before EU-cAMP tracer (Perkin Elmer; TRF0263) was added to each well and further incubated at room temperature protected from light for 1 hour. After the final incubation, the assay plates were read using Envision plate reader with EU615 and APC 665 Emission filters. The data was analysed using calculation of: TR-FRET(665 nm/615 nm)×10 000. Lower value (lower FRET) was interpreted as stronger inhibition of CB1 and the data was used to calculated IC50. Antagonist activity of clones obtained from Examples 2 and 3 are shown in Table 15A (n=1 and n=2) and Table 15B (n=3).

TABLE 15A
IC50 values of cognate and LC-swapped affinity matured variants

| | | | n = 1 | | n = 2 | | Mean |
|---|---|---|---|---|---|---|---|
| | | | IC50, - | | IC50, - | | IC50, - |
| | | | fold vs | | fold vs | | fold vs |
| | | n = 1 | Rimo | n = 2 | Rimo | Mean | Rimo |
| | | IC50 | (<1 | IC50 | (<1 | IC50 | (<1 |
| Clone | Source | (nM) | desired) | (nM) | desired) | (nM) | desired) |
| LIBC528116-1 | LC swap | 3.93 | 0.93 | 0.88 | 0.49 | 2.40 | 0.80 |
| LIBC527814-1 | LC swap | 4.72 | 1.12 | 1.22 | 0.68 | 2.97 | 0.99 |
| LIBC527984-1 | LC swap | 7.24 | 1.72 | 1.27 | 0.71 | 4.25 | 1.42 |
| LIBC527997-1 | LC swap | 4.37 | 1.04 | 1.57 | 0.88 | 2.97 | 0.99 |
| LIBC527879-1 | LC swap | 6.12 | 1.45 | 1.78 | 0.99 | 3.95 | 1.32 |
| LIBC527919-1 | LC swap | 5.09 | 1.21 | 1.80 | 1.00 | 3.44 | 1.15 |
| LIBC528131-1 | LC swap | 6.63 | 1.57 | 1.83 | 1.02 | 4.23 | 1.41 |
| LIBC528141-1 | LC swap | 6.26 | 1.49 | 1.84 | 1.03 | 4.05 | 1.35 |
| LIBC527968-1 | LC swap | 6.10 | 1.45 | 1.95 | 1.09 | 4.03 | 1.34 |
| LIBC527912-1 | LC swap | 5.91 | 1.40 | 1.99 | 1.11 | 3.95 | 1.32 |
| LIBC527906-1 | LC swap | 7.66 | 1.82 | 2.64 | 1.48 | 5.15 | 1.72 |
| LIBC528148-1 | LC swap | 9.10 | 2.16 | 2.80 | 1.56 | 5.95 | 1.98 |
| LIBC528169-1 | LC swap | 12.30 | 2.92 | 3.31 | 1.85 | 7.81 | 2.60 |
| LIBC527827-1 | LC swap | 32.03 | 7.60 | 15.29 | 8.54 | 23.66 | 7.89 |
| LIBC527869-1 | LC swap | 57.82 | 13.73 | 36.16 | 20.20 | 46.99 | 15.66 |
| LIBC524049-1 | cognate | 4.41 | 1.05 | 1.34 | 0.75 | 2.87 | 0.96 |
| LIBC523797-1 | cognate | 4.70 | 1.12 | 1.44 | 0.80 | 3.07 | 1.02 |
| LIBC523661-1 | cognate | 5.24 | 1.24 | 1.35 | 0.75 | 3.30 | 1.10 |
| LIBC523844-1 | cognate | 6.20 | 1.47 | 1.46 | 0.82 | 3.83 | 1.28 |
| LIBC523969-1 | cognate | 6.45 | 1.53 | 1.26 | 0.70 | 3.86 | 1.29 |
| LIBC523760-1 | cognate | 6.12 | 1.45 | 2.44 | 1.36 | 4.28 | 1.43 |
| LIBC523603-1 | cognate | 7.29 | 1.73 | 2.16 | 1.20 | 4.72 | 1.57 |
| LIBC523813-1 | cognate | 8.96 | 2.13 | 2.94 | 1.64 | 5.95 | 1.98 |
| LIBC523670-1 | cognate | 8.72 | 2.07 | 3.52 | 1.97 | 6.12 | 2.04 |
| LIBC523748-1 | cognate | 9.57 | 2.27 | 4.43 | 2.48 | 7.00 | 2.33 |
| LIBC523862-1 | cognate | 11.20 | 2.66 | 3.42 | 1.91 | 7.31 | 2.44 |
| LIBC523868-1 | cognate | 11.86 | 2.81 | 3.32 | 1.86 | 7.59 | 2.53 |
| LIBC523857-1 | cognate | 13.40 | 3.18 | 2.48 | 1.39 | 7.94 | 2.65 |
| LIBC523596-1 | cognate | 13.98 | 3.32 | 4.00 | 2.23 | 8.99 | 3.00 |
| 10D10 HC | control | 10.86 | 2.58 | 9.81 | 5.48 | 10.33 | 3.44 |
| D83K/LC | (previous | | | | | | |
| N35Y (26970-1) | benchmark) | | | | | | |
| 10D10 | controls | 118.11 | 28.04 | 43.44 | 24.27 | 80.77 | 26.92 |
| Rimonabant | controls | 4.21 | 1.00 | 1.79 | 1.00 | 3.00 | 1.00 |

TABLE 15B
IC50 values of exemplary 10D10 variants

| | Mean IC50 |
|---|---|
| Name | (nM, n = 3) |
| LIBC528116-1 | 1.77 |
| LIBC527814-1 | 2.07 |
| LIBC527997-1 | 2.54 |
| LIBC527919-1 | 3.01 |
| LIBC523797-1 | 3.42 |
| LIBC524049-1 | 3.71 |
| LIBC523661-1 | 3.88 |
| 10D10 HC D83K/LC | 14.07 |
| N35Y (26970-1) | |
| 10D10 | 120.28 |
| rimonabant | 2.83 |

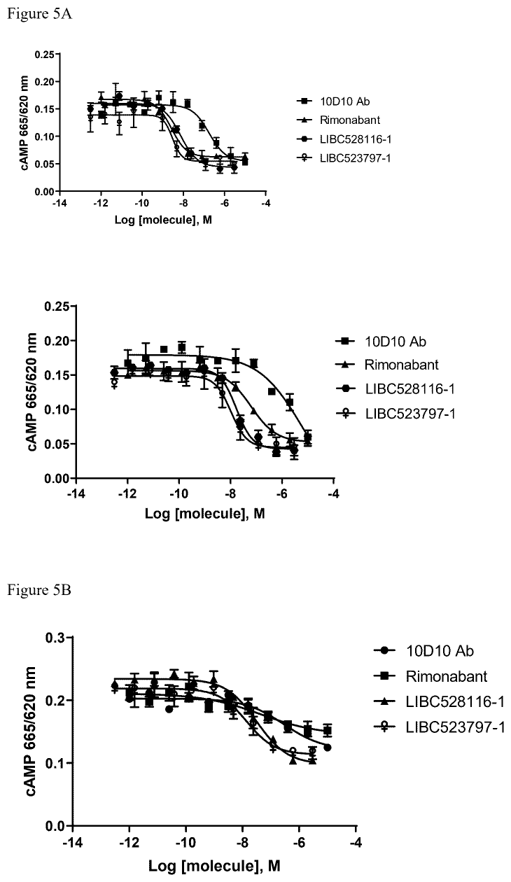

In addition, antagonist activity of exemplary 10D10 variants and a benchmark anti-CB1I antibody was measured in the presence of endogenous huCB1 ligands anandamide and 2-Arachidonoylglycerol (2-AG). Specifically, CHOK1 human CB1 receptor cells were plated overnight in DMEM with 0.5% FBS, P/S glutamine, 25 mM HEPES, NEAA, Na Pyruvate, and 400 μg/ml G418 (Invitrogen) at a density of 20,000 cells per well in a 96-well white flat bottom plate (Costar, catalog #3917) and placed in a humidified incubator maintained at 37° C. with 5% C02. Cells were treated sequentially with a dose response of antibodies, then CB1 receptor ligands at concentrations of 100 nM WIN 55,212, 10 μM anandamide or 500 nM 2-AG (Sigma), then lastly with 35 μM forskolin (Sigma) in the assay buffer of DMEM with 0.1% BSA. After a 30 minute incubation, d2-cAMP and Eu3+-Cryptate-cAMP antibody (Cisbio, catalog #62AM4PEC) were added, followed by incubation at room temperature for 1 hour. Wavelengths at 665 nm and 620 nm were measured using the EnVision Multilabel Reader and the 665/620 ratio was calculated. The graphs were generated by plotting the concentration of CB1 antibody on the x-axis, against the average of the two replicate values for cAMP levels (665/620 ratio). The data points were then fit using a log (agonist) versus response, variable slope (4 parameters) in GraphPad Prism 7.04, to obtain values for IC50. The results are shown in Tables 15C and 15D and FIG. 5A.

Inverse agonist activity. Inverse agonist activity of exemplary 10D10 variants was also measured. CHOK1 human CB1 receptor cells were plated overnight in DMEM with 0.5% FBS, P/S glutamine, 25 mM HEPES, NEAA, Na Pyruvate, and 400 μg/ml G418 at a density of 20,000 cells per well in a 96-well white flat bottom plate and placed in a humidified incubator maintained at 37° C. with 5% CO2. Cells were treated sequentially with a dose response of antibodies and then with 2 μM forskolin in the assay buffer of DMEM with 0.1% BSA. After a 30 minute incubation, d2-cAMP and Eu3+-Cryptate-cAMP antibody were added, followed by incubation at room temperature for 1 hour. Wavelengths at 665 nm and 620 nm were measured using the EnVision Multilabel Reader and the 665/620 ratio was calculated. The graphs were generated by plotting the concentration of CB1 antibody on the x-axis, against the average of the two replicate values for cAMP levels (665/620 ratio). The data points were then fit using a log (agonist) versus response, variable slope (4 parameters) in GraphPad Prism 7.04, to obtain values for IC50. The results are shown in Table 15C and FIG. 5B.

TABLE 15C
IC50 values of exemplary variants in
antagonist and inverse agonist assays

| | | | | Inverse agonist assay |
|---|---|---|---|---|
| | Antagonist assay IC50 (nM) | | | IC50 (nM) |
| Name | CP 55,940 | 2-AG | Anandamide | — |
| Rimonabant | 2.8 | 2.6 | 62.5 | 44.2 |
| 10D10 | 120.3 | 177.8 | 6044.0 | 294.9 |
| LIBC528116-1 | 1.8 | 7.2 | 17.0 | 31.7 |
| LIBC523797-1 | 3.4 | 2.9 | 10.4 | 14.1 |

TABLE 15D
IC50 of exemplary variants and a benchmark
antibody an antagonist assay

| Name | IC50 (nM) |
|---|---|
| LIBC527814-1 | 3.8 |
| LIBC528116-1 | 3.4 |
| 10D10 | 143.8 |
| Benchmark mAb* | 95.8 |

*a benchmark mAb in the clinic

Potency of the affinity matured antibodies (both cognate and LC-swapped affinity matured antibodies) were analyzed using in vitro cAMP assays and the results are summarized in Tables 15A-15D and FIGS. 5A and 5B. Most variants from the affinity maturations were more potent than 26970-1, a 10D10 variant that was obtained in previous efforts and used as a benchmark in the assays (Tables 15A and 15B). 26970-1 contains HC D83K/LC N35Y substitutions and was the best available benchmark antibody. Some affinity matured variants exhibited similar or slightly better potency than rimonabant in multiple runs of the functional assay. The most potent antibodies were about 30-66× and 3.6-8× more potent than 10D10 and 26970-1, respectively (Table 15A). The LIBC528116-1 antibody was consistently the best antagonist in the CP 55,940 assay (Tables 15A-15C), and the LIBC523797-1 antibody was the most potent in assays run in the presence of endogenous CB1 receptor agonists 2-AG and anandamide (Table 15C). In addition, the LIBC523797-1 antibody was the most superior inverse agonist (Table 15C), its potency was equivalent to rimonabant in the 2-AG assay and 3-4× more potent in the anandamide and inverse agonist assays (Table 15C). Finally, the most potent 10D10 variants were shown to be ˜30× more potent than a benchmark anti-CB1 antibody in the clinic when the antibodies were tested side-by-side (Table 15D). The potency results demonstrated that the affinity maturation efforts achieved the lofty design goal of obtaining antibodies with potency in the single digit nM range, a goal that had remained elusive for many years.

### Binding Affinity

Affinity assay using Octet. The Octet@HTX (Sartorius, Germany) instrument was used to determine affinities of anti-CB1 antibodies. Prior to experiment, Streptavidin (SA) biosensors (ForteBio, #18-5021) were pre-hydrated for 10 min in Octet assay buffer containing 10 mM Tris (Fisher, #BP152-1), 150 mM NaCl (Fisher, #5271-10), 1 mM CaCl₂) (Fisher, #BP510-500), 0.1 mg/mL BSA (BioShop, #ALB001.1), and 0.1% Triton-X 100 (Calbiochem, #9410-OP), pH7.4. SA biosensors were loaded with biotinylated anti-human Fc antibody (Invitrogen, #A18827) followed by one baseline step of 60 s in Octet assay buffer. Anti-CB1 antibodies were prepared in Octet assay buffer and captured by the anti-human Fc antibody prior to being submerged in wells containing different concentrations of CB1-nanodisc (#CB1-008, in-house) for 5 min, followed by 10 min dissociation. CB1-nanodisc was titrated 1 to 2 from 400 nM to 6.25 nM and prepared in Octet assay buffer. SA biosensors were used once without regeneration. For data evaluation, ForteBio Data Analysis v11.0 software was used. The kinetic rate constants, association rate constant (ka, M−1s−1), dissociation rate constant (kd, s−1), and the equilibrium rate constant (KD, M) were determined by using a 1:1 Langmuir model.

On-cell ranking using KinExA The KinExA® 3200 (Sapidyne, Boise, ID) instrument was used to rank binding of anti-CB1 antibodies to full length CB1 (DNA391968-1, in-house) transiently expressed on HEK 293T cells. Prior to the on-cell ranking experiment, on-cell affinity was determined to a benchmark anti-CB1 antibody, LIBC523661. The affinity experiment was performed with HEK 293T CB1 cells one day post transfection and titrated 1 to 3 from 2.5 million cells/mL to ˜51 cells/mL. The HEK 293T CB1 cells were equilibrated with a final antibody concentration (binding arm concentration is two times the antibody concentration) of either 5 μM, 100 μM, or 1 nM of LIBC523661 in cell culture media containing FreeStyle 293T culture media (Gibco, #12338-018), 2% Ultra Low IgG FBS (Invitrogen, #16250-078), 50 μg/mL G418 (Sigma, #G8168), and 0.05% w/v/sodium azide (Sigma, #S2002), for 24 hours at RT. Cells were separated by centrifugation and the free antibody in the supernatant at equilibrium was collected and measured by KinExA® technology by passing the supernatant over PMMA beads (Sapidyne) pre-coated with goat anti-human Fc (Invitrigen, #31125) and detected with goat anti-human IgG (H+L) AlexFluor649 (Jackson Immunoresearch, #109-605-088). The KD was obtained from nonlinear regression of the curves using a one site homogeneous binding model provided in the KinExA® Pro software Version 4.2.12. The KD for LIBC523661 was determined to be 64.0 μM with 95% CI 46.6-88.2 μM and receptor number per cell equal to 1.1 million copies/cell. It was determined using the KD-control curve that ˜12 thousand cells/mL are required to achieve 50% KD for LIBC523661. Therefore, on-cell ranking of anti-CB1 antibodies was performed using 20 μM of each antibody with 12 thousand cells/mL. Anti-CB1 antibodies were prepared in 293 culture media without cells or with HEK 293T CB1 cells and incubated for 24 hours at RT. Similarly, to affinity experiment, cells were separated by centrifugation and free antibody in supernatant was collected and measured by KinExA® technology. The binding of each anti-CB1 antibody is reported as percent inhibited free fraction (% IFF) with is calculated as antibody signal obtained in presence of 12 thousand cells/mL minus signal from blank divided by antibody signal obtained in the absence of cells minus signal from blank (the lower the % IFF, the higher the affinity, the higher the % IFF, the lower the affinity). Relative binding of all anti-CB1 antibodies was compared to LIBC523661 by taking the % IFF of LIBC523661 divided by the % IFF of antibody.

Octet and Kinexa binding assays of the affinity matured 10D10 variants confirm that potency enhancements came with improvements in binding affinities to huCB1 (Table 16). Octet revealed that faster on-rates and slower off-rates contributed to 10-50× higher huCB1-ND binding affinities vs 10D10, whereas Kinexa validated binding improvements to wt huCB1 expressed on cells.

TABLE 16
Octet and Kinexa of exemplary variants

| | | | | KinExA* | |
|---|---|---|---|---|---|
| | Octet (nanodisc) | | | (on cell) | IC50, nM |
| | k on, | k off, | KD, | Relative | (CP |
| Name | M⁻¹s⁻¹ | s⁻¹ | nM | binding* | agonist) |
| Benchmark# | 9.46E+03 | 1.25E−02 | 1326 | 0.5 | 95 |
| 10D10 | 1.64E+3 | 1.25E−02 | 755 | 0.5 | 121 |
| 26970-1 | 7.33E+04 | 5.53E−03 | 76 | 0.5 | 14 |
| LIBC527814 | 5.66E+04 | 1.98E−03 | 35 | 1.9 | 2.1 |
| LIBC528116 | 4.31E+04 | 1.23E−03 | 29 | 2.0 | 1.8 |
| LIBC523797 | 5.15E+04 | 9.43E−04 | 18 | 2.1 | 3.4 |
| LIBC523661 | 3.57E+04 | 2.75E−03 | 77 | 1.0 | 3.9 |
| LIBC524049 | 5.90E+04 | 9.04E−04 | 15 | 1.5 | 3.7 |
| LIBC527997 | 5.18E+04 | 1.36E−03 | 26 | 2.1 | 2.5 |
| LIBC527919 | 5.62E+04 | 3.39E−03 | 60 | 2.9 | 3.0 |

*Calculated from fraction of free mAb vs LIBC523661 mAb
#a benchmark mAb in the clinic

### Example 6. Improved Expression of the Affinity Matured Variants

The cognate and LC-swapped affinity matured variants were tested for transient and stable expression in HEK293 and CHO cells. Four out of the five most potent cognate 10D10 variants expressed transiently in HEK 293-6E cells at 100-200 mg/L (Table 17), representing >3× or >7× higher titer than 10D10 or a benchmark variant. Examination of the LC sequences suggests that reduction of hydrophobicity at key positions (especially L58Q) could be tied to improved expression titer (Table 17). The most potent LC-swapped 10D10 HC variants expressed transiently at 15-80 mg/L and expressed well at >670 mg/L in stable production runs from CHO-K1.

TABLE 17
Improved expression of exemplary
cognate affinity matured variants

| Name | Harvest (mg/L) |
|---|---|
| 10D10 | 34.6 |
| 10D10 HC D83K/LC | 14.4 |
| N35Y | |
| LIBC524049-1 | 26.2 |
| LIBC523797-1 | 91.7 |
| LIBC523661-1 | 177.1 |
| LIBC523844-1 | 100.9 |
| LIBC523969-1 | 121.9 |

### Example 7. Effects of Affinity Matured Antibodies in Animal Models

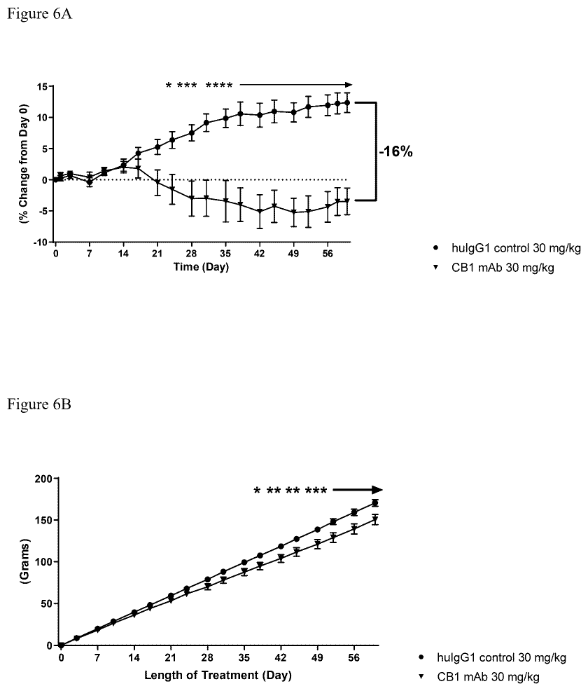

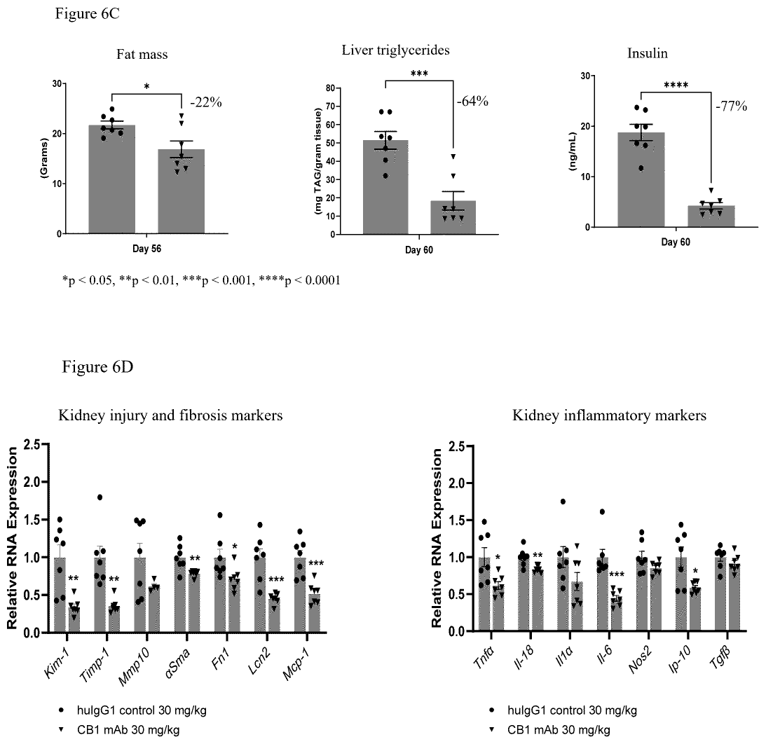

The LIBC528116-1 (37940-4) antibody in SEFL2 format (See Jacobsen F. W. et al., J Biol Chem. 2017 Feb. 3; 292(5): 1865-1875) was scaled up for testing in diet-induced obese (DIO) transgenic mice with huCB1 donor DNA knocked in using CRISPR-Cas9 technology to mediate the replacement of mouse Cnr1 coding sequence with the human CNR1 counterpart. Specifically, 6- to 10-week-old huCB1 knock in (KI) mouse was fed with high fat diet (HFD, Research Diets D12492 60% kcal from fat) for 13 weeks to induce obesity. The LIBC528116-1 antibody and a huIgG1 control antibody were administered to the mice at 30 mg/kg weekly (QW) for 60 days (n=7 mice/group), and the body weight and food intake were measured every 3-4 days. Total fat mass was measured by MRI on day 58 of treatment, and triglyceride and insulin levels of the animals were analyzed and the results are shown in Table 18 and FIGS. 6A-6C. At day 60, the affinity-matured 10D10 variant reduced body weight by 15.9% vs huIgG1 control, and reduced fat mass by 22%, liver weight by 47%, liver triglycerides by 64%, and plasma insulin by 77% (Table 18, FIGS. 6A and 6C). By contrast, an earlier study with the D83K/N35Y variant of 10D10 (26970-2) resulted in no impact on weight gain compared to isotype control. These results showed that potent peripherally-acting anti-CB1 antibody descried herein was an effective treatment for anti-obesity, hyperinsulinemia, and non-alcoholic steatohepatitis (NASH).

TABLE 18
Impact of affinity matured antibody on body weight,
fat mass and liver weight in huCB1 KI mouse

| | | | | BW % | BW % | | Liver Weight |
|---|---|---|---|---|---|---|---|
| | IC50 | | BW % | Change | Change | Fat Mass | % Change |
| Name | (nM) | Mouse Model | Change | Day 45 | Day 60 | % Change | (Day) |
| Rimonabant | 2.3 | JAX WT DIO | −12.9% | N/A | N/A | −23% | −7% |
| 3 mg/kg (6.5 | | (12 weeks | (day 17) | | | (Day 15) | (Day 17) |
| μmol/kg, QD) | | HFD) | | | | | |
| 26970-2 mAb | 9.5 | huCNR1 TG | −1.9% | N/A | N/A | −2% | −19% |
| 30 mg/kg | | (12 weeks | (Day 31-32) | | | (Day 30) | (Day 32) |
| (0.2 μmol/kg, | | HFD) | | | | | |
| QW) | | | | | | | |
| 37940-4 mAb | 2.7 | huCNR1 TG | −12.1% | −15.2% | −15.9% | −22% | −47% |
| 30 mg/kg | | (13 weeks | (Day 31-32) | | | (Day 58) | (Day 60) |
| (0.2 μmol/kg, | | HFD) | | | | | |
| QW) | | | | | | | |

Fibrosis and inflammatory markers in kidney were also measured in the animals. Specifically, RNA was extracted and isolated from mouse kidney tissue via TRIzol extraction. 50-100 mg of tissue and a 5 mm stainless steel bead (Qiagen, #69989) was placed in a microcentrifuge tube with 1 mL of TRIzol (Ambion by life technologies, #15596018). The TissueLyser II machine was used at frequency (1/S) 30.0 for 3 minutes and repeated once. Lysates were then incubated for 5 minutes followed by a quick spin down. 200 μL of chloroform (Sigma-Aldrich, #372978-100 mL) was then added to each tube and vortexed for 30 seconds. The tubes were then incubated for 5 minutes before spinning down the tubes at 4° C. at 12,000 g for 15 minutes. The top aqueous layer was removed and placed into a new deep well plate and mixed with equal parts 70% EtOH. Upon mixing, the solution was transferred to the RNA Plate from the RNeasy Plus 96 Kit and RNA isolation was done per kit instructions (Qiagen, #74192) Qiagen DNase step was performed per kit instructions (Qiagen, #79254). RNA was diluted in RNase/DNase free water and the concentration of RNA was confirmed via NanoDrop 8000. RNA was then diluted to 50 ng/8.5 uL of water to be then run on Quantstudio 7 Flex Real-Time PCR system. TaqMan RNA- to Ct one-Step Kit (Applied Biosystems, #4392938) was used per instruction with TaqMan gene primer probe sets. CT values were used to calculate relative RNA expression levels. Treatment with the anti-CB1 antibody reduced the expression levels of the fibrosis and inflammatory markers in kidney compared to control IgG (FIG. 6D).

### Example 8. New Xenomouse Antibody Campaign

Mouse immunization: Several xenomouse groups were immunized with different combinations of immunogens. Immunogens were: Plasmid DNA encoding full length huCB1 with E3k epitope for T-cell activation. DNA was coated on gold particles and delivered intradermally; HEK293T cells transiently transfected with full length huCB1 with E3k epitope. Cells were delivered subcutaneously; and Nanoparticles (nanodiscs) containing huCB1 protein engineered for enhanced stability. Nanoparticles were delivered subcutaneously.

Immunizations took several months; blood samples were collected from animals and CB1 antibody titers measured periodically. Because previous experience and published CB1 structures suggested that large N-terminal domain of CB1 was likely to be irrelevant for drug function, only animals showing titers to both full length and truncated CB1 (i.e., devoid of the 1-98 aa N-term) were chosen for harvest to improve chances of finding functional antibodies during screening. Titer measurements were done by FACS on CHO—S cells transiently transfected with full length or truncated huCB1 or an empty vector.

Plasma B cell screening: Traditional hybridoma generation relies on immortalization of B cells by means of fusion with myeloma cells. Because of low fusion efficiency, valuable B cell clones can be difficult to recover through this process if they are rare. To complement hybridoma generation, direct screening of antibody secreting plasma B cells was implemented. The latter approach focuses on memory B cells and was used to increase the chance of finding valuable clones by sampling a different cell population.

B cells for screening were obtained from spleen, lymph nodes and bone marrow of sacrificed animals. Plasma B cells were separated from memory B cells according to CD138 cell surface marker expression using a commercial CD138 enrichment kit (Stemcell Technologies 17877). CD138+ cells were then combined with HEK293T cells transfected with truncated huCB1 and HEK293T cells transfected with an empty vector. The three types of cells were combined together in B cell media, and then mixed with a warm agarose solution (Sigma A5030, 1% final concentration), placed into fluorinated oil (RAN Biotechnologies 008-FluoroSurfactant-2 wtH-50G) and emulsified by vortexing. The result was a medium-in-oil emulsion that contained microdroplets with cells trapped in them. After a 1 h incubation and a 10 min cooldown down, the agarose microgels were extracted from oil, washed with 1% FBS in PBS and screened using fluorescently labeled anti-human antibodies as a secondary detection reagent (Jackson 109-545-098). Microgels were examined under a fluorescence microscope and those containing fluorescent signal were manually picked using an Eppendorf micromanipulator. Isolated microgels were placed in individual PCR tubes with lysis buffer and subjected to RT-PCR reaction to rescue antibody sequences from B cells trapped in microgels for cloning and expression.

Molecular rescue and sequencing of CB1 antibodies from B cells: RNA (total or mRNA) was purified from samples containing CB1 antibody-producing single B cells using Agencourt RNA Clean XP magnetic beads. Purified RNA was used as template in a template-switching PCR to generate first strand cDNA via reverse transcription, followed by cDNA amplification. The cDNA was cleaned up using Agencourt AMPure XP PCR clean up kit and used as a template to amplify the antibody heavy and light chain variable region (V) genes. The fully human antibody gamma heavy chain was obtained using the cDNA as template and amplifying the variable region of the gamma heavy chain using multiplex PCR. The 5′ gamma chain-specific primer annealed to the signal sequence of the antibody heavy chain, while the 3′ primer annealed to a region of the gamma constant domain. The fully human antibody kappa light chain was obtained using the cDNA as template and amplifying the variable region of the kappa light chain using multiplex PCR. The 5′ kappa light chain-specific primer annealed to the signal sequence of the antibody light chain while the 3′ primer annealed to a region of the kappa constant domain. The fully human antibody lambda light chain was obtained using the cDNA as template and amplifying the variable region of the lambda light chain using multiplex PCR. The 5′ lambda light chain-specific primer annealed to the signal sequence of light chain while the 3′ primer annealed to a region of the lambda constant domain. The primers used in the multiplex PCR reactions also have overhangs that facilitate Golden Gate cloning into an expression vector. The amplicons from the multiplex PCR reactions were purified using Agencourt AMPure XP PCR clean up kit. The clean PCR amplicons were then cloned into an expression vector using standard Golden Gate cloning methods followed by transformation into chemically competent E. coli bacterial cells. Bacterial colonies from the transformations were cultured and then sequenced directly. Amino acid sequences were deduced from the corresponding nucleic acid sequences bioinformatically. The derived amino acid sequences were then analyzed to determine the germline sequence origin of the antibodies and to identify deviations from the germline sequence.

Hybridoma generation and screening: hybridoma was generated by immortalization of B cells with mouse myeloma cells using standard technique. Hybridomas were first plated at a density of several clones per well and secreted antibodies were screened for binding to both full length and truncated CB1 transiently expressed on HEK293T or CHO—S cells. Polyclonal wells showing CB1 binding were single-cell sorted to obtain individual antibody-secreting clones.

A total number of 1325 Xenomouse clones were screened, of which 71 bound to both full length and truncated huCB1. Most of the 71 clones were non-functional and only 9 clones met potency standards set out for the mouse campaign. Three unique antibodies were isolated from the nine clones. The sequences of the antibodies (Tables 19 and 20) are similar to the sequence of 10D10. Notably, the Y57H mutation in HCDR2 that emerged after affinity maturation antibodies is also present in these antibodies (Table 19).

TABLE 19
CDR sequence of antibodies from the mouse campaign

| | Seq | | Seq | | Seq | |
|---|---|---|---|---|---|---|
| Name | No. | CDR1 | No. | CDR2 | No. | CDR3 |
| LIBC529593-1 | 471 | RSSQSLLHRSGYNYLD | 472 | LGSNRAS | 473 | MQSLOTPRT |
| LV | | | | | | |
| LIBC529593-1 | 474 | RGGDYWS | 475 | HIYYSGSTNYNPSLRS | 476 | DYDILTGYSYYYYGLDV |
| HV | | | | | | |
| LIBC560340-1 | 477 | RSSQSLLYSNGHNFLD | 478 | LGSNRAS | 479 | MQALQTPRT |
| LV | | | | | | |
| LIBC560340-1 | 480 | RGGDYWN | 481 | HIYYSGSKNYNPSLKS | 482 | GYDSSGYSYYYYGMDV |
| HV | | | | | | |
| LIBC560657-1 | 483 | RSSQSLLYSNGHNYLD | 484 | LGSNRAP | 485 | MQALQTPRT |
| LV | | | | | | |
| LIBC560657-1 | 486 | RGGDYWN | 487 | HIYYTGTKYYNPSLKS | 488 | GYDSSGYSYYYYGMDV |
| HV | | | | | | |
| 10D10 LV | 91 | RSSQSLLHSNGYNYLD | 92 | LGSNRAS | 93 | MQALQTPRT |
| 10D10 HV | 94 | RGGDYWS | 95 | YIYYSGSTNYNPSLKS | 96 | DYDILTGYSYYYYGMDV |

TABLE 20
Variable region sequence of antibodies from the mouse campaign

| | Seq | | Seq | |
|---|---|---|---|---|
| Name | No | HC HV | No. | LC LV |
| LIBC529593- | 489 | QVQLQESGPGLVKPSQTLSLTCTVSGGSIRRGGDY | 490 | DIVMTQSPLSLPVTPGEPASISCRSSQSLLHRSGY |
| 1 | | WSWIRQHPGKGLEWIGHIYYSGSTNYNPSLRSRVRI | | NYLDWYLQKPGQSPQLLIYLGSNRASGVPDRFS |
| | | SVDTSKNQFSLKLSSVTAADTAVYYCARDYDILTGYS | | GSGSGTDFTLKISRVEAEDVGVYYCMQSLQTPR |
| | | YYYYGLDVWGQGTTVTVSS | | TFGQGTKVEIKR |
| LIBC560340- | 491 | QVQLQESGPGLVKPSQTLSLICIVSGGSIRRGGDYW | 492 | DIVMTQSPLSLPVTPGEPASISCRSSQSLLYSNGH |
| 1 | | NWIRQHPGKGLEWIGHIYYSGSKNYNPSLKSRVTIS | | NFLDWYLQKPGQSPQLLIYLGSNRASGVPDRFS |
| | | VDTSKNQFSLKLSSVTAADTAVYYCARGYDSSGYSY | | GSGSGTDFTLKISRVEAEDVGVYYCMQALQTPR |
| | | YYYGMDVWGQGTTVTVSS | | TFGGGTKVEIKR |
| LIBC560657- | 493 | QVQLRESGPGLVKPSQTLSLICIVSGGSIRRGGDYW | 494 | DIVMTQSPLSLPVTPGEPASISCRSSQSLLYSNGH |
| 1 | | NWIRQHPGKGLEWIGHIYYTGTKYYNPSLKSRVTIS | | NYLDWYLQKPGQSPQLLIYLGSNRAPGVPDRFS |
| | | VDTSKNQFSLKLSSVTAADTAVYYCARGYDSSGYSY | | GSGSGTDFTLKISRVEAEDVGVYFCMQALQTPR |
| | | YYYGMDVWGQGTTVTVSS | | TFGGGTKVEIKR |
| 10D10 | 127 | QVQLQESGPGLVKPSQTLSLTCTVSGGSIRRGGDY | 128 | DIVMTQSPLSLPVTPGEPASISCRSSQSLLHSNGY |
| | | WSWIRQHPGKGLEWIGYIYYSGSTNYNPSLKSRATI | | NYLDWYLQKPGQSPQLLIYLGSNRASGVPDRFS |
| | | SVDTSKNQFSLKLSSVTAADTAVYYCARDYDILTGYS | | GSGSGTDFTLKIRRVEAEDVGVYYCMQALQTPR |
| | | YYYYGMDVWGQGTTVTVSS | | TFGQGTKVEIKR |

Antibody characterization: Antibody clones identified above were tested for binding to full length human CB1 and N-term truncated human CB1 to confirm that antibodies bind to extracellular loops ECL1-3 and not to the CB1 N-terminus. Binding to human CB2 was also tested to make sure that there would be no side effects due to cross-reactivity to CB2. Finally, binding to mouse CB1 was tested. Following binding studies, functional screening using a TR-FRET cAMP assay was performed to measure antibody inhibitory effect on CB1.

To assess target specificity, binding was measured by FACS on HEK293T cells transiently transfected with the protein of interest and compared to nonspecific binding to cells transfected with an empty vector. Binding results are presented as ratios of Mean Fluorescence Intensities of target- and mock-transfected cells. The results showed that all three antibodies bound to full length and truncated human CB1, but not to human CB2 or murine CB1 (Table 21).

The inhibitory effect of the antibodies on CB1 function was assessed using a Time-Resolved Forster Resonance Energy Transfer (TR-FRET) cAMP assay. Antibodies that inhibit CB1 cause an increase of cAMP which, in turn, results in a decrease of FRET signal in the assay. Hence, lower assay signal means better inhibition of CB1 by the antibody. To improve assay sensitivity CP55,940 and Forskolin were used to activate cellular CB1 and adenylyl cyclase, respectively. Rimonabant was used as a positive control.

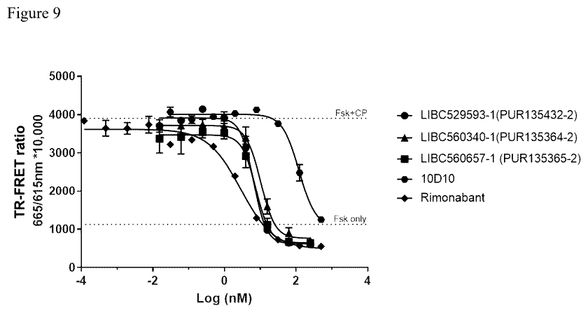

Briefly, stably transfected CB1 CHO cells were cultured in DMEM (Sigma Cat #D5671) with 0.5% FBS, 25 mM HEPES overnight at 37° C./5% CO2. On the day of assay, CB1 antibodies and Rimonabant were titrated 1 in 4 at 4 times final concentration using DMEM (Sigma Cat #D5671) with 0.1% BSA (from PE kit), 25 mM HEPES, the titrated antibodies were transferred to the 384 well assay plate at a volume of 7.5 μL per well. The cells were harvested using Accutase (Sigma) and neutralized with DMEM (Sigma Cat #D5671) with 0.5% FBS, 25 mM HEPES. These cells were pelleted and resuspended to 1.33×106 cells/mL using DMEM (Sigma Cat #D5671) with 0.1% BSA (from PE kit), 25 mM HEPES. The anti cAMP antibody from the Lance Ultra cAMP Detection kit (Perkin Elmer; TRF0263) was added to the cells at a 1 in 150 ratio of antibody to cell volume, 3-Isobutyl-1-methylxanthine (IBMX; Sigma) was also added to the cells at a final concentration of 1 mM. The cells were transferred to the assay plate at a volume of 15 μL per well or 20,000 cells per well. Forskolin and CP55,940 cocktail was diluted to 4 times final concentration using DMEM (Sigma Cat #D5671) with 0.1% BSA (from PE kit), 25 mM HEPES and transferred to the assay plate at a volume of 7.5 μL per well. The assay plate was incubated at 37° C./5% CO2 for ½ hour before EU-cAMP tracer (Perkin Elmer; TRF0263) was added to each well and further incubated at room temperature protected from light for 1 hour. After the final incubation, the assay plates were read using Envision plate reader with EU615 and APC 665 Emission filters. The data was analyzed using calculation of: (665 nm/615 nm)×10,000. Lower value (lower FRET) indicated stronger inhibition of CB1 and the data was used to calculated IC50. The results showed that all three antibodies were much superior to 10D10 in terms of potency (Table 22 and FIG. 9).

TABLE 21
Binding data of the antibodies

| | Antibody binding, MFI ratio | | |
|---|---|---|---|
| Target | LIBC529593 | LIBC560340 | LIBC560657 |
| Full length hu CB1 | 422 | 578 | 440 |
| Truncated hu CB1 | 495 | 705 | 444 |
| Full length hu CB2 | 1.1 | 1.0 | 1.0 |
| Full length mu CB1 | 1.0 | 1.0 | 1.0 |

TABLE 22
Potency data of the antibodies

| Mole- | | | | | Rimona- |
|---|---|---|---|---|---|
| cule | LIBC529593 | LIBC560340 | LIBC560657 | 10D10 | bant |
| IC50, | 6.4 | 10.2 | 7.5 | 120 | 2.8 |
| nM | | | | | |

### Example 9. Further Affinity Maturation of Anti-CB1 Antibodies

Another round of affinity maturation was carried out to further improve functional potency. Analyses of the affinity matured antibodies from the previous round (Examples 1-3 above) showed that there was good correlation between GFP-tagged huCB1-SMALP binding and IC50 for the cognate 10D10 variants, and huCB1-GFP-SMALP binding predicted a majority of the most potent LC-swapped variants. Thus, improving binding to huCB1-GFP-SMALP was the focus during screening and sorting in this round of affinity maturation to obtain antibodies with subnanomolar potencies.

Two strategies were used in this affinity maturation. First, the VK1 LC shared by the top LC-swapped variants fortuitously belongs to a well-studied germline where there is high confidence in the LCDR positions predicted to be solvent-exposed and tolerant to mutation. Thus, to further optimize their LCs we sought to pair the HCs of LIBC528116-1 and other top LC-swapped variants with VK1/O2 LCDR libraries previously utilized in internal affinity maturation projects. While the co-crystal structure suggests that mutations at solvent-exposed LC positions are unlikely to create contacts with huCB1, it is hypothesized that LC mutations may provide more optimal structural support of HC loop conformations to enhance binding and potency. To increase selection stringency, monovalent yeast-display Fab libraries were constructed and sorted/screened for improved binding against wtCB1-SMALP while retaining huCB1-ND binding (vs LIBC58116-1 Fab).

Second, we leveraged the surprising discovery of more 10D10-like antibodies from the new mouse campaigns (see Example 8) to utilize better starting points for affinity maturation. The antibodies from the new mouse campaign indicated certain positions in HCDR2 and HC FR3 could be further explored to improve binding and potency. In addition, the new antibodies cluster into two highly-related HCDR3s that differ in length by one amino acid (e.g., LIBC560657-1 and LIBC529593-1 of Example 8) indicating that the length of HCDR3 could be varied. Yeast display libraries for further cognate affinity maturation merged HC and LC mutations from the most potent cognate 10D10 variants from Example 2 into the LIBC529593-1 sequence context and incorporated both HCDR3 lengths. HC library 2 incorporated the shorter HCDR3 sequence and additionally contained fixes at HCDR3 position 112 for the predicted DS isomerization site. The design of the further cognate affinity-maturation libraries is summarized in Tables 23-25.

TABLE 23
Design of 10D10-like HC library 1 for further cognate AM*

| 10D10 residue | Location | Diversification strategy |
|---|---|---|
| Y57 | HCDR2 | H^{#} |
| I58 | HCDR2 | I, V |
| Y60 | HCDR2 | Y, Q |
| S65 | HCDR2 | E, A, K, T |
| S67 | HCDR2 | S, Q |
| N69 | HCDR2 | E, A, K, T |
| S73 | HCDR2 | S, N |
| L74 | HCDR2 | L, F |
| K75 | HCDR2 | K, R^{#} |
| S76 | HCDR2 | S, G, N, D |
| A78 | HFR3 | V^{#} |
| T79 | HFR3 | T, R^{#} |
| D83 | de loop (HFR3) | N |
| S85 | de loop_(HFR3) | Y |
| M136 | HCDR3 | M, L^{#} |

^{#}mutation found in LIBC529593-1
*Theoretical combinatorial diversity: 1.6E4

TABLE 24
Design of 10D10-like HC library 2 for further cognate AM*

| 10D10 | | Diversification | 10D10 | | Diversification |
|---|---|---|---|---|---|
| residue | location | strategy | residue | location | strategy |
| Y57 | HCDR2 | H^{#} | A78 | HFR3 | V# |
| I58 | HCDR2 | I, V | T79 | HFR3 | T, R# |
| Y60 | HCDR2 | Y, Q | D83 | de loop | N |
| | | | | (HFR3) | |
| S65 | HCDR2 | E, A, K, T | S85 | de loop | Y |
| | | | | (HFR3) | |
| S67 | HCDR2 | S, Q | D109 | HCDR3 | G |
| N69 | HCDR2 | E, A, K, T | I112 | HCDR3 | I, A, T, V |
| S73 | HCDR2 | S, N | L113 | HCDR3 | deletion |
| L74 | HCDR2 | L, F | T114 | HCDR3 | S |
| K75 | HCDR2 | K, R^{#} | M136 | HCDR3 | M, L# |
| S76 | HCDR2 | S, G, N, D | | | |

#mutation found in LIBC529593-1
*Theoretical combinatorial diversity: 6.5E4

TABLE 25
Design of 10D10-like LC library for further cognate AM*

| 10D10 | | Diversification |
|---|---|---|
| residue | location | strategy |
| S34 | LCDR1 | R^{#} |
| N35 | LCDR1 | S^{#} |
| L58 | LCDR2 | L, Q |
| R94 | LC FR3 | S^{#} |
| M107 | LCDR3 | R |
| A109 | LCDR3 | A, S^{#} |
| L110 | LCDR3 | V, R |
| Q111 | LCDR3 | A, G |
| T135 | LCDR3 | I, L |

^{#}mutation found in LIBC529593-1
*Theoretical combinatorial diversity: 33 (32 + 1)

The 10D10 HC and LC library designs were combinatorially paired to construct Fab-yeast libraries to select for increased binding stringency. The cognate libraries for further affinity maturation were sorted/screened for improved binding against wtCB1-SMALP while retaining huCB1-ND binding (vs LIBC523797-1 Fab).

The libraries constructed for both further affinity maturation campaigns were sorted against successively lower concentrations of huCB1-GFP-SMALP, starting at 25 or 50 nM. We conducted clonal binding screens at huCB1-SMALP concentrations as low as 2-5 nM, representing a 20-50× lower concentration than what was employed in the previous affinity maturation campaign (Examples 2-4). The most improved binders exhibiting minimal non-specific binding and predicted sequence liabilities from the further VK1 LC optimization campaign and the further cognate affinity maturation campaign were then selected based on similar principles as described before (Examples 2 and 3). Eight clones were obtained from the VK1 LC optimization, and 18 clones were obtained from the further cognate affinity maturation. These top clones showed improved huCB1-SMALP binding, similar huCB1-ND binding, and low levels of empty-ND binding versus the LIBC528116-1 and LIBC523797-1 Fab-yeast benchmarks from both LC-swapped and cognate campaigns, respectively.

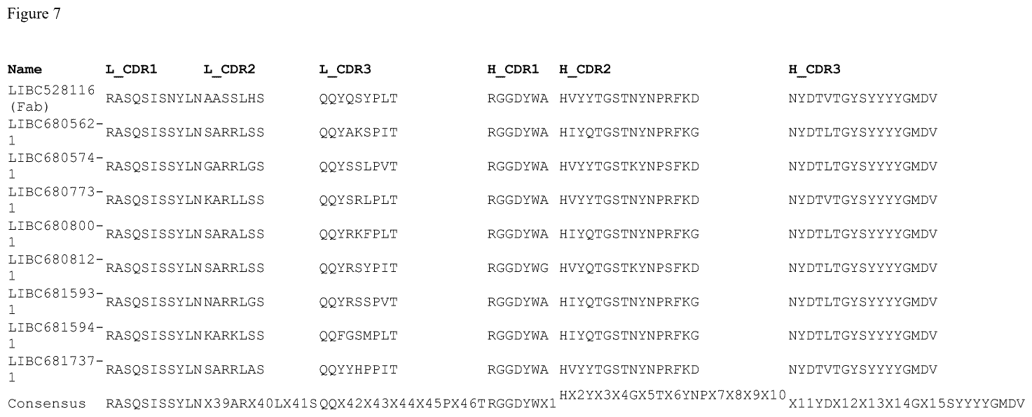

CDR sequences, germline information and binding data of the top 8 further LC-swapped affinity maturation variants are summarized in Tables 26 and 27 and FIG. 7. All top clones showed improved binding against CB1-SMALP and CB1-ND (Table 28) while exhibiting minimal binding to irrelevant XCR1-SMALP and empty-ND. For LIBC681737-1, the LC P12S germline reversion mutation was included when making IgG1 SEFL2 mAb for functional testing.

TABLE 26
CDR sequence of top 8 further VK1 LC-optimized variants

| | Seq | | Seq | | Seq | |
|---|---|---|---|---|---|---|
| Name | No. | CDR1 | No. | CDR2 | No. | CDR3 |
| LIBC680812-1_LC huCB1 | 405 | RASQSISSYLN | 406 | SARRLSS | 407 | QQYRSYPIT |
| LV | | | | | | |
| LIBC680812-1_ | 408 | RGGDYWG | 409 | HVYQTGSTKYNPSFKD | 410 | NYDTLTGYSYYYYGMDV |
| mol:fdChaincf huCB1 | | | | | | |
| HV | | | | | | |
| LIBC680562-1_LC huCB1 | 411 | RASQSISSYLN | 412 | SARRLSS | 413 | QQYAKSPIT |
| LV | | | | | | |
| LIBC680562-1_ | 414 | RGGDYWA | 415 | HIYQTGSTNYNPRFKG | 416 | NYDTLTGYSYYYYGMDV |
| mol:fdChaincf huCB1 | | | | | | |
| HV | | | | | | |
| LIBC680800-1_LC huCB1 | 417 | RASQSISSYLN | 418 | SARALSS | 419 | QQYRKFPLT |
| LV | | | | | | |
| LIBC680800-1_ | 420 | RGGDYWA | 421 | HIYQTGSTNYNPRFKG | 422 | NYDTLTGYSYYYYGMDV |
| mol:fdChaincf huCB1 | | | | | | |
| HV | | | | | | |
| LIBC680574-1_LC huCB1 | 423 | RASQSISSYLN | 424 | GARRLGS | 425 | QQYSSLPVT |
| LV | | | | | | |
| LIBC680574-1_ | 426 | RGGDYWA | 427 | HVYYTGSTKYNPSFKD | 428 | NYDTLTGYSYYYYGMDV |
| mol:fdChaincf huCB1 | | | | | | |
| HV | | | | | | |
| LIBC680773-1_LC huCB1 | 429 | RASQSISSYLN | 430 | KARLLSS | 431 | QQYSRLPLT |
| LV | | | | | | |
| LIBC680773-1_ | 432 | RGGDYWA | 433 | HVYYTGSTNYNPRFKD | 434 | NYDTVTGYSYYYYGMDV |
| mol:fdChaincf huCB1 | | | | | | |
| HV | | | | | | |
| LIBC681594-1_LC huCB1 | 435 | RASQSISSYLN | 436 | KARKLSS | 437 | QQFGSMPLT |
| LV | | | | | | |
| LIBC681594-1_ | 438 | RGGDYWA | 439 | HIYQTGSTNYNPRFKG | 440 | NYDTLTGYSYYYYGMDV |
| mol:fdChaincf huCB1 | | | | | | |
| HV | | | | | | |
| LIBC681737-1 | 441 | RASQSISSYLN | 442 | SARRLAS | 443 | QQYYHPPIT |
| (LC:P12S)_LC huCB1 LV | | | | | | |
| LIBC681737-1(LC:P12S)_ | 444 | RGGDYWA | 445 | HVYYTGSTNYNPRFKD | 446 | NYDTVTGYSYYYYGMDV |
| mol:fdChaincf | | | | | | |
| huCB1 HV | | | | | | |
| LIBC681593-1_LC huCB1 | 447 | RASQSISSYLN | 448 | NARRLGS | 449 | QQYRSSPVT |
| LV | | | | | | |
| LIBC681593-1_ | 450 | RGGDYWA | 451 | HIYQTGSTNYNPRFKG | 452 | NYDTLTGYSYYYYGMDV |
| mol:fdChaincf huCB1 | | | | | | |
| HV | | | | | | |
| LIBC681737-1_LC huCB1 | 441 | RASQSISSYLN | 442 | SARRLAS | 443 | QQYYHPPIT |
| LV | | | | | | |
| LIBC681737-1_ | 444 | RGGDYWA | 445 | HVYYTGSTNYNPRFKD | 446 | NYDTVTGYSYYYYGMDV |
| mol:fdChaincf huCB1 | | | | | | |
| HV | | | | | | |

TABLE 27
Variable region sequence of top 8 further VK1 LC-optimized variants

| | Seq | | Seq | |
|---|---|---|---|---|
| Name | No. | LC LV | No. | mol:fdChaincf HV |
| LIBC680812- | 453 | KPGKAPKLLIYSARRLSSGVPSRFSGSGSGTDFTLTISSL | 454 | QVQLQESGPGLVKPSQTLSLTCTVSGGSIRRGGDYWGWIRQHP |
| 1 | | DIQMTQSPSSLSASVGDRVTITCRASQSISSYLNWYQQ | | GKGLEWIGHVYQTGSTKYNPSFKDRATISVNTYKNQFSLKLSSVT |
| | | QPEDFATYYCQQYRSYPITFGGGTKVEIKR | | AADTAVYYCARNYDTLTGYSYYYYGMDVWGQGTTVTVSS |
| LIBC680562- | 455 | DIQMTQSPSSLSASVGDRVTITCRASQSISSYLNWYQQ | 456 | QVQLQESGPGLVKPSQTLSLTCTVSGGSIRRGGDYWAWIRQHP |
| 1 | | KPGKAPKLLIYSARRLSSGVPSRFSGSGSGTDFTLTISSL | | GKGLEWIGHIYQTGSTNYNPRFKGRATISVNTYKNQFSLKLSSVT |
| | | QPEDFATYYCQQYAKSPITFGGGTKVEIKR | | AADTAVYYCARNYDTLTGYSYYYYGMDVWGQGTTVTVSS |
| LIBC680800- | 457 | DIQMTQSPSSLSASVGDRVTITCRASQSISSYLNWYQQ | 458 | QVQLQESGPGLVKPSQTLSLTCTVSGGSIRRGGDYWAWIRQHP |
| 1 | | KPGKAPKLLIYSARALSSGVPSRFSGSGSGTDFTLTISSL | | GKGLEWIGHIYQTGSTNYNPRFKGRATISVNTYKNQFSLKLSSVT |
| | | QPEDFATYYCQQYRKFPLTFGGGTKVEIKR | | AADTAVYYCARNYDTLTGYSYYYYGMDVWGQGTTVTVSS |
| LIBC680574- | 459 | DIQMTQSPSSLSASVGDRVTITCRASQSISSYLNWYQQ | 460 | QVQLQESGPGLVKPSQTLSLTCTVSGGSIRRGGDYWAWIRQHP |
| 1 | | KPGKAPKLLIYGARRLGSGVPSRFSGSGSGTDFTLTISSL | | GKGLEWIGHVYYTGSTKYNPSFKDRATISVNTYKNQFSLKLSSVT |
| | | QPEDFATYYCQQYSSLPVTFGGGTKVEIKR | | AADTAVYYCARNYDTLTGYSYYYYGMDVWGQGTTVTVSS |
| LIBC680773- | 461 | DIQMTQSPSSLSASVGDRVTITCRASQSISSYLNWYQQ | 462 | GKGLEWIGHVYYTGSTNYNPRFKDRATISVNTYKNQFSLKLSSVT |
| 1 | | KPGKAPKLLIYKARLLSSGVPSRFSGSGSGTDFTLTISSL | | QVQLQESGPGLVKPSQTLSLTCTVSGGSIRRGGDYWAWIRQHP |
| | | QPEDFATYYCQQYSRLPLTFGGGTKVEIKR | | AADTAVYYCARNYDTVTGYSYYYYGMDVWGQGTTVTVSS |
| LIBC681594- | 463 | DIQMTQSPSSLSASVGDRVTITCRASQSISSYLNWYQQ | 464 | QVQLQESGPGLVKPSQTLSLTCTVSGGSIRRGGDYWAWIRQHP |
| 1 | | KPGKAPKLLIYKARKLSSGVPSRFSGSGSGTDFTLTISSL | | GKGLEWIGHIYQTGSTNYNPRFKGRATISVNTYKNQFSLKLSSVT |
| | | QPEDFATYYCQQFGSMPLTFGGGTKVEIKR | | AADTAVYYCARNYDTLTGYSYYYYGMDVWGQGTTVTVSS |
| LIBC681737- | 465 | DIQMTQSPSSLSASVGDRVTITCRASQSISSYLNWYQQ | 466 | QVQLQESGPGLVKPSQTLSLTCTVSGGSIRRGGDYWAWIRQHP |
| 1(LC:P12S) | | KPGKAPKLLIYSARRLASGVPSRFSGSGSGTDFTLTISSL | | GKGLEWIGHVYYTGSTNYNPRFKDRATISVNTYKNQFSLKLSSVT |
| | | QPEDFATYYCQQYYHPPITFGGGTKVEIKR | | AADTAVYYCARNYDTVTGYSYYYYGMDVWGQGTTVTVSS |
| LIBC681593- | 467 | DIQMTQSPSSLSASVGDRVTITCRASQSISSYLNWYQQ | 468 | QVQLQESGPGLVKPSQTLSLTCTVSGGSIRRGGDYWAWIRQHP |
| 1 | | KPGKAPKLLIYNARRLGSGVPSRFSGSGSGTDFTLTISSL | | GKGLEWIGHIYQTGSTNYNPRFKGRATISVNTYKNQFSLKLSSVT |
| | | QPEDFATYYCQQYRSSPVTFGGGTKVEIKR | | AADTAVYYCARNYDTLTGYSYYYYGMDVWGQGTTVTVSS |
| LIBC681737- | 469 | DIQMTQSPSSLPASVGDRVTITCRASQSISSYLNWYQQ | 470 | QVQLQESGPGLVKPSQTLSLTCTVSGGSIRRGGDYWAWIRQHP |
| 1 | | KPGKAPKLLIYSARRLASGVPSRFSGSGSGTDFTLTISSL | | GKGLEWIGHVYYTGSTNYNPRFKDRATISVNTYKNQFSLKLSSVT |
| | | QPEDFATYYCQQYYHPPITFGGGTKVEIKR | | AADTAVYYCARNYDTVTGYSYYYYGMDVWGQGTTVTVSS |

TABLE 28
Germline information of top 8 further VK1 LC-optimized variants

| Name | VH Germline | VL Germline |
|---|---|---|
| LIBC680562-1 | VH4\|4-31/D3\|3-9\|RF2/JH6 | VK1\|O2/JK4 |
| LIBC680574-1 | VH4\|4-31/D3\|3-9\|RF2/JH6 | VK1\|O2/JK4 |
| LIBC680773-1 | VH4\|4-31/D3\|3-9\|RF2/JH6 | VK1\|O2/JK4 |
| LIBC680800-1 | VH4\|4-31/D3\|3-9\|RF2/JH6 | VK1\|O2/JK4 |
| LIBC680812-1 | VH4\|4-31/D3\|3-9\|RF2/JH6 | VK1\|O2/JK4 |
| LIBC681593-1 | VH4\|4-31/D3\|3-9\|RF2/JH6 | VK1\|O2/JK4 |
| LIBC681594-1 | VH4\|4-31/D3\|3-9\|RF2/JH6 | VK1\|O2/JK4 |
| LIBC681737-1 | VH4\|4-31/D3\|3-9\|RF2/JH6 | VK1\|O2/JK4 |

TABLE 29
Binding data of top 8 further VK1 LC-optimized variants

| | 2.5 nM | 25 nM | 0.2 | 50 nM |
|---|---|---|---|---|
| | CB1- | XCR1- | nM | Empty |
| Name | SMALP-GFP* | SMALP-GFP* | CB1-ND* | ND* |
| LIBC680562-1 | 1.94 | 0.64 | 3.34 | 0.68 |
| LIBC680574-1 | 1.72 | 0.56 | NA | 0.72 |
| LIBC680773-1 | 1.9 | 0.84 | 2.4 | 0.75 |
| LIBC680800-1 | 1.76 | 0.47 | 2.52 | 0.78 |
| LIBC680812-1 | 1.71 | 0.59 | 2.23 | 0.74 |
| LIBC681593-1 | 2.37 | 0.72 | NA | 0.91 |
| LIBC681594-1 | 2.57 | 1.04 | 3.56 | 0.83 |
| LIBC681737-1 | 1.94 | 0.62 | 1.89 | 0.73 |
| LIBC528116 | 1 | 1 | 1 | 1 |
| (Fab) | | | | |

*Relative binding fold change compared to LIBC528116 (Fab), calculated after background subtraction

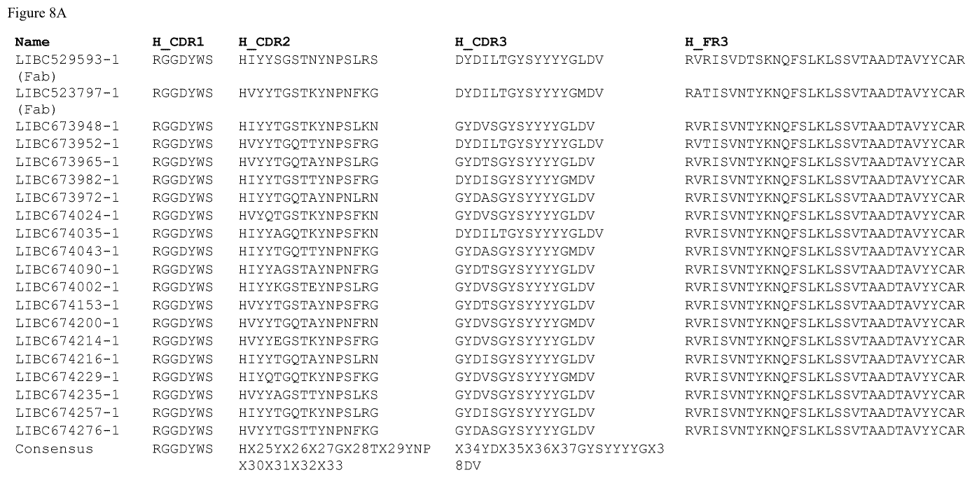

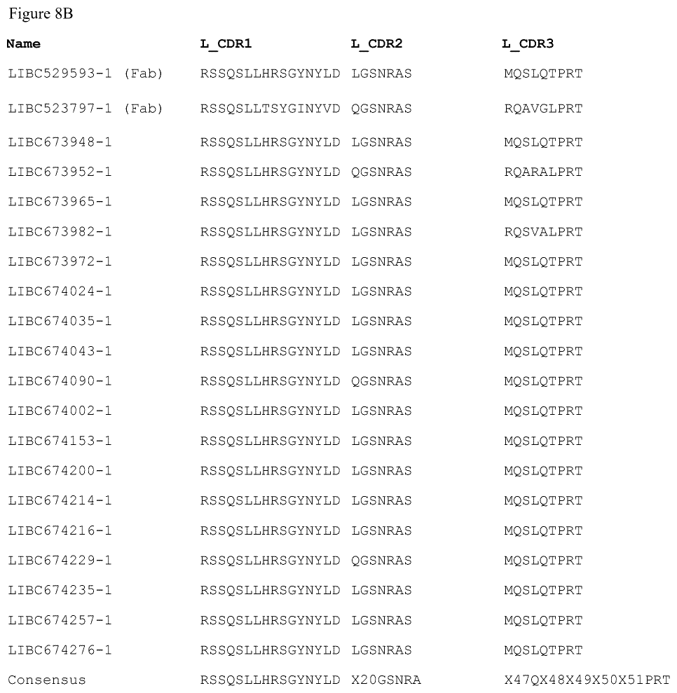

CDR sequences, germline information and binding data of the top 18 further cognate affinity maturation variants are summarized in Tables 30 and 31 and FIGS. 8A and 8B. Most of the 18 binders utilize the shorter HCDR3 and possess the HC T79R FR3 mutation in the library design for HC library 2, highlighting the benefit of rationally merging features from all available sequence-function relationships. All eighteen cognate variants met the stringent binding improvement threshold against huCB1I-GFP-SMALP and had minimal binding to irrelevant XCR1-SMALP and empty-ND (Table 32). These variants also exhibited better binding to CB 1 SMALP relative to LIBC523797-1. For LIBC674043-1 and LIBC674200-1, VH R149S and VL G76D germline reversion mutations were also included in the panel of IgG1 SEFL2 mAbs produced for functional testing.

TABLE 30
CDR sequence of top 18 further cognate variants

| | Seq | | Seq | | Seq | |
|---|---|---|---|---|---|---|
| Name | No. | CDR1 | No. | CDR2 | No. | CDR3 |
| LIBC673972-1_LC huCB1 LV | 257 | RSSQSLLHRSGYNYLD | 258 | LGSNRAS | 259 | MQSLQTPRT |
| LIBC673972-1_mol: | 260 | RGGDYWS | 261 | HIYYTGQTAYNPNLRN | 262 | GYDASGYSYYYYGLDV |
| fdChaincf huCB1 HV | | | | | | |
| LIBC673952-1_LC huCB1 LV | 263 | RSSQSLLHRSGYNYLD | 264 | QGSNRAS | 265 | RQARALPRT |
| LIBC673952-1_mol: | 266 | RGGDYWS | 267 | HVYYTGQTTYNPSFRG | 268 | DYDILTGYSYYYYGLDV |
| fdChaincf huCB1 HV | | | | | | |
| LIBC673982-1_LC huCB1 LV | 269 | RSSQSLLHRSGYNYLD | 270 | LGSNRAS | 271 | RQSVALPRT |
| LIBC673982-1_mol: | 272 | RGGDYWS | 273 | HIYYTGSTTYNPSFRG | 274 | DYDISGYSYYYYGMDV |
| fdChaincf huCB1 HV | | | | | | |
| LIBC674043-1_LC huCB1 LV | 275 | RSSQSLLHRSGYNYLD | 276 | LGSNRAS | 277 | MQSLOTPRT |
| LIBC674043-1_mol: | 278 | RGGDYWS | 279 | HIYYTGQTTYNPNFKG | 280 | GYDASGYSYYYYGMDV |
| fdChaincf huCB1 HV | | | | | | |
| LIBC673948-1_LC huCB1 LV | 281 | RSSQSLLHRSGYNYLD | 282 | LGSNRAS | 283 | MQSLQTPRT |
| LIBC673948-1_mol: | 284 | RGGDYWS | 285 | HIYYTGSTKYNPSLKN | 286 | GYDVSGYSYYYYGLDV |
| fdChaincf huCB1 HV | | | | | | |
| LIBC674090-1_LC huCB1 LV | 287 | RSSQSLLHRSGYNYLD | 288 | QGSNRAS | 289 | MQSLQTPRT |
| LIBC674090-1_mol: | 290 | RGGDYWS | 291 | HIYYAGSTAYNPNFRG | 292 | GYDTSGYSYYYYGLDV |
| fdChaincf huCB1 HV | | | | | | |
| LIBC674200-1(LC:G76D)_ | 293 | RSSQSLLHRSGYNYLD | 294 | LGSNRAS | 295 | MQSLQTPRT |
| LC huCB1 LV | | | | | | |
| LIBC674200- | 296 | RGGDYWS | 297 | HVYYTGQTAYNPNFRN | 298 | GYDVSGYSYYYYGMDV |
| 1(LC:G76D)_mol: | | | | | | |
| fdChaincf huCB1 HV | | | | | | |
| LIBC673965-1_LC huCB1 LV | 299 | RSSQSLLHRSGYNYLD | 300 | LGSNRAS | 301 | MQSLQTPRT |
| LIBC673965-1_mol: | 302 | RGGDYWS | 303 | HVYYTGQTAYNPSLRG | 304 | GYDTSGYSYYYYGLDV |
| fdChaincf huCB1 HV | | | | | | |
| LIBC674216-1_LC huCB1 LV | 305 | RSSQSLLHRSGYNYLD | 306 | LGSNRAS | 307 | MQSLQTPRT |
| LIBC674216-1_mol: | 308 | RGGDYWS | 309 | HIYYTGQTAYNPSLRN | 310 | GYDISGYSYYYYGLDV |
| fdChaincf huCB1 HV | | | | | | |
| LIBC674043-1 | 275 | RSSQSLLHRSGYNYLD | 276 | LGSNRAS | 277 | MQSLQTPRT |
| (mol:fdChaincf:R149S)_ | | | | | | |
| LC huCB1 LV | | | | | | |
| LIBC674043-1 | 278 | RGGDYWS | 279 | HIYYTGQTTYNPNFKG | 280 | GYDASGYSYYYYGMDV |
| (mol:fdChaincf:R149S)_ | | | | | | |
| mol:fdChaincf huCB1 HV | | | | | | |
| LIBC674214-1_LC huCB1 LV | 311 | RSSQSLLHRSGYNYLD | 312 | LGSNRAS | 313 | MQSLQTPRT |
| LIBC674214-1_mol: | 314 | RGGDYWS | 315 | HVYYEGSTKYNPSFRG | 316 | GYDVSGYSYYYYGLDV |
| fdChaincf huCB1 HV | | | | | | |
| LIBC674235-1_LC huCB1 LV | 317 | RSSQSLLHRSGYNYLD | 318 | LGSNRAS | 319 | MQSLQTPRT |
| LIBC674235-1_mol: | 320 | RGGDYWS | 321 | HVYYAGSTTYNPSLKS | 322 | GYDVSGYSYYYYGLDV |
| fdChaincf huCB1 HV | | | | | | |
| LIBC674257-1_LC huCB1 LV | 323 | RSSQSLLHRSGYNYLD | | 324LGSNRAS | 325 | MQSLOTPRT |
| LIBC674257-1_ | 326 | RGGDYWS | 327 | HIYYTGQTKYNPSLRG | 328 | GYDISGYSYYYYGLDV |
| mol:fdChaincf huCB1 HV | | | | | | |
| LIBC674200-1_LC huCB1 LV | 293 | RSSQSLLHRSGYNYLD | 294 | LGSNRAS | 295 | MQSLOTPRT |
| LIBC674200-1_mol: | 296 | RGGDYWS | 297 | HVYYTGQTAYNPNFRN | 298 | GYDVSGYSYYYYGMDV |
| fdChaincf huCB1 HV | | | | | | |
| LIBC674153-1_LC huCB1 LV | 329 | RSSQSLLHRSGYNYLD | 330 | LGSNRAS | 331 | MQSLQTPRT |
| LIBC674153-1_mol: | 332 | RGGDYWS | 333 | HVYYTGSTAYNPSFRG | 334 | GYDTSGYSYYYYGLDV |
| fdChaincf huCB1 HV | | | | | | |
| LIBC674002-1_LC huCB1 LV | 335 | RSSQSLLHRSGYNYLD | 336 | LGSNRAS | 337 | MQSLQTPRT |
| LIBC674002-1_mol: | 338 | RGGDYWS | 339 | HIYYKGSTEYNPSLRG | 340 | GYDVSGYSYYYYGLDV |
| fdChaincf huCB1 HV | | | | | | |
| LIBC674276-1_LC huCB1 LV | 341 | RSSQSLLHRSGYNYLD | 342 | LGSNRAS | 343 | MQSLQTPRT |
| LIBC674276-1_mol: | 344 | RGGDYWS | 345 | HVYYTGSTTYNPNFKG | 346 | GYDASGYSYYYYGLDV |
| fdChaincf huCB1 HV | | | | | | |
| LIBC674024-1_LC huCB1 LV | 347 | RSSQSLLHRSGYNYLD | 348 | LGSNRAS | 349 | MQSLQTPRT |
| LIBC674024-1_mol: | 350 | RGGDYWS | 351 | HVYQTGSTKYNPSFKN | 352 | GYDVSGYSYYYYGLDV |
| fdChaincf huCB1 HV | | | | | | |
| LIBC674035-1_LC huCB1 LV | 353 | RSSQSLLHRSGYNYLD | 354 | LGSNRAS | 355 | MQSLQTPRT |
| LIBC674035-1_mol: | 356 | RGGDYWS | 357 | HIYYAGQTKYNPSFKN | 358 | GYDISGYSYYYYGLDV |
| fdChaincf huCB1 HV | | | | | | |
| LIBC674229-1_LC huCB1 LV | 359 | RSSQSLLHRSGYNYLD | 360 | QGSNRAS | 361 | MQSLQTPRT |
| LIBC674229-1_mol: | 362 | RGGDYWS | 363 | HIYQTGQTKYNPSFKG | 364 | GYDVSGYSYYYYGMDV |
| fdChaincf huCB1 HV | | | | | | |

TABLE 31
Variable regions sequence of top 18 further cognate variants

| | Seq | | Seq | |
|---|---|---|---|---|
| Name | No. | LC LV | No. | mol:fdChaincf HV |
| LIBC67 | 365 | DIVMTQSPLSLPVTPGEPASISCRSSQSLLHRSGY | 366 | QVQLQESGPGLVKPSQTLSLTCTVSGGSIRRGGDYWSWI |
| 3972-1 | | NYLDWYLQKPGQSPQLLIYLGSNRASGVPDRFSG | | RQHPGKGLEWIGHIYYTGQTAYNPNLRNRVRISVNTYKN |
| | | SGSGTDFTLKISRVEAEDVGVYYCMQSLQTPRTF | | QFSLKLSSVTAADTAVYYCARGYDASGYSYYYYGLDVWG |
| | | GQGTKVEIKR | | QGTTVTVSS |
| LIBC67 | 367 | DIVMTQSPLSLPVTPGEPASISCRSSQSLLHRSGY | 368 | QVQLQESGPGLVKPSQTLSLTCTVSGGSIRRGGDYWSWI |
| 3952-1 | | NYLDWYLQKPGQSPQLLIYQGSNRASGVPDRFS | | RQHPGKGLEWIGHVYYTGQTTYNPSFRGRVTISVNTYKN |
| | | GSGSGTDFTLKISRVEAEDVGVYYCRQARALPRTF | | QFSLKLSSVTAADTAVYYCARDYDILTGYSYYYYGLDVWG |
| | | GQGTKVEIKR | | QGTTVTVSS |
| LIBC67 | 369 | DIVMTQSPLSLPVTPGEPASISCRSSQSLLHRSGY | 370 | QVQLQESGPGLVKPSQTLSLTCTVSGGSIRRGGDYWSWI |
| 3982-1 | | NYLDWYLQKPGQSPQLLIYLGSNRASGVPDRFSG | | RQHPGKGLEWIGHIYYTGSTTYNPSFRGRVRISVNTYKNQ |
| | | SGSGTDFTLKISRVEAEDVGVYYCRQSVALPRTFG | | FSLKLSSVTAADTAVYYCARDYDISGYSYYYYGMDVWGQ |
| | | QGTKVEIKR | | GTTVTVSS |
| LIBC67 | 371 | DIVMTQSPLSLPVTPGEPASISCRSSQSLLHRSGY | 372 | QVQLQESGPGLVKPSQTLSLTCTVSGGSIRRGGDYWSWI |
| 4043-1 | | NYLDWYLQKPGQSPQLLIYLGSNRASGVPDRFSG | | RQHPGKGLEWIGHIYYTGQTTYNPNFKGRVRISVNTYKN |
| | | SGSGTDFTLKISRVEAEDVGVYYCMQSLQTPRTF | | QFSLKLSSVTAADTAVYYCARGYDASGYSYYYYGMDVWG |
| | | GQGTKVEIKR | | QGTTVTVSR |
| LIBC67 | 373 | DIVMTQSPLSLPVTPGEPASISCRSSQSLLHRSGY | 374 | QVQLQESGPGLVKPSQTLSLTCTVSGGSIRRGGDYWSWI |
| 3948-1 | | NYLDWYLQKPGQSPQLLIYLGSNRASGVPDRFSG | | RQHPGKGLEWIGHIYYTGSTKYNPSLKNRVRISVNTYKNQ |
| | | SGSGTDFTLKISRVEAEDVGVYYCMQSLQTPRTF | | FSLKLSSVTAADTAVYYCARGYDVSGYSYYYYGLDVWGQ |
| | | GQGTKVEIKR | | GTTVTVSS |
| LIBC67 | 375 | DIVMTQSPLSLPVTPGEPASISCRSSQSLLHRSGY | 376 | QVQLQESGPGLVKPSQTLSLTCTVSGGSIRRGGDYWSWI |
| 4090-1 | | NYLDWYLQKPGQSPQLLIYQGSNRASGVPDRFS | | RQHPGKGLEWIGHIYYAGSTAYNPNFRGRVRISVNTYKN |
| | | GSGSGTDFTLKISRVEAEDVGVYYCMQSLOTPRT | | QFSLKLSSVTAADTAVYYCARGYDTSGYSYYYYGLDVWG |
| | | FGQGTKVEIKR | | QGTTVTVSS |
| LIBC67 | 377 | DIVMTQSPLSLPVTPGEPASISCRSSQSLLHRSGY | 378 | QVQLQESGPGLVKPSQTLSLTCTVSGGSIRRGGDYWSWI |
| 4200-1 | | NYLDWYLQKPGQSPQLLIYLGSNRASGVPDRFSG | | RQHPGKGLEWIGHVYYTGQTAYNPNFRNRVRISVNTYKN |
| (LC: | | SGSGTDFTLKISRVEAEDVGVYYCMQSLQTPRTF | | QFSLKLSSVTAADTAVYYCARGYDVSGYSYYYYGMDVWG |
| G76D) | | GQGTKVEIKR | | QGTTVTVSS |
| LIBC67 | 379 | DIVMTQSPLSLPVTPGEPASISCRSSQSLLHRSGY | 380 | QVQLQESGPGLVKPSQTLSLTCTVSGGSIRRGGDYWSWI |
| 3965-1 | | NYLDWYLQKPGQSPQLLIYLGSNRASGVPDRFSG | | RQHPGKGLEWIGHVYYTGQTAYNPSLRGRVRISVNTYKN |
| | | SGSGTDFTLKISRVEAEDVGVYYCMQSLQTPRTF | | QFSLKLSSVTAADTAVYYCARGYDTSGYSYYYYGLDVWG |
| | | GQGTKVEIKR | | QGTTVTVSS |
| LIBC67 | 381 | DIVMTQSPLSLPVTPGEPASISCRSSQSLLHRSGY | 382 | QVQLQESGPGLVKPSQTLSLTCTVSGGSIRRGGDYWSWI |
| 4216-1 | | NYLDWYLQKPGQSPQLLIYLGSNRASGVPDRFSG | | RQHPGKGLEWIGHIYYTGQTAYNPSLRNRVRISVNTYKN |
| | | SGSGTDFTLKISRVEAEDVGVYYCMQSLQTPRTF | | QFSLKLSSVTAADTAVYYCARGYDISGYSYYYYGLDVWGQ |
| | | GQGTKVEIKR | | GTTVTVSS |
| LIBC67 | 383 | DIVMTQSPLSLPVTPGEPASISCRSSQSLLHRSGY | 384 | QVQLQESGPGLVKPSQTLSLTCTVSGGSIRRGGDYWSWI |
| 4043-1 | | NYLDWYLQKPGQSPQLLIYLGSNRASGVPDRFSG | | RQHPGKGLEWIGHIYYTGQTTYNPNFKGRVRISVNTYKN |
| (mol: | | SGSGTDFTLKISRVEAEDVGVYYCMQSLOTPRTF | | QFSLKLSSVTAADTAVYYCARGYDASGYSYYYYGMDVWG |
| fdChain | | GQGTKVEIKR | | QGTTVTVSS |
| cf: | | | | |
| R149S) | | | | |
| LIBC67 | 385 | DIVMTQSPLSLPVTPGEPASISCRSSQSLLHRSGY | 386 | QVQLQESGPGLVKPSQTLSLTCTVSGGSIRRGGDYWSWI |
| 4214-1 | | NYLDWYLQKPGQSPQLLIYLGSNRASGVPDRFSG | | RQHPGKGLEWIGHVYYEGSTKYNPSFRGRVRISVNTYKN |
| | | SGSGTDFTLKISRVEAEDVGVYYCMQSLQTPRTF | | QFSLKLSSVTAADTAVYYCARGYDVSGYSYYYYGLDVWG |
| | | GQGTKVEIKR | | QGTTVTVSS |
| LIBC67 | 387 | DIVMTQSPLSLPVTPGEPASISCRSSQSLLHRSGY | 388 | QVQLQESGPGLVKPSQTLSLTCTVSGGSIRRGGDYWSWI |
| 4235-1 | | NYLDWYLQKPGQSPQLLIYLGSNRASGVPDRFSG | | RQHPGKGLEWIGHVYYAGSTTYNPSLKSRVRISVNTYKN |
| | | SGSGTDFTLKISRVEAEDVGVYYCMQSLQTPRTF | | QFSLKLSSVTAADTAVYYCARGYDVSGYSYYYYGLDVWG |
| | | GQGTKVEIKR | | QGTTVTVSS |
| LIBC67 | 389 | DIVMTQSPLSLPVTPGEPASISCRSSQSLLHRSGY | 390 | QVQLQESGPGLVKPSQTLSLTCTVSGGSIRRGGDYWSWI |
| 4257-1 | | NYLDWYLQKPGQSPQLLIYLGSNRASGVPDRFSG | | RQHPGKGLEWIGHIYYTGQTKYNPSLRGRVRISVNTYKN |
| | | SGSGTDFTLKISRVEAEDVGVYYCMQSLQTPRTF | | QFSLKLSSVTAADTAVYYCARGYDISGYSYYYYGLDVWGQ |
| | | GQGTKVEIKR | | GTTVTVSS |
| LIBC67 | 391 | DIVMTQSPLSLPVTPGEPASISCRSSQSLLHRSGY | 392 | QVQLQESGPGLVKPSQTLSLTCTVSGGSIRRGGDYWSWI |
| 4200-1 | | NYLDWYLQKPGQSPQLLIYLGSNRASGVPGRFSG | | RQHPGKGLEWIGHVYYTGQTAYNPNFRNRVRISVNTYKN |
| | | SGSGTDFTLKISRVEAEDVGVYYCMQSLQTPRTF | | QFSLKLSSVTAADTAVYYCARGYDVSGYSYYYYGMDVWG |
| | | GQGTKVEIKR | | QGTTVTVSS |
| LIBC67 | 393 | DIVMTQSPLSLPVTPGEPASISCRSSQSLLHRSGY | 394 | QVQLQESGPGLVKPSQTLSLTCTVSGGSIRRGGDYWSWI |
| 4153-1 | | NYLDWYLQKPGQSPQLLIYLGSNRASGVPDRFSG | | RQHPGKGLEWIGHVYYTGSTAYNPSFRGRVRISVNTYKN |
| | | SGSGTDFTLKISRVEAEDVGVYYCMQSLQTPRTF | | QFSLKLSSVTAADTAVYYCARGYDTSGYSYYYYGLDVWG |
| | | GQGTKVEIKR | | QGTTVTVSS |
| LIBC67 | 395 | DIVMTQSPLSLPVTPGEPASISCRSSQSLLHRSGY | 396 | QVQLQESGPGLVKPSQTLSLTCTVSGGSIRRGGDYWSWI |
| 4002-1 | | NYLDWYLQKPGQSPQLLIYLGSNRASGVPDRFSG | | RQHPGKGLEWIGHIYYKGSTEYNPSLRGRVRISVNTYKNQ |
| | | SGSGTDFTLKISRVEAEDVGVYYCMQSLQTPRTF | | FSLKLSSVTAADTAVYYCARGYDVSGYSYYYYGLDVWGQ |
| | | GQGTKVEIKR | | GTTVTVSS |
| LIBC67 | 397 | DIVMTQSPLSLPVTPGEPASISCRSSQSLLHRSGY | 398 | QVQLQESGPGLVKPSQTLSLTCTVSGGSIRRGGDYWSWI |
| 4276-1 | | NYLDWYLQKPGQSPQLLIYLGSNRASGVPDRFSG | | RQHPGKGLEWIGHVYYTGSTTYNPNFKGRVRISVNTYKN |
| | | SGSGTDFTLKISRVEAEDVGVYYCMQSLQTPRTF | | QFSLKLSSVTAADTAVYYCARGYDASGYSYYYYGLDVWG |
| | | GQGTKVEIKR | | QGTTVTVSS |
| LIBC67 | 399 | DIVMTQSPLSLPVTPGEPASISCRSSQSLLHRSGY | 400 | QVQLQESGPGLVKPSQTLSLTCTVSGGSIRRGGDYWSWI |
| 4024-1 | | NYLDWYLQKPGQSPQLLIYLGSNRASGVPDRFSG | | RQHPGKGLEWIGHVYQTGSTKYNPSFKNRVRISVNTYKN |
| | | SGSGTDFTLKISRVEAEDVGVYYCMQSLQTPRTF | | QFSLKLSSVTAADTAVYYCARGYDVSGYSYYYYGLDVWG |
| | | GQGTKVEIKR | | QGTTVTVSS |
| LIBC67 | 401 | DIVMTQSPLSLPVTPGEPASISCRSSQSLLHRSGY | 402 | QVQLQESGPGLVKPSQTLSLTCTVSGGSIRRGGDYWSWI |
| 4035-1 | | NYLDWYLQKPGQSPQLLIYLGSNRASGVPDRFSG | | RQHPGKGLEWIGHIYYAGQTKYNPSFKNRVRISVNTYKN |
| | | SGSGTDFTLKISRVEAEDVGVYYCMQSLQTPRTF | | QFSLKLSSVTAADTAVYYCARGYDISGYSYYYYGLDVWGQ |
| | | GQGTKVEIKR | | GTTVTVSS |
| LIBC67 | 403 | DIVMTQSPLSLPVTPGEPASISCRSSQSLLHRSGY | 404 | QVQLQESGPGLVKPSQTLSLTCTVSGGSIRRGGDYWSWI |
| 4229-1 | | NYLDWYLQKPGQSPQLLIYQGSNRASGVPDRFS | | RQHPGKGLEWIGHIYQTGQTKYNPSFKGRVRISVNTYKN |
| | | GSGSGTDFTLKISRVEAEDVGVYYCMQSLOTPRT | | QFSLKLSSVTAADTAVYYCARGYDVSGYSYYYYGMDVWG |
| | | FGQGTKVEIKR | | QGTTVTVSS |

TABLE 32
Binding data of top 18 further cognate variants

| | 10 nM | 5 nM | 50 nM | 0.5 | 50 nM |
|---|---|---|---|---|---|
| | CB1 | CB1 | XCR1 | nM | Empty |
| HTB Barcode | SMALP* | SMALP* | SMALP* | CB1 ND* | ND* |
| LIBC529593-1 | 1.06 | 1.42 | 1.50 | 0.38 | 1.14 |
| (Fab) | | | | | |
| LIBC523797-1 | 1.00 | 1.00 | 1.00 | 1.00 | 1.00 |
| (Fab) | | | | | |
| LIBC673948-1 | 2.46 | 1.37 | 0.88 | 0.64 | 0.48 |
| LIBC673952-1 | 1.91 | 1.23 | 0.59 | 0.77 | 0.62 |
| LIBC673965-1 | 2.50 | 1.30 | 0.74 | 0.65 | 0.43 |
| LIBC673982-1 | 2.16 | 1.24 | 0.79 | 0.78 | 0.60 |
| LIBC673972-1 | 2.46 | 1.46 | 0.80 | 0.63 | 0.40 |
| LIBC674024-1 | 2.39 | 1.24 | 0.86 | 0.49 | 0.55 |
| LIBC674035-1 | 2.74 | 1.29 | 0.91 | 0.52 | 0.56 |
| LIBC674043-1 | 3.23 | 1.20 | 0.62 | 1.85 | 0.75 |
| LIBC674090-1 | 2.85 | 1.20 | 0.74 | 1.35 | 0.99 |
| LIBC674002-1 | 2.59 | 1.49 | 0.91 | 0.46 | 0.03 |
| LIBC674153-1 | 2.62 | 1.35 | 0.68 | 1.83 | 0.33 |
| LIBC674200-1 | 3.31 | 1.33 | 0.79 | 2.08 | 0.74 |
| LIBC674214-1 | 2.10 | 1.17 | 0.68 | 1.76 | 1.09 |
| LIBC674216-1 | 1.96 | 1.10 | 0.52 | 1.66 | 0.65 |
| LIBC674229-1 | 3.17 | 1.11 | 0.80 | 2.22 | 0.75 |
| LIBC674235-1 | 2.05 | 1.30 | 0.68 | 1.28 | 0.72 |
| LIBC674257-1 | 3.34 | 1.19 | 0.77 | 1.86 | 0.73 |
| LIBC674276-1 | 2.06 | 1.17 | 0.54 | 2.04 | 0.51 |

*Relative binding fold change compared to LIBC523797-1(Fab), calculated after background subtraction

### Example 10. Functional Analysis of Further Affinity Matured Variants

Clones isolated from Example 9 above were cloned and expressed as IgG1 SEFL2 mAbs for functional analysis. Antagonist activity of the antibodies was analyzed using the TR-FRET cAMP assay as described above (e.g., Example 8) and potency of the antibodies are summarized in Table 33. The potency of all the antibodies was in single digit nM, with several having potency of less than 1 nM in all four experiments.

TABLE 33
IC50 values of exemplary antibodies
from further affinity maturation

| | IC50 (nM, | | IC50 (nM, |
|---|---|---|---|
| Molecule | n = 4) | Molecule | n = 4) |
| LIBC673952-1 | 2.48 | LIBC680562-1 | 0.71 |
| LIBC673965-1 | 2.21 | LIBC680574-1 | 0.75 |
| LIBC673972-1 | 2.43 | LIBC680773-1 | 0.74 |
| LIBC673982-1 | 2.11 | LIBC680800-1 | 1.57 |
| LIBC674090-1 | 1.76 | LIBC680812-1 | 0.68 |
| LIBC674200-1 | 2.39 | LIBC681593-1 | 0.61 |
| LIBC674200-1 | 2.23 | LIBC681594-1 | 1.24 |
| (LC: G76D) | | LIBC681737-1 | 0.55 |
| LIBC674229-1 | 4.16 | | |
| 26970-1 | 14.41 | | |
| 10D10 | 346.54 | | |
| Rimonabant | 3.40 | | |
| LIBC528116-1 | 2.02 | | |

### Example 11. Effects of Further Affinity Matured Variants in Animal Model

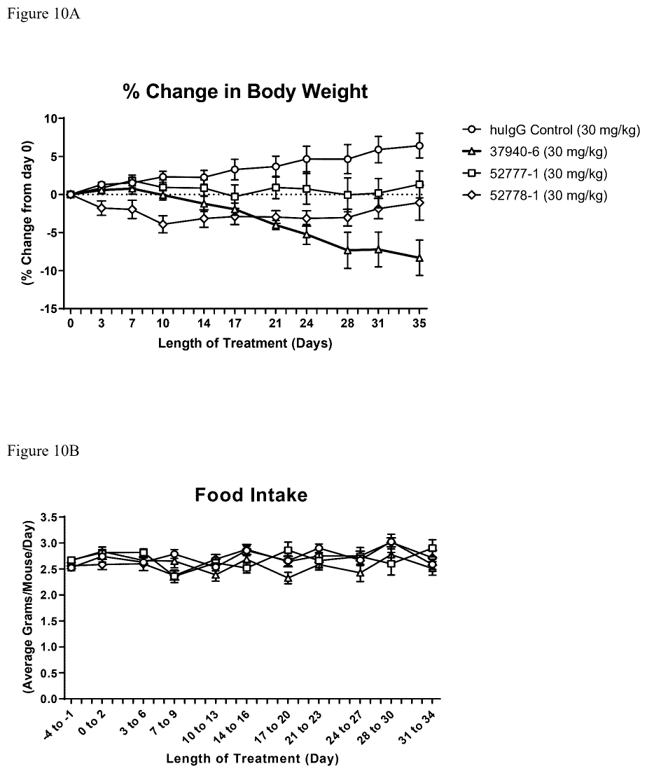

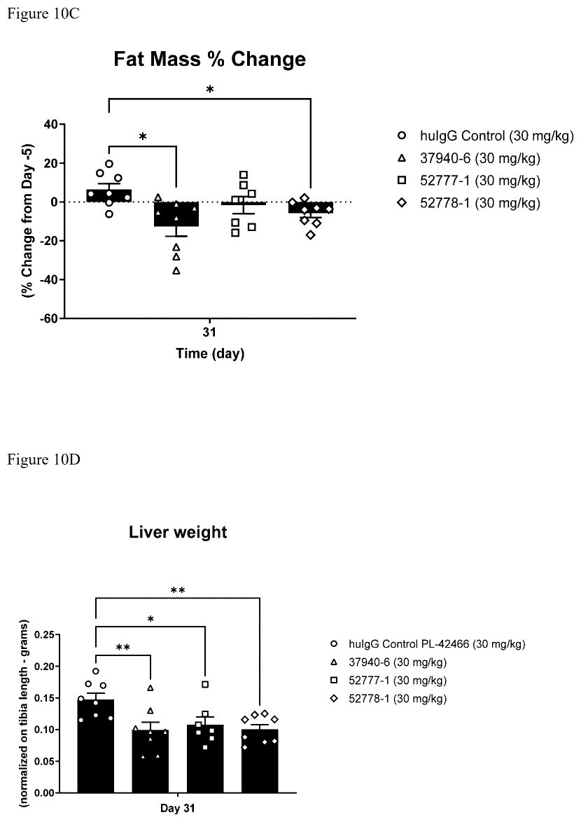

The further affinity matured antibodies LIBC673952 (52777) and LIBC680574 (52778) in YTE format (Dall'Acqua et al., The Journal of Immunology, 169: 5171-5180 (2002)) was scaled up for testing in diet-induced obese (DIO) transgenic mice with huCB1 knocked in. 6- to 10-week-old huCB1 knock in (KI) mice were fed with high fat diet (HFD) for 15 weeks to induce obesity. The antibodies (52777, 52778, and 37940) and a control antibody (huIgG control) were administered to the mice at 30 mg/kg QW for 35 days, and the body weight and food intake of the animals were measured every 3-4 days and the results are shown in FIGS. 10A and 10B. The administration of the further affinity matured antibodies reduced body weight of the animals (FIG. 10A), while the average daily food intake did not change significantly (FIG. 10B). Fat mass and liver weight of the animals were also measured, and the results are shown in FIGS. 10C and 10D. These results showed that potent peripherally-acting anti-CB1 antibody descried herein was an effective treatment for anti-obesity, hyperinsulinemia, and NASH.

### Example 12. Effects of Anti-CB1 mAb in Energy Expenditure and Insulin Tolerance Test

Materials and Methods: All studies using mice complied with all relevant ethical regulations and approval for the studies performed were obtained from Amgen's IACUC institutional review board (Thousand Oaks, CA). Lighting in animal holding rooms was maintained on 12:12 hr light:dark cycle, and the ambient temperature and humidity range was at 68-79° F. and 30-70%, respectively. Animals had ad libitum access to irradiated pelleted feed and reverse-osmosis chlorinated (0.3 to 0.5 ppm) water via an automatic watering system or by water bottles as indicated. Cages were changed weekly.

Male huCNR1 TG mice (4-7 weeks old) were shipped from Charles River Laboratories (Hollister, CA) to Amgen (Thousand Oaks, CA), singly house upon arrival, and started high-fat diet (HFD) feeding (60% kcal fat, Research Diets D12492i) for 12 weeks. After 12 weeks HFD feeding, mice were acclimated to water bottles for one week and then n=24 mice were transferred to the Comprehensive Laboratory Animal Monitoring System (CLAMS, Columbus Instruments, Columbus, Ohio—Oxymax Model 2018, 0233-004M, Oxymax for Windows v5.53 software, Hardware configuration 190395) for an additional two weeks of acclimation. Volume of oxygen consumption (V02), carbon dioxide production (VCO2), respiratory exchange ratio (RER), and energy expenditure (HEAT) were measured during that period. At the start of treatment, mice had been fed HFD for 15 weeks total and n=23 mice were randomized into two groups based on body weight, age, V02, VCO2, RER, and Heat during baseline measurements from days −3 to −1. Mice were dosed with huIgG1 control PL-42466 (n=11 mice) or CB1 mAb 37940-17 (n=12 mice) at 30 mg/kg (2 mL/kg) IP weekly for a total of 5 injections. Mice were housed in the CLAMS system from days −14 to day 7, were returned to home caging on days 7-14, returned to the CLAMS system from days 14 to 28, and were returned to home caging on day 28 throughout the duration of the study. Body weight and food intake were measured weekly. For simplicity, data is displayed as lights on and lights off cycles (12 hours on: 12 hours off).

For the insulin tolerance test (ITT), on day 32 of treatment (4 days post 5th IP injection), mice were fasted at 6 am for 6 hours. At 12 pm, blood glucose was measured by retroorbital bleed using a glucometer (AlphaTrak) and mice were immediately IP dosed with insulin (0.75 U/kg). Blood glucose was measured by retroorbital bleed at 30-, 60-, 90-, and 120-minutes post insulin administration. Area under the curve (AUC) was calculated for each mouse using GraphPad Prism (version 9.5.1).

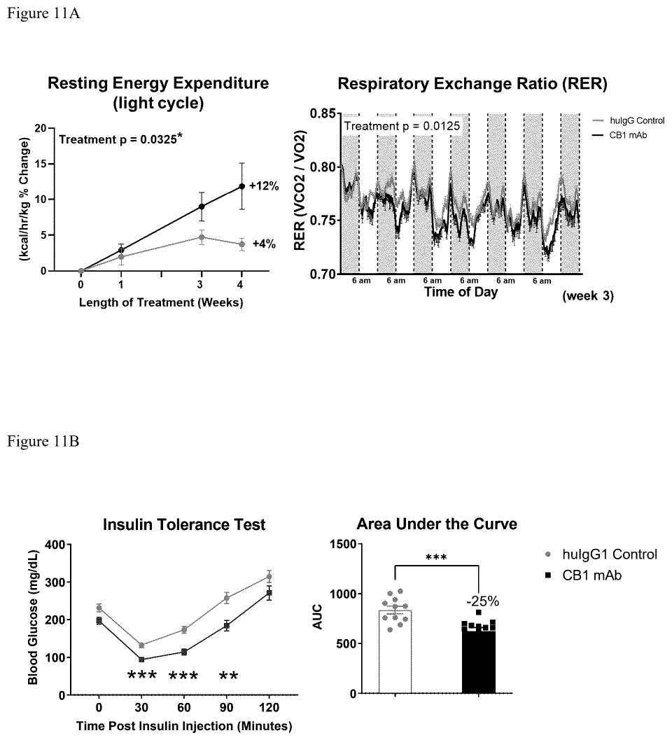

Results: Energy expenditure measured as heat (kcal/hr/kg) and respiratory exchange ratio (RER) were averaged per week (acclimation, week 1, week 3, and week 4) for the light cycle (6 am-6 pm) and the dark cycle (6 pm-6 am). In the light cycle when mice are resting, energy expenditure during week 4 of treatment was significantly higher in the CB1 mAb treated mice (+12% compared to baseline for CB1 vs. +4% compared to baseline for control, p=0.0325; two-way repeated measures ANOVA with Sidak's test for multiple comparisons, FIG. 11A). Additionally, RER was significantly lower in the CB1 mAb treatment group during week 3 of treatment (treatment p=0.0125; two-way repeated measures ANOVA with Sidak's test for multiple comparisons), indicating increased lipid oxidation (FIG. 11A).

To assess insulin sensitivity, an insulin tolerance test (ITT) was conducted. After a 6-hour fast, mice did not differ in baseline blood glucose measurements; however, after insulin administration, CB1 mAb treated mice had statistically lower blood glucose at 30-, 60-, and 90-mins post-insulin injection (p<0.01, two-way repeated measures ANOVA with Sidak's test for multiple comparisons) and 25% lower AUC (p=0.0006; Student's unpaired t-test), indicating significantly improved insulin sensitivity after CB1 mAb treatment (FIG. 11B).

### Example 13. Brain Uptake Study of Ant-CB1 Mab in Tg32 Mice

This study was carried out to characterize the single-dose plasma pharmacokinetics and brain uptake of an exemplary anti-CB1 antagonist mAb following intravenous bolus administration in male huFcRn Tg 32 homozygous mice.

Materials and Methods: Food and water were provided ad libitum throughout the study duration. On the day before dosing, the stock test articles were removed from −70° C. (±10° C.) storage and placed at 2-8° C. to thaw overnight. On the morning of dose administration, the stock test article was removed from 2-8° C. storage, mixed by gentle inversion, and placed on wet ice for the dilution procedures. The vehicle was removed from 2-8° C. storage and mixed by gentle inversion and placed on wet ice for the dilution procedures. For all groups, the stock test articles were diluted in the appropriate formulation buffer to obtain the final dose solution concentrations listed in the experimental design. Following dose solution preparation, all remaining vehicles (if applicable) were returned to 2-8° C. storage. Following dose administration, all remaining stock test articles (if applicable) and remaining dosing solutions (if applicable) were placed at −70° C. (±10° C.) and transferred to the Bioanalytical Principal Investigator for subsequent analysis and storage. Test animals received an intravenous bolus dose of the appropriate dosing solution via the lateral tail vein.

SARSTEDT Microvette® K3 EDTA plasma separation tubes were used to collect approximately 0.05 mL of whole blood per test system at each serial time point via submandibular venipuncture. After putting the whole blood into the SARSTEDT Microvette® plasma tubes, the tubes were mixed by 8-10 gentle inversions and placed on wet ice. The specimens were centrifuged at 2-8° C. at approximately 14,000 rcf for 5 minutes using a calibrated Eppendorf 5417R Centrifuge System (Brinkmann Instruments, Inc., Westbury, NY 11590). All plasma specimens were stored at approximately −70° C. (±10° C.) until analysis.

Transcardial perfusion was performed at the end of the study. Each test system was deeply anesthetized with isoflurane (5% in O2(g) at a rate of 1.5 L/min) and remained under a surgical plane of anesthesia throughout the perfusion procedure. A cut was made in the skin right below the sternum and the tip of the sternum was located. Grasping the sternum, a cut was made through the diaphragm and along both sides of the rib cage to expose the heart, giving the animal a pneumothorax. The heart was steadied, and a 20 gauge, ½″ needle was placed in the left ventricle. The needle was connected via tubing to a perfusion/syringe pump, and 0.9% saline was allowed to run at a rate of approximately 4 mL/minute for approximately 5 minutes. Once flow was established, the right atrium was incised to allow fluid to flow out of the animal, preventing over-pressurizing of the vasculature. After 5 minutes of perfusion with saline, 10% neutral buffered formalin was perfused for an additional 5 minutes. Once perfusion was complete, the needle was removed from the left ventricle and the appropriate tissue(s) was harvested for subsequent analysis.

Tissues were harvested from each test system as outlined below immediately following exsanguination at the terminal time point. Tissues were rinsed with 0.9% sterile saline upon extraction, blotted with dry gauze, immediately placed into cryovials (Eppendorf Protein LoBind, cat. #0030108116), weighed, and submerged in liquid nitrogen to snap freeze the tissues. Once the tissues were snapped frozen, they were stored on dry ice during necropsy procedures and at −70° C. (±10° C.) for subsequent analysis.

Plasma and tissue specimens for PK test article concentration determination were analyzed using an Enzyme-Linked ImmunoSorbant Assay (ELISA). Specimens were maintained at −70° C. (±10° C.) prior to analysis. Noncompartmental analysis was performed on the mean plasma test article concentration vs. time data from all mice at each sampling time per dose group. Parameters of interest include tissue-to-plasma percentage ratio on day 10, and half-life (t_{1/2}).

Results: the PK and brain exposure of the antibody are shown in Table 34 below. The results show that brain exposure of the anti-CB1 mAb is 0.2% of plasma concentration of the antibody. Transgenic Tg32 mice expressing human neonatal Fc receptor (hFcRn) are known to be predictive of human PK for mAb therapeutics (Avery et. al. mAbs 20161). These results show that a 0.002 fraction of mAb plasma concentration can distribute to the brain. These results are also consistent with the previously published literature on Immunoglobulin G1 (IgG1) mAbs in other mouse models (e.g., Avery L. B. et al., MAbs. 2016 August-September; 8(6):1064-78. doi: 10.1080/19420862.2016.1193660. Epub 2016 May 27. PMID: 27232760; PMCID: PMC4968115; Shah D. K. et al., MAbs. 2013 March-April; 5(2):297-305. doi: 10.4161/mabs.23684. Epub 2013 Feb. 13. PMID: 23406896; PMCID: PMC3893240; and Garg A. and Balthasar J. P., AAPS J. 2009 September; 11(3):553-7. doi: 10.1208/s⁻¹²²⁴⁸-009-9129-9. Epub 2009 Jul. 28. PMID: 19636712; PMCID: PMC2758122).

TABLE 34
PK and brain exposure of the anti-CB1 mAb

| | Animal system | Anti-CB1 mAb |
|---|---|---|
| PK (T_{1/2}) | Tg32 mice | 10.37 days |
| | (huFcRn) | |
| Brain exposure (% of plasma | Tg32 mice | 0.21 ± 0.07% |
| concentration on day 10) | (huFcRn) | |

### Example 14. Cryo-Em Structures of Cb1-Fab Complexes

Cryo-EM structures of huCB1 in complex with Fab of 10D10 and four affinity matured (AM) antibodies were resolved in a resolution of from ˜3.1 to 3.7 Å, which is sufficient to determine epitope/paratope interactions. The potency of the antibodies ranges from 1.77 to 47 nM. The cryo-EM structures confirmed that only heavy chain of the antibodies is involved in binding and ECL2 of CB1 is the major epitope. In addition, HCDR1 plays a major role in binding of CB1 ECL2, this is consistent with the fact that HCDR1 sequences of AM antibodies are highly conserved. R32 (AHo numbering) that is adjacent to HCDR1 is also involved in epitope interaction. HCDR2 are diverse in sequence and certain residues (e.g., Y59, AHo numbering) in HCDR2 appear to contribute to binding affinity. Y131 and Y132 (AHo numbering) of HCDR3 are conserved among the tested antibodies and bind to CB1.

TABLE 35
Consensus sequences of affinity matured variants

| Seq. | | |
|---|---|---|
| No. | Name | Sequence |
| 530 | Consensus sequence | RGGDYWX1 |
| | affinity matured variants HV | X1 is A, S, or G |
| | CDR1 | |
| 531 | Consensus sequence | HX2YX3X4GX5TX6YNPX7X8X9X10 |
| | affinity matured variants HV | X2 is I or V; X3 is H, Y, or Q; X4 is E, T, or S; X5 is K, |
| | CDR2 | S, or Q; X6 is A, K or N; X7 is N, S, K, or R; X8 is F or |
| | | L; X9 is E or K; and X10 is G, D, S, or N |
| 532 | Consensus sequence | X11YDX12X13X14GX15SYYYYGMDV |
| | affinity matured variants HV | X11 is D, E, or N; X12 is A, I, P, T, or V; X13 is F, L, |
| | CDR3 | or V; X14 is S or T; and X15 is H, N, or Y |
| 533 | Consensus sequence | RSSQSLLX16SX17GX18NYX19D |
| | affinity matured variants LV | X16 is H, S, or T; X17 is S, T, or Y; X18 is A, N, I or Y; |
| | CDR1 | and X19 is L or V |
| 534 | Consensus sequence | X20GSNRA |
| | affinity matured variants LV | X20 is L or Q |
| | CDR2 | |
| 535 | Consensus sequence | X21QAX22X23X24PRT |
| | affinity matured variants LV | X21 is Mor R; X22 is L, I, R, or V; X23 is Q, E, T, A, or |
| | CDR3 | G; and X24 is T, I, L, or Q |
| 564 | Consensus sequence_further | RGGDYWX1 |
| | affinity matured LC swapped | X1 is A or G |
| | variants HV CDR1 | |
| 565 | Consensus sequence_further | HX2YX3X4GX5TX6YNPX7X8X9X10 |
| | affinity matured LC swapped | X2 is I or V; X3 is Y, or Q; X4 is T; X5 is S; X6 is K or |
| | variants HV CDR2 | N; X7 is S, or R; X8 is F; X9 is K; and X10 is G or D |
| 566 | Consensus sequence_further | X11YDX12X13X14GX15SYYYYGMDV |
| | affinity matured LC swapped | X11 is N; X12 is T; X13 is L, or V; X14 is T; and X15 is |
| | variants HV CDR3 | Y |
| 405 | Consensus sequence_further | RASQSISSYLN |
| | affinity matured LC swapped | |
| | variants LV CDR1 | |
| 536 | Consensus sequence_further | X39ARX40LX41S |
| | affinity matured LC swapped | X39 is N, S, K or G; X40 is R, K, L or A; and X41 is A, |
| | variants LV CDR2 | G or S |
| 537 | Consensus sequence_further | QQX42X43X44X45PX46T |
| | affinity matured LC swapped | X42 is Y or F, X43 is R, A, S, G, or Y; X44 is S, K, R or |
| | variants LV CDR3 | H; X45 is S, L, F, Y, P, or M; and X46 is L, I or V |
| 1 | Consensus sequence_further | RGGDYWS |
| | affinity matured cognate | |
| | variants HV CDR1 | |
| 538 | Consensus sequence_further | HX25YX26X27GX28TX29YNPX30X31X32X33 |
| | affinity matured cognate | X25 is I or V; X26 is Y or Q; X27 is A, E, K, or T; X28 |
| | variants HV CDR2 | is S or Q; X29 is A, E, K, or T; X30 is N or S; X31 is F |
| | | or L, X32 is K or R; and X33 is G, S, or |
| 539 | Consensus sequence_further | X34YDX35X36X37GYSYYYYGX38DV |
| | affinity matured cognate | X34 is D or G; X35 is A, I, T, or V; X36 is L or absent; |
| | variants HV CDR3 | X37 is S or T; and X38 is M or L |
| 257 | Consensus sequence_further | RSSQSLLHRSGYNYLD |
| | affinity matured cognate | |
| | variants LV CDR1 | |
| 534 | Consensus sequence_further | X20GSNRA |
| | affinity matured cognate | X20 is L or Q |
| | variants LV CDR2 | |
| 540 | Consensus sequence_further | X47QX48X49X50X51PRT |
| | affinity matured cognate | X47 is M or R; X48 is A or S; X49 is L, R, or V; X50 is |
| | variants LV CDR3 | Q or A; and X51 is T or L |

While this invention has been described with an emphasis upon preferred aspects, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

1. An isolated antibody that binds to human cannabinoid receptor 1 (huCB1), wherein the antibody comprises a heavy chain variable region (HV) comprising heavy chain complimentary regions (CDR) HCDR1, HCDR2, and HCDR3, and a light chain variable region (LV) comprising light chain complimentary regions (CDR) LCDR1, LCDR2, and LCDR3, wherein:
(a) HCDR1, HCDR2 and HCDR3 comprise the amino acid sequence of SEQ ID NOS: 135-137, respectively, and LCDR1, LCDR2 and LCDR3 comprise the amino acid sequence of SEQ ID NOS: 138-140, respectively;
(b) HCDR1, HCDR2 and HCDR3 comprise the amino acid sequence of SEQ ID NOS: 426-428, respectively and LCDR1, LCDR2 and LCDR3 comprise the amino acid sequence of SEQ ID NOS: 423-425, respectively;
(c) HCDR1, HCDR2 and HCDR3 comprise the amino acid sequence of SEQ ID NOS: 266-268, respectively and LCDR1, LCDR2 and LCDR3 comprise the amino acid sequence of SEQ ID NOS: 263-235, respectively;
(d) HCDR1, HCDR2 and HCDR3 comprise the amino acid sequence of SEQ ID NOS: 129-131, respectively and LCDR1, LCDR2 and LCDR3 comprise the amino acid sequence of SEQ ID NOS: 132-134, respectively;
(e) HCDR1, HCDR2 and HCDR3 comprise the amino acid sequence of SEQ ID NOS: 159-161, respectively and LCDR1, LCDR2 and LCDR3 comprise the amino acid sequence of SEQ ID NOS: 162-164, respectively;
(f) HCDR1, HCDR2 and HCDR3 comprise the amino acid sequence of SEQ ID NOS: 207-209, respectively and LCDR1, LCDR2 and LCDR3 comprise the amino acid sequence of SEQ ID NOS: 210-212, respectively;
(g) HCDR1, HCDR2 and HCDR3 comprise the amino acid sequence of SEQ ID NOS: 1-3, respectively and LCDR1, LCDR2 and LCDR3 comprise the amino acid sequence of SEQ ID NOS: 4-6, respectively; or
(h) HCDR1, HCDR2 and HCDR3 comprise the amino acid sequence of SEQ ID NOS: 55-57, respectively and LCDR1, LCDR2 and LCDR3 comprise the amino acid sequence of SEQ ID NOS: 58-60, respectively.

2. The antibody of claim 1, wherein HV comprises an amino acid sequence selected from SEQ ID NO: 228, SEQ 1-N-DO ID NO: 460, SEQ ID NO: 368, SEQ ID NO: 100, SEQ ID NO: 236, SEQ ID NO: 226, SEQ ID NO: 252, SEQ ID NO: 98, and SEQ ID NO: 116.

3. The antibody of claim 1, wherein LV comprises an amino acid sequence selected from SEQ ID NO: 227, SEQ ID NO: 459, SEQ ID NO: 367, SEQ ID NO: 99, SEQ ID NO: 235, SEQ ID NO: 225, SEQ ID NO: 251, SEQ ID NO: 97, and SEQ ID NO: 115.

4. The antibody of claim 1, wherein
i) HV comprises the amino acid sequence of SEQ ID NO: 228, and LV comprises the amino acid sequence of SEQ ID NO: 227;
ii) HV comprises the amino acid sequence of SEQ ID NO: 460, and LV comprises the amino acid sequence of SEQ ID NO: 459;
iii) HV comprises the amino acid sequence of SEQ ID NO: 368, and LV comprises the amino acid sequence of SEQ ID NO: 367;
iv) HV comprises the amino acid sequence of SEQ ID NO: 100, and LV comprises the amino acid sequence of SEQ ID NO: 99;
v) HV comprises the amino acid sequence of SEQ ID NO: 236, and LV comprises the amino acid sequence of SEQ ID NO: 235;
vi) HV comprises the amino acid sequence of SEQ ID NO: 226, and LV comprises the amino acid sequence of SEQ ID NO: 225;
vii) HV comprises the amino acid sequence of SEQ ID NO: 252, and LV comprises the amino acid sequence of SEQ ID NO: 251;
viii) HV comprises the amino acid sequence of SEQ ID NO: 98, and LV comprises the amino acid sequence of SEQ ID NO: 97; or
ix) HV comprises the amino acid sequence of SEQ ID NO: 116, and LV comprises the amino acid sequence of SEQ ID NO: 115.

5. An isolated antibody that binds to huCB1, wherein the antibody comprises a HV and a LV, wherein
a) the HV comprises HCDR1, HCDR2 and HCDR3, wherein
HCDR1 comprises the amino acid sequence of RGGDYWX1 (SEQ ID NO: 530), wherein X1 is A, S, or G;
HCDR2 comprises the amino acid sequence of HX2YX3X4GX5TX6YNPX7X8X9X10 (SEQ ID NO: 531), wherein X2 is I or V; X3 is H, Y, or Q; X4 is E, T, or S; X5 is K, S, or Q; X6 is A, K or N; X7 is N, S, K, or R; X8 is F or L; X9 is E or K; and X10 is G, D, S, or N; and
HCDR3 comprises the amino acid sequence of X11YDX12X13X14GX15SYYYYGMDV (SEQ ID NO: 532), wherein X11 is D, E, or N; X12 is A, I, P, T, or V; X13 is F, L, or V; X14 is S or T; and X15 is H, N, or Y;
and
b) the LV is of a VK1/O2/JK4, a VK2/A19/JK1, or a VK3/A27/JK1 germline.

6. The antibody of claim 5, wherein
a) HCDR1 comprises the amino acid sequence of RGGDYWX1 (SEQ ID NO: 530), wherein X1 is A or S;
HCDR2 comprises the amino acid sequence of HX2YX3X4GX5TX6YNPX7X8X9X10 (SEQ ID NO: 531), wherein X2 is I or V; X3 is Y or Q; X4 is E, T, or S; X5 is S or Q; X6 is A or K; X7 is N, S, K, or R; X8 is F or L; X9 is E or K; and X10 is G, D, S, or N; and
HCDR3 comprises the amino acid sequence of X11YDX12X13X14GX15SYYYYGMDV (SEQ ID NO: 532), wherein X11 is D, E, or N; X12 is A, I, P, or V; X13 is L or V; X14 is S or T; and X15 is H or Y; and
b) the LV is of a VK2/A19/JK1 germline.

7. The antibody of claim 6, wherein X4 is T, X15 is Y.

8. The antibody of claim 5, wherein
b) the LV comprises LCDR1, LCDR2 and LCDR3, and wherein
LCDR1 comprises the amino acid sequence RSSQSLLX16SX17GX18NYX19D (SEQ ID NO: 533), wherein X16 is H, S, or T; X17 is S, T, or Y; X18 is A, N, I or Y; and X19 is L or V;
LCDR2 comprises the amino acid sequence of X20GSNRA (SEQ ID NO: 534), wherein X20 is L or Q; and
LCDR3 comprises the amino acid sequence of X21QAX22X23X24PRT (SEQ ID NO: 535), wherein X21 is M or R; X22 is L, I, R, or V; X23 is Q, E, T, A, or G; and X24 is T, I, L, or Q.

9. The antibody of claim 8, wherein X24 is L.

10. The antibody of claim 5, wherein
a) HCDR1 comprises the amino acid sequence of RGGDYWX1 (SEQ ID NO: 530), wherein X1 is A, G or S;
HCDR2 comprises the amino acid sequence of HX2YX3X4GX5TX6YNPX7X8X9X10 (SEQ ID NO: 531), wherein X2 is I or V; X3 is H, Y, or Q; X4 is T or S; X5 is K, S or Q; X6 is A, K or N; X7 is N, S, K, or R; X8 is F or L; X9 is E or K; and X10 is G, D, S, or N; and
HCDR3 comprises the amino acid sequence of X11YDX12X13X14GX15SYYYYGMDV (SEQ ID NO: 532), wherein X11 is D or N; X12 is A, I, or T; X13 is F, L, or V; X14 is S or T; and X15 is H, N or Y; and
b) the LV is of a VK1/O2/JK4 or a VK3/A27/JK1 germline.

11. The antibody of claim 10, wherein a) X1 is A or G; X3 is Q or Y, X4 is T, X8 is F, X14 is T; and X15 is Y.

12. The antibody of claim 10 wherein b) the LV is of a VK1/O2/JK4 germline.

13. The antibody of claim 11, wherein
b) the LV is of a VK1/O2/JK4 germline and comprises light chain LCDR1, LCDR2 and LCDR3, and wherein
LCDR1 comprises the amino acid sequence of RASQSISNYLN (SEQ ID NO: 132), RASQSIISYLN (SEQ ID NO: 150), or RASQSISSYLN (SEQ ID NO: 186);
LCDR2 comprises the amino acid sequence of AASSLHS (SEQ ID NO: 133) or AASSLRS (SEQ ID NO: 151); and
LCDR3 comprises the amino acid sequence of QQYQSYPLT (SEQ ID NO: 134) or QQYSNYPLT (SEQ ID NO: 152);
or
b) the LV is of a VK3/A27/JK1 germline and comprises light chain LCDR1, LCDR2 and LCDR3, and wherein
LCDR1 comprises the amino acid sequence of RASQSVSSYLG (SEQ ID NO: 168);
LCDR2 comprises the amino acid sequence of GASSRAT (SEQ ID NO: 169); and
LCDR3 comprises the amino acid sequence of QQYGSSPRT (SEQ ID NO: 170).

14. The antibody of claim 13, wherein LCDR1 comprises the amino acid sequence of RASQSISNYLN (SEQ ID NO: 132), LCDR2 comprises the amino acid sequence of AASSLHS (SEQ ID NO: 133), and LCDR3 comprises the amino acid sequence of QQYQSYPLT (SEQ ID NO: 134).

15. The antibody of claim 10, wherein
a) HCDR1 comprises the amino acid sequence of RGGDYWX1 (SEQ ID NO: 530), wherein X1 is A or G,
HCDR2 comprises the amino acid sequence of HX2YX3X4GX5TX6YNPX7X8X9X10 (SEQ ID NO: 531), wherein X2 is I or V; X3 is Y or Q; X4 is T; X5 is S; X6 is K or N; X7 is S or R; X8 is F; X9 is K; and X10 is G or D; and
HCDR3 comprises the amino acid sequence of X11YDX12X13X14GX15SYYYYGMDV (SEQ ID NO: 532), wherein X11 is N; X12 is T; X13 is L or V; X14 is T; and X15 is Y, and
b) the LV is of a VK1/O2/JK4 germline.

16. The antibody of claim 15, wherein
a) HCDR1 comprises the amino acid sequence of RGGDYWA (SEQ ID NO: 414) or RGGDYWG (SEQ ID NO: 408);
HCDR2 comprises the amino acid sequence of HVYYTGSTKYNPSFKD (SEQ ID NO: 427), HVYYTGSTNYNPRFKD (SEQ ID NO: 445), HIYQTGSTNYNPRFKG (SEQ ID NO: 415), or HVYQTGSTKYNPSFKD (SEQ ID NO: 409); and
HCDR3 comprises the amino acid sequence of NYDTLTGYSYYYYGMDV (SEQ ID NO: 410) or NYDTVTGYSYYYYGMDV (SEQ ID NO: 446).

17. The antibody of claim 15, wherein b) the LV comprises LCDR1, LCDR2 and LCDR3, and wherein LCDR1 comprises the sequence of RASQSISSYLN (SEQ ID NO: 405); LCDR2 comprises the sequence of X39ARX40LX41S (SEQ ID NO: 536), wherein X39 is N, S, K or G; X40 is R, K, L or A; and X41 is A, G or S; and LCDR3 comprises the sequence of QQX42X43X44X45PX46T (SEQ ID NO: 537), wherein X42 is Y or F, X43 is R, A, S, G, or Y; X44 is S, K, R or H; X45 is S, L, F, Y, P, or M; and X46 is L, I or V.

18. The antibody of claim 17, wherein X42 is Y.

19. The antibody of claim 5, wherein the amino acid at position 83 of the HV is N and the amino acid at position 85 of the HV is Y.

20. The antibody of claim 19, wherein the amino acid at position 94 of the LV is S.

21. An isolated antibody that binds to huCB1, the antibody comprises a HV and a LV, wherein
a) the HV comprises CDRH1, CDRH2 and CDRH3, wherein
CDRH1 comprises the amino acid sequence of RGGDYWS (SEQ ID NO: 1);
CDRH2 comprises the amino acid sequence of HX25YX26X27GX28TX29YNPX30X31X32X33 (SEQ ID NO: 538), wherein X25 is I or V; X26 is Y or Q; X27 is A, E, K, or T; X28 is S or Q; X29 is A, E, K, or T; X30 is N or S; X31 is F or L, X32 is K or R; and X33 is G, S, or N; and
CDRH3 comprises the amino acid sequence of X34YDX35X36X37GYSYYYYGX38DV (SEQ ID NO: 539), wherein X34 is D or G; X35 is A, I, T, or V; X36 is L or absent; X37 is S or T; and X38 is M or L; and
b) the LV is of a VK2/A19/JK1 germline.

22. The antibody of claim 21, wherein a) X26 is Y, X34 is G, X36 is absent, and X37 is S.

23. The antibody of claim 21, wherein b) the LV comprises LCDR1, LCDR2 and LCDR3, wherein LCDR1 comprises the amino acid sequence of RSSQSLLHRSGYNYLD (SEQ ID NO: 257); LCDR2 comprises the amino acid sequence of LGSNRAS (SEQ ID NO: 258) or QGSNRAS (SEQ ID NO: 264); and LCDR3 comprises the amino acid sequence of MQSLQTPRT (SEQ ID NO: 259), RQSVALPRT (SEQ ID NO: 271), or RQARALPRT (SEQ ID NO: 265).

24. The antibody of claim 21, wherein the amino acid at position 83 of the HV is N and the amino acid at position 85 of the HV is Y.

25. The antibody of claim 24, wherein the amino acid at position 79 of the HV is R.

26. The antibody of claim 21, wherein the amino acid at position 94 of the LV is S, or the amino acid at position 76 is D, or both.

27. The antibody of claim 21, wherein HCDR2 of the antibody comprises 5 or fewer mutations compared to HVYYTGSTNYNPRFKD (SEQ ID NO: 136).

28. The antibody of 27, wherein HCDR3 of the antibody comprises 3 or fewer mutations compared to NYDTVTGYSYYYYGMDV (SEQ ID NO: 137).

29. The antibody of claim 21, wherein HCDR2 of the antibody comprises 5 or fewer mutations compared to HVYYTGSTKYNPNFKG (SEQ ID NO: 8).

30. The antibody of claim 29, wherein HCDR3 of the antibody comprises 4 or fewer mutations compared to DYDILTGYSYYYYGMDV (SEQ ID NO: 9).

31. An isolated antibody or antigen binding fragment thereof that binds to huCB1, wherein the antigen binding molecule comprises a HV comprising HCDR1, HCDR2 and HCDR3 and a LV comprising LCDR1, LCDR2 and LCDR3, wherein HCDR1 comprises the amino acid sequence of RGGDYWS (SEQ ID NO: 474) or RGGDYWN (SEQ ID NO: 480), HCDR2 comprises the amino acid sequence of HIYYSGSTNYNPSLRS (SEQ ID NO: 475), HIYYSGSKNYNPSLKS (SEQ ID NO: 481), or HIYYTGTKYYNPSLKS (SEQ ID NO: 487), and HCDR3 comprises the amino acid sequence of DYDILYGYSYYYYGLDV (SEQ ID NO: 476), or GYDSSGYSYYYYGMDV (SEQ ID NO: 475), and wherein LCDR1 comprises the amino acid sequence of RSSQSLLHRSGYNYLD (SEQ ID NO: 471), RSSQSLLYSNGHNFLD (SEQ ID NO: 477), or RSSQSLLYSNGHNYLD (SEQ ID NO: 483), LCDR2 comprises the amino acid sequence of LGSNRAS (SEQ ID NO: 472) or LGSNRAP (SEQ ID NO: 484), and LCDR3 comprises the amino acid sequence of MQSLQTPRT (SEQ ID NO: 473) or MQALQTPRT (SEQ ID NO: 479).

32. The antibody of claim 31, wherein the HV comprises the amino acid sequence of SEQ ID NO: 489, SEQ ID NO: 491 or SEQ ID NO: 493, and the LV comprises the amino acid sequence of SEQ ID NO: 490, SEQ ID NO: 492 or SEQ ID NO 494.

33. The antibody of claim 21, wherein the amino acid at position 32 according to AHo numbering in the heavy chain is an R.

34. The antibody of claim 1, wherein the antibody is a monoclonal antibody.

35. (canceled)

36. The antibody of claim 34, wherein the monoclonal antibody is an antagonist antibody of huCB1.

37. The antibody of claim 34, wherein the monoclonal antibody is an inverse agonist antibody of huCB1.

38. The antibody of claim 36, wherein the antibody has a binding affinity for huCB1 that is at least 3 times higher compared to an antibody comprising a heavy chain variable region having the amino acid sequence of SEQ ID NO: 256 and a light chain variable region having the amino acid sequence of SEQ ID NO: 255 (10D10 LC N35Y).

39. The antibody of claim 36, wherein the antibody has an IC50 of 10 nM or less as determined using a cell-based cAMP assay.

40. The antibody of claim 39, wherein the antibody has an IC50 of 5 nM or less, or of 1 nM or less as determined using a cell-based cAMP assay.

41. (canceled)

42. The antibody of claim 41, wherein the monoclonal antibody is a human IgG1 antibody.

43. The antibody of claim 42, wherein the antibody comprises a mutation at amino acid position N297 according to EU numbering in its heavy chain.

44. The antibody of claim 43, wherein the mutation is N297G.

45. The antibody of claim 43, wherein the antibody further comprises R292C and V302C mutations according to EU numbering in its heavy chain.

46. The antibody of claim 42, wherein the antibody comprises mutations at amino acid positions M252, S254, and T256 according to EU numbering in its heavy chain.

47. The antibody of claim 46, wherein the mutations are M252Y, S254T, and T256E.

48. The antibody of claim 40, wherein the antibody comprises a heavy chain constant region amino acid sequence selected from SEQ ID NOs: 520-529 and a light chain constant region amino acid sequence selected from SEQ ID NOs: 513-519.

49. The antibody of claim 40, wherein the antibody comprises:
i) a heavy chain constant region amino acid sequence of SEQ ID NOs: 525, 526 or 527 and a light chain constant region amino acid sequence of SEQ ID NOs: 518 or 519; or
ii) a heavy chain constant region amino acid sequence of SEQ ID NOs: 525, 526 or 527 and a light chain constant region amino acid sequence of SEQ ID NOs: 518.

50. A pharmaceutical composition comprising the antibody of claim 1 and a pharmaceutically acceptable excipient.

51. An isolated polynucleotide that encodes the antibody of claim 1.

52. An expression vector comprising the polynucleotide of claim 51.

53. A host cell comprising the expression vector of claim 52.

54. A method of producing an antibody that binds to huCB1 comprising culturing the host cell of claim 53 under conditions that allow expression of the antibody and recovering the antibody from the culture medium or host cell.

55. A method for treating a subject in need of antagonizing or inverse agonizing the CB1 receptor, the method comprises administering an effective amount of the pharmaceutical composition of claim 50 to the subject.

56. A method for treating a disease or disorder in a subject responsive to antagonizing or inverse agonizing the CB1 receptor, the method comprises administering to the subject the antibody of claim 1.

57. The method of claim 56, wherein the CB1 receptor is a peripheral CB1 receptor.

58. The method of claim 57, wherein the administration results in one or more of reduced body weight, reduced appetite, improved metabolic parameters, reduced blood glucose levels, reduced insulin levels, reduced triglyceride levels, reduced kidney injury, reduced kidney fibrosis, reduced kidney inflammation, and improved kidney function.

59. The method of claim 57, wherein the disease or disorder is selected from obesity, diabetes, dyslipidemia, metabolic diseases, liver disease, fibrosis, non-alcoholic steatohepatitis (NASH), primary biliary cirrhosis, renal disease, kidney fibrosis, chronic kidney disease, IgA nephropathy, osteoporosis, atherosclerosis, cardiovascular disease, cancer, inflammatory disease, and a combination thereof.

60. A method for treating a disease or disorder in a subject in need thereof, the method comprises administering to the subject the antibody of claim 1, wherein the disease or disorder is obesity, diabetes, dyslipidemia, metabolic diseases, liver disease, fibrosis, NASH, primary biliary cirrhosis, renal disease, kidney fibrosis, chronic kidney disease, IgA nephropathy, osteoporosis, atherosclerosis, cardiovascular disease, cancer, inflammatory disease or a combination thereof.

61. (canceled)

62. The method of claim 60, wherein the administration results in one or more of reduced body weight, reduced appetite, improved metabolic parameters, reduced blood glucose levels, reduced insulin levels, reduced triglyceride levels, reduced kidney injury, reduced kidney fibrosis, reduced kidney inflammation, and improved kidney function.

63. The method of claim 56, wherein the subject is a human.

64.-66. (canceled)

67. The method of claim 59, wherein the disease or disorder is obesity.

68. The method of claim 56, wherein the subject has a BMI of at least 27 kg/m2.

69. The method or antibody of claim 68, wherein the subject has a BMI of at least 30 kg/m2.


SEQUENCE LISTING (566 sequences)

SEQ ID NO: 1 - protein, 7 aa, synthetic construct
RGGDYWS

SEQ ID NO: 2 - protein, 16 aa, synthetic construct
HIYYEGSTKYNPSFKG

SEQ ID NO: 3 - protein, 17 aa, synthetic construct
DYDPVTGHSYYYYGMDV

SEQ ID NO: 4 - protein, 16 aa, synthetic construct
RSSQSLLTSSGYNYVD

SEQ ID NO: 5 - protein, 7 aa, synthetic construct
QGSNRAS

SEQ ID NO: 6 - protein, 9 aa, synthetic construct
RQARAIPRT

SEQ ID NO: 7 - protein, 7 aa, synthetic construct
RGGDYWS

SEQ ID NO: 8 - protein, 16 aa, synthetic construct
HVYYTGSTKYNPNFKG

SEQ ID NO: 9 - protein, 17 aa, synthetic construct
DYDILTGYSYYYYGMDV

SEQ ID NO: 10 - protein, 16 aa, synthetic construct
RSSQSLLTSYGINYVD

SEQ ID NO: 11 - protein, 7 aa, synthetic construct
QGSNRAS

SEQ ID NO: 12 - protein, 9 aa, synthetic construct
RQAVGLPRT

SEQ ID NO: 13 - protein, 7 aa, synthetic construct
RGGDYWS

SEQ ID NO: 14 - protein, 16 aa, synthetic construct
HIYQTGQTKYNPNLKG

SEQ ID NO: 15 - protein, 17 aa, synthetic construct
DYDILTGYSYYYYGMDV

SEQ ID NO: 16 - protein, 16 aa, synthetic construct
RSSQSLLSSTGANYLD

SEQ ID NO: 17 - protein, 7 aa, synthetic construct
QGSNRAS

SEQ ID NO: 18 - protein, 9 aa, synthetic construct
RQAVGLPRT

SEQ ID NO: 19 - protein, 7 aa, synthetic construct
RGGDYWS

SEQ ID NO: 20 - protein, 16 aa, synthetic construct
HVYYTGSTKYNPSFEG

SEQ ID NO: 21 - protein, 17 aa, synthetic construct
DYDILTGYSYYYYGMDV

SEQ ID NO: 22 - protein, 16 aa, synthetic construct
RSSQSLLHSYGANYLD

SEQ ID NO: 23 - protein, 7 aa, synthetic construct
LGSNRAS

SEQ ID NO: 24 - protein, 9 aa, synthetic construct
RQARALPRT

SEQ ID NO: 25 - protein, 7 aa, synthetic construct
RGGDYWS

SEQ ID NO: 26 - protein, 16 aa, synthetic construct
HIYQTGQTAYNPRLEG

SEQ ID NO: 27 - protein, 17 aa, synthetic construct
DYDILTGYSYYYYGMDV

SEQ ID NO: 28 - protein, 16 aa, synthetic construct
RSSQSLLSSTGANYLD

SEQ ID NO: 29 - protein, 7 aa, synthetic construct
LGSNRAS

SEQ ID NO: 30 - protein, 9 aa, synthetic construct
RQAVELPRT

SEQ ID NO: 31 - protein, 7 aa, synthetic construct
RGGDYWS

SEQ ID NO: 32 - protein, 16 aa, synthetic construct
HVYYEGSTAYNPSFED

SEQ ID NO: 33 - protein, 17 aa, synthetic construct
DYDILTGYSYYYYGMDV

SEQ ID NO: 34 - protein, 16 aa, synthetic construct
RSSQSLLTSYGINYVD

SEQ ID NO: 35 - protein, 7 aa, synthetic construct
LGSNRAS

SEQ ID NO: 36 - protein, 9 aa, synthetic construct
MQARQIPRT

SEQ ID NO: 37 - protein, 7 aa, synthetic construct
RGGDYWS

SEQ ID NO: 38 - protein, 16 aa, synthetic construct
HVYYTGSTKYNPSFKD

SEQ ID NO: 39 - protein, 17 aa, synthetic construct
DYDILTGYSYYYYGMDV

SEQ ID NO: 40 - protein, 16 aa, synthetic construct
RSSQSLLSSSGANYVD

SEQ ID NO: 41 - protein, 7 aa, synthetic construct
QGSNRAS

SEQ ID NO: 42 - protein, 9 aa, synthetic construct
RQARALPRT

SEQ ID NO: 43 - protein, 7 aa, synthetic construct
RGGDYWS

SEQ ID NO: 44 - protein, 16 aa, synthetic construct
HVYQTGSTKYNPKFKG

SEQ ID NO: 45 - protein, 17 aa, synthetic construct
DYDILTGYSYYYYGMDV

SEQ ID NO: 46 - protein, 16 aa, synthetic construct
RSSQSLLTSSGANYVD

SEQ ID NO: 47 - protein, 7 aa, synthetic construct
LGSNRAS

SEQ ID NO: 48 - protein, 9 aa, synthetic construct
RQAVGTPRT

SEQ ID NO: 49 - protein, 7 aa, synthetic construct
RGGDYWS

SEQ ID NO: 50 - protein, 16 aa, synthetic construct
HIYQTGSTAYNPSLKG

SEQ ID NO: 51 - protein, 17 aa, synthetic construct
DYDILTGYSYYYYGMDV

SEQ ID NO: 52 - protein, 16 aa, synthetic construct
RSSQSLLHSTGANYVD

SEQ ID NO: 53 - protein, 7 aa, synthetic construct
LGSNRAS

SEQ ID NO: 54 - protein, 9 aa, synthetic construct
RQAIALPRT

SEQ ID NO: 55 - protein, 7 aa, synthetic construct
RGGDYWS

SEQ ID NO: 56 - protein, 16 aa, synthetic construct
HIYYTGSTAYNPSLKS

SEQ ID NO: 57 - protein, 17 aa, synthetic construct
DYDILTGYSYYYYGMDV

SEQ ID NO: 58 - protein, 16 aa, synthetic construct
RSSQSLLHSYGANYLD

SEQ ID NO: 59 - protein, 7 aa, synthetic construct
LGSNRAS

SEQ ID NO: 60 - protein, 9 aa, synthetic construct
RQARTLPRT

SEQ ID NO: 61 - protein, 7 aa, synthetic construct
RGGDYWS

SEQ ID NO: 62 - protein, 16 aa, synthetic construct
HVYYTGQTAYNPKFKD

SEQ ID NO: 63 - protein, 17 aa, synthetic construct
DYDILTGYSYYYYGMDV

SEQ ID NO: 64 - protein, 16 aa, synthetic construct
RSSQSLLSSSGANYVD

SEQ ID NO: 65 - protein, 7 aa, synthetic construct
LGSNRAS

SEQ ID NO: 66 - protein, 9 aa, synthetic construct
MQARGIPRT

SEQ ID NO: 67 - protein, 7 aa, synthetic construct
RGGDYWS

SEQ ID NO: 68 - protein, 16 aa, synthetic construct
HIYQSGQTKYNPSLKD

SEQ ID NO: 69 - protein, 17 aa, synthetic construct
DYDILTGYSYYYYGMDV

SEQ ID NO: 70 - protein, 16 aa, synthetic construct
RSSQSLLTSSGANYVD

SEQ ID NO: 71 - protein, 7 aa, synthetic construct
LGSNRAS

SEQ ID NO: 72 - protein, 9 aa, synthetic construct
RQAREQPRT

SEQ ID NO: 73 - protein, 7 aa, synthetic construct
RGGDYWA

SEQ ID NO: 74 - protein, 16 aa, synthetic construct
HVYQSGQTAYNPNFEN

SEQ ID NO: 75 - protein, 17 aa, synthetic construct
DYDAVSGYSYYYYGMDV

SEQ ID NO: 76 - protein, 16 aa, synthetic construct
RSSQSLLSSYGNNYVD

SEQ ID NO: 77 - protein, 7 aa, synthetic construct
QGSNRAS

SEQ ID NO: 78 - protein, 9 aa, synthetic construct
RQAVGLPRT

SEQ ID NO: 79 - protein, 7 aa, synthetic construct
RGGDYWS

SEQ ID NO: 80 - protein, 16 aa, synthetic construct
HVYQSGQTAYNPNFED

SEQ ID NO: 81 - protein, 17 aa, synthetic construct
EYDVVSGYSYYYYGMDV

SEQ ID NO: 82 - protein, 16 aa, synthetic construct
RSSQSLLTSSGYNYVD

SEQ ID NO: 83 - protein, 7 aa, synthetic construct
QGSNRAS

SEQ ID NO: 84 - protein, 9 aa, synthetic construct
MQAVKIPRT

SEQ ID NO: 85 - protein, 7 aa, synthetic construct
RGGDYWA

SEQ ID NO: 86 - protein, 16 aa, synthetic construct
HVYQTGQTAYNPNLES

SEQ ID NO: 87 - protein, 17 aa, synthetic construct
NYDVLSGYSYYYYGMDV

SEQ ID NO: 88 - protein, 16 aa, synthetic construct
RSSQSLLTSYGANYLD

SEQ ID NO: 89 - protein, 7 aa, synthetic construct
QGSNRAS

SEQ ID NO: 90 - protein, 9 aa, synthetic construct
MQALQIPRT

SEQ ID NO: 91 - protein, 7 aa, synthetic construct
RGGDYWS

SEQ ID NO: 92 - protein, 16 aa, synthetic construct
YIYYSGSTNYNPSLKS

SEQ ID NO: 93 - protein, 17 aa, synthetic construct
DYDILTGYSYYYYGMDV

SEQ ID NO: 94 - protein, 16 aa, synthetic construct
RSSQSLLHSNGYNYLD

SEQ ID NO: 95 - protein, 7 aa, synthetic construct
LGSNRAS

SEQ ID NO: 96 - protein, 9 aa, synthetic construct
MQALQTPRT

SEQ ID NO: 97 - protein, 113 aa, synthetic construct
DIVMTQSPLSLPVTPGEPASISCRSSQSLLTSSGYNYVDWYLQKPGQSPQLLIYQGSNRASGVPDRFSGSGSGTDFTLKISRVEAEDVGVYYCRQARAIPRTFGQGTKVEIKR

SEQ ID NO: 98 - protein, 127 aa, synthetic construct
QVQLQESGPGLVKPSQTLSLTCTVSGGSIRRGGDYWSWIRQHPGKGLEWIGHIYYEGSTKYNPSFKGRATISVNTYKNQFSLKLSSVTAADTAVYYCARDYDPVTGHSYYYYGMDVWGQGTTVTVSS

SEQ ID NO: 99 - protein, 113 aa, synthetic construct
DIVMTQSPLSLPVTPGEPASISCRSSQSLLTSYGINYVDWYLQKPGQSPQLLIYQGSNRASGVPDRFSGSGSGTDFTLKISRVEAEDVGVYYCRQAVGLPRTFGQGTKVEIKR

SEQ ID NO: 100 - protein, 127 aa, synthetic construct
QVQLQESGPGLVKPSQTLSLTCTVSGGSIRRGGDYWSWIRQHPGKGLEWIGHVYYTGSTKYNPNFKGRATISVNTYKNQFSLKLSSVTAADTAVYYCARDYDILTGYSYYYYGMDVWGQGTTVTVSS

SEQ ID NO: 101 - protein, 113 aa, synthetic construct
DIVMTQSPLSLPVTPGEPASISCRSSQSLLSSTGANYLDWYLQKPGQSPQLLIYQGSNRASGVPDRFSGSGSGTDFTLKISRVEAEDVGVYYCRQAVGLPRTFGQGTKVEIKR

SEQ ID NO: 102 - protein, 127 aa, synthetic construct
QVQLQESGPGLVKPSQTLSLTCTVSGGSIRRGGDYWSWIRQHPGKGLEWIGHIYQTGQTKYNPNLKGRATISVNTYKNQFSLKLSSVTAADTAVYYCARDYDILTGYSYYYYGMDVWGQGTTVTVSS

SEQ ID NO: 103 - protein, 113 aa, synthetic construct
DIVMTQSPLSLPVTPGEPASISCRSSQSLLHSYGANYLDWYLQKPGQSPQLLIYLGSNRASGVPDRFSGSGSGTDFTLKISRVEAEDVGVYYCRQARALPRTFGQGTKVEIKR

SEQ ID NO: 104 - protein, 127 aa, synthetic construct
QVQLQESGPGLVKPSQTLSLTCTVSGGSIRRGGDYWSWIRQHPGKGLEWIGHVYYTGSTKYNPSFEGRATISVKTYKNQFSLKLSSVTAADTAVYYCARDYDILTGYSYYYYGMDVWGQGTTVTVSS

SEQ ID NO: 105 - protein, 113 aa, synthetic construct
DIVMTQSPLSLPVTPGEPASISCRSSQSLLSSTGANYLDWYLQKPGQSPQLLIYLGSNRASGVPDRFSGSGSGTDFTLKISRVEAEDVGVYYCRQAVELPRTFGQGTKVEIKR

SEQ ID NO: 106 - protein, 127 aa, synthetic construct
QVQLQESGPGLVKPSQTLSLTCTVSGGSIRRGGDYWSWIRQHPGKGLEWIGHIYQTGQTAYNPRLEGRATISVNTYKNQFSLKLSSVTAADTAVYYCARDYDILTGYSYYYYGMDVWGQGTTVTVSS

SEQ ID NO: 107 - protein, 113 aa, synthetic construct
DIVMTQSPLSLPVTPGEPASISCRSSQSLLTSYGINYVDWYLQKPGQSPQLLIYLGSNRASGVPDRFSGSGSGTDFTLKISRVEAEDVGVYYCMQARQIPRTFGQGTKVEIKR

SEQ ID NO: 108 - protein, 127 aa, synthetic construct
QVQLQESGPGLVKPSQTLSLTCTVSGGSIRRGGDYWSWIRQHPGKGLEWIGHVYYEGSTAYNPSFEDRATISVNTYKNQFSLKLSSVTAADTAVYYCARDYDILTGYSYYYYGMDVWGQGTTVTVSS

SEQ ID NO: 109 - protein, 113 aa, synthetic construct
DIVMTQSPLSLPVTPGEPASISCRSSQSLLSSSGANYVDWYLQKPGQSPQLLIYQGSNRASGVPDRFSGSGSGTDFTLKISRVEAEDVGVYYCRQARALPRTFGQGTKVEIKR

SEQ ID NO: 110 - protein, 127 aa, synthetic construct
QVQLQESGPGLVKPSQTLSLTCTVSGGSIRRGGDYWSWIRQHPGKGLEWIGHVYYTGSTKYNPSFKDRATISVNTYKNQFSLKLSSVTAADTAVYYCARDYDILTGYSYYYYGMDVWGQGTTVTVSS

SEQ ID NO: 111 - protein, 113 aa, synthetic construct
DIVMTQSPLSLPVTPGEPASISCRSSQSLLTSSGANYVDWYLQKPGQSPQLLIYLGSNRASGVPDRFSGSGSGTDFTLKISRVEAEDVGVYYCRQAVGTPRTFGQGTKVEIKR

SEQ ID NO: 112 - protein, 127 aa, synthetic construct
QVQLQESGPGLVKPSQTLSLTCTVSGGSIRRGGDYWSWIRQHPGKGLEWIGHVYQTGSTKYNPKFKGRATISVNTYKNQFSLKLSSVTAADTAVYYCARDYDILTGYSYYYYGMDVWGQGTTVTVSS

SEQ ID NO: 113 - protein, 113 aa, synthetic construct
DIVMTQSPLSLPVTPGEPASISCRSSQSLLHSTGANYVDWYLQKPGQSPQLLIYLGSNRASGVPDRFSGSGSGTDFTLKISRVEAEDVGVYYCRQAIALPRTFGQGTKVEIKR

SEQ ID NO: 114 - protein, 127 aa, synthetic construct
QVQLQESGPGLVKPSQTLSLTCTVSGGSIRRGGDYWSWIRQHPGKGLEWIGHIYQTGSTAYNPSLKGRATISVNTYKNQFSLKLSSVTAADTAVYYCARDYDILTGYSYYYYGMDVWGQGTTVTVSS

SEQ ID NO: 115 - protein, 113 aa, synthetic construct
DIVMTQSPLSLPVTPGEPASISCRSSQSLLHSYGANYLDWYLQKPGQSPQLLIYLGSNRASGVPDRFSGSGSGTDFTLKISRVEAEDVGVYYCRQARTLPRTFGQGTKVEIKR

SEQ ID NO: 116 - protein, 127 aa, synthetic construct
QVQLQESGPGLVKPSQTLSLTCTVSGGSIRRGGDYWSWIRQHPGKGLEWIGHIYYTGSTAYNPSLKSRATISVNTYKNQFSLKLSSVTAADTAVYYCARDYDILTGYSYYYYGMDVWGQGTTVTVSS

SEQ ID NO: 117 - protein, 113 aa, synthetic construct
DIVMTQSPLSLPVTPGEPASISCRSSQSLLSSSGANYVDWYLQKPGQSPQLLIYLGSNRASGVPDRFSGSGSGTDFTLKISRVEAEDVGVYYCMQARGIPRTFGQGTKVEIKR

SEQ ID NO: 118 - protein, 127 aa, synthetic construct
QVQLQESGPGLVKPSQTLSLTCTVSGGSIRRGGDYWSWIRQHPGKGLEWIGHVYYTGQTAYNPKFKDRATISVKTYKNQFSLKLSSVTAADTAVYYCARDYDILTGYSYYYYGMDVWGQGTTVTVSS

SEQ ID NO: 119 - protein, 113 aa, synthetic construct
DIVMTQSPLSLPVTPGEPASISCRSSQSLLTSSGANYVDWYLQKPGQSPQLLIYLGSNRASGVPDRFSGSGSGTDFTLKISRVEAEDVGVYYCRQAREQPRTFGQGTKVEIKR

SEQ ID NO: 120 - protein, 127 aa, synthetic construct
QVQLQESGPGLVKPSQTLSLTCTVSGGSIRRGGDYWSWIRQHPGKGLEWIGHIYQSGQTKYNPSLKDRATISVNTYKNQFSLKLSSVTAADTAVYYCARDYDILTGYSYYYYGMDVWGQGTTVTVSS

SEQ ID NO: 121 - protein, 113 aa, synthetic construct
DIVMTQSPLSLPVTPGEPASISCRSSQSLLSSYGNNYVDWYLQKPGQSPQLLIYQGSNRASGVPDRFSGSGSGTDFTLKISRVEAEDVGVYYCRQAVGLPRTFGQGTKVEIKR

SEQ ID NO: 122 - protein, 127 aa, synthetic construct
QVQLQESGPGLVKPSQTLSLTCTVSGGSIRRGGDYWAWIRQHPGKGLEWIGHVYQSGQTAYNPNFENRATISVNTYKNQFSLKLSSVTAADTAVYYCARDYDAVSGYSYYYYGMDVWGQGTTVTVSS

SEQ ID NO: 123 - protein, 113 aa, synthetic construct
DIVMTQSPLSLPVTPGEPASISCRSSQSLLTSSGYNYVDWYLQKPGQSPQLLIYQGSNRASGVPDRFSGSGSGTDFTLKISRVEAEDVGVYYCMQAVKIPRTFGQGTKVEIKR

SEQ ID NO: 124 - protein, 127 aa, synthetic construct
QVQLQESGPGLVKPSQTLSLTCTVSGGSIRRGGDYWSWIRQHPGKGLEWIGHVYQSGQTAYNPNFEDRATISVNTYKNQFSLKLSSVTAADTAVYYCAREYDVVSGYSYYYYGMDVWGQGTTVTVSS

SEQ ID NO: 125 - protein, 113 aa, synthetic construct
DIVMTQSPLSLPVTPGEPASISCRSSQSLLTSYGANYLDWYLQKPGQSPQLLIYQGSNRASGVPDRFSGSGSGTDFTLKISRVEAEDVGVYYCMQALQIPRTFGQGTKVEIKR

SEQ ID NO: 126 - protein, 127 aa, synthetic construct
QVQLQESGPGLVKPSQTLSLTCTVSGGSIRRGGDYWAWIRQHPGKGLEWIGHVYQTGQTAYNPNLESRATISVNTYKNQFSLKLSSVTAADTAVYYCARNYDVLSGYSYYYYGMDVWGQGTTVTVSS

SEQ ID NO: 127 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKIRRVEAEDVGVYYCMQALQTPRTFGQGTKVEIKR

SEQ ID NO: 128 - protein, 127 aa, synthetic construct
QVQLQESGPGLVKPSQTLSLTCTVSGGSIRRGGDYWSWIRQHPGKGLEWIGYIYYSGSTNYNPSLKSRATISVDTSKNQFSLKLSSVTAADTAVYYCARDYDILTGYSYYYYGMDVWGQGTTVTVSS

SEQ ID NO: 129 - protein, 7 aa, synthetic construct
RGGDYWA

SEQ ID NO: 130 - protein, 16 aa, synthetic construct
HIYQTGSTNYNPRFKG

SEQ ID NO: 131 - protein, 17 aa, synthetic construct
NYDTLTGYSYYYYGMDV

SEQ ID NO: 132 - protein, 11 aa, synthetic construct
RASQSISNYLN

SEQ ID NO: 133 - protein, 7 aa, synthetic construct
AASSLHS

SEQ ID NO: 134 - protein, 9 aa, synthetic construct
QQYQSYPLT

SEQ ID NO: 135 - protein, 7 aa, synthetic construct
RGGDYWA

SEQ ID NO: 136 - protein, 16 aa, synthetic construct
HVYYTGSTNYNPRFKD

SEQ ID NO: 137 - protein, 17 aa, synthetic construct
NYDTVTGYSYYYYGMDV

SEQ ID NO: 138 - protein, 11 aa, synthetic construct
RASQSISNYLN

SEQ ID NO: 139 - protein, 7 aa, synthetic construct
AASSLHS

SEQ ID NO: 140 - protein, 9 aa, synthetic construct
QQYQSYPLT

SEQ ID NO: 141 - protein, 7 aa, synthetic construct
RGGDYWS

SEQ ID NO: 142 - protein, 16 aa, synthetic construct
HIYQSGSTKYNPSFED

SEQ ID NO: 143 - protein, 17 aa, synthetic construct
DYDILTGYSYYYYGMDV

SEQ ID NO: 144 - protein, 11 aa, synthetic construct
RASQSISNYLN

SEQ ID NO: 145 - protein, 7 aa, synthetic construct
AASSLHS

SEQ ID NO: 146 - protein, 9 aa, synthetic construct
QQYQSYPLT

SEQ ID NO: 147 - protein, 7 aa, synthetic construct
RGGDYWG

SEQ ID NO: 148 - protein, 16 aa, synthetic construct
HIYQSGSTNYNPKLKS

SEQ ID NO: 149 - protein, 17 aa, synthetic construct
DYDIFTGYSYYYYGMDV

SEQ ID NO: 150 - protein, 11 aa, synthetic construct
RASQSIISYLN

SEQ ID NO: 151 - protein, 7 aa, synthetic construct
AASSLRS

SEQ ID NO: 152 - protein, 9 aa, synthetic construct
QQYSNYPLT

SEQ ID NO: 153 - protein, 7 aa, synthetic construct
RGGDYWS

SEQ ID NO: 154 - protein, 16 aa, synthetic construct
HVYQTGSTKYNPRFKG

SEQ ID NO: 155 - protein, 17 aa, synthetic construct
DYDILTGYSYYYYGMDV

SEQ ID NO: 156 - protein, 11 aa, synthetic construct
RASQSISNYLN

SEQ ID NO: 157 - protein, 7 aa, synthetic construct
AASSLHS

SEQ ID NO: 158 - protein, 9 aa, synthetic construct
QQYQSYPLT

SEQ ID NO: 159 - protein, 7 aa, synthetic construct
RGGDYWG

SEQ ID NO: 160 - protein, 16 aa, synthetic construct
HVYQTGSTKYNPSFKD

SEQ ID NO: 161 - protein, 17 aa, synthetic construct
NYDTLTGYSYYYYGMDV

SEQ ID NO: 162 - protein, 11 aa, synthetic construct
RASQSISNYLN

SEQ ID NO: 163 - protein, 7 aa, synthetic construct
AASSLHS

SEQ ID NO: 164 - protein, 9 aa, synthetic construct
QQYQSYPLT

SEQ ID NO: 165 - protein, 7 aa, synthetic construct
RGGDYWS

SEQ ID NO: 166 - protein, 16 aa, synthetic construct
HIYQTGQTAYNPNLEG

SEQ ID NO: 167 - protein, 17 aa, synthetic construct
DYDILTGYSYYYYGMDV

SEQ ID NO: 168 - protein, 11 aa, synthetic construct
RASQSVSSYLG

SEQ ID NO: 169 - protein, 7 aa, synthetic construct
GASSRAT

SEQ ID NO: 170 - protein, 9 aa, synthetic construct
QQYGSSPRT

SEQ ID NO: 171 - protein, 7 aa, synthetic construct
RGGDYWS

SEQ ID NO: 172 - protein, 16 aa, synthetic construct
HIYQTGKTNYNPNFKS

SEQ ID NO: 173 - protein, 17 aa, synthetic construct
DYDILTGYSYYYYGMDV

SEQ ID NO: 174 - protein, 11 aa, synthetic construct
RASQSISNYLN

SEQ ID NO: 175 - protein, 7 aa, synthetic construct
AASSLHS

SEQ ID NO: 176 - protein, 9 aa, synthetic construct
QQYQSYPLT

SEQ ID NO: 177 - protein, 7 aa, synthetic construct
RGGDYWG

SEQ ID NO: 178 - protein, 16 aa, synthetic construct
HIYQTGSTNYNPNFKD

SEQ ID NO: 179 - protein, 17 aa, synthetic construct
DYDILTGYSYYYYGMDV

SEQ ID NO: 180 - protein, 11 aa, synthetic construct
RASQSISNYLN

SEQ ID NO: 181 - protein, 7 aa, synthetic construct
AASSLHS

SEQ ID NO: 182 - protein, 9 aa, synthetic construct
QQYQSYPLT

SEQ ID NO: 183 - protein, 7 aa, synthetic construct
RGGDYWS

SEQ ID NO: 184 - protein, 16 aa, synthetic construct
YIYQTGQTKYNPNFEG

SEQ ID NO: 185 - protein, 17 aa, synthetic construct
DYDILTGYSYYYYGMDV

SEQ ID NO: 186 - protein, 11 aa, synthetic construct
RASQSISSYLN

SEQ ID NO: 187 - protein, 7 aa, synthetic construct
AASSLRS

SEQ ID NO: 188 - protein, 9 aa, synthetic construct
QQYSNYPLT

SEQ ID NO: 189 - protein, 7 aa, synthetic construct
RGGDYWS

SEQ ID NO: 190 - protein, 16 aa, synthetic construct
HIYQSGQTKYNPKLKG

SEQ ID NO: 191 - protein, 17 aa, synthetic construct
DYDILTGYSYYYYGMDV

SEQ ID NO: 192 - protein, 11 aa, synthetic construct
RASQSISNYLN

SEQ ID NO: 193 - protein, 7 aa, synthetic construct
AASSLHS

SEQ ID NO: 194 - protein, 9 aa, synthetic construct
QQYQSYPLT

SEQ ID NO: 195 - protein, 7 aa, synthetic construct
RGGDYWS

SEQ ID NO: 196 - protein, 16 aa, synthetic construct
HIYQSGSTKYNPSFKD

SEQ ID NO: 197 - protein, 17 aa, synthetic construct
DYDILTGYSYYYYGMDV

SEQ ID NO: 198 - protein, 11 aa, synthetic construct
RASQSISNYLN

SEQ ID NO: 199 - protein, 7 aa, synthetic construct
AASSLHS

SEQ ID NO: 200 - protein, 9 aa, synthetic construct
QQYQSYPLT

SEQ ID NO: 201 - protein, 7 aa, synthetic construct
RGGDYWA

SEQ ID NO: 202 - protein, 16 aa, synthetic construct
HIYHTGKTNYNPSFKG

SEQ ID NO: 203 - protein, 17 aa, synthetic construct
NYDTLSGNSYYYYGMDV

SEQ ID NO: 204 - protein, 11 aa, synthetic construct
RASQSISNYLN

SEQ ID NO: 205 - protein, 7 aa, synthetic construct
AASSLHS

SEQ ID NO: 206 - protein, 9 aa, synthetic construct
QQYQSYPLT

SEQ ID NO: 207 - protein, 7 aa, synthetic construct
RGGDYWA

SEQ ID NO: 208 - protein, 16 aa, synthetic construct
HVYQTGSTAYNPNFKD

SEQ ID NO: 209 - protein, 17 aa, synthetic construct
NYDALSGYSYYYYGMDV

SEQ ID NO: 210 - protein, 11 aa, synthetic construct
RASQSIISYLN

SEQ ID NO: 211 - protein, 7 aa, synthetic construct
AASSLRS

SEQ ID NO: 212 - protein, 9 aa, synthetic construct
QQYSNYPLT

SEQ ID NO: 213 - protein, 7 aa, synthetic construct
RGGDYWG

SEQ ID NO: 214 - protein, 16 aa, synthetic construct
HVYQSGQTAYNPKFEN

SEQ ID NO: 215 - protein, 17 aa, synthetic construct
NYDTLTGHSYYYYGMDV

SEQ ID NO: 216 - protein, 11 aa, synthetic construct
RASQSISNYLN

SEQ ID NO: 217 - protein, 7 aa, synthetic construct
AASSLHS

SEQ ID NO: 218 - protein, 9 aa, synthetic construct
QQYQSYPLT

SEQ ID NO: 219 - protein, 7 aa, synthetic construct
RGGDYWS

SEQ ID NO: 220 - protein, 16 aa, synthetic construct
YIYYSGSTNYNPSLKS

SEQ ID NO: 221 - protein, 17 aa, synthetic construct
DYDILTGYSYYYYGMDV

SEQ ID NO: 222 - protein, 16 aa, synthetic construct
RSSQSLLHSYGYNYLD

SEQ ID NO: 223 - protein, 7 aa, synthetic construct
LGSNRAS

SEQ ID NO: 224 - protein, 9 aa, synthetic construct
MQALQTPRT

SEQ ID NO: 225 - protein, 108 aa, synthetic construct
DIQMTQSPSSLSASVGDRVTITCRASQSISNYLNWYQQKPGKAPKLLIYAASSLHSGVPSRFSGSGSGTDFTLTISSLQPEDFASYYCQQYQSYPLTFGQGTKVEIKR

SEQ ID NO: 226 - protein, 127 aa, synthetic construct
QVQLQESGPGLVKPSQTLSLTCTVSGGSIRRGGDYWAWIRQHPGKGLEWIGHIYQTGSTNYNPRFKGRATISVNTYKNQFSLKLSSVTAADTAVYYCARNYDTLTGYSYYYYGMDVWGQGTTVTVSS

SEQ ID NO: 227 - protein, 108 aa, synthetic construct
DIQLTQSPSTLSASVGDRVTITCRASQSISNYLNWYQQKPGKAPKLLIYAASSLHSGVPSRFSGSGSGTDFTLTISSLQPEDFASYYCQQYQSYPLTFGQGTKVEIKR

SEQ ID NO: 228 - protein, 127 aa, synthetic construct
QVQLQESGPGLVKPSQTLSLTCTVSGGSIRRGGDYWAWIRQHPGKGLEWIGHVYYTGSTNYNPRFKDRATISVNTYKNQFSLKLSSVTAADTAVYYCARNYDTVTGYSYYYYGMDVWGQGTTVTVSS

SEQ ID NO: 229 - protein, 108 aa, synthetic construct; identical to SEQ ID NO: 227

SEQ ID NO: 230 - protein, 127 aa, synthetic construct
QVQLQESGPGLVKPSQTLSLTCTVSGGSIRRGGDYWSWIRQHPGKGLEWIGHIYQSGSTKYNPSFEDRATISVNTYKNQFSLKLSSVTAADTAVYYCARDYDILTGYSYYYYGMDVWGQGTTVTVSS

SEQ ID NO: 231 - protein, 108 aa, synthetic construct
DIQLTQSPSSLSASVGDRVTITCRASQSIISYLNWYQQKPGKAPKLLIYAASSLRSGVPSRFSGSGSGTDFTLTISSLQPEDFATYYCQQYSNYPLTFGGGTRVEIKR

SEQ ID NO: 232 - protein, 127 aa, synthetic construct
QVQLQESGPGLVKPSQTLSLTCTVSGGSIRRGGDYWGWIRQHPGKGLEWIGHIYQSGSTNYNPKLKSRATISVDTYKNQFSLKLSSVTAADTAVYYCARDYDIFTGYSYYYYGMDVWGQGTTVTVSS

SEQ ID NO: 233 - protein, 108 aa, synthetic construct; identical to SEQ ID NO: 227

SEQ ID NO: 234 - protein, 127 aa, synthetic construct
QVQLQESGPGLVKPSQTLSLTCTVSGGSIRRGGDYWSWIRQHPGKGLEWIGHVYQTGSTKYNPRFKGRATISVNTYKNQFSLKLSSVTAADTAVYYCARDYDILTGYSYYYYGMDVWGQGTTVTVSS

SEQ ID NO: 235 - protein, 108 aa, synthetic construct
DIQMTQSPSTLSASVGDRVTITCRASQSISNYLNWYQQKPGKAPKLLIYAASSLHSGVPSRFSGSGSGTDFTLTISSLQPEDFASYYCQQYQSYPLTFGQGTKVEIKR

SEQ ID NO: 236 - protein, 127 aa, synthetic construct
QVQLQESGPGLVKPSQTLSLTCTVSGGSIRRGGDYWGWIRQHPGKGLEWIGHVYQTGSTKYNPSFKDRATISVNTYKNQFSLKLSSVTAADTAVYYCARNYDTLTGYSYYYYGMDVWGQGTTVTVSS

SEQ ID NO: 237 - protein, 108 aa, synthetic construct
EIVLTQSPATLSLSPGERATLSCRASQSVSSYLGWYQQKPGQAPRLLIYGASSRATGIPDRFSGSGSGTDFTLTISRLEPEDFAVYYCQQYGSSPRTFGQGTKVEIKR

SEQ ID NO: 238 - protein, 127 aa, synthetic construct
QVQLQESGPGLVKPSQTLSLTCTVSGGSIRRGGDYWSWIRQHPGKGLEWIGHIYQTGQTAYNPNLEGRATISVNTYKNQFSLKLSSVTAADTAVYYCARDYDILTGYSYYYYGMDVWGQGTTVTVSS

SEQ ID NO: 239 - protein, 108 aa, synthetic construct; identical to SEQ ID NO: 235

SEQ ID NO: 240 - protein, 127 aa, synthetic construct
QVQLQESGPGLVKPSQTLSLTCTVSGGSIRRGGDYWSWIRQHPGKGLEWIGHIYQTGKTNYNPNFKSRATISVNTYKNQFSLKLSSVTAADTAVYYCARDYDILTGYSYYYYGMDVWGQGTTVTVSS

SEQ ID NO: 241 - protein, 108 aa, synthetic construct; identical to SEQ ID NO: 227

SEQ ID NO: 242 - protein, 127 aa, synthetic construct
QVQLQESGPGLVKPSQTLSLTCTVSGGSIRRGGDYWGWIRQHPGKGLEWIGHIYQTGSTNYNPNFKDRATISVDTYKNQFSLKLSSVTAADTAVYYCARDYDILTGYSYYYYGMDVWGQGTTVTVSS

SEQ ID NO: 243 - protein, 108 aa, synthetic construct
DIQLTQSPSSLSASVGDRVTITCRASQSISSYLNWYQQKPGKAPKLLIYAASSLRSGVPSRFSGSGSGTDFTLTISSLQPEDFATYYCQQYSNYPLTFGGGTRVEIKR

SEQ ID NO: 244 - protein, 127 aa, synthetic construct
QVQLQESGPGLVKPSQTLSLTCTVSGGSIRRGGDYWSWIRQHPGKGLEWIGYIYQTGQTKYNPNFEGRATISVNTYKNQFSLKLSSVTAADTAVYYCARDYDILTGYSYYYYGMDVWGQGTTVTVSS

SEQ ID NO: 245 - protein, 108 aa, synthetic construct; identical to SEQ ID NO: 227

SEQ ID NO: 246 - protein, 127 aa, synthetic construct
QVQLQESGPGLVKPSQTLSLTCTVSGGSIRRGGDYWSWIRQHPGKGLEWIGHIYQSGQTKYNPKLKGRATISVDTYKNQFSLKLSSVTAADTAVYYCARDYDILTGYSYYYYGMDVWGQGTTVTVSS

SEQ ID NO: 247 - protein, 108 aa, synthetic construct; identical to SEQ ID NO: 227

SEQ ID NO: 248 - protein, 127 aa, synthetic construct
QVQLQESGPGLVKPSQTLSLTCTVSGGSIRRGGDYWSWIRQHPGKGLEWIGHIYQSGSTKYNPSFKDRATISVNTYKNQFSLKLSSVTAADTAVYYCARDYDILTGYSYYYYGMDVWGQGTTVTVSS

SEQ ID NO: 249 - protein, 108 aa, synthetic construct; identical to SEQ ID NO: 227

SEQ ID NO: 250 - protein, 127 aa, synthetic construct
QVQLQESGPGLVKPSQTLSLTCTVSGGNIRRGGDYWAWIRQHPGKGLEWIGHIYHTGKTNYNPSFKGRATISVKTYKNQFSLKLSSVTAADTAVYYCARNYDTLSGNSYYYYGMDVWGQGTTVTVSS

SEQ ID NO: 251 - protein, 108 aa, synthetic construct; identical to SEQ ID NO: 231

SEQ ID NO: 252 - protein, 127 aa, synthetic construct
QVQLQESGPGLVKPSQTLSLTCTVSGGSIRRGGDYWAWIRQHPGKGLEWIGHVYQTGSTAYNPNFKDRATISVNTYKNQFSLKLSSVTAADTAVYYCARNYDALSGYSYYYYGMDVWGQGTTVTVSS

SEQ ID NO: 253 - protein, 108 aa, synthetic construct; identical to SEQ ID NO: 235

SEQ ID NO: 254 - protein, 127 aa, synthetic construct
QVQLQESGPGLVKPSQTLSLTCTVSGGSIRRGGDYWGWIRQHPGKGLEWIGHVYQSGQTAYNPKFENRATISVKTYKNQFSLKLSSVTAADTAVYYCARNYDTLTGHSYYYYGMDVWGQGTTVTVSS

SEQ ID NO: 255 - protein, 113 aa, synthetic construct
DIVMTQSPLSLPVTPGEPASISCRSSQSLLHSYGYNYLDWYLQKPGQSPQLLIYLGSNRASGVPDRFSGSGSGTDFTLKIRRVEAEDVGVYYCMQALQTPRTFGQGTKVEIKR

SEQ ID NO: 256 - protein, 127 aa, synthetic construct; identical to SEQ ID NO: 128

SEQ ID NO: 257 - protein, 16 aa, synthetic construct
RSSQSLLHRSGYNYLD

SEQ ID NO: 258 - protein, 7 aa, synthetic construct
LGSNRAS

SEQ ID NO: 259 - protein, 9 aa, synthetic construct
MQSLQTPRT

SEQ ID NO: 260 - protein, 7 aa, synthetic construct
RGGDYWS

SEQ ID NO: 261 - protein, 16 aa, synthetic construct
HIYYTGQTAYNPNLRN

SEQ ID NO: 262 - protein, 16 aa, synthetic construct
GYDASGYSYYYYGLDV

SEQ ID NO: 263 - protein, 16 aa, synthetic construct
RSSQSLLHRSGYNYLD

SEQ ID NO: 264 - protein, 7 aa, synthetic construct
QGSNRAS

SEQ ID NO: 265 - protein, 9 aa, synthetic construct
RQARALPRT

SEQ ID NO: 266 - protein, 7 aa, synthetic construct
RGGDYWS

SEQ ID NO: 267 - protein, 16 aa, synthetic construct
HVYYTGQTTYNPSFRG

SEQ ID NO: 268 - protein, 17 aa, synthetic construct
DYDILTGYSYYYYGLDV

SEQ ID NO: 269 - protein, 16 aa, synthetic construct
RSSQSLLHRSGYNYLD

SEQ ID NO: 270 - protein, 7 aa, synthetic construct
LGSNRAS

SEQ ID NO: 271 - protein, 9 aa, synthetic construct
RQSVALPRT

SEQ ID NO: 272 - protein, 7 aa, synthetic construct
RGGDYWS

SEQ ID NO: 273 - protein, 16 aa, synthetic construct
HIYYTGSTTYNPSFRG

SEQ ID NO: 274 - protein, 16 aa, synthetic construct
DYDISGYSYYYYGMDV

SEQ ID NO: 275 - protein, 16 aa, synthetic construct
RSSQSLLHRSGYNYLD

SEQ ID NO: 276 - protein, 7 aa, synthetic construct
LGSNRAS

SEQ ID NO: 277 - protein, 9 aa, synthetic construct
MQSLQTPRT

SEQ ID NO: 278 - protein, 7 aa, synthetic construct
RGGDYWS

SEQ ID NO: 279 - protein, 16 aa, synthetic construct
HIYYTGQTTYNPNFKG

SEQ ID NO: 280 - protein, 16 aa, synthetic construct
GYDASGYSYYYYGMDV

SEQ ID NO: 281 - protein, 16 aa, synthetic construct
RSSQSLLHRSGYNYLD

SEQ ID NO: 282 - protein, 7 aa, synthetic construct
LGSNRAS

SEQ ID NO: 283 - protein, 9 aa, synthetic construct
MQSLQTPRT

SEQ ID NO: 284 - protein, 7 aa, synthetic construct
RGGDYWS

SEQ ID NO: 285 - protein, 16 aa, synthetic construct
HIYYTGSTKYNPSLKN

SEQ ID NO: 286 - protein, 16 aa, synthetic construct
GYDVSGYSYYYYGLDV

SEQ ID NO: 287 - protein, 16 aa, synthetic construct
RSSQSLLHRSGYNYLD

SEQ ID NO: 288 - protein, 7 aa, synthetic construct
QGSNRAS

SEQ ID NO: 289 - protein, 9 aa, synthetic construct
MQSLQTPRT

SEQ ID NO: 290 - protein, 7 aa, synthetic construct
RGGDYWS

SEQ ID NO: 291 - protein, 16 aa, synthetic construct
HIYYAGSTAYNPNFRG

SEQ ID NO: 292 - protein, 16 aa, synthetic construct
GYDTSGYSYYYYGLDV

SEQ ID NO: 293 - protein, 16 aa, synthetic construct
RSSQSLLHRSGYNYLD

SEQ ID NO: 294 - protein, 7 aa, synthetic construct
LGSNRAS

SEQ ID NO: 295 - protein, 9 aa, synthetic construct
MQSLQTPRT

SEQ ID NO: 296 - protein, 7 aa, synthetic construct
RGGDYWS

SEQ ID NO: 297 - protein, 16 aa, synthetic construct
HVYYTGQTAYNPNFRN

SEQ ID NO: 298 - protein, 16 aa, synthetic construct
GYDVSGYSYYYYGMDV

SEQ ID NO: 299 - protein, 16 aa, synthetic construct
RSSQSLLHRSGYNYLD

SEQ ID NO: 300 - protein, 7 aa, synthetic construct
LGSNRAS

SEQ ID NO: 301 - protein, 9 aa, synthetic construct
MQSLQTPRT

SEQ ID NO: 302 - protein, 7 aa, synthetic construct
RGGDYWS

SEQ ID NO: 303 - protein, 16 aa, synthetic construct
HVYYTGQTAYNPSLRG

SEQ ID NO: 304 - protein, 16 aa, synthetic construct
GYDTSGYSYYYYGLDV

SEQ ID NO: 305 - protein, 16 aa, synthetic construct
RSSQSLLHRSGYNYLD

SEQ ID NO: 306 - protein, 7 aa, synthetic construct
LGSNRAS

SEQ ID NO: 307 - protein, 9 aa, synthetic construct
MQSLQTPRT

SEQ ID NO: 308 - protein, 7 aa, synthetic construct
RGGDYWS

SEQ ID NO: 309 - protein, 16 aa, synthetic construct
HIYYTGQTAYNPSLRN

SEQ ID NO: 310 - protein, 16 aa, synthetic construct
GYDISGYSYYYYGLDV

SEQ ID NO: 311 - protein, 16 aa, synthetic construct
RSSQSLLHRSGYNYLD

SEQ ID NO: 312 - protein, 7 aa, synthetic construct
LGSNRAS

SEQ ID NO: 313 - protein, 9 aa, synthetic construct
MQSLQTPRT

SEQ ID NO: 314 - protein, 7 aa, synthetic construct
RGGDYWS

SEQ ID NO: 315 - protein, 16 aa, synthetic construct
HVYYEGSTKYNPSFRG

SEQ ID NO: 316 - protein, 16 aa, synthetic construct
GYDVSGYSYYYYGLDV

SEQ ID NO: 317 - protein, 16 aa, synthetic construct
RSSQSLLHRSGYNYLD

SEQ ID NO: 318 - protein, 7 aa, synthetic construct
LGSNRAS

SEQ ID NO: 319 - protein, 9 aa, synthetic construct
MQSLQTPRT

SEQ ID NO: 320 - protein, 7 aa, synthetic construct
RGGDYWS

SEQ ID NO: 321 - protein, 16 aa, synthetic construct
HVYYAGSTTYNPSLKS

SEQ ID NO: 322 - protein, 16 aa, synthetic construct
GYDVSGYSYYYYGLDV

SEQ ID NO: 323 - protein, 16 aa, synthetic construct
RSSQSLLHRSGYNYLD

SEQ ID NO: 324 - protein, 7 aa, synthetic construct
LGSNRAS

SEQ ID NO: 325 - protein, 9 aa, synthetic construct
MQSLQTPRT

SEQ ID NO: 326 - protein, 7 aa, synthetic construct
RGGDYWS

SEQ ID NO: 327 - protein, 16 aa, synthetic construct
HIYYTGQTKYNPSLRG

SEQ ID NO: 328 - protein, 16 aa, synthetic construct
GYDISGYSYYYYGLDV

SEQ ID NO: 329 - protein, 16 aa, synthetic construct
RSSQSLLHRSGYNYLD

SEQ ID NO: 330 - protein, 7 aa, synthetic construct
LGSNRAS

SEQ ID NO: 331 - protein, 9 aa, synthetic construct
MQSLQTPRT

SEQ ID NO: 332 - protein, 7 aa, synthetic construct
RGGDYWS

SEQ ID NO: 333 - protein, 16 aa, synthetic construct
HVYYTGSTAYNPSFRG

SEQ ID NO: 334 - protein, 16 aa, synthetic construct
GYDTSGYSYYYYGLDV

SEQ ID NO: 335 - protein, 16 aa, synthetic construct
RSSQSLLHRSGYNYLD

SEQ ID NO: 336 - protein, 7 aa, synthetic construct
LGSNRAS

SEQ ID NO: 337 - protein, 9 aa, synthetic construct
MQSLQTPRT

SEQ ID NO: 338 - protein, 7 aa, synthetic construct
RGGDYWS

SEQ ID NO: 339 - protein, 16 aa, synthetic construct
HIYYKGSTEYNPSLRG

SEQ ID NO: 340 - protein, 16 aa, synthetic construct
GYDVSGYSYYYYGLDV

SEQ ID NO: 341 - protein, 16 aa, synthetic construct
RSSQSLLHRSGYNYLD

SEQ ID NO: 342 - protein, 7 aa, synthetic construct
LGSNRAS

SEQ ID NO: 343 - protein, 9 aa, synthetic construct
MQSLQTPRT

SEQ ID NO: 344 - protein, 7 aa, synthetic construct
RGGDYWS

SEQ ID NO: 345 - protein, 16 aa, synthetic construct
HVYYTGSTTYNPNFKG

SEQ ID NO: 346 - protein, 16 aa, synthetic construct
GYDASGYSYYYYGLDV

SEQ ID NO: 347 - protein, 16 aa, synthetic construct
RSSQSLLHRSGYNYLD

SEQ ID NO: 348 - protein, 7 aa, synthetic construct
LGSNRAS

SEQ ID NO: 349 - protein, 9 aa, synthetic construct
MQSLQTPRT

SEQ ID NO: 350 - protein, 7 aa, synthetic construct
RGGDYWS

SEQ ID NO: 351 - protein, 16 aa, synthetic construct
HVYQTGSTKYNPSFKN

SEQ ID NO: 352 - protein, 16 aa, synthetic construct
GYDVSGYSYYYYGLDV

SEQ ID NO: 353 - protein, 16 aa, synthetic construct
RSSQSLLHRSGYNYLD

SEQ ID NO: 354 - protein, 7 aa, synthetic construct
LGSNRAS

SEQ ID NO: 355 - protein, 9 aa, synthetic construct
MQSLQTPRT

SEQ ID NO: 356 - protein, 7 aa, synthetic construct
RGGDYWS

SEQ ID NO: 357 - protein, 16 aa, synthetic construct
HIYYAGQTKYNPSFKN

SEQ ID NO: 358 - protein, 16 aa, synthetic construct
GYDISGYSYYYYGLDV

SEQ ID NO: 359 - protein, 16 aa, synthetic construct
RSSQSLLHRSGYNYLD

SEQ ID NO: 360 - protein, 7 aa, synthetic construct
QGSNRAS

SEQ ID NO: 361 - protein, 9 aa, synthetic construct
MQSLQTPRT

SEQ ID NO: 362 - protein, 7 aa, synthetic construct
RGGDYWS

SEQ ID NO: 363 - protein, 16 aa, synthetic construct
HIYQTGQTKYNPSFKG

SEQ ID NO: 364 - protein, 16 aa, synthetic construct
GYDVSGYSYYYYGMDV

SEQ ID NO: 365 - protein, 113 aa, synthetic construct
DIVMTQSPLSLPVTPGEPASISCRSSQSLLHRSGYNYLDWYLQKPGQSPQLLIYLGSNRASGVPDRFSGSGSGTDFTLKISRVEAEDVGVYYCMQSLQTPRTFGQGTKVEIKR

SEQ ID NO: 366 - protein, 126 aa, synthetic construct
QVQLQESGPGLVKPSQTLSLTCTVSGGSIRRGGDYWSWIRQHPGKGLEWIGHIYYTGQTAYNPNLRNRVRISVNTYKNQFSLKLSSVTAADTAVYYCARGYDASGYSYYYYGLDVWGQGTTVTVSS

SEQ ID NO: 367 - protein, 113 aa, synthetic construct
DIVMTQSPLSLPVTPGEPASISCRSSQSLLHRSGYNYLDWYLQKPGQSPQLLIYQGSNRASGVPDRFSGSGSGTDFTLKISRVEAEDVGVYYCRQARALPRTFGQGTKVEIKR

SEQ ID NO: 368 - protein, 127 aa, synthetic construct
QVQLQESGPGLVKPSQTLSLTCTVSGGSIRRGGDYWSWIRQHPGKGLEWIGHVYYTGQTTYNPSFRGRVTISVNTYKNQFSLKLSSVTAADTAVYYCARDYDILTGYSYYYYGLDVWGQGTTVTVSS

SEQ ID NO: 369 - protein, 113 aa, synthetic construct
DIVMTQSPLSLPVTPGEPASISCRSSQSLLHRSGYNYLDWYLQKPGQSPQLLIYLGSNRASGVPDRFSGSGSGTDFTLKISRVEAEDVGVYYCRQSVALPRTFGQGTKVEIKR

SEQ ID NO: 370 - protein, 126 aa, synthetic construct
QVQLQESGPGLVKPSQTLSLTCTVSGGSIRRGGDYWSWIRQHPGKGLEWIGHIYYTGSTTYNPSFRGRVRISVNTYKNQFSLKLSSVTAADTAVYYCARDYDISGYSYYYYGMDVWGQGTTVTVSS

SEQ ID NO: 371 - protein, 113 aa, synthetic construct; identical to SEQ ID NO: 365

SEQ ID NO: 372 - protein, 126 aa, synthetic construct
QVQLQESGPGLVKPSQTLSLTCTVSGGSIRRGGDYWSWIRQHPGKGLEWIGHIYYTGQTTYNPNFKGRVRISVNTYKNQFSLKLSSVTAADTAVYYCARGYDASGYSYYYYGMDVWGQGTTVTVSR

SEQ ID NO: 373 - protein, 113 aa, synthetic construct; identical to SEQ ID NO: 365

SEQ ID NO: 374 - protein, 126 aa, synthetic construct
QVQLQESGPGLVKPSQTLSLTCTVSGGSIRRGGDYWSWIRQHPGKGLEWIGHIYYTGSTKYNPSLKNRVRISVNTYKNQFSLKLSSVTAADTAVYYCARGYDVSGYSYYYYGLDVWGQGTTVTVSS

SEQ ID NO: 375 - protein, 113 aa, synthetic construct
DIVMTQSPLSLPVTPGEPASISCRSSQSLLHRSGYNYLDWYLQKPGQSPQLLIYQGSNRASGVPDRFSGSGSGTDFTLKISRVEAEDVGVYYCMQSLQTPRTFGQGTKVEIKR

SEQ ID NO: 376 - protein, 126 aa, synthetic construct
QVQLQESGPGLVKPSQTLSLTCTVSGGSIRRGGDYWSWIRQHPGKGLEWIGHIYYAGSTAYNPNFRGRVRISVNTYKNQFSLKLSSVTAADTAVYYCARGYDTSGYSYYYYGLDVWGQGTTVTVSS

SEQ ID NO: 377 - protein, 113 aa, synthetic construct; identical to SEQ ID NO: 365

SEQ ID NO: 378 - protein, 126 aa, synthetic construct
QVQLQESGPGLVKPSQTLSLTCTVSGGSIRRGGDYWSWIRQHPGKGLEWIGHVYYTGQTAYNPNFRNRVRISVNTYKNQFSLKLSSVTAADTAVYYCARGYDVSGYSYYYYGMDVWGQGTTVTVSS

SEQ ID NO: 379 - protein, 113 aa, synthetic construct; identical to SEQ ID NO: 365

SEQ ID NO: 380 - protein, 126 aa, synthetic construct
QVQLQESGPGLVKPSQTLSLTCTVSGGSIRRGGDYWSWIRQHPGKGLEWIGHVYYTGQTAYNPSLRGRVRISVNTYKNQFSLKLSSVTAADTAVYYCARGYDTSGYSYYYYGLDVWGQGTTVTVSS

SEQ ID NO: 381 - protein, 113 aa, synthetic construct; identical to SEQ ID NO: 365

SEQ ID NO: 382 - protein, 126 aa, synthetic construct
QVQLQESGPGLVKPSQTLSLTCTVSGGSIRRGGDYWSWIRQHPGKGLEWIGHIYYTGQTAYNPSLRNRVRISVNTYKNQFSLKLSSVTAADTAVYYCARGYDISGYSYYYYGLDVWGQGTTVTVSS

SEQ ID NO: 383 - protein, 113 aa, synthetic construct; identical to SEQ ID NO: 365

SEQ ID NO: 384 - protein, 126 aa, synthetic construct
QVQLQESGPGLVKPSQTLSLTCTVSGGSIRRGGDYWSWIRQHPGKGLEWIGHIYYTGQTTYNPNFKGRVRISVNTYKNQFSLKLSSVTAADTAVYYCARGYDASGYSYYYYGMDVWGQGTTVTVSS

SEQ ID NO: 385 - protein, 113 aa, synthetic construct; identical to SEQ ID NO: 365

SEQ ID NO: 386 - protein, 126 aa, synthetic construct
QVQLQESGPGLVKPSQTLSLTCTVSGGSIRRGGDYWSWIRQHPGKGLEWIGHVYYEGSTKYNPSFRGRVRISVNTYKNQFSLKLSSVTAADTAVYYCARGYDVSGYSYYYYGLDVWGQGTTVTVSS

SEQ ID NO: 387 - protein, 113 aa, synthetic construct; identical to SEQ ID NO: 365

SEQ ID NO: 388 - protein, 126 aa, synthetic construct
QVQLQESGPGLVKPSQTLSLTCTVSGGSIRRGGDYWSWIRQHPGKGLEWIGHVYYAGSTTYNPSLKSRVRISVNTYKNQFSLKLSSVTAADTAVYYCARGYDVSGYSYYYYGLDVWGQGTTVTVSS

SEQ ID NO: 389 - protein, 113 aa, synthetic construct; identical to SEQ ID NO: 365

SEQ ID NO: 390 - protein, 126 aa, synthetic construct
QVQLQESGPGLVKPSQTLSLTCTVSGGSIRRGGDYWSWIRQHPGKGLEWIGHIYYTGQTKYNPSLRGRVRISVNTYKNQFSLKLSSVTAADTAVYYCARGYDISGYSYYYYGLDVWGQGTTVTVSS

SEQ ID NO: 391 - protein, 113 aa, synthetic construct
DIVMTQSPLSLPVTPGEPASISCRSSQSLLHRSGYNYLDWYLQKPGQSPQLLIYLGSNRASGVPGRFSGSGSGTDFTLKISRVEAEDVGVYYCMQSLQTPRTFGQGTKVEIKR

SEQ ID NO: 392 - protein, 126 aa, synthetic construct; identical to SEQ ID NO: 378

SEQ ID NO: 393 - protein, 113 aa, synthetic construct; identical to SEQ ID NO: 365

SEQ ID NO: 394 - protein, 126 aa, synthetic construct
QVQLQESGPGLVKPSQTLSLTCTVSGGSIRRGGDYWSWIRQHPGKGLEWIGHVYYTGSTAYNPSFRGRVRISVNTYKNQFSLKLSSVTAADTAVYYCARGYDTSGYSYYYYGLDVWGQGTTVTVSS

SEQ ID NO: 395 - protein, 113 aa, synthetic construct; identical to SEQ ID NO: 365

SEQ ID NO: 396 - protein, 126 aa, synthetic construct
QVQLQESGPGLVKPSQTLSLTCTVSGGSIRRGGDYWSWIRQHPGKGLEWIGHIYYKGSTEYNPSLRGRVRISVNTYKNQFSLKLSSVTAADTAVYYCARGYDVSGYSYYYYGLDVWGQGTTVTVSS

SEQ ID NO: 397 - protein, 113 aa, synthetic construct; identical to SEQ ID NO: 365

SEQ ID NO: 398 - protein, 126 aa, synthetic construct
QVQLQESGPGLVKPSQTLSLTCTVSGGSIRRGGDYWSWIRQHPGKGLEWIGHVYYTGSTTYNPNFKGRVRISVNTYKNQFSLKLSSVTAADTAVYYCARGYDASGYSYYYYGLDVWGQGTTVTVSS

SEQ ID NO: 399 - protein, 113 aa, synthetic construct; identical to SEQ ID NO: 365

SEQ ID NO: 400 - protein, 126 aa, synthetic construct
QVQLQESGPGLVKPSQTLSLTCTVSGGSIRRGGDYWSWIRQHPGKGLEWIGHVYQTGSTKYNPSFKNRVRISVNTYKNQFSLKLSSVTAADTAVYYCARGYDVSGYSYYYYGLDVWGQGTTVTVSS

SEQ ID NO: 401 - protein, 113 aa, synthetic construct; identical to SEQ ID NO: 365

SEQ ID NO: 402 - protein, 126 aa, synthetic construct
QVQLQESGPGLVKPSQTLSLTCTVSGGSIRRGGDYWSWIRQHPGKGLEWIGHIYYAGQTKYNPSFKNRVRISVNTYKNQFSLKLSSVTAADTAVYYCARGYDISGYSYYYYGLDVWGQGTTVTVSS

SEQ ID NO: 403 - protein, 113 aa, synthetic construct; identical to SEQ ID NO: 375

SEQ ID NO: 404 - protein, 126 aa, synthetic construct
QVQLQESGPGLVKPSQTLSLTCTVSGGSIRRGGDYWSWIRQHPGKGLEWIGHIYQTGQTKYNPSFKGRVRISVNTYKNQFSLKLSSVTAADTAVYYCARGYDVSGYSYYYYGMDVWGQGTTVTVSS

SEQ ID NO: 405 - protein, 11 aa, synthetic construct
RASQSISSYLN

SEQ ID NO: 406 - protein, 7 aa, synthetic construct
SARRLSS

SEQ ID NO: 407 - protein, 9 aa, synthetic construct
QQYRSYPIT

SEQ ID NO: 408 - protein, 7 aa, synthetic construct
RGGDYWG

SEQ ID NO: 409 - protein, 16 aa, synthetic construct
HVYQTGSTKYNPSFKD

SEQ ID NO: 410 - protein, 17 aa, synthetic construct
NYDTLTGYSYYYYGMDV

SEQ ID NO: 411 - protein, 11 aa, synthetic construct
RASQSISSYLN

SEQ ID NO: 412 - protein, 7 aa, synthetic construct
SARRLSS

SEQ ID NO: 413 - protein, 9 aa, synthetic construct
QQYAKSPIT

SEQ ID NO: 414 - protein, 7 aa, synthetic construct
RGGDYWA

SEQ ID NO: 415 - protein, 16 aa, synthetic construct
HIYQTGSTNYNPRFKG

SEQ ID NO: 416 - protein, 17 aa, synthetic construct
NYDTLTGYSYYYYGMDV

SEQ ID NO: 417 - protein, 11 aa, synthetic construct
RASQSISSYLN

SEQ ID NO: 418 - protein, 7 aa, synthetic construct
SARALSS

SEQ ID NO: 419 - protein, 9 aa, synthetic construct
QQYRKFPLT

SEQ ID NO: 420 - protein, 7 aa, synthetic construct
RGGDYWA

SEQ ID NO: 421 - protein, 16 aa, synthetic construct
HIYQTGSTNYNPRFKG

SEQ ID NO: 422 - protein, 17 aa, synthetic construct
NYDTLTGYSYYYYGMDV

SEQ ID NO: 423 - protein, 11 aa, synthetic construct
RASQSISSYLN

SEQ ID NO: 424 - protein, 7 aa, synthetic construct
GARRLGS

SEQ ID NO: 425 - protein, 9 aa, synthetic construct
QQYSSLPVT

SEQ ID NO: 426 - protein, 7 aa, synthetic construct
RGGDYWA

SEQ ID NO: 427 - protein, 16 aa, synthetic construct
HVYYTGSTKYNPSFKD

SEQ ID NO: 428 - protein, 17 aa, synthetic construct
NYDTLTGYSYYYYGMDV

SEQ ID NO: 429 - protein, 11 aa, synthetic construct
RASQSISSYLN

SEQ ID NO: 430 - protein, 7 aa, synthetic construct
KARLLSS

SEQ ID NO: 431 - protein, 9 aa, synthetic construct
QQYSRLPLT

SEQ ID NO: 432 - protein, 7 aa, synthetic construct
RGGDYWA

SEQ ID NO: 433 - protein, 16 aa, synthetic construct
HVYYTGSTNYNPRFKD

SEQ ID NO: 434 - protein, 17 aa, synthetic construct
NYDTVTGYSYYYYGMDV

SEQ ID NO: 435 - protein, 11 aa, synthetic construct
RASQSISSYLN

SEQ ID NO: 436 - protein, 7 aa, synthetic construct
KARKLSS

SEQ ID NO: 437 - protein, 9 aa, synthetic construct
QQFGSMPLT

SEQ ID NO: 438 - protein, 7 aa, synthetic construct
RGGDYWA

SEQ ID NO: 439 - protein, 16 aa, synthetic construct
HIYQTGSTNYNPRFKG

SEQ ID NO: 440 - protein, 17 aa, synthetic construct
NYDTLTGYSYYYYGMDV

SEQ ID NO: 441 - protein, 11 aa, synthetic construct
RASQSISSYLN

SEQ ID NO: 442 - protein, 7 aa, synthetic construct
SARRLAS

SEQ ID NO: 443 - protein, 9 aa, synthetic construct
QQYYHPPIT

SEQ ID NO: 444 - protein, 7 aa, synthetic construct
RGGDYWA

SEQ ID NO: 445 - protein, 16 aa, synthetic construct
HVYYTGSTNYNPRFKD

SEQ ID NO: 446 - protein, 17 aa, synthetic construct
NYDTVTGYSYYYYGMDV

SEQ ID NO: 447 - protein, 11 aa, synthetic construct
RASQSISSYLN

SEQ ID NO: 448 - protein, 7 aa, synthetic construct
NARRLGS

SEQ ID NO: 449 - protein, 9 aa, synthetic construct
QQYRSSPVT

SEQ ID NO: 450 - protein, 7 aa, synthetic construct
RGGDYWA

SEQ ID NO: 451 - protein, 16 aa, synthetic construct
HIYQTGSTNYNPRFKG

SEQ ID NO: 452 - protein, 17 aa, synthetic construct
NYDTLTGYSYYYYGMDV

SEQ ID NO: 453 - protein, 108 aa, synthetic construct
DIQMTQSPSSLSASVGDRVTITCRASQSISSYLNWYQQKPGKAPKLLIYSARRLSSGVPSRFSGSGSGTDFTLTISSLQPEDFATYYCQQYRSYPITFGGGTKVEIKR

SEQ ID NO: 454 - protein, 127 aa, synthetic construct; identical to SEQ ID NO: 236

SEQ ID NO: 455 - protein, 108 aa, synthetic construct
DIQMTQSPSSLSASVGDRVTITCRASQSISSYLNWYQQKPGKAPKLLIYSARRLSSGVPSRFSGSGSGTDFTLTISSLQPEDFATYYCQQYAKSPITFGGGTKVEIKR

SEQ ID NO: 456 - protein, 127 aa, synthetic construct; identical to SEQ ID NO: 226

SEQ ID NO: 457 - protein, 108 aa, synthetic construct
DIQMTQSPSSLSASVGDRVTITCRASQSISSYLNWYQQKPGKAPKLLIYSARALSSGVPSRFSGSGSGTDFTLTISSLQPEDFATYYCQQYRKFPLTFGGGTKVEIKR

SEQ ID NO: 458 - protein, 127 aa, synthetic construct; identical to SEQ ID NO: 226

SEQ ID NO: 459 - protein, 108 aa, synthetic construct
DIQMTQSPSSLSASVGDRVTITCRASQSISSYLNWYQQKPGKAPKLLIYGARRLGSGVPSRFSGSGSGTDFTLTISSLQPEDFATYYCQQYSSLPVTFGGGTKVEIKR

SEQ ID NO: 460 - protein, 127 aa, synthetic construct
QVQLQESGPGLVKPSQTLSLTCTVSGGSIRRGGDYWAWIRQHPGKGLEWIGHVYYTGSTKYNPSFKDRATISVNTYKNQFSLKLSSVTAADTAVYYCARNYDTLTGYSYYYYGMDVWGQGTTVTVSS

SEQ ID NO: 461 - protein, 108 aa, synthetic construct
DIQMTQSPSSLSASVGDRVTITCRASQSISSYLNWYQQKPGKAPKLLIYKARLLSSGVPSRFSGSGSGTDFTLTISSLQPEDFATYYCQQYSRLPLTFGGGTKVEIKR

SEQ ID NO: 462 - protein, 127 aa, synthetic construct; identical to SEQ ID NO: 228

SEQ ID NO: 463 - protein, 108 aa, synthetic construct
DIQMTQSPSSLSASVGDRVTITCRASQSISSYLNWYQQKPGKAPKLLIYKARKLSSGVPSRFSGSGSGTDFTLTISSLQPEDFATYYCQQFGSMPLTFGGGTKVEIKR

SEQ ID NO: 464 - protein, 127 aa, synthetic construct; identical to SEQ ID NO: 226

SEQ ID NO: 465 - protein, 108 aa, synthetic construct
DIQMTQSPSSLSASVGDRVTITCRASQSISSYLNWYQQKPGKAPKLLIYSARRLASGVPSRFSGSGSGTDFTLTISSLQPEDFATYYCQQYYHPPITFGGGTKVEIKR

SEQ ID NO: 466 - protein, 127 aa, synthetic construct; identical to SEQ ID NO: 228

SEQ ID NO: 467 - protein, 108 aa, synthetic construct
DIQMTQSPSSLSASVGDRVTITCRASQSISSYLNWYQQKPGKAPKLLIYNARRLGSGVPSRFSGSGSGTDFTLTISSLQPEDFATYYCQQYRSSPVTFGGGTKVEIKR

SEQ ID NO: 468 - protein, 127 aa, synthetic construct; identical to SEQ ID NO: 226

SEQ ID NO: 469 - protein, 108 aa, synthetic construct
DIQMTQSPSSLPASVGDRVTITCRASQSISSYLNWYQQKPGKAPKLLIYSARRLASGVPSRFSGSGSGTDFTLTISSLQPEDFATYYCQQYYHPPITFGGGTKVEIKR

SEQ ID NO: 470 - protein, 127 aa, synthetic construct; identical to SEQ ID NO: 228

SEQ ID NO: 471 - protein, 16 aa, synthetic construct
RSSQSLLHRSGYNYLD

SEQ ID NO: 472 - protein, 7 aa, synthetic construct
LGSNRAS

SEQ ID NO: 473 - protein, 9 aa, synthetic construct
MQSLQTPRT

SEQ ID NO: 474 - protein, 7 aa, synthetic construct
RGGDYWS

SEQ ID NO: 475 - protein, 16 aa, synthetic construct
HIYYSGSTNYNPSLRS

SEQ ID NO: 476 - protein, 17 aa, synthetic construct
DYDILTGYSYYYYGLDV

SEQ ID NO: 477 - protein, 16 aa, synthetic construct
RSSQSLLYSNGHNFLD

SEQ ID NO: 478 - protein, 7 aa, synthetic construct
LGSNRAS

SEQ ID NO: 479 - protein, 9 aa, synthetic construct
MQALQTPRT

SEQ ID NO: 480 - protein, 7 aa, synthetic construct
RGGDYWN

SEQ ID NO: 481 - protein, 16 aa, synthetic construct
HIYYSGSKNYNPSLKS

SEQ ID NO: 482 - protein, 16 aa, synthetic construct
GYDSSGYSYYYYGMDV

SEQ ID NO: 483 - protein, 16 aa, synthetic construct
RSSQSLLYSNGHNYLD

SEQ ID NO: 484 - protein, 7 aa, synthetic construct
LGSNRAP

SEQ ID NO: 485 - protein, 9 aa, synthetic construct
MQALQTPRT

SEQ ID NO: 486 - protein, 7 aa, synthetic construct
RGGDYWN

SEQ ID NO: 487 - protein, 16 aa, synthetic construct
HIYYTGTKYYNPSLKS

SEQ ID NO: 488 - protein, 16 aa, synthetic construct
GYDSSGYSYYYYGMDV

SEQ ID NO: 489 - protein, 127 aa, synthetic construct
QVQLQESGPGLVKPSQTLSLTCTVSGGSIRRGGDYWSWIRQHPGKGLEWIGHIYYSGSTNYNPSLRSRVRISVDTSKNQFSLKLSSVTAADTAVYYCARDYDILTGYSYYYYGLDVWGQGTTVTVSS

SEQ ID NO: 490 - protein, 113 aa, synthetic construct; identical to SEQ ID NO: 365

SEQ ID NO: 491 - protein, 126 aa, synthetic construct
QVQLQESGPGLVKPSQTLSLICIVSGGSIRRGGDYWNWIRQHPGKGLEWIGHIYYSGSKNYNPSLKSRVTISVDTSKNQFSLKLSSVTAADTAVYYCARGYDSSGYSYYYYGMDVWGQGTTVTVSS

SEQ ID NO: 492 - protein, 113 aa, synthetic construct
DIVMTQSPLSLPVTPGEPASISCRSSQSLLYSNGHNFLDWYLQKPGQSPQLLIYLGSNRASGVPDRFSGSGSGTDFTLKISRVEAEDVGVYYCMQALQTPRTFGGGTKVEIKR

SEQ ID NO: 493 - protein, 126 aa, synthetic construct
QVQLRESGPGLVKPSQTLSLICIVSGGSIRRGGDYWNWIRQHPGKGLEWIGHIYYTGTKYYNPSLKSRVTISVDTSKNQFSLKLSSVTAADTAVYYCARGYDSSGYSYYYYGMDVWGQGTTVTVSS

SEQ ID NO: 494 - protein, 113 aa, synthetic construct
DIVMTQSPLSLPVTPGEPASISCRSSQSLLYSNGHNYLDWYLQKPGQSPQLLIYLGSNRAPGVPDRFSGSGSGTDFTLKISRVEAEDVGVYFCMQALQTPRTFGGGTKVEIKR

SEQ ID NO: 495 - protein, 12 aa, synthetic construct
RASQSVSSSYLA

SEQ ID NO: 496 - protein, 7 aa, synthetic construct
GASSRAT

SEQ ID NO: 497 - protein, 9 aa, synthetic construct
QQGYSNPRT

SEQ ID NO: 498 - protein, 11 aa, synthetic construct
RASQSIISYLN

SEQ ID NO: 499 - protein, 7 aa, synthetic construct
AASSLRS

SEQ ID NO: 500 - protein, 9 aa, synthetic construct
QQYSNYPLT

SEQ ID NO: 501 - protein, 11 aa, synthetic construct
RASQSVSNYLA

SEQ ID NO: 502 - protein, 7 aa, synthetic construct
DASNRAT

SEQ ID NO: 503 - protein, 9 aa, synthetic construct
QQYGSLPRT

SEQ ID NO: 504 - protein, 11 aa, synthetic construct
RASQSVSSYLG

SEQ ID NO: 505 - protein, 7 aa, synthetic construct
GASSRAT

SEQ ID NO: 506 - protein, 9 aa, synthetic construct
QQYGSSPRT

SEQ ID NO: 507 - protein, 11 aa, synthetic construct
RASQSVSSYLA

SEQ ID NO: 508 - protein, 7 aa, synthetic construct
DASNRAT

SEQ ID NO: 509 - protein, 9 aa, synthetic construct
QQYGSLPRT

SEQ ID NO: 510 - protein, 11 aa, synthetic construct
RASQSISNYLN

SEQ ID NO: 511 - protein, 7 aa, synthetic construct
AASSLHS

SEQ ID NO: 512 - protein, 9 aa, synthetic construct
QQYQSYPLT

SEQ ID NO: 513 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 514 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 515 - protein, 105 aa, synthetic construct
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 516 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 517 - protein, 106 aa, synthetic construct
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 518 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - protein, 107 aa, synthetic construct [sequence omitted]

SEQ ID NO: 520 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 521 - protein, 330 aa, synthetic construct [sequence omitted]

SEQ ID NO: 522 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 523 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 524 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 526 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 527 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 528 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 529 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 530 - protein, 7 aa, synthetic construct
  VARIANT 7: note = Xaa is A, S or G
RGGDYWX

SEQ ID NO: 531 - protein, 16 aa, synthetic construct
  VARIANT 2: note = Xaa is I or V
  VARIANT 4: note = Xaa is H, Y or Q
  VARIANT 5: note = Xaa is E, T, or S
  VARIANT 7: note = Xaa is K, S, or Q
  VARIANT 9: note = Xaa is A, K or N
  VARIANT 13: note = Xaa is N, S, K, or R
  VARIANT 14: note = Xaa is F or L
  VARIANT 15: note = Xaa is E or K
  VARIANT 16: note = Xaa is G, D, S, or N
HXYXXGXTXYNPXXXX

SEQ ID NO: 532 - protein, 17 aa, synthetic construct
  VARIANT 1: note = Xaa is N
  VARIANT 4: note = Xaa is T
  VARIANT 5: note = Xaa is L, or V
  VARIANT 6: note = Xaa is T
  VARIANT 8: note = Xaa is Y
XYDXXXGXSYYYYGMDV

SEQ ID NO: 533 - protein, 16 aa, synthetic construct
  VARIANT 8: note = Xaa is H, S, or T
  VARIANT 10: note = Xaa is S, T, or Y;
  VARIANT 12: note = Xaa is A, N, I or Y
  VARIANT 15: note = Xaa is L or V
RSSQSLLXSXGXNYXD

SEQ ID NO: 534 - protein, 6 aa, synthetic construct
  NON_STD 1: note = Xaa is L or V
XGSNRA

SEQ ID NO: 535 - protein, 9 aa, synthetic construct
  VARIANT 1: note = Xaa is M or R
  VARIANT 4: note = Xaa is L, I, R, or V
  VARIANT 5: note = X is Q, E, T, A, or G
  VARIANT 6: note = Xaa is T, I, L, or Q
XQAXXXPRT

SEQ ID NO: 536 - protein, 7 aa, synthetic construct
  VARIANT 1: note = X is N, S, K or G
  VARIANT 4: note = Xaa is R, K, L or A
  VARIANT 6: note = Xaa is A, G or S
XARXLXS

SEQ ID NO: 537 - protein, 9 aa, synthetic construct
  VARIANT 3: note = Xaa is Y or F
  VARIANT 4: note = Xaa is R, A, S, G, or Y
  VARIANT 5: note = Xaa is S, K, R or H
  VARIANT 6: note = Xaa is S, L, F, Y, P, or M
  VARIANT 8: note = Xaa is L, I or V
QQXXXXPXT

SEQ ID NO: 538 - protein, 16 aa, synthetic construct
  VARIANT 2: note = Xaa is I or V
  VARIANT 4: note = Xaa is Y or Q
  VARIANT 5: note = Xaa is A, E, K, or T
  VARIANT 7: note = Xaa is S or Q
  VARIANT 9: note = Xaa is A, E, K, or T
  VARIANT 13: note = Xaa is N or S
  VARIANT 14: note = X is F or L
  VARIANT 15: note = Xaa is K or R
  VARIANT 16: note = Xaa is G, S, or N
HXYXXGXTXYNPXXXX

SEQ ID NO: 539 - protein, 17 aa, synthetic construct
  VARIANT 1: note = Xaa is D or G
  VARIANT 4: note = Xaa is A, I, T, or V
  VARIANT 5: note = Xaa is L or absent
  VARIANT 6: note = Xaa is S or T
  VARIANT 15: note = Xaa is M or L
XYDXXXGYSYYYYGXDV

SEQ ID NO: 540 - protein, 9 aa, synthetic construct
  VARIANT 1: note = Xaa is M or R
  VARIANT 3: note = Xaa is A or S
  VARIANT 4: note = Xaa is L, R, or V
  VARIANT 5: note = Xaa is Q or A
  VARIANT 6: note = Xaa is T or L
XQXXXXPRT

SEQ ID NO: 541 - protein, 22 aa, synthetic construct
MDMRVPAQLLGLLLLWLRGARC

SEQ ID NO: 542 - protein, 19 aa, synthetic construct
MAWALLLLTLLTQGTGSWA

SEQ ID NO: 543 - protein, 19 aa, synthetic construct
MTCSPLLLTLLIHCTGSWA

SEQ ID NO: 544 - protein, 20 aa, synthetic construct
MEAPAQLLFLLLLWLPDTTG

SEQ ID NO: 545 - protein, 19 aa, synthetic construct
MEWTWRVLFLVAAATGAHS

SEQ ID NO: 546 - protein, 20 aa, synthetic construct
METPAQLLFLLLLWLPDTTG

SEQ ID NO: 547 - protein, 20 aa, synthetic construct
METPAQLLFLLLLWLPDTTG

SEQ ID NO: 548 - protein, 19 aa, synthetic construct
MKHLWFFLLLVAAPRWVLS

SEQ ID NO: 549 - protein, 19 aa, synthetic construct
MEWSWVFLFFLSVTTGVHS

SEQ ID NO: 550 - protein, 22 aa, synthetic construct
MDIRAPTQLLGLLLLWLPGAKC

SEQ ID NO: 551 - protein, 22 aa, synthetic construct
MDIRAPTQLLGLLLLWLPGARC

SEQ ID NO: 552 - protein, 22 aa, synthetic construct
MDTRAPTQLLGLLLLWLPGATF

SEQ ID NO: 553 - protein, 22 aa, synthetic construct
MDTRAPTQLLGLLLLWLPGARC

SEQ ID NO: 554 - protein, 19 aa, synthetic construct
METGLRWLLLVAVLKGVQC

SEQ ID NO: 555 - protein, 21 aa, synthetic construct
METGLRWLLLVAVLKGVQCQE

SEQ ID NO: 556 - protein, 22 aa, synthetic construct
MDMRAPTQLLGLLLLWLPGARC

SEQ ID NO: 557 - protein, 19 aa, synthetic construct
MKILILGIFLFLCSTPAWA

SEQ ID NO: 558 - protein, 19 aa, synthetic construct
MRTLAILAAILLVALQAQA

SEQ ID NO: 559 - protein, 472 aa, synthetic construct
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 560 - protein, 359 aa, synthetic construct
MEECWVTEIANGSKDGLDSNPMKDYMILSGPQKTAVAVLCTLLGLLSALENVAVLYLILSSHQLRRKPSYLFIGSLAGADFLASVVFACSFVNFHVFHGVDSKAVFLLKIGSVTMTFTASVGSLLLTAIDRYLCLRYPPSYKALLTRGRALVTLGIMWVLSALVSYLPLMGWTCCPRPCSELFPLIPNDYLLSWLLFIAFLFSGIIYTYGHVLWKAHQHVASLSGHQDRQVGMARMRLDVRLAKTLGLVLAVLLICWFPVLALMAHSLATTLSDQVKKAFAFCSMLCLINSMVNPVIYALRSGEIRSSAHHCLAHWKKCVRGLGSEAKEEAPRSSVTETEADGKITPWPDSRDLDLSDC

SEQ ID NO: 561 - protein, 473 aa, synthetic construct
MKSILDGLADTTFRTITTDLLYVGSNDIQYEDIKGDMASKLGYFPQKFPLTSFRGSPFQEKMTAGDNSPLVPAGDTTNITEFYNKSLSSFKENEDNIQCGENFMDMECFMILNPSQQLAIAVLSLTLGTFTVLENLLVLCVILHSRSLRCRPSYHFIGSLAVADLLGSVIFVYSFVDFHVFHRKDSPNVFLFKLGGVTASFTASVGSLFLTAIDRYISIHRPLAYKRIVTRPKAVVAFCLMWTIAIVIAVLPLLGWNCKKLQSVCSDIFPLIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNTASMHRAAESCIKSTVKIAKVTMSVSTDTSAEAL

SEQ ID NO: 562 - protein, 457 aa, synthetic construct
QVQLQESGPGLVKPSQTLSLTCTVSGGSIRRGGDYWAWIRQHPGKGLEWIGHVYYTGSTNYNPRFKDRATISVNTYKNQFSLKLSSVTAADTAVYYCARNYDTVTGYSYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 563 - protein, 214 aa, synthetic construct
DIQLTQSPSTLSASVGDRVTITCRASQSISNYLNWYQQKPGKAPKLLIYAASSLHSGVPSRFSGSGSGTDFTLTISSLQPEDFASYYCQQYQSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 564 - protein, 7 aa, synthetic construct
  VARIANT 7: note = Xaa is A or G
RGGDYWX

SEQ ID NO: 565 - protein, 16 aa, synthetic construct
  VARIANT 2: note = Xaa is I or V
  VARIANT 4: note = Xaa is H, Y, or Q
  VARIANT 5: note = Xaa is E, T, or S
  VARIANT 7: note = Xaa is K, S, or Q
  VARIANT 9: note = Xaa is A, K or N
  VARIANT 13: note = Xaa is N, S, K, or R
  VARIANT 14: note = Xaa is F or L
  VARIANT 15: note = Xaa is E or K
  VARIANT 16: note = Xaa is G, D, S, or N
HXYXXGXTXYNPXXXX

SEQ ID NO: 566 - protein, 17 aa, synthetic construct
  VARIANT 1: note = Xaa is N
  VARIANT 4: note = Xaa is T
  VARIANT 5: note = Xaa is L, or V
  VARIANT 6: note = Xaa is T
  VARIANT 8: note = Xaa is Y
XYDXXXGXSYYYYGMDV